(19) United States
(12) Patent Application Publication
(10) Pub. No.: US 2026/0102298 A1
(43) Pub. Date: Apr. 16, 2026

(54) SYSTEMS AND METHODS FOR TEMPERATURE REGULATION OF AN ARTICLE OF FURNITURE

(71) Applicant: Eight Sleep Inc., New York, NY (US)

(72) Inventors: Nicole Elizabeth MOYEN, Natick, MA (US); David Da HE, San Carlos, CA (US); Massimo ANDREASI BASSI, Union City, NJ (US); Matteo FRANCESCHETTI, Miami Shores, FL (US); Alexandra ZATARAIN, Miami Shores, FL (US)

(21) Appl. No.: 19/380,102

(22) Filed: Nov. 5, 2025

Related U.S. Application Data
(63) Continuation of application No. PCT/US24/29903, filed on May 17, 2024.
(60) Provisional application No. 63/467,733, filed on May 19, 2023.
(60) Provisional application No. 63/534,795, filed on Aug. 25, 2023.
(60) Provisional application No. 63/606,146, filed on Dec. 5, 2023.

(51) Int. Cl. A61G 7/05; A47C 21/04

(52) U.S. Cl. CPC A61G 7/05; A47C 21/044; A47C 21/048; A61G 2200/12; A61G 2200/32; A61G 2203/30; A61G 2210/70; A61G 2210/90

(57) ABSTRACT

Described herein are systems and methods (e.g., computer-implemented methods) for regulating the temperature of a bed device. The methods (e.g., computer-implemented methods) can regulate the temperature of the bed device based on biological sex, age, time of night, season, geolocation, and women's health.

### CROSS-REFERENCE TO RELATED APPLICATIONS

This application is a bypass continuation of International Patent Application No. PCT/US24/29903, filed May 17, 2024, which claims the benefit of U.S. Provisional Application No. 63/467,733, filed on May 19, 2023, U.S. Provisional Application No. 63/534,795, filed on Aug. 25, 2023, and U.S. Provisional Application No. 63/606,146, filed on Dec. 5, 2023; each of which applications is incorporated by reference herein in its entirety for all purposes.

### BACKGROUND

Conditions of an article of furniture can be adjusted during use. For example, temperatures of a bed can be adjusted during an individual's sleep to, for example, help the individual sleep better.

### SUMMARY

The present disclosure describes technologies relating to regulating a condition (e.g., temperature) of an article of furniture (e.g., a bed device, such as a mattress or a mattress cover). The present disclosure describes using a fluid (e.g., a liquid or gas) and one or more temperature regulators of the fluid to regulate the condition of at least a portion of the article of furniture.

In one aspect, the present disclosure provides a method comprising: providing a control signal for a temperature regulator that is operatively coupled to an article of furniture, wherein the control signal comprises an instruction for the temperature regulator to change a temperature of a portion of the article of furniture from an initial temperature to a final temperature over a plurality of incremental temperature changes, wherein the plurality of incremental temperature changes comprises a predetermined number of incremental temperature changes.

In some embodiments, the article of furniture is a bed device.

In some embodiments of any one of the preceding embodiments, the predetermined number of the plurality of incremental temperature changes comprises at least 2 incremental changes, at least 3 incremental changes, or at least 4 incremental changes.

In some embodiments of any one of the preceding embodiments, the control signal further comprises an instruction for the temperature regulator to maintain the portion of the article of furniture at an average temperature between two adjacent incremental temperature changes of the plurality of incremental temperature changes. In some embodiments, the average temperature is maintained for at least about 10 minutes, at least about 20 minutes, at least about 30 minutes, at least about 60 minutes, or at least about 80 minutes.

In some embodiments of any one of the preceding embodiments, the initial temperature is lower than the final temperature.

In some embodiments of any one of the preceding embodiments, the control signal further comprises an instruction for the temperature regulator to set the temperature of the portion of the article of furniture to a bedtime temperature prior to changing the temperature from the initial temperature to the final temperature, wherein the bedtime temperature and the initial temperature are different. In some embodiments, the bedtime temperature is greater than the initial temperature. In some embodiments, the bedtime temperature, the initial temperature, and the final temperature are (i) provided by a user of the article of furniture or (ii) automatically generated by a computer processor.

In some embodiments of any one of the preceding embodiments, the plurality of incremental temperature changes is implemented within at most about 60 minutes, at most about 30 minutes, at most about 20 minutes, or at most about 10 minutes of the bedtime.

In some embodiments of any one of the preceding embodiments, a characteristic of the plurality of incremental temperature changes is different for different users, wherein the characteristic comprises (i) the predetermined number or (ii) a target temperature of the article of furniture or a duration thereof between two adjacent incremental changes.

In some embodiments of any one of the preceding embodiments, the initial temperature corresponds to a deep sleep temperature.

In some embodiments of any one of the preceding embodiments, the final temperature corresponds to a rapid eye movement (REM) sleep temperature.

In some embodiments of any one of the preceding embodiments, at least a portion of the temperature regulator is contained within the portion of the article of furniture.

In one aspect, the present disclosure provides a method comprising: providing a control signal for a temperature regulator that is operatively coupled to an article of furniture, wherein the control signal comprises an instruction for the temperature regulator to sequentially change a temperature of a portion of the article of furniture to each target temperature of a combination of predetermined target temperatures during a single use of the article of furniture by a user, wherein the combination of predetermined target temperatures is determined prior to the single use of the article of furniture by the user, wherein each target temperature of the combination of target temperatures are different from one another, and wherein the combination of target temperatures is selected from a database comprising a plurality of combinations of target temperatures based on one or more members selected from the group consisting of a geolocation of the user, biological sex of the user, and age of the user.

In some embodiments, the article of furniture is a bed device.

In some embodiments of any one of the preceding embodiments, the combination of predetermined target temperatures comprises at least two different target temperatures or at least three different target temperatures.

In some embodiments of any one of the preceding embodiments, the combination of predetermined target temperatures comprises at most five different target temperatures or at most four different target temperatures.

In some embodiments of any one of the preceding embodiments, the combination of predetermined target temperatures comprises a bedtime temperature.

In some embodiments of any one of the preceding embodiments, the combination of predetermined target temperatures does not comprise a wake-up temperature.

In some embodiments of any one of the preceding embodiments, each combination of the combinations of target predetermined temperatures is different based on the one or more members selected from the group consisting of a geolocation of the user, biological sex of the user, and age of the user. In some embodiments, the one or more members comprises two members or all three members elected from the group consisting of the geolocation of the user, the biological sex of the user, and the age of the user. In some embodiments, the one or more members comprises the geolocation of the user. In some embodiments, the one or more members comprises the biological sex of the user. In some embodiments, the one or more members comprises the age of the user.

In some embodiments of any one of the preceding embodiments, the method further comprises subsequent to use of the article of furniture by the user based on the combination of the predetermined target temperatures, modifying one or more target temperatures of the combination of predetermined target temperatures, to effect enhanced user experience.

In some embodiments of any one of the preceding embodiments, the user is not permitted or recommended to change the combination of predetermined target temperatures for at least one use of the article of furniture, optionally wherein the at least one use comprises at least two uses, at least five uses, or at least seven uses. In some embodiments, the method further comprises subsequent to using the article of furniture based on the combination of target predetermined temperatures for at least one day, permitting the user to modify one or more target temperatures of the combination of target predetermined temperatures via a graphical user interface (GUI) operatively coupled to the article of furniture, to effect enhanced user experience. In some embodiments, the at least one day comprises at least two days, at least five days, or at least seven days. In some embodiments, the enhanced user experience is enhanced sleep. In some embodiments, the one or more target temperatures of the combination of target predetermined temperatures are changed (i) based on feedback from the user via a graphical user interface (GUI) operatively coupled to the article of furniture or (ii) automatically by a computer processor in absence of any user feedback.

In some embodiments of any one of the preceding embodiments, the combination of target predetermined temperatures is provided when the user is using the article of furniture for the first time.

In some embodiments of any one of the preceding embodiments, the combination of target predetermined temperatures is provided when an identity of the user is undetermined.

In some embodiments of any one of the preceding embodiments, the providing comprises recommending the combination of target predetermined temperatures to the user.

In some embodiments of any one of the preceding embodiments, at least a portion of the temperature regulator is contained within the portion of the article of furniture.

In one aspect, the present disclosure provides a method comprising: providing a control signal for a temperature regulator that is operatively coupled to an article of furniture, wherein the control signal comprises an instruction for the temperature regulator to change a temperature of a portion of the article of furniture to a predetermined target temperature during use of the article of furniture by a user, wherein the predetermined target temperature is determined prior to a single use of the article of furniture by the user, wherein the target temperature is determined based on biological sex of the user, such that a first provided temperature for a female user and a second provided temperature for a male user are different.

In some embodiments, the article of furniture is a bed device.

In some embodiments of any one of the preceding embodiments, the first provided temperature for the female user is greater than the second provided temperature for the male user. In some embodiments, the first provided temperature for the female user is greater than the second provided temperature for the male user by at least or up to about 1 degree Celsius, at least or up to about 1.5 degrees Celsius, at least or up to about 2 degrees Celsius, at least or up to about 3 degrees Celsius, at least or up to about 4 degrees Celsius, or at least or up to about 5 degrees Celsius.

In some embodiments of any one of the preceding embodiments, the user is the female user, and wherein the predetermined target temperature is determined based on a biological state of the female user, wherein the biological state is women-specific. In some embodiments, the biological state of the female user comprises one or more members selected from the group consisting of (i) schedule of menstrual cycle, a time within the menstrual cycle, or lack of menstrual cycle, (ii) use or birth control or lack thereof, (iii) menopause or lack thereof, (iv) use of hormonal therapy or lack thereof, (v) pregnancy or lack thereof, (vi) post-partum period, (vii) breast-feeding period, (viii) polycystic ovarian syndrome, (xi) experience of hot flash, and (x) other women-specific conditions or disease. In some embodiments, the one or more members are provided or selected by the user via a graphical user interface (GUI) operatively coupled to the article of furniture. In some embodiments, the one or more members comprises two or more members selected from the group consisting of (i)-(ix). In some embodiments, the predetermined target temperature is selected from a database comprising two or more different target temperatures for two or more members selected from the group consisting of (i)-(ix).

In some embodiments of any one of the preceding embodiments, the predetermined target temperature is selected from a database comprising the first provided temperature and the second provided temperature.

In some embodiments of any one of the preceding embodiments, the method further comprises permitting the user to modify the predetermined target temperature via a graphical user interface (GUI) operatively coupled to the article of furniture.

In some embodiments of any one of the preceding embodiments, the method further comprises receiving feedback from the user via a graphical user interface (GUI) operatively coupled to the article of furniture after use of the article of furniture based on the predetermined target temperature, and subsequently modifying the predetermined target temperature based on the feedback.

In some embodiments of any one of the preceding embodiments, the biological sex of the user is provided by the user via a graphical user interface (GUI) operatively coupled to the article of furniture.

In some embodiments of any one of the preceding embodiments, at least a portion of the temperature regulator is contained within the portion of the article of furniture.

In some embodiments of any one of the preceding embodiments, the first provided temperature for a male user during a sleep stage is lower than the first provided temperature for a female user during the sleep stage.

In one aspect, the present disclosure provides a method comprising: (a) retrieving an input from a user or a user device, wherein the input is indicative of a biological condition of a female individual selected from the group consisting of (i) schedule of menstrual cycle, a time within the menstrual cycle, or lack of menstrual cycle, (ii) use of birth control or lack thereof, (iii) menopause or lack thereof, (iv) use of hormonal therapy or lack thereof, (v) pregnancy or lack thereof, (vi) post-partum period, (vii) breast-feeding period, (viii) polycystic ovarian syndrome, (ix) experience of hot flash, and (x) other women-specific conditions or diseases, and (b) based on the input, providing a control signal for a temperature regulator that is operatively coupled to an article of furniture, wherein the control signal comprises an instruction for the temperature regulator to change a temperature of a portion of the article of furniture to a target temperature specific for the biological condition of the female individual, wherein the target temperature is selected from a database comprising a plurality of different target temperatures associated with the plurality of different biological conditions selected from the group consisting of (i)-(x).

In some embodiments, the article of furniture is a bed device.

In some embodiments of any one of the preceding embodiments, the target temperature is for enhancing user experience of the user while using the article of furniture.

In some embodiments of any one of the preceding embodiments, the target temperature is for enhancing sleep quality of the user during or subsequent to sleeping on the article of furniture at about the target temperature.

In some embodiments of any one of the preceding embodiments, the target temperature is for enhancing a health condition of the user during or subsequent to during or subsequent to sleeping on the article of furniture at about the target temperature.

In some embodiments of any one of the preceding embodiments, the input is provided by the user via a graphical user interface (GUI) of a user device that is operatively coupled to the article of furniture.

In some embodiments of any one of the preceding embodiments, the biological condition of the female individual comprises a plurality of members selected from the group consisting of (i)-(x). In some embodiments of any one of the preceding embodiments, the instruction is for the temperature regulator to change the temperature of the portion of the article of furniture to the target temperature (i) for a predetermined duration of time and/or (ii) at a predetermined time.

In some embodiments of any one of the preceding embodiments, the predetermined period of time is retrieved from the database.

In some embodiments of any one of the preceding embodiments, the method further comprises permitting the user to modify the target temperature specific for the biological condition of the female individual via a graphical user interface (GUI) of a user device operatively coupled to the article of furniture, and storing the modified target temperature in a database operatively coupled to the article of furniture. In some embodiments, the method further comprises (i) receiving a feedback from the user via a graphical user interface (GUI) of a user device operatively coupled to the article of furniture subsequent to the user's use of the article of furniture at the target temperature, and (ii) automatically modifying the target temperature based on the feedback for the user's future use of the article of furniture.

In some embodiments of any one of the preceding embodiments, a biological sex of the user is provided by the user via a graphical user interface (GUI) of a user device operatively coupled to the article of furniture.

In some embodiments of any one of the preceding embodiments, at least a portion of the temperature regulator is contained within the portion of the article of furniture.

In some embodiments of any one of the preceding embodiments, the article of furniture further comprises a sensor disclosed in a surface of the article of furniture.

In some embodiments of any one of the preceding embodiments, the biological condition of the female individual is a schedule of a menstrual cycle, a time within the menstrual cycle, or a lack of a menstrual cycle.

In some embodiments of any one of the preceding embodiments, the biological condition of the female individual is use of birth control or lack thereof.

In some embodiments of any one of the preceding embodiments, the biological condition of the female individual is menopause or lack thereof.

In some embodiments of any one of the preceding embodiments, the biological condition of the female individual is use of hormonal therapy or lack thereof.

In some embodiments of any one of the preceding embodiments, the biological condition of the female individual is pregnancy or lack thereof.

In some embodiments of any one of the preceding embodiments, the biological condition of the female individual is post-partum period.

In some embodiments of any one of the preceding embodiments, the biological condition of the female individual is breast-feeding period.

In some embodiments of any one of the preceding embodiments, the biological condition of the female individual is polycystic ovarian syndrome.

In some embodiments of any one of the preceding embodiments, the biological condition of the female individual is experience of hot flashes.

In some embodiments of any one of the preceding embodiments, the biological condition of the female individual is other women-specific conditions or diseases.

In one aspect, the present disclosure provides a method comprising: (a) receiving a plurality of data points of a biological signal of a user, wherein the plurality of data points is collected over time, (b) analyzing the plurality of data points to determine a time or an estimated time when a sign of a rate of change of a temperature of the user becomes different, and (c) based on the determining, providing a control signal for a temperature regulator that is operatively coupled to an article of furniture, wherein the control signal comprises an instruction to change an operation of the temperature regulator for regulating a temperature of a portion of the article of furniture. In some embodiments, the article of furniture is a bed device.

In some embodiments of any one of the preceding embodiments, the plurality of data points is collected over time while the user is using the article of furniture.

In some embodiments of any one of the preceding embodiments, the biological signal is not a temperature of the user, optionally wherein the biological signal is a heart signal. In some embodiments, the biological signal is a temperature of the user.

In some embodiments of any one of the preceding embodiments, the analyzing occurs substantially in real-time after the receiving of the plurality of data points.

In some embodiments of any one of the preceding embodiments, the determining occurs substantially in real-time after the analyzing.

In some embodiments of any one of the preceding embodiments, the providing occurs substantially in real-time after the determining.

In some embodiments of any one of the preceding embodiments, the time or the estimated time is when a negative rate of change becomes a positive rate of change, or vice versa. In some embodiments, the time or the estimated time is when a positive or negative rate of change becomes substantially zero, or vice versa.

In some embodiments of any one of the preceding embodiments, the time or the estimated time is when a core temperature nadir (or a circadian rhythm temperature) of the user occur or is predicted to occur.

In some embodiments of any one of the preceding embodiments, the plurality of data points is measured by a sensor, optionally wherein the sensor is (i) a part of a user device that the user is wearing or (ii) a part of the article of furniture.

In some embodiments of any one of the preceding embodiments, the instruction directs the temperature regulator to change the temperature of the portion of the article of furniture.

In some embodiments of any one of the preceding embodiments, the instruction comprises a future time when the temperature regulator is to change the temperature of the portion of the article of furniture.

In some embodiments of any one of the preceding embodiments, the instruction comprises a change in a duration of time that the temperature regulator is to maintain the temperature of the portion of the article of furniture substantially constant before changing the temperature of the portion of the article of furniture.

In some embodiments of any one of the preceding embodiments, the temperature regulator is configured to change the temperature of the portion of the article of furniture from an initial temperature to a final temperature over a plurality of incremental temperature changes, and wherein the instruction comprises a change in a condition of the plurality of incremental temperature changes.

In some embodiments of any one of the preceding embodiments, at least a portion of the temperature regulator is contained within the portion of the article of furniture.

In some embodiments of any one of the preceding embodiments, the method is a computer-implemented method.

In some embodiments of any one of the preceding embodiments, the instruction is generated via an artificial intelligence algorithm or a machine learning algorithm.

In some embodiments of any one of the preceding embodiments, the control signal comprises an instruction for the temperature regulator to change a temperature of a portion of the article of furniture to a predetermined target temperature during use of the article of furniture by the user. In some embodiments, the predetermined target temperature is determined prior to a single use of the article of furniture by the user.

In some embodiments of any one of the preceding embodiments, the control signal comprises an instruction for the temperature regulator to change a temperature of a portion of the article of furniture from an initial temperature to a final temperature over a plurality of incremental temperature changes. In some embodiments, the plurality of incremental temperature changes comprises a predetermined number of incremental temperature changes.

In one aspect, the present disclosure provides a method comprising: providing a control signal for a temperature regulator that is operatively coupled to an article of furniture, wherein the control signal comprises an instruction for the temperature regulator to sequentially change a temperature of a portion of the article of furniture to each target temperature of a combination of target temperatures during use of the article of furniture by a user, wherein each target temperature of the combination of target temperatures are different from one another, and wherein the combination of target temperatures is selected from a database comprising a plurality of combinations of target temperatures based on one or more members selected from the group consisting of a geolocation of the user, biological sex of the user, and age of the user, wherein the user is not permitted to change the combination of target temperatures for at least one use of the article of furniture.

In some embodiments, the article of furniture is a bed device.

In some embodiments of any one of the preceding embodiments, the combination of target temperatures comprises at least two different target temperatures or at least three different target temperatures.

In some embodiments of any one of the preceding embodiments, the combination of target temperatures comprises at most five different target temperatures or at most four different target temperatures.

In some embodiments of any one of the preceding embodiments, the combination of target temperatures comprises a bedtime temperature.

In some embodiments of any one of the preceding embodiments, the combination of target temperatures does not comprise a wake-up temperature.

In some embodiments of any one of the preceding embodiments, each combination of the combinations of target temperatures is different based on the one or more members selected from the group consisting of a geolocation of the user, biological sex of the user, and age of the user. In some embodiments, the one or more members comprises two members or all three members elected from the group consisting of the geolocation of the user, the biological sex of the user, and the age of the user. In some embodiments, the one or more members comprises the geolocation of the user. In some embodiments, the one or more members comprises the biological sex of the user. In some embodiments, the one or more members comprises the age of the user.

In some embodiments of any one of the preceding embodiments, the method further comprises subsequent to use of the article of furniture by the user based on the combination of the target temperatures, modifying one or more target temperatures of the combination of target temperatures, to effect enhanced user experience.

In some embodiments of any one of the preceding embodiments, the user is not permitted to change the combination of target temperatures for at least three uses.

In some embodiments of any one of the preceding embodiments, the user is not permitted to change the combination of target temperatures for at least five uses.

In some embodiments of any one of the preceding embodiments, the user is not permitted to change the combination of target temperatures for at least seven uses.

In some embodiments of any one of the preceding embodiments, the method further comprises, subsequent to using the article of furniture based on the combination of target temperatures for the at least one use, permitting the user to modify one or more target temperatures of the combination of target temperatures via a graphical user interface (GUI) operatively coupled to the article of furniture, to effect enhanced user experience. In some embodiments, the enhanced user experience is enhanced sleep.

In some embodiments of any one of the preceding embodiments, the one or more target temperatures of the combination of target temperatures are changed (i) based on feedback from the user via a graphical user interface (GUI) operatively coupled to the article of furniture or (ii) automatically by a computer processor in absence of any user feedback.

In some embodiments of any one of the preceding embodiments, the combination of target temperatures is provided when the user is using the article of furniture for the first time.

In some embodiments of any one of the preceding embodiments, the combination of target temperatures is provided when an identity of the user is undetermined.

In some embodiments of any one of the preceding embodiments, the providing comprises recommending the combination of target temperatures to the user.

In some embodiments of any one of the preceding embodiments, at least a portion of the temperature regulator is contained within the portion of the article of furniture.

In one aspect, the present disclosure provides a method comprising: (a) determining a target temperature of at least a portion a bed device, and (b) sending an instruction to a temperature regulator coupled to the bed device, wherein the instruction is for the temperature regulator to subject at least a portion of the bed device to a plurality of temperature fluctuation cycles, wherein a temperature fluctuation cycle of the plurality comprises (i) a temperature increase and (ii) a temperature decrease, wherein an average temperature of the at least the portion of the bed device over a duration of the plurality is not different than the target temperature by more than 5 degrees Celsius.

In some embodiments, the average temperature is not different than the target temperature by more than 2 degrees Celsius.

In some embodiments of any one of the preceding claims, the temperature increase is by at least about 0.5 degrees Celsius.

In some embodiments of any one of the preceding claims, the temperature decrease is by at least about 0.5 degrees Celsius.

In some embodiments of any one of the preceding claims, the duration is at most about 30 minutes.

In some embodiments of any one of the preceding claims, the plurality comprises at least 5 temperature fluctuation cycles.

In some embodiments of any one of the preceding claims, the plurality comprises at least 10 temperature fluctuation cycles.

In some embodiments of any one of the preceding claims, the target temperature is associated with a sleep phase of a user of the bed device.

In some embodiments of any one of the preceding claims, the method further comprises, in (a), determining a target duration of time during which to set a temperature of the at least the portion of the bed device to about the target temperature.

In some embodiments of any one of the preceding claims, wherein the temperature regulator is capable of directing a fluid that is temperature regulated on or adjacent to the at least the portion of the bed device, wherein the instruction is for the temperature regulator to (i) subject the fluid to the plurality of temperature fluctuation cycles and (ii) direct the fluid to the at least the portion of the bed device. In some embodiments, the fluid is liquid. In some embodiments, the fluid is air.

In one aspect, the present disclosure provides a method comprising: receiving input from a user of an article of furniture, based on the input from the user, providing a control signal for a temperature regulator that is operatively coupled to the article of furniture, wherein the control signal comprises an instruction for the temperature regulator to adjust a temperature of at least a portion of the article of furniture from a predetermined temperature profile to a preset temperature in real time to adjust the current temperature of the article of furniture.

In some embodiments, the input from the user is provided via a graphical user interface (GUI). In some embodiments, the GUI is present on a user device.

In some embodiments, the input from the user is provided via one or more sensors disposed in the article of furniture. In some embodiments, the one or more sensors are disposed in the top of the article of furniture. In some embodiments, the one or more sensors detects an increase in a body temperature of the user.

In some embodiments of any one of the preceding embodiments, the input provided is that the user is having a hot flash.

In some embodiments of any one of the preceding embodiments, the predetermined temperature profile is stored in a database.

In some embodiments of any one of the preceding embodiments, the preset temperature is preprogrammed by the user prior to use of the article of furniture.

In some embodiments of any one of the preceding embodiments, the article of furniture remains at the preset temperature for at least about 30 minutes.

In some embodiments of any one of the preceding embodiments, the article of furniture remains at the present temperature for at least about 60 minutes.

In some embodiments of any one of the preceding embodiments, the temperature of the at least the portion of the article of furniture is adjusted to the preset temperature at a rate of at most about 10° C./min.

In some embodiments of any one of the preceding embodiments, the method comprises: (a) directing a temperature regulator to maintain the temperature of the at least the portion of an article of furniture substantially at a target temperature based on the predetermined temperature profile for the article of furniture, the predetermined temperature profile comprising the target temperature, (b) while the at least the portion of the article of furniture is maintained substantially at the target temperature, receiving the input from the user, and (c) based on the input, providing the control signal to the temperature regulator, the control signal comprising the instruction to change the temperature of the article of furniture to the preset temperature, wherein the target temperature and the present temperature are different.

In some embodiments, the instruction is to change the temperature of the article of furniture to the preset temperature transiently for a predetermined duration of time. In some embodiments, the predetermined duration of time is less than about 30 minutes, less than about 20 minutes, or less than about 10 minutes. In some embodiments, the method further comprises, subsequent to (c), directing the temperature regulator to regulate the temperature of the at least the portion of the article of furniture based on the predetermined temperature profile. In some embodiments, the method further comprises, subsequent to (c), directing the temperature regulator to change the temperature of the at least the portion of the article of furniture back to the target temperature. In some embodiments, the method further comprises, subsequent to (c), directing the temperature regulator to change the temperature of the at least the portion of the article of furniture to an additional target temperature of the predetermined temperature profile.

In some embodiments of any one of the preceding embodiments, the preset temperature and the target temperature are different by at least about 0.5 degrees Celsius (° C.), at least about 1° C., at least about 2° C., at least about 3° C., at least about 4° C., at least about 5° C., or more.

In some embodiments of any one of the preceding embodiments, the preset temperature and the target temperature are different by at most about 5° C., at most about 4° C., at most about 3° C., at most about 2° C., at most about 1° C., at most about 0.5° C., or less.

In some embodiments of any one of the preceding embodiments, the preset temperature is predetermined and stored in a database prior to (a).

In some embodiments of any one of the preceding embodiments, a system comprising one or more computer processors and computer memory coupled thereto, the computer memory comprising a machine executable code that, upon execution by the one or more computer processors, implements the method of any one of the preceding claims.

In some embodiments, the system further comprises the article of furniture.

In some embodiments of any one of the preceding embodiments, the system further comprises the temperature regulator of any one of the preceding claims.

In one aspect, the present disclosure provides a method (e.g., a computer-implemented method) comprising providing a control signal for a temperature regulator that is operatively coupled to an article of furniture, wherein the control signal comprises an instruction for the temperature regulator to change a temperature of a portion of the article of furniture from an initial temperature to a final temperature over a plurality of incremental temperature changes, wherein the plurality of incremental temperature changes comprises a predetermined number of incremental temperature changes.

In one aspect, the present disclosure provides a method (e.g., a computer-implemented method) comprising providing a control signal for a temperature regulator that is operatively coupled to an article of furniture, wherein the control signal comprises an instruction for the temperature regulator to sequentially change a temperature of a portion of the article of furniture to each target temperature of a combination of target temperatures during use of the article of furniture by a user, wherein each target temperature of the combination of target temperatures are different from one another, and wherein the combination of target temperatures is selected from a database comprising a plurality of combinations of target temperatures based on one or more members selected from the group consisting of a geolocation of the user, biological sex of the user, and age of the user.

In one aspect, the present disclosure provides a method (e.g., a computer-implemented method) comprising providing a control signal for a temperature regulator that is operatively coupled to an article of furniture, wherein the control signal comprises an instruction for the temperature regulator to change a temperature of a portion of the article of furniture to a target temperature during use of the article of furniture by a user, wherein the target temperature is determined based on biological sex of the user, such that a first provided temperature for a female user and a second provided temperature for a male user are different.

In one aspect, the present disclosure provides a method (e.g., a computer-implemented method) comprising (a) receiving a plurality of data points of a biological signal of a user, wherein the plurality of data points is collected over time; (b) analyzing the plurality of data points to determine substantially when a sign of a rate of change of a temperature of the user becomes different; and (c) based on the determining, providing a control signal for a temperature regulator that is operatively coupled to an article of furniture, wherein the control signal comprises an instruction to change an operation of the temperature regulator for regulating a temperature of a portion of the article of furniture.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 is a diagram of a bed device, according to one embodiment.

FIG. 2 illustrates an example of a bed device, according to one embodiment.

FIG. 3 illustrates an example of layers comprising a bed pad device, according to one embodiment.

FIG. 4 illustrates an example of a system for regulating temperatures of a plurality of portions of an article of furniture.

FIG. 5 shows a computer system that is programmed or otherwise configured to implement methods provided herein.

FIG. 6 depicts the incremental changes (e.g., increases) in the bed device between the initial temperature and the final temperature. The temperature of the bed device can change (e.g., increases) every 90 minutes to correlate with average length of a sleep cycle (SC). If the subject has over four sleep cycles, then the bed device can be maintained at the final bed device temperature. SC1: Sleep Cycle 1, SC2: Sleep Cycle 2; SC3: Sleep Cycle 3; SC4: Sleep Cycle 4; SC5: Sleep Cycle 5

FIG. 7 shows the percent of dial updates per subject per week tested in the incremental changes (e.g., increases) between initial and final temperature group (New AP (Exp 2)) and the no incremental changes (e.g., increases) between initial and final temperature group (Baseline (current AP)). The x-axis shows the Subject ID number for the subjects included in the study.

FIG. 8A illustrates an example graphical user interface (GUI) depicting the incremental temperature changes of the bed device.

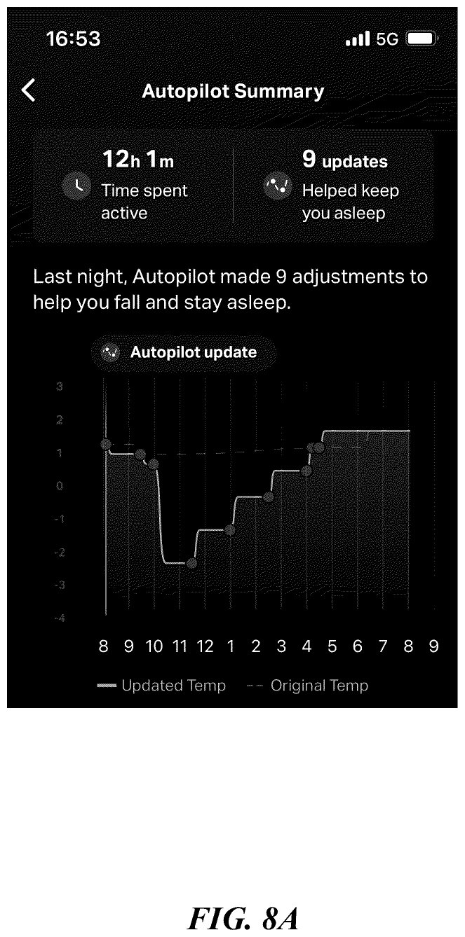

FIG. 8B illustrates an example graphical user interface (GUI) depicting the incremental temperature changes of the bed device, including temperature fluctuations of the final temperature.

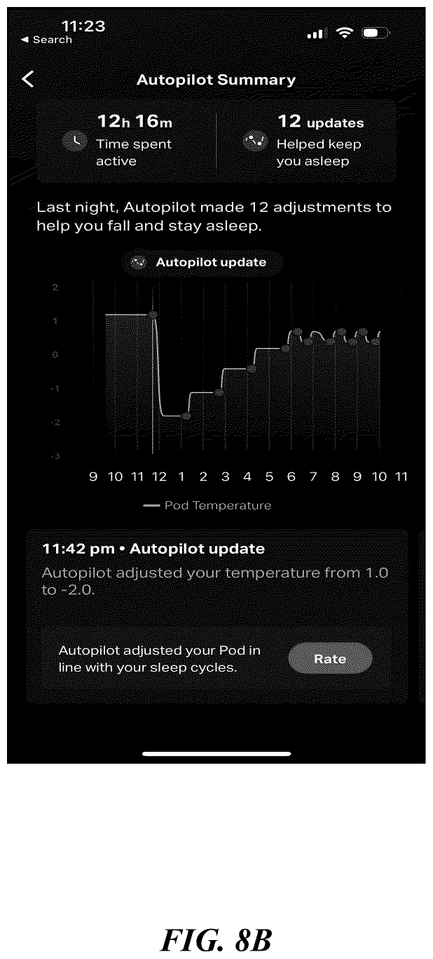

FIG. 8C illustrates an example GUI depicting the four temperatures that the subject can set in the bed device app. Bed Time-bedtime temperature; Deep-initial temperature; REM-final temperature; Wakeup-wake-up temperature.

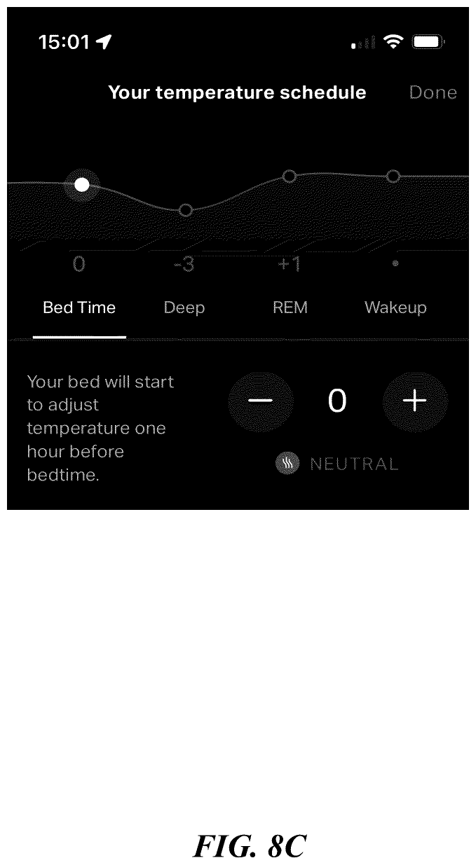

FIG. 9A depicts the bed device temperature dial values for the control, T1, and T2 groups for the initial temperature profile recommended based on age, biological sex, and seasonal information.

FIG. 9B depicts the bed device temperature dial values for the control, T1, and T2 groups for the initial temperature profile recommended based on age, biological sex, and geographical location.

FIG. 10 illustrates the group sizes for each initial temperature profile group (control, T1, and T2), number of male/female subjects, number of subjects in each age group (less than 36 years old, between 36 to 50 years old, greater than 50 years old), and winter/summer profiles.

FIG. 11 depicts the difference in temperature level of female subject's bed device during the subject's follicular phase and luteal phase. The difference in temperature level was calculated by taking the median temperature across each user/period/phase, taking the temperature difference within each month between bed device temperatures for the luteal phase minus the follicular phase. For a given subject, the mean temperature differences were calculated across all months. The y-axis represents the temperature difference on the bed device temperature dial. Age bin 0.0 represents female users younger than 35 years old; age bin 1.0 represents female users between the age of 35 to 60 years old. The (1) boxes represent the bed device temperature during follicular phase; the (2) boxes represent the bed device temperature during luteal phase. The triangle depicts the mean difference for each group and the black link inside the box depicts the median of each group.

FIG. 12 depicts the temperature levels of the bed device for the female subject during follicular phase and luteal phase of the subject's menstrual cycle. The (1) boxes represent the bed device temperatures during the subject's follicular phase; the (2) boxes represent the bed device temperatures during the subject's luteal phase. The open circles on the plot represent the mean for each group; the black horizontal line within each box in the boxplot represents the median of each group. Age-bin 0.0 represent female subjects under 35 years of age; age bin 1.0 represent female subjects between the age of 35 to 60 years old.

FIG. 13 depicts the percentage dial updates during the follicular and the luteal phase of the subject's menstrual cycle. The (1) boxes represent the follicular phase; the (2) boxes represent the luteal phase. The open circles on the plot represent the mean for each group; the black horizontal line within each box in the boxplot represents the median of each group. Age-bin 0.0 represent female subjects under 35 years of age; age bin 1.0 represent female subjects between the age of 35 to 60 years old.

FIG. 14 depicts the percentage of nights that subject adjusted the temperature dial values (e.g., override) for male subjects and female subjects. The data was normalized by taking the total number of nights with a bed device temperature for each individual and calculating the percentage of nights the subject made a dial value adjustment. The (1) boxes represent male subjects; the (2) boxes represent female subjects. The open circles on the plot represent the mean for each group; the black horizontal line within each box in the boxplot represents the median of each group.

FIG. 15 depicts normal core body temperature curve throughout the night. Women generally can have an earlier core temperature nadir compared to men. During a 24-hour period, the core temperature of the subject can fluctuate between about 1° C. (1.8° F.). The red line illustrates the core temperature nadir.

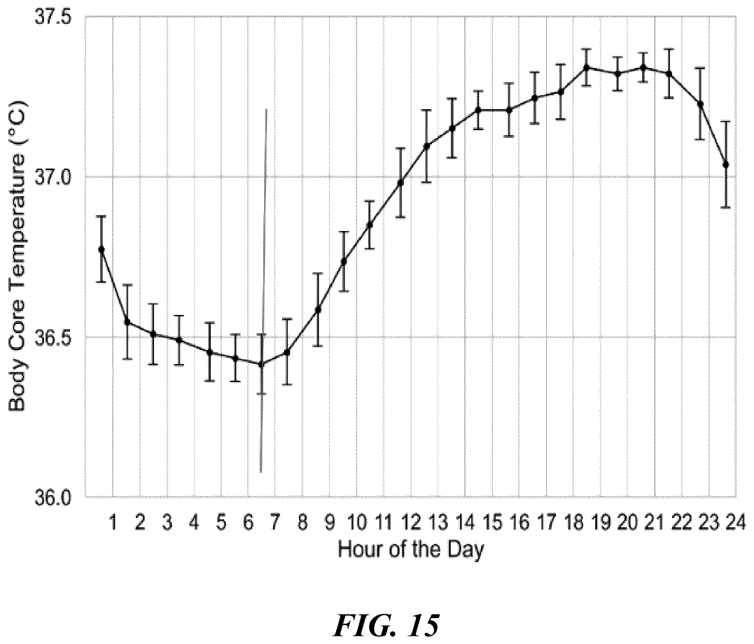

FIG. 16 depicts a schematic for the experimental design testing the temperature regulation of the bed device. Subjects (n=54) spent the first seven nights with temperature regulation off (bed device OFF Baseline), and then spent seven nights with temperature regulation on (bed device ON), followed by the last two nights with temperature regulation off (bed device OFF End). Sleep stages, sleep onset latency (SOL), sleep efficiency (SE), and total sleep time (TST) were recorded on Nights 6-9 and 13-16 through a home sleep test device (HST). Throughout the entire study, sleeping HR and HRV along with daily exercise were recorded via a Fitbit Activity Track or Smartwatch available from Alphabet Inc. of Mountain View, CA. Ratings of perceptual comfort and sleep quality were recorded via a daily morning survey.

FIG. 17 shows changes in HR and HRV based on bed device temperature status in female users (1) and male users (2). Means±SE for minimum sleeping HR and median sleeping HRV at bed device OFF Baseline, bed device ON, and Post-bed device OFF stratified by sex. * Indicates significant difference in linear mixed models from bed device OFF Baseline at p<0.05.

FIG. 18 shows changes in sleep latency metrics from bed device OFF for cool (1) vs warm (2) bedtime temperatures. Mean difference and 95% confidence intervals for the change in sleep onset latency (SOL) and Deep SOL metrics from bed device OFF Baseline vs. bed device ON sleeping at cool or warm Bedtime temperatures, stratified by sex. REM SOL represents the mean difference from bed device OFF to cool vs. warm initial Phase temperatures. * Indicates significant difference from bed device OFF Baseline at P<0.10.

FIG. 19 shows changes in absolute sleep stage duration based on cool (1) vs. warm (2) sleepers in initial and final phases. Boxplots depicting distributions of changes in time (min) spent in each sleep stage for bed device OFF vs. Cool or Warm temperatures during initial and final Phase, for females (A) and males (B). Each individual dot represents the weighted mean change in time spent in that sleep stage for each individual. The black diamonds denote means and the horizontal lines within the boxes denote medians. The dotted line at 0 indicates no change from bed device OFF baseline to bed device ON. * Indicates significant difference in linear mixed models from bed device OFF Baseline at p<0.10.

FIG. 20 shows changes in the percentage of total sleep time spent in each sleep stage on cool (1) vs. warm (2) sleepers in initial and final phases. Boxplots depicting distributions of changes in the percentage of time spent in each sleep stage for bed device OFF vs. Cool or Warm temperatures. Initial Phase (left-hand panels) and final Phase (righthand panels) are separated for females (A) and males (B). Each individual dot represents the weighted mean change in time spent in that sleep stage for each individual. The black diamonds denote means and the horizontal lines within the boxes denote medians. The dotted line at 0 indicates no change from bed device OFF baseline to bed device ON. * Indicates significant difference in linear mixed models from bed device OFF Baseline at p<0.10.

FIG. 21 depicts a Confusion matrix showing the sleep staging algorithm's accuracy.

### DETAILED DESCRIPTION

While preferred embodiments of the present disclosure have been shown and described herein, it will be obvious to those skilled in the art that such embodiments are provided by way of example only. Numerous variations, changes, and substitutions will now occur to those skilled in the art without departing from the scope present disclosure. It should be understood that various alternatives to the embodiments of the present disclosure described herein may be employed in practicing the embodiments present disclosure. It is intended that the below claims define the scope of the invention and that methods and structures within the scope of these claims and their equivalents be covered thereby.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the inventions herein can be described in the context of a single embodiment, the features can also be provided separately or in any suitable combination. Conversely, although the inventions herein can be described herein in the context of separate embodiments for clarity, the inventions herein can also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting. In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the inventions.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the invention, and vice versa. Furthermore, compositions of the present disclosure can be used to achieve methods of the invention.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

The terms “subject” and “patient,” as used herein, include animals (e.g., vertebrates, amphibians, fish, mammals, cats, dogs, horses, pigs, cows, sheep, rodents, rabbits, squirrels, bears, primates (e.g., chimpanzees, gorillas, and humans)), which are capable of suffering from cancer. “Subject” may also be a cell, a population of cells, a tissue, an organ, or an organism, preferably to human and constituents thereof. In a preferred embodiment, the mammal is a human.

A “control” or “standard control” refers to a sample, measurement, or value that serves as a reference, usually a known reference, for comparison to a test sample, measurement, or value.

The terms “furniture,” “article of furniture,” or “piece of furniture,” as used interchangeably herein, can refer to a bed, crib, bassinet, chair, seat, loveseat, sofa, couch, head rest, stool, ottoman, bench, or any panel intended to be covered with a fabric. The article of furniture can be intended for use in a home, an office, a medical facility (e.g., a hospital), or on a vehicle of transportation such as a car, truck, boat, bus, train or the like. The article of furniture can be intended for use for at least one person (and/or at least one animal, such as a pet). The article of furniture can be intended for use for at least about 1, 2, 3, 4, 5, 6, 7, 8, 9, 10, or more persons. The article of furniture can be intended for use for at most about 10, 9, 8, 7, 6, 5, 4, 3, 2, or 1 person. In an example, the article of furniture may be a bed, and the bed may comprise a plurality of sizes comprising single, single extra-long, double, queen, king, super king, etc. In another example, the article of furniture may be an infant warmer (e.g., a babytherm) to provide heat at one or more temperatures to an infant.

The terms “bed” or “bed device,” as used interchangeably herein, may be an article of furniture used for sleep or rest. The bed may comprise a mattress, a mattress pad, and/or a covering (e.g., a blanket). One or more users may sleep or rest on and/or adjacent to a surface of the bed. The surface may be a top surface of the bed. The top surface of the bed may be flat or textured. The bed may be the mattress. The bed may be the mattress pad that covers at least a portion of a surface of a mattress or at least a surface of the mattress. Alternatively or in addition to, the user(s) may sleep under a surface of the bed. The surface may be one or more surfaces of a covering, such as, for example, a blanket. The blanket may be disposed on top of at least a part of the user(s). The bed may be the blanket. The terms “biological signal,” “vital signal,” and “bio signal” can be used interchangeably. Examples of the biological signal can include a heart signal (e.g., heart rate or sound), a respiration (breathing) signal (e.g., respiration rate or sound), a motion, a temperature, a movement, perspiration, sound, neural activity, etc. The article of furniture (e.g., the bed) may be capable of detecting one or more biological signals of the user(s). The article of furniture may be capable of adjusting a property of the article of furniture (e.g., temperature or movement of the article of furniture, such as vibration, geometric configuration, etc.) to control (e.g., increase, decrease, or maintain) the biological signal(s) of the user(s) of the article of furniture.

### Systems and Methods for Temperature Regulation of an Article of Furniture

Various aspects of the present disclosure provide systems and methods (e.g., computer-implemented methods) for providing or regulating a setting of an article of furniture. Non-limiting examples of a setting of an article of furniture can include temperature, shape, movement (e.g., vibration), etc. The article of furniture can be, for example, a bed device, chair, or sofa. Thus, any disclosures directed to one example of an article of furniture (e.g., a bed device) may be interchangeable with another example of an article of furniture (e.g., chair, sofa, etc.).

Various aspects of the systems and methods (e.g., computer-implemented methods) provided herein can be utilized to tailor a setting of the article of furniture (e.g., temperature of a bed device) based on one or more parameters specific for a user of the article of furniture. Non-limiting examples of such parameter(s) can include, age, biological sex, geolocation, time (e.g., with respect to the day, with respect to the week, with respect to the month, with respect to the year, with respect to the season) of use of the article of furniture, sleep habits or patterns (e.g., average bedtime), health condition or state (e.g., healthy, a disease type, pregnancy, menstruation cycle, menopause, hot flashes, night sweats, etc.). The setting of the article of furniture can be tailored prior to, during, and/or subsequent to the user's use of the article of furniture. For example, a temperature of a bed device can be adjusted, via the system and methods provided herein, prior to the user's getting into the bed device, prior to the user's falling asleep on the bed device, during the user's sleep on the bed device, prior to the user's waking up while sleeping on the bed device, during the user's waking up on the bed device, and/or subsequent to the user's use of the bed device.

Without wishing to be bound by theory, a same setting of the article of furniture can feel different or have different effects (e.g., health effects, different experiences) on different people, and adjusting a setting of the article of furniture based on one or more parameters of an individual can enhance the individual's user experience (e.g., can enhance quality of sleep of the user on a bed device). For example, a setting that is beneficial for a biological male user's experience in using the article of furniture (e.g., sleeping on a bed device) may not be beneficial for a biological female user's experience in using the same, or vice versa. Thus, various aspects of the systems and methods, for example, can fulfill the unmet need of providing an optimal and/or personalized setting of the article of furniture for the user and/or assisting the user to determine such optimal and/or personalized setting. The setting of the article of furniture can be adjusted prior to the user's use (e.g., prior to the user's first use of the article of furniture, prior to user's use of the day, etc.), during the user's use, and/or subsequent to the user's use. In some cases, such parameter(s) can be estimated based on detection data from one or more detectors such as sensors operatively coupled to the article of furniture (e.g., sensor(s) in the article of furniture). Alternatively or in addition to, such parameter(s) can be provided by the user (e.g., via graphical user interface (GUI) of a user device, such as a smart watch or smart phone).

The systems and methods (e.g., computer-implemented methods) of the present disclosure can be utilized for regulating temperature of a surface. In some embodiments, the surface is a bed device. In some embodiments, the bed device changes temperature. In some embodiments, the bed device changes temperature while a subject is on the surface of the bed device. In some embodiments, the bed device is operatively coupled to one or more sensors. The one or more sensors can be part of the bed device, part of a room or space comprising the bed device there within, and/or a part of a wearable device that the user of the bed device is wearing. The sensor(s) can be configured to measure one or more biological signals comprising heart signal, breathing signal, respiration rate, temperature, movement, presence, etc. Non-limiting examples of a sensor can include a capacitance sensor (e.g., for detecting presence), a piezo sensor (e.g., for measuring a biological signal such as heart signal, breathing signal, respiration signal, etc.), a temperature sensor (e.g., for measuring temperature of the user, or that of the bed), pressure sensor, optical sensor, humidity sensors, etc.

In some embodiments, the bed device further comprises a processor. In some embodiments, the processor analyzes the data collected by the one or more sensors. In some embodiments, the bed device further comprises a temperature regulator. In some embodiments, the temperature regulator is operatively connected to the processor. In some embodiments, the processor can provide a control signal to the temperature regulator. In some embodiments, the control signal can be an instruction for the temperature regulator to change a temperature of the article of furniture. In some embodiments, the control signal can be an instruction for the temperature regulator to change a temperature of a portion of the article of furniture (e.g., bed device). In some embodiments, the temperature regulator is capable of directing a fluid that is temperature regulated on at least a portion of the bed device. In some embodiments, the temperature regulator is capable of directing a fluid that is temperature regulated adjacent to the at least the portion of the bed device. In some cases, the fluid is liquid. In some cases, the fluid is a gas. In some cases, the gas is air. In some cases, the temperature regulator is located within the bed device. In some cases, the temperature regulator is located separate from the bed device.

In some embodiments, the bed device can further comprise a device comprising a memory. In some cases, the device can be coupled to the processor. In some cases, the device can be a RAM device, a solid-state drive (SSD), a hard disc, a flash drive, a flash memory, a compact disc, a pen drive, a hard disc drive, or any combination thereof.

In some embodiments, the one or more sensors detects one or more biometrics from the subject when on the surface of the bed device. In some embodiments, the one or more sensors measures one or more biometrics from the subject when on the surface of the bed device. In some embodiments, the one or more sensors collect data of the one or more biometrics from the subject when on the surface of the bed device. In some embodiments, the one or more biometrics is heart rate (HR). Heart rate can measure the number of times each minute a heart beats. In some embodiments, the one or more biometrics is heart rate variability (HRV). Heart rate variability can measure the variation in time between each heartbeat (e.g., the time difference between the peaks of R-waves over time in cardiac cycle). In some embodiments, the one or more biometrics is the skin temperature of the subject when on the surface of the bed device. In some embodiments, the one or more biometrics is respiration rate. The respiration rate can be the number of breaths the subject takes per minute. In some embodiments, the one or more biometrics is the perspiration of the subject when on the bed device. In some embodiments, the one or more sensors detect moisture or changes in humidity from the subject's perspiration. In some embodiments, the one or more biometrics are HR, HRV, respiration rate, and skin temperature. In some embodiments, the one or more biometrics are HR and HRV. In some embodiments, the one or more biometrics are HR and skin temperature. In some embodiments, the one or more biometrics are HRV and skin temperature. In some embodiments, the one or more biometrics can be an electrocardiogram. In some embodiments, the one or more biometrics can be an electromyography. In some embodiments, the one or more biometrics can be an electroencephalography. In some embodiments, the one or more biometrics are percent light sleep, percent deep sleep, percent REM sleep, total sleep time, sleep cycle length, wake time, total net sleep score, HR, HRV, skin temperature, respiration rate, electrocardiogram, electromyography, electroencephalography, or any combination thereof.

In some embodiments, the one or more sensors are in a wearable device that the subject wears. In some embodiments, the wearable device measures the one or more biometrics of the subject. In some cases, the wearable device may measure any of the biometrics disclosed herein. In some embodiments, the wearable device syncs with the bed device app and the bed device app collects data from the wearable device. Non-limiting examples of wearable devices include: 3P smartwatches available from 3P Experts of Addison, TX, Apple Smartwatches available from Apple Inc. of Cupertino, CA, Fitbit smartwatches, Garmin smartwatches available from Garmin Ltd. of Olathe, KS, Oura smartring available from Oura Health Oy of Oulu, Finland, WHOOP smartbands available from WHOOP Inc. of Boston, MA, WHOOP smartwatches available from WHOOP Inc. of Boston, MA, Biostrap monitor available from Biostrap USA, LLC of Duarte, CA, a chest wore heart monitoring band, a sensor patch, a sensor headband, and Go2sleeper smartring available from SLEEPON of Shenzhen, China.

In some embodiments, the user of the bed device can wake up during the use of the bed device. In some embodiments, the bed device app shows the user the length of time the user was asleep, the sleep phrase the user was in when the user woke up, and/or the type of sleep (e.g., light sleep, deep sleep, REM-sleep) the user was in when the user woke up.

In some embodiments, the one or more sensors measures the one or more biometrics while the subject on the bed device. In some embodiments, the wearable device measures the one or more biometrics while the subject is not on the bed device. In some embodiments, the bed device app sends a summary to the subject through the phone. In some embodiments, the bed device app sends the summary to the subject between every 24 hours to about every 31 days. In some embodiments, the bed device app sends the summary to the subject every about 24 hours, about 48 hours, about 3 days, about 4 days, about 5 days, about 6 days, about 7 days, about 14 days, about 28 days, about 30 days, or about 31 days.

In some embodiments, the summary has information collected while the subject is on the bed device. In some embodiments, the summary has information collected from the one or more sensors. Non-limiting information in the summary can include: HR, HRV, respiration rate, occurrence of perspiration events, humidity, timing of perspiration events, skin temperature, total sleep time, percent of light sleep, percent of deep sleep, percent of REM sleep, wake time, and net sleep score. In some embodiments, the summary includes recommendations to improve sleep. In some embodiments, the summary includes exercise suggestions to improve sleep (e.g., light vs heavy exercise). In some embodiments, the summary includes suggested bedtimes to improve sleep. In some embodiments, the summary includes whether the subject needs more sleep in one or more sleep stages (e.g., deep sleep, light sleep, REM sleep). In some embodiments, the summary has information collected over between about 24 hours to about ten years. In some embodiments, the summary has information collected over about 24 hours, about 48 hours, about 3 days, about 4 days, about 5 days, about 7 days, about 14 days, about 28 days, about 30 days, about 31 days, about two months, about three months, about four months, about five months, about six months, about seven months, about eight months, about nine months, about ten months, about 11 months, about one year, about two years, about three years, about four years, about five years, about six years, about seven years, about eight years, about nine years, or about ten years.

### Regulating Temperatures Based on Sleep Cycles

One aspect of the present disclosure provides systems and methods (e.g., a computer-implemented method) for regulating the temperature of a bed device when a subject is on the bed device. In some embodiments, the regulating the temperature of the bed device is based on an individual's sleep cycles and sleep cycle length. In some embodiments, the regulating the temperature of the bed device based on the individual's sleep cycles and sleep cycle length is performed retroactively. In some embodiments, the regulating the temperature of the bed device based on the individual's sleep cycles and sleep cycle length is performed in real time during use of the bed device. In some embodiments, the regulating the temperature of the bed device can occur between about 1 second to about 10 minutes after determining the individual's sleep cycles or sleep cycle length. In some cases, the regulating the temperature of the bed device can occur between about 1 second to about 10 seconds, between about 10 seconds to about 30 seconds, between about 30 seconds to 60 seconds, between about 60 seconds to about 2 minutes, between about 2 minutes to about 3 minutes, between about 3 minutes to about 4 minutes, between about 4 minutes to about 5 minutes, between about 5 minutes to about 6 minutes, between about 6 minutes to about 7 minutes, between about 7 minutes to about 8 minutes, between about 8 minutes to about 9 minutes, or between about 9 minutes to about 10 minutes after determining the individual's sleep cycles or sleep cycle length. In some cases, the regulating the temperature of the bed device can occur at least about 1 second, at least about 5 seconds, at least about 10 seconds, at least about 15 seconds, at least about 20 seconds, at least about 25 seconds, at least about 30 seconds, at least about 35 seconds, at least about 40 seconds, at least about 45 seconds, at least about 50 seconds, at least about 55 seconds, at least about 60 seconds, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, or more than 10 minutes after determining the individual's sleep cycles or sleep cycle length. In some cases, the regulating the temperature of the bed device can occur at most about 10 minutes, at most about 9 minutes, at most about 8 minutes, at most about 7 minutes, at most about 6 minutes, at most about 5 minutes, at most about 4 minutes, at most about 3 minutes, at most about 2 minutes, at most about 60 seconds, at most about 55 seconds, at most about 50 seconds, at most about 45 seconds, at most about 40 seconds, at most about 35 seconds, at most about 30 seconds, at most about 25 seconds, at most about 20 seconds, at most about 15 seconds, at most about 10 seconds, at most about 5 seconds, at most about 1 second, or less than 1 second after determining the individual's sleep cycles or sleep cycle length. In some cases, the regulating the temperature of the bed device can occur about 1 second, about 5 seconds, about 10 seconds, about 15 seconds, about 20 seconds, about 25 seconds, about 30 seconds, about 35 seconds, about 40 seconds, about 45 seconds, about 50 seconds, about 55 seconds, about 60 seconds, about 2 minutes, about 3 minutes, about 4 minutes, about 5 minutes, about 6 minutes, about 7 minutes, about 8 minutes, about 9 minutes, or about 10 minutes after determining the individual's sleep cycles or sleep cycle length.

In some embodiments, the respiration rate and/or the heart rate (HR) of the user can decrease when the user is asleep on the bed device. In some cases, the respiration rate and/or the heart rate of the user can be lower when the user is asleep on the bed device than when the user is awake on the bed device.

In some cases, a sleep cycle can have a plurality of stages of sleep from the following: stage one of non-rapid eye movement (REM) sleep, stage two of non-REM sleep, stage three of non-REM sleep, and REM sleep. In stage one of non-REM sleep, a subject's eyes are closed and the subject can be easily woken up. This stage may last between about 1 minute to about 10 minutes. In some embodiments, stage one of non-REM sleep lasts about one minute, about two minutes, about three minutes, about four minutes, about five minutes, about six minutes, about seven minutes, about eight minutes, about nine minutes, or about ten minutes. In some embodiments, the stage one of non-REM sleep lasts about five minutes. In some embodiments, the stage one of non-REM sleep lasts about ten minutes. Stage one of non-REM sleep can be considered light sleep.

In stage two of non-REM sleep, a subject may be in light sleep. The subject's HR can slow and the subject's body temperature can decrease. Stage two of non-REM sleep can be considered light sleep. In some embodiments, the first cycle of stage two non-REM sleep can last between about 10 minutes to about 25 minutes. In some embodiments the first cycle of stage two non-REM sleep can last about 10 minutes, about 15 minutes, about 20 minutes, or about 25 minutes. In some embodiments, each subsequent cycle of stage two non-REM sleep increases from the previous cycle.

Stage three of non-REM sleep can be the deep sleep stage. A subject in stage three of non-REM sleep may be difficult to awaken. Stage three of non-REM sleep can be considered deep sleep. In stage three of non-REM sleep, a subject's respiration rate and heart rate decreases. In some embodiments, stage three of non-REM sleep lasts between about 20 minutes to about 90 minutes. In some embodiments, stage three of non-REM sleep lasts about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, or about 90 minutes.

REM sleep can be the final stage of a sleep cycle of a subject. During REM sleep, a subject's heart rate and respiration rate can increase. A subject can dream during REM sleep. The first period of REM sleep may last about 10 minutes. Each subsequent cycle of REM sleep can increase from the length of time in REM sleep in the previous cycle. The final period of REM sleep may last about 60 minutes. Following the REM sleep stage, there can a period of the subject being awake. In some embodiments, the period of wake is between about 0 minutes to about 20 minutes. In some embodiments, the period of wake is about 0 minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. After completion of all stages of the sleep cycle, the sleep cycle starts over again.

In some embodiments, a subject has between about 3 to about 6 sleep cycles. In some embodiments, a subject has about four sleep cycles. In some embodiments, a subject has about five sleep cycles. In some embodiments, the subject has about six sleep cycles. In some embodiments, the first sleep cycle lasts between about 60 to 100 minutes. In some embodiments, the first sleep cycle lasts about 60 minutes, about 65 minutes, about 70 minutes, about 75 minutes, about 80 minutes, about 85 minutes, about 90 minutes, about 95 minutes, or about 100 minutes. In some embodiments, subsequent sleep cycles after the first sleep cycle last between about 90 to about 130 minutes. In some embodiments, the subsequent sleep cycles last about 90 minutes, about 95 minutes, about 100 minutes, about 105 minutes, about 110 minutes, about 115 minutes, about 120 minutes, about 125 minutes, or about 130 minutes.

In some embodiments, the bed device further comprises a processor. In some embodiments, the processor analyzes the data collected by the one or more sensors. In some embodiments, the bed device further comprises a temperature regulator. In some embodiments, the temperature regulator is operatively connected to the processor. In some embodiments, the processor can provide a control signal to the temperature regulator. In some embodiments, the control signal can be an instruction for the temperature regulator to change a temperature of the article of furniture. In some embodiments, the control signal can be an instruction for the temperature regulator to change a temperature of a portion of the article of furniture (e.g., bed device). In some embodiments, the control signal can be an instruction to change the temperature to the bedtime temperature. In some embodiments, the control signal can be an instruction to change the bedtime temperature to the initial temperature. In some embodiments, the control signal can be an instruction to change the temperature to the initial temperature. In some embodiments, the control signal can be an instruction to change the initial temperature to the final temperature. In some embodiments, the control signal can be an instruction to change the temperature to the final temperature. In some embodiments, the control signal can be an instruction to change the temperature to the wake-up temperature. In some embodiments, the control signal can be an instruction to change the final temperature to the wake-up temperature.

In some embodiments, the sleep cycle(s) provided herein can be predicted by the bed device (e.g., by a computer processor operatively coupled to the bed device). Such prediction can be based on information from other users and/or sensing data collected from the user while using the bed device.

In some embodiments, the subject (or user) selects a plurality of temperatures (e.g., via GUI operatively coupled to the system provided herein). For example, the subject can select at least four temperature settings: a first temperature (e.g., Bedtime or a bedtime temperature), a second temperature (e.g., Early Phase or an initial temperature), a third temperature (e.g., Late Phase or a final temperature), and a fourth temperature (e.g., a wake-up temperature).

In some embodiments, the bedtime temperature is the temperature of the bed device when the subject falls asleep while on the surface of the bed device. In some embodiments, the bed device maintains at the bedtime temperature for a period of time after the subject falls asleep on the surface of the bed device. The period of time can be at least or up to about 5 minutes, at least or up to about 10 minutes, at least or up to about 15 minutes, at least or up to about 20 minutes, at least or up to about 25 minutes, at least or up to about 30 minutes, at least or up to about 40 minutes, at least or up to about 50 minutes, at least or up to about 60 minutes, at least or up to about 90 minutes, or at least or up to about 120 minutes. In some embodiments, the initial temperature is the temperature of the bed device when the subject starts the first sleep cycle on the surface of the bed device. In some embodiments, the initial temperature of the bed device promotes deep sleep. In some embodiments, the initial temperature corresponds to a deep sleep temperature. In some embodiments, the final temperature is the temperature of the bed device following the initial temperature. In some embodiments, the final temperature of the bed device promotes REM sleep. In some embodiments, the final temperature corresponds to a rapid eye movement (REM) sleep temperature. In some embodiments, the final temperature of the bed device is the temperature that the subject will awaken to on the subject of the bed device. Alternatively, the bedtime temperature can be the temperature of the bed device that is preset prior to but near a bedtime (e.g., a predesignated bedtime, average bedtime) of the user prior to the user's use of the bed device.

In some embodiments, the initial temperature of the bed device is colder (e.g., lower) than the bedtime temperature of the bed device. In some embodiments, the initial temperature of the bed device is between about 0.5° Celsius (C.) to about 5° C. colder than the bedtime temperature of the bed device. In some embodiments, the initial temperature of the bed device is at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 6° C., at least or up to about 7° C., at least or up to about 8° C., at least or up to about 9° C., or at least or up to about 10° C. colder than the bedtime temperature of the bed device.

In some embodiments, the bedtime temperature of the bed device is colder (e.g., lower) than the initial temperature of the bed device. In some embodiments, the bedtime temperature of the bed device is between about 0.5° Celsius (C.) to about 5° C. colder than the initial temperature of the bed device. In some embodiments, the bedtime temperature of the bed device is at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 6° C., at least or up to about 7° C., at least or up to about 8° C., at least or up to about 9° C., or at least or up to about 10° C. colder than the initial temperature of the bed device.

In some embodiments, the initial temperature of the bed device is warmer (e.g., higher) than the bedtime temperature of the bed device. In some embodiments, the initial temperature of the bed device is between about 0.5° Celsius (C.) to about 5° C. colder than the bedtime temperature of the bed device. In some embodiments, the initial temperature of the bed device is at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 6° C., at least or up to about 7° C., at least or up to about 8° C., at least or up to about 9° C., or at least or up to about 10° C. colder than the bedtime temperature of the bed device.

In some embodiments, the bedtime temperature of the bed device is warmer (e.g., greater) than the initial temperature of the bed device. In some embodiments, the bedtime temperature of the bed device is between about 0.5° Celsius (C.) to about 5° C. warmer than the bedtime temperature of the bed device. In some embodiments, the bedtime temperature of the bed device is at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 6° C., at least or up to about 7° C., at least or up to about 8° C., at least or up to about 9° C., or at least or up to about 10° C. warmer than the initial temperature of the bed device.

In some embodiments, the initial temperature of the bed device is colder (e.g., lower) than the final temperature of the bed device. In some embodiments, the initial temperature of the bed device is between about 0.5° to about 5° C. colder than the final temperature of the bed device. In some embodiments, the initial temperature of the bed device is at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., or at least or up to about 5° C., at least or up to about 6° C., at least or up to about 7° C., at least or up to about 8° C., at least or up to about 9° C., or at least or up to about 10° C. colder than the final temperature of the bed device.

In some embodiments, the final temperature of the bed device is colder (e.g., lower) than the initial temperature of the bed device. In some embodiments, the final temperature of the bed device is between about 0.5° to about 5° C. colder than the initial temperature of the bed device. In some embodiments, the final temperature of the bed device is at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., or at least or up to about 5° C., at least or up to about 6° C., at least or up to about 7° C., at least or up to about 8° C., at least or up to about 9° C., or at least or up to about 10° C. colder than the initial temperature of the bed device.

In some embodiments, the initial temperature of the bed device is warmer (e.g., greater) than the final temperature of the bed device. In some embodiments, the initial temperature of the bed device is between about 0.5° to about 5° C. colder than the final temperature of the bed device. In some embodiments, the initial temperature of the bed device is at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., or at least or up to about 5° C., at least or up to about 6° C., at least or up to about 7° C., at least or up to about 8° C., at least or up to about 9° C., or at least or up to about 10° C. colder than the final temperature of the bed device.

In some embodiments, the final temperature of the bed device is warmer (e.g., greater) than the initial temperature of the bed device. In some embodiments, the final temperature of the bed device is between about 0.5° to about 5° C. colder than the initial temperature of the bed device. In some embodiments, the final temperature of the bed device is at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., or at least or up to about 5° C., at least or up to about 6° C., at least or up to about 7° C., at least or up to about 8° C., at least or up to about 9° C., or at least or up to about 10° C. colder than the initial temperature of the bed device.

In some embodiments, the bedtime temperature of the bed device is colder (e.g., lower) than the final temperature of the bed device. In some embodiments, the bedtime temperature of the bed device is between about 0.5° to about 5° C. colder than the final temperature of the bed device. In some embodiments, the bedtime temperature of the bed device is at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., or at least or up to about 5° C., at least or up to about 6° C., at least or up to about 7° C., at least or up to about 8° C., at least or up to about 9° C., or at least or up to about 10° C. colder than the final temperature of the bed device.

In some embodiments, the final temperature of the bed device is colder (e.g., lower) than the bedtime temperature of the bed device. In some embodiments, the final temperature of the bed device is between about 0.5° to about 5° C. colder than the bedtime temperature of the bed device. In some embodiments, the final temperature of the bed device is at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., or at least or up to about 5° C., at least or up to about 6° C., at least or up to about 7° C., at least or up to about 8° C., at least or up to about 9° C., or at least or up to about 10° C. colder than the bedtime temperature of the bed device.

In some embodiments, the bedtime temperature of the bed device is warmer (e.g., greater) than the final temperature of the bed device. In some embodiments, the bedtime temperature of the bed device is between about 0.5° to about 5° C. colder than the final temperature of the bed device. In some embodiments, the bedtime temperature of the bed device is at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., or at least or up to about 5° C., at least or up to about 6° C., at least or up to about 7° C., at least or up to about 8° C., at least or up to about 9° C., or at least or up to about 10° C. colder than the final temperature of the bed device.

In some embodiments, the final temperature of the bed device is warmer (e.g., greater) than the bedtime temperature of the bed device. In some embodiments, the final temperature of the bed device is between about 0.5° to about 5° C. colder than the bedtime temperature of the bed device. In some embodiments, the final temperature of the bed device is at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., or at least or up to about 5° C., at least or up to about 6° C., at least or up to about 7° C., at least or up to about 8° C., at least or up to about 9° C., or at least or up to about 10° C. colder than the bedtime temperature of the bed device.

In some embodiments, the bedtime temperature of the bed device can be at a steady temperature setting. In some cases, the steady temperature setting can be when the temperature of the bed device remains at the same degrees Celsius during the duration of the bedtime temperature of the bed device. In some embodiments, the bedtime temperature of the bed device can be at a varying temperature setting. In some cases, the varying temperature setting can be when the temperature of the bed device remains at a temperature within a range of temperatures during duration of the bedtime temperature of the bed device. In some examples, the average of temperatures that occurred during the varying temperature setting during the duration of the bedtime temperature of the bed device can be the temperature of the bed device during the steady temperature setting of the bedtime temperature. In some cases, the average of the temperatures that occurred during the varying temperature setting during the duration of the bedtime temperature of the bed device can be referred to as the average temperature. In some embodiments, the range of temperatures during the duration of the bedtime temperature of the bed device can be at least or up to about 10° C. lower than the set temperature of the bedtime temperature. In some cases, the range of temperature during the duration of the bedtime temperature of the bed device can be at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., or at least or up to about 20° C. lower than the set temperature of the bedtime temperature. In some embodiments, the range of temperatures during the duration of the bedtime temperature of the bed device can be at least or up to about 20° C. higher than the set temperature of the bedtime temperature. In some cases, the range of temperature during the duration of the bedtime temperature of the bed device can at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., or at least or up to about 20° C. higher than the set temperature of the bedtime temperature.

In some embodiments, the initial temperature of the bed device can be at a steady temperature setting. In some cases, the steady temperature setting can be when the temperature of the bed device remains at the same degrees Celsius during the duration of the initial temperature of the bed device. In some embodiments, the initial temperature of the bed device can be at a varying temperature setting. In some cases, the varying temperature setting can be when the temperature of the bed device remains at a temperature within a range of temperatures during duration of the initial temperature of the bed device. In some examples, the average of temperatures that occurred during the varying temperature setting during the duration of the initial temperature of the bed device can be the temperature of the bed device during the steady temperature setting of the initial temperature. In some cases, the average of the temperatures that occurred during the varying temperature setting during the duration of the initial temperature of the bed device can be referred to as the average temperature. In some embodiments, the range of temperatures during the duration of the initial temperature of the bed device can be at least or up to about 10° C. lower than the set temperature of the initial temperature. In some cases, the range of temperature during the duration of the initial temperature of the bed device can be at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., or at least or up to about 20° C. lower than the set temperature of the initial temperature. In some embodiments, the range of temperatures during the duration of the initial temperature of the bed device can be at least or up to about 20° C. higher than the set temperature of the initial temperature. In some cases, the range of temperature during the duration of the initial temperature of the bed device can at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., or at least or up to about 20° C. higher than the set temperature of the initial temperature.

In some embodiments, the final temperature of the bed device can be at a steady temperature setting. In some cases, the steady temperature setting can be when the temperature of the bed device remains at the same degrees Celsius during the duration of the final temperature of the bed device. In some embodiments, the final temperature of the bed device can be at a varying temperature setting. In some cases, the varying temperature setting can be when the temperature of the bed device remains at a temperature within a range of temperatures during duration of the final temperature of the bed device. In some examples, the average of temperatures that occurred during the varying temperature setting during the duration of the final temperature of the bed device can be the temperature of the bed device during the steady temperature setting of the final temperature. In some cases, the average of the temperatures that occurred during the varying temperature setting during the duration of the final temperature of the bed device can be referred to as the average temperature. In some embodiments, the range of temperatures during the duration of the final temperature of the bed device can be at least or up to about 10° C. lower than the set temperature of the final temperature. In some cases, the range of temperature during the duration of the final temperature of the bed device can be at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., or at least or up to about 20° C. lower than the set temperature of the final temperature. In some embodiments, the range of temperatures during the duration of the final temperature of the bed device can be at least or up to about 20° C. higher than the set temperature of the final temperature. In some cases, the range of temperature during the duration of the final temperature of the bed device can at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., or at least or up to about 20° C. higher than the set temperature of the final temperature.

In some embodiments, the wakeup temperature of the bed device can be at a steady temperature setting. In some cases, the steady temperature setting can be when the temperature of the bed device remains at the same degrees Celsius during the duration of the wakeup temperature of the bed device. In some embodiments, the wakeup temperature of the bed device can be at a varying temperature setting. In some cases, the varying temperature setting can be when the temperature of the bed device remains at a temperature within a range of temperatures during duration of the wakeup temperature of the bed device. In some examples, the average of temperatures that occurred during the varying temperature setting during the duration of the wakeup temperature of the bed device can be the temperature of the bed device during the steady temperature setting of the wakeup temperature. In some cases, the average of the temperatures that occurred during the varying temperature setting during the duration of the wakeup temperature of the bed device can be referred to as the average temperature. In some embodiments, the range of temperatures during the duration of the wakeup temperature of the bed device can be at least or up to about 10° C. lower than the set temperature of the wakeup temperature. In some cases, the range of temperature during the duration of the wakeup temperature of the bed device can be at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., or at least or up to about 20° C. lower than the set temperature of the wakeup temperature. In some embodiments, the range of temperatures during the duration of the wakeup temperature of the bed device can be at least or up to about 20° C. higher than the set temperature of the wakeup temperature. In some cases, the range of temperature during the duration of the wakeup temperature of the bed device can at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., or at least or up to about 20° C. higher than the set temperature of the wakeup temperature.

In some embodiments, the temperature of the bed device incrementally changes (e.g., increases) from one temperature setting to another temperature setting (e.g., from the initial temperature to the final temperature). A number of the incremental changes can be determined based on, for example, a number of incremental changes desired between the two temperature settings, a duration between two adjacent incremental changes of a plurality of incremental changes to be performed between the two temperature settings, consistency or variability of the incremental changes of a plurality of incremental changes between the two temperature settings, etc.

In some embodiments, the temperature of the bed device can comprise a plurality of incremental temperature changes. In some cases, the temperature of the bed device can comprise a plurality of incremental temperature changes during use of the bed device. In some examples, the temperature of the bed device can comprise a plurality of incremental temperature changes during a single use of the bed device.

In some embodiments, the plurality of incremental temperature changes can comprise a predetermined number of incremental temperature changes. In some cases, the predetermined number of incremental temperature changes can comprise between about 0 incremental temperatures changes to about 100 incremental temperatures changes. In some cases, the predetermined number of incremental temperature changes can comprise at least or up to about 0 incremental temperature changes, at least or up to about 1 incremental temperature change, at least or up to about 2 incremental temperature changes, at least or up to about 3 incremental temperature changes, at least or up to about 4 incremental temperature changes, at least or up to about 5 incremental temperature changes, at least or up to about 6 incremental temperature changes, at least or up to about 7 incremental temperature changes, at least or up to about 8 incremental temperature changes, at least or up to about 9 incremental temperature changes, at least or up to about 10 incremental temperature changes, at least or up to about 11 incremental temperature changes, at least or up to about 12 incremental temperature changes, at least or up to about 13 incremental temperature changes, at least or up to about 14 incremental temperature changes, at least or up to about 15 incremental temperature changes, at least or up to about 16 incremental temperature changes, at least or up to about 17 incremental temperature changes, at least or up to about 18 incremental temperature changes, at least or up to about 19 incremental temperature changes, at least or up to about 20 incremental temperature changes, at least or up to about 21 incremental temperature changes, at least or up to about 22 incremental temperature changes, at least or up to about 23 incremental temperature changes, at least or up to about 24 incremental temperature changes, at least or up to about 25 incremental temperature changes, at least or up to about 26 incremental temperature changes, at least or up to about 27 incremental temperature changes, at least or up to about 28 incremental temperature changes, at least or up to about 29 incremental temperature changes, at least or up to about 30 incremental temperature changes, at least or up to about 31 incremental temperature changes, at least or up to about 32 incremental temperature changes, at least or up to about 33 incremental temperature changes, at least or up to about 34 incremental temperature changes, at least or up to about 35 incremental temperature changes, at least or up to about 36 incremental temperature changes, at least or up to about 37 incremental temperature changes, at least or up to about 38 incremental temperature changes, at least or up to about 39 incremental temperature changes, at least or up to about 40 incremental temperature changes, at least or up to about 41 incremental temperature changes, at least or up to about 42 incremental temperature changes, at least or up to about 43 incremental temperature changes, at least or up to about 44 incremental temperature changes, at least or up to about 45 incremental temperature changes, at least or up to about 46 incremental temperature changes, at least or up to about 47 incremental temperature changes, at least or up to about 48 incremental temperature changes, at least or up to about 49 incremental temperature changes, at least or up to about 50 incremental temperature changes, at least or up to about 51 incremental temperature changes, at least or up to about 52 incremental temperature changes, at least or up to about 53 incremental temperature changes, at least or up to about 54 incremental temperature changes, at least or up to about 55 incremental temperature changes, at least or up to about 56 incremental temperature changes, at least or up to about 57 incremental temperature changes, at least or up to about 58 incremental temperature changes, at least or up to about 59 incremental temperature changes, at least or up to about 60 incremental temperature changes, at least or up to about 61 incremental temperature changes, at least or up to about 62 incremental temperature changes, at least or up to about 63 incremental temperature changes, at least or up to about 64 incremental temperature changes, at least or up to about 65 incremental temperature changes, at least or up to about 66 incremental temperature changes, at least or up to about 67 incremental temperature changes, at least or up to about 68 incremental temperature changes, at least or up to about 69 incremental temperature changes, at least or up to about 70 incremental temperature changes, at least or up to about 71 incremental temperature changes, at least or up to about 72 incremental temperature changes, at least or up to about 73 incremental temperature changes, at least or up to about 74 incremental temperature changes, at least or up to about 75 incremental temperature changes, at least or up to about 76 incremental temperature changes, at least or up to about 77 incremental temperature changes, at least or up to about 78 incremental temperature changes, at least or up to about 79 incremental temperature changes, at least or up to about 80 incremental temperature changes, at least or up to about 81 incremental temperature changes, at least or up to about 82 incremental temperature changes, at least or up to about 83 incremental temperature changes, at least or up to about 84 incremental temperature changes, at least or up to about 85 incremental temperature changes, at least or up to about 86 incremental temperature changes, at least or up to about 87 incremental temperature changes, at least or up to about 88 incremental temperature changes, at least or up to about 89 incremental temperature changes, at least or up to about 90 incremental temperature changes, at least or up to about 91 incremental temperature changes, at least or up to about 92 incremental temperature changes, at least or up to about 93 incremental temperature changes, at least or up to about 94 incremental temperature changes, at least or up to about 95 incremental temperature changes, at least or up to about 96 incremental temperature changes, at least or up to about 97 incremental temperature changes, at least or up to about 98 incremental temperature changes, at least or up to about 99 incremental temperature changes, or at least or up to about 100 incremental temperature changes.

In some embodiments, the plurality of incremental temperature changes can comprise one or more consecutive temperature increases. In some cases, the plurality incremental temperatures changes can comprise two or more consecutive temperature increases. In some cases, the plurality incremental temperatures changes can comprise three or more consecutive temperature increases. In some cases, the plurality incremental temperatures changes can comprise four or more consecutive temperature increases. In some cases, the plurality incremental temperatures changes can comprise five or more consecutive temperature increases.

In some embodiments, the plurality of incremental temperature changes can comprise one or more consecutive temperature decreases. In some cases, the plurality incremental temperatures changes can comprise two or more consecutive temperature decreases. In some cases, the plurality incremental temperatures changes can comprise three or more consecutive temperature decreases. In some cases, the plurality of incremental temperature changes can comprise four or more consecutive temperature decreases. In some cases, the plurality incremental temperatures changes can comprise five or more consecutive temperature decreases.

In some embodiments, an incremental temperature change of the plurality of incremental temperature changes of the bed device can be at a steady temperature setting. In some cases, the steady temperature setting can be when the temperature of the bed device remains at the same degrees Celsius during the duration of the incremental temperature change of the bed device. In some embodiments, an incremental temperature change of the plurality of incremental temperature changes of the bed device can be at a varying temperature setting. In some cases, the varying temperature setting can be when the temperature of the bed device remains at a temperature within a range of temperatures during duration of the incremental temperature change of the bed device. In some examples, the average of temperatures that occurred during the varying temperature setting during the duration of the incremental temperature change of the bed device can be the temperature of the bed device during the steady temperature setting of the incremental temperature change. In some cases, the average of the temperatures that occurred during the varying temperature setting during the duration of the incremental temperature change of the bed device can be referred to as the average temperature. In some embodiments, the range of temperatures during the duration of the incremental temperature change of the bed device can be at least or up to about 10° C. lower than the set temperature of the incremental temperature change. In some cases, the range of temperature during the duration of the incremental temperature change of the bed device can be at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., or at least or up to about 10° lower than the set temperature of the incremental temperature change. In some embodiments, the range of temperatures during the duration of the incremental temperature change of the bed device can be at least or up to about 10° C. higher than the set temperature of the incremental temperature change. In some cases, the range of temperature during the duration of the incremental temperature change of the bed device can be at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., or at least or up to about 10° higher than the set temperature of the incremental temperature change.

The number of the incremental changes (e.g., increase) between two temperature settings (e.g., two adjacent temperature settings) can be at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 11, at least or up to about 12, at least or up to about 13, at least or up to about 14, at least or up to about 15, at least or up to about 16, at least or up to about 17, at least or up to about 18, at least or up to about 19, at least or up to about 20, at least or up to about 21, at least or up to about 22, at least or up to about 23, at least or up to about 24, at least or up to about 25, at least or up to about 26, at least or up to about 27, at least or up to about 28, at least or up to about 29, at least or up to about 30, at least or up to about 31, at least or up to about 32, at least or up to about 33, at least or up to about 34, at least or up to about 35, at least or up to about 36, at least or up to about 37, at least or up to about 38, at least or up to about 39, at least or up to about 40, at least or up to about 41, at least or up to about 42, at least or up to about 43, at least or up to about 44, at least or up to about 45, at least or up to about 46, at least or up to about 47, at least or up to about 48, at least or up to about 49, at least or up to about 50, at least or up to about 51, at least or up to about 52, at least or up to about 53, at least or up to about 54, at least or up to about 55, at least or up to about 56, at least or up to about 57, at least or up to about 58, at least or up to about 59, at least or up to about 60, at least or up to about 61, at least or up to about 62, at least or up to about 63, at least or up to about 64, at least or up to about 65, at least or up to about 66, at least or up to about 67, at least or up to about 68, at least or up to about 69, at least or up to about 70, at least or up to about 71, at least or up to about 72, at least or up to about 73, at least or up to about 74, at least or up to about 75, at least or up to about 76, at least or up to about 77, at least or up to about 78, at least or up to about 79, at least or up to about 80, at least or up to about 81, at least or up to about 82, at least or up to about 83, at least or up to about 84, at least or up to about 85, at least or up to about 86, at least or up to about 87, at least or up to about 88, at least or up to about 89, at least or up to about 90, at least or up to about 91, at least or up to about 92, at least or up to about 93, at least or up to about 94, at least or up to about 95, at least or up to about 96, at least or up to about 97, at least or up to about 98, at least or up to about 99, or at least or up to about 100.

The length of time between two adjacent incremental changes can be between about 0 minutes to about 30 minutes. The length of time between two adjacent increment changes can at least or up to about 0 seconds, at least or up to about 1 second, at least or up to about 2 seconds, at least or up to about 3 seconds, at least or up to about 3 seconds, at least or up to about 4 seconds, at least or up to about 5 seconds, at least or up to about 6 seconds, at least or up to about 7 seconds, at least or up to about 8 seconds, at least or up to about 9 seconds, at least or up to about 10 seconds, at least or up to about 11 seconds, at least or up to about 12 seconds, at least or up to about 13 seconds, at least or up to about 14 seconds, at least or up to about 15 seconds, at least or up to about 16 seconds, at least or up to about 17 seconds, at least or up to about 18 seconds, at least or up to about 19 seconds, at least or up to about 20 seconds, at least or up to about 21 seconds, at least or up to about 22 seconds, at least or up to about 23 seconds, at least or up to about 24 seconds, at least or up to about 25 seconds, at least or up to about 26 seconds, at least or up to about 27 seconds, at least or up to about 28 seconds, at least or up to about 29 seconds, at least or up to about 30 seconds, at least or up to about 31 seconds, at least or up to about 32 seconds, at least or up to about 33 seconds, at least or up to about 34 seconds, at least or up to about 35 seconds, at least or up to about 36 seconds, at least or up to about 37 seconds, at least or up to about 38 seconds, at least or up to about 39 seconds, at least or up to about 40 seconds, at least or up to about 41 seconds, at least or up to about 42 seconds, at least or up to about 43 seconds, at least or up to about 44 seconds, at least or up to about 45 seconds, at least or up to about 46 seconds, at least or up to about 47 seconds, at least or up to about 48 seconds, at least or up to about 49 seconds, at least or up to about 50 seconds, at least or up to about 51 seconds, at least or up to about 52 seconds, at least or up to about 53 seconds, at least or up to about 54 seconds, at least or up to about 55 seconds, at least or up to about 56 seconds, at least or up to about 57 seconds, at least or up to about 58 seconds, at least or up to about 59 seconds, at least or up to about 60 seconds, at least or up to about 61 seconds, at least or up to about 62 seconds, at least or up to about 63 seconds, at least or up to about 64 seconds, at least or up to about 65 seconds, at least or up to about 66 seconds, at least or up to about 67 seconds, at least or up to about 68 seconds, at least or up to about 69 seconds, at least or up to about 70 seconds, at least or up to about 71 seconds, at least or up to about 72 seconds, at least or up to about 73 seconds, at least or up to about 74 seconds, at least or up to about 75 seconds, at least or up to about 76 seconds, at least or up to about 77 seconds, at least or up to about 78 seconds, at least or up to about 79 seconds, at least or up to about 80 seconds, at least or up to about 81 seconds, at least or up to about 82 seconds, at least or up to about 83 seconds, at least or up to about 84 seconds, at least or up to about 85 seconds, at least or up to about 86 seconds, at least or up to about 87 seconds, at least or up to about 88 seconds, at least or up to about 89 seconds, at least or up to about 90 seconds, at least or up to about 91 seconds, at least or up to about 92 seconds, at least or up to about 93 seconds, at least or up to about 94 seconds, at least or up to about 95 seconds, at least or up to about 96 seconds, at least or up to about 97 seconds, at least or up to about 98 seconds, at least or up to about 99 seconds, at least or up to about 100 seconds, at least or up to about 101 seconds, at least or up to about 102 seconds, at least or up to about 103 seconds, at least or up to about 104 seconds, at least or up to about 105 seconds, at least or up to about 106 seconds, at least or up to about 107 seconds, at least or up to about 108 seconds, at least or up to about 109 seconds, at least or up to about 110 seconds, at least or up to about 111 seconds, at least or up to about 112 seconds, at least or up to about 113 seconds, at least or up to about 114 seconds, at least or up to about 115 seconds, at least or up to about 116 seconds, at least or up to about 117 seconds, at least or up to about 118 seconds, at least or up to about 119 seconds, at least or up to about 2 minutes, at least or up to about 3 minutes, at least or up to about 4 minutes, at least or up to about 5 minutes, at least or up to about 6 minutes, at least or up to about minutes, at least or up to about 7 minutes, at least or up to about 8 minutes, at least or up to about 9 minutes, at least or up to about 10 minutes, at least or up to about 11 minutes, at least or up to about 12 minutes, at least or up to about 13 minutes, at least or up to about 14 minutes, at least or up to about 15 minutes, at least or up to about 16 minutes, at least or up to about 17 minutes, at least or up to about 18 minutes, at least or up to about 19 minutes, at least or up to about 20 minutes, at least or up to about 21 minutes, at least or up to about 22 minutes, at least or up to about 23 minutes, at least or up to about 24 minutes, at least or up to about 25 minutes, at least or up to about 26 minutes, at least or up to about 27 minutes, at least or up to about 28 minutes, at least or up to about 29 minutes, or at least or up to about 30 minutes.

The number of the incremental changes (e.g., decrease) between bedtime temperature and initial temperature can be at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 11, at least or up to about 12, at least or up to about 13, at least or up to about 14, at least or up to about 15, at least or up to about 16, at least or up to about 17, at least or up to about 18, at least or up to about 19, at least or up to about 20, at least or up to about 21, at least or up to about 22, at least or up to about 23, at least or up to about 24, at least or up to about 25, at least or up to about 26, at least or up to about 27, at least or up to about 28, at least or up to about 29, at least or up to about 30, at least or up to about 31, at least or up to about 32, at least or up to about 33, at least or up to about 34, at least or up to about 35, at least or up to about 36, at least or up to about 37, at least or up to about 38, at least or up to about 39, at least or up to about 40, at least or up to about 41, at least or up to about 42, at least or up to about 43, at least or up to about 44, at least or up to about 45, at least or up to about 46, at least or up to about 47, at least or up to about 48, at least or up to about 49, at least or up to about 50, at least or up to about 51, at least or up to about 52, at least or up to about 53, at least or up to about 54, at least or up to about 55, at least or up to about 56, at least or up to about 57, at least or up to about 58, at least or up to about 59, at least or up to about 60, at least or up to about 61, at least or up to about 62, at least or up to about 63, at least or up to about 64, at least or up to about 65, at least or up to about 66, at least or up to about 67, at least or up to about 68, at least or up to about 69, at least or up to about 70, at least or up to about 71, at least or up to about 72, at least or up to about 73, at least or up to about 74, at least or up to about 75, at least or up to about 76, at least or up to about 77, at least or up to about 78, at least or up to about 79, at least or up to about 80, at least or up to about 81, at least or up to about 82, at least or up to about 83, at least or up to about 84, at least or up to about 85, at least or up to about 86, at least or up to about 87, at least or up to about 88, at least or up to about 89, at least or up to about 90, at least or up to about 91, at least or up to about 92, at least or up to about 93, at least or up to about 94, at least or up to about 95, at least or up to about 96, at least or up to about 97, at least or up to about 98, at least or up to about 99, or at least or up to about 100.

The number of the incremental changes (e.g., increase) between the initial temperature and the final temperature can be at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 11, at least or up to about 12, at least or up to about 13, at least or up to about 14, at least or up to about 15, at least or up to about 16, at least or up to about 17, at least or up to about 18, at least or up to about 19, at least or up to about 20, at least or up to about 21, at least or up to about 22, at least or up to about 23, at least or up to about 24, at least or up to about 25, at least or up to about 26, at least or up to about 27, at least or up to about 28, at least or up to about 29, at least or up to about 30, at least or up to about 31, at least or up to about 32, at least or up to about 33, at least or up to about 34, at least or up to about 35, at least or up to about 36, at least or up to about 37, at least or up to about 38, at least or up to about 39, at least or up to about 40, at least or up to about 41, at least or up to about 42, at least or up to about 43, at least or up to about 44, at least or up to about 45, at least or up to about 46, at least or up to about 47, at least or up to about 48, at least or up to about 49, at least or up to about 50, at least or up to about 51, at least or up to about 52, at least or up to about 53, at least or up to about 54, at least or up to about 55, at least or up to about 56, at least or up to about 57, at least or up to about 58, at least or up to about 59, at least or up to about 60, at least or up to about 61, at least or up to about 62, at least or up to about 63, at least or up to about 64, at least or up to about 65, at least or up to about 66, at least or up to about 67, at least or up to about 68, at least or up to about 69, at least or up to about 70, at least or up to about 71, at least or up to about 72, at least or up to about 73, at least or up to about 74, at least or up to about 75, at least or up to about 76, at least or up to about 77, at least or up to about 78, at least or up to about 79, at least or up to about 80, at least or up to about 81, at least or up to about 82, at least or up to about 83, at least or up to about 84, at least or up to about 85, at least or up to about 86, at least or up to about 87, at least or up to about 88, at least or up to about 89, at least or up to about 90, at least or up to about 91, at least or up to about 92, at least or up to about 93, at least or up to about 94, at least or up to about 95, at least or up to about 96, at least or up to about 97, at least or up to about 98, at least or up to about 99, or at least or up to about 100.

The number of the incremental changes (e.g., increase or decrease) between the final temperature and the wakeup temperature can be at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 11, at least or up to about 12, at least or up to about 13, at least or up to about 14, at least or up to about 15, at least or up to about 16, at least or up to about 17, at least or up to about 18, at least or up to about 19, at least or up to about 20, at least or up to about 21, at least or up to about 22, at least or up to about 23, at least or up to about 24, at least or up to about 25, at least or up to about 26, at least or up to about 27, at least or up to about 28, at least or up to about 29, at least or up to about 30, at least or up to about 31, at least or up to about 32, at least or up to about 33, at least or up to about 34, at least or up to about 35, at least or up to about 36, at least or up to about 37, at least or up to about 38, at least or up to about 39, at least or up to about 40, at least or up to about 41, at least or up to about 42, at least or up to about 43, at least or up to about 44, at least or up to about 45, at least or up to about 46, at least or up to about 47, at least or up to about 48, at least or up to about 49, at least or up to about 50, at least or up to about 51, at least or up to about 52, at least or up to about 53, at least or up to about 54, at least or up to about 55, at least or up to about 56, at least or up to about 57, at least or up to about 58, at least or up to about 59, at least or up to about 60, at least or up to about 61, at least or up to about 62, at least or up to about 63, at least or up to about 64, at least or up to about 65, at least or up to about 66, at least or up to about 67, at least or up to about 68, at least or up to about 69, at least or up to about 70, at least or up to about 71, at least or up to about 72, at least or up to about 73, at least or up to about 74, at least or up to about 75, at least or up to about 76, at least or up to about 77, at least or up to about 78, at least or up to about 79, at least or up to about 80, at least or up to about 81, at least or up to about 82, at least or up to about 83, at least or up to about 84, at least or up to about 85, at least or up to about 86, at least or up to about 87, at least or up to about 88, at least or up to about 89, at least or up to about 90, at least or up to about 91, at least or up to about 92, at least or up to about 93, at least or up to about 94, at least or up to about 95, at least or up to about 96, at least or up to about 97, at least or up to about 98, at least or up to about 99, or at least or up to about 100.

In some embodiments, the temperature of the bed device changes (e.g., increases) periodically, e.g., between every about 70 minutes to every about 120 minutes. A duration of time between two adjacent periodic changes of the temperature of the bed device can be at least or up to about 1 minute, at least or up to about 2 minutes, at least or up to about 3 minutes, at least or up to about 4 minutes, at least or up to about 5 minutes, at least or up to about 6 minutes, at least or up to about 7 minutes, at least or up to about 8 minutes, at least or up to about 9 minutes, at least or about to about 10 minutes, at least or up to about 15 minutes, at least or up to about 20 minutes, at least or up to about 25 minutes, at least or up to about 30 minutes, at least or up to about 35 minutes, at least or up to about 40 minutes, at least or up to about 45 minutes, at least or up to about 50 minutes, at least or up to about 55 minutes, at least or up to about 60 minutes, at least or up to about 65 minutes, at least or up to about 70 minutes, at least or up to about 75 minutes, at least or up to about 80 minutes, at least or up to about 85 minutes, at least or up to about 90 minutes, at least or up to about 95 minutes, at least or up to about 100 minutes, at least or up to about 105 minutes, at least or up to about 110 minutes, at least or up to about 115 minutes, at least or up to about 120 minutes, at least or up to about 125 minutes, at least or up to about 130 minutes, at least or up to about 135 minutes, at least or up to about 140 minutes, at least or up to about 145 minutes, or at least or up to about 150 minutes. In some embodiments, the temperature of the bed device changes (e.g., increases) about every 5 minutes, about every 10 minutes, about every 20 minutes, about every 30 minutes, about every 40 minutes, about every 50 minutes, about every 60 minutes, about every 70 minutes, about every 75 minutes, about every 80 minutes, about every 85 minutes, about every 90 minutes, about every 95 minutes, about every 100 minutes, about every 110 minutes, about every 115 minutes, or about every 120 minutes. In some embodiments, the temperature of the bed device changes (e.g., increases) about every 90 minutes.

In some embodiments, a difference in temperature (or the temperature change) between the two adjacent periodic changes can be at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3° C., at least or up to about 3.1° C., at least or up to about 3.2° C., at least or up to about 3.3° C., at least or up to about 3.4° C., at least or up to about 3.5° C., at least or up to about 3.6° C., at least or up to about 0.7° C., at least or up to about 3.8° C., at least or up to about 0.9° C., at least or up to about 4° C., at least or up to about 4.5° C., at least or up to about 5° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.9° C., or at least or up to about 10° C.

In some embodiments, the plurality of incremental changes can comprise two or more incremental changes. The durations of time between the plurality of incremental changes can be substantially the same or different. The temperature changes of the two or more incremental changes can be substantially the same or different.

In some embodiments, a temperature of the article of furniture (e.g., the bed device) between the plurality of incremental changes may remain substantially constant. In some embodiments, the substantial constant temperature is maintained for between about 0 minutes to about 240 minutes. In some embodiments, the substantial constant temperature is maintained for at least or up to about 0 minutes, at least or up to about 5 minutes, at least or up to about 10 minutes, at least or up to about 15 minutes, at least or up to about 20 minutes, at least or up to about 25 minutes, at least or up to about 30 minutes, at least or up to about 35 minutes, at least or up to about 40 minutes, at least or up to about 45 minutes, at least or up to about 50 minutes, at least or up to about 55 minutes, at least or up to about 60 minutes, at least or up to about 65 minutes, at least or up to about 70 minutes, at least or up to about 75 minutes, at least or up to about 80 minutes, at least or up to about 85 minutes, at least or up to about 90 minutes, at least or up to about 95 minutes, at least or up to about 100 minutes, at least or up to about 110 minutes, at least or up to about 120 minutes, at least or up to about 130 minutes, at least or up to about 140 minutes, at least or up to about 150 minutes, at least or up to about 160 minutes, at least or up to about 170 minutes, at least or up to about 180 minutes, at least or up to about 190 minutes, at least or up to about 200 minutes, at least or up to about 210 minutes, at least or up to about 220 minutes, at least or up to about 230 minutes, or at least or up to about 240 minutes.

In some embodiments, any change in temperature of the article of furniture as provided herein (e.g., as directed by the temperature regulator) can be at a rate of at least or up to about 0.01° C. per minute (° C./min), at least or up to about 0.02° C./min, at least or up to about 0.03° C./min, at least or up to about 0.04° C./min, at least or up to about 0.05° C./min, at least or up to about 0.06° C./min, at least or up to about 0.07° C./min, at least or up to about 0.08° C./min, at least or up to about 0.09° C./min, at least or up to about 0.1° C./min, at least or up to about 0.2° C./min, at least or up to about 0.3° C./min, at least or up to about 0.4° C./min, at least or up to about 0.5° C./min, at least or up to about 0.6° C./min, at least or up to about 0.7° C./min, at least or up to about 0.8° C./min, at least or up to about 0.9° C./min, at least or up to about 1° C./min, at least or up to about 1.1° C./min, at least or up to about 1.2° C./min, at least or up to about 1.3° C./min, at least or up to about 1.4° C./min, at least or up to about 1.5° C./min, at least or up to about 1.6° C./min, at least or up to about 1.7° C./min, at least or up to about 1.8° C./min, at least or up to about 1.9° C./min, at least or up to about 2° C./min, at least or up to about 2.5° C./min, at least or up to about 3° C./min, at least or up to about 4° C./min, or at least or up to about 5° C./min.

In some embodiments, the plurality of incremental changes can start after a user's bedtime, e.g., within at least or up to about 1 minute, within at least or up to about 2 minutes, within at least or up to about 3 minutes, within at least or up to about 4 minutes, within at least or up to about 5 minutes, within at least or up to about 6 minutes, within at least or up to about 7 minutes, within at least or up to about 8 minutes, within at least or up to about 9 minutes, within at least or up to about 10 minutes, within at least or up to about 15 minutes, within at least or up to about 20 minutes, within at least or up to about 25 minutes, within at least or up to about 30 minutes, within at least or up to about 35 minutes, within at least or up to about 40 minutes, within at least or up to about 45 minutes, within at least or up to about 50 minutes, within at least or up to about 55 minutes, within at least or up to about 60 minutes of the bedtime.

The bedtime as provided herein can be when the user starts using the article of furniture (e.g., gets on the article of furniture) or when the user is expected to use the article of furniture (e.g., based on an average value of previous uses, or a predetermined time provided by the user via the GUI).

One or more conditions of the plurality of incremental changes (e.g., the number of the plurality of incremental changes, the duration of time between two adjacent periodic changes of the plurality of incremental changes, the temperature change between the two adjacent periodic changes, the rate of the temperature change, the time of implementing the plurality of incremental changes, etc.) can be determined prior to use of the article of furniture by the user, or during use of the article of furniture by the user (e.g., based on biological sensing data generated during the use by the user). In some embodiments, a characteristic of the plurality of incremental temperature changes is different for different users, wherein the characteristic comprises (i) a number of incremental changes, (ii) a target temperature of the article of furniture, or (iii) a duration thereof between two adjacent incremental changes. Alternatively or in addition to, such one or more conditions of the plurality of incremental changes can be changed in accordance with any of the methods provided herein.

In some embodiments, the bed device can be adjusted to a plurality of target temperatures. In some cases, the plurality of target temperatures can comprise between about one target temperature to about 100 target temperatures. In some cases, the plurality of target temperatures can comprise between about one target temperature to about ten target temperatures, between about ten target temperatures to about 20 target temperatures, between about 20 target temperatures to about 30 target temperatures, between about 30 target temperatures to about 40 target temperatures, between about 40 target temperatures to about 50 target temperatures, between about 50 target temperatures to about 60 target temperatures, between about 60 target temperatures to about 70 target temperatures, between about 70 target temperatures to about 80 target temperatures, between about 80 target temperatures to about 90 target temperatures, or between about 90 target temperatures to about 100 target temperatures. In some cases, the plurality of temperatures can comprise at least about one target temperature, at least about two target temperatures, at least about three target temperatures, at least about four target temperatures, at least about five target temperatures, at least about six target temperatures, at least about seven target temperatures, at least about eight target temperatures, at least about nine target temperatures, at least about ten target temperatures, at least about 11 target temperatures, at least about 12 target temperatures, at least about 13 target temperatures, at least about 14 target temperatures, at least about 15 target temperatures, at least about 16 target temperatures, at least about 17 target temperatures, at least about 18 target temperatures, at least about 19 target temperatures, at least about 20 target temperatures, at least about 21 target temperatures, at least about 22 target temperatures, at least about 23 target temperatures, at least about 24 target temperatures, at least about 25 target temperatures, at least about 26 target temperatures, at least about 27 target temperatures, at least about 28 target temperatures, at least about 29 target temperatures, at least about 30 target temperatures, at least about 31 target temperatures, at least about 32 target temperatures, at least about 33 target temperatures, at least about 34 target temperatures, at least about 35 target temperatures, at least about 36 target temperatures, at least about 37 target temperatures, at least about 38 target temperatures, at least about 39 target temperatures, at least about 40 target temperatures, at least about 41 target temperatures, at least about 42 target temperatures, at least about 43 target temperatures, at least about 44 target temperatures, at least about 45 target temperatures, at least about 46 target temperatures, at least about 47 target temperatures, at least about 48 target temperatures, at least about 49 target temperatures, at least about 50 target temperatures, at least about 51 target temperatures, at least about 52 target temperatures, at least about 53 target temperatures, at least about 54 target temperatures, at least about 55 target temperatures, at least about 56 target temperatures, at least about 57 target temperatures, at least about 58 target temperatures, at least about 59 target temperatures, at least about 60 target temperatures, at least about 61 target temperatures, at least about 62 target temperatures, at least about 63 target temperatures, at least about 64 target temperatures, at least about 65 target temperatures, at least about 66 target temperatures, at least about 67 target temperatures, at least about 68 target temperatures, at least about 69 target temperatures, at least about 70 target temperatures, at least about 71 target temperatures, at least about 72 target temperatures, at least about 73 target temperatures, at least about 74 target temperatures, at least about 75 target temperatures, at least about 76 target temperatures, at least about 77 target temperatures, at least about 78 target temperatures, at least about 79 target temperatures, at least about 80 target temperatures, at least about 81 target temperatures, at least about 82 target temperatures, at least about 83 target temperatures, at least about 84 target temperatures, at least about 85 target temperatures, at least about 86 target temperatures, at least about 87 target temperatures, at least about 88 target temperatures, at least about 89 target temperatures, at least about 90 target temperatures, at least about 91 target temperatures, at least about 92 target temperatures, at least about 93 target temperatures, at least about 94 target temperatures, at least about 95 target temperatures, at least about 96 target temperatures, at least about 97 target temperatures, at least about 98 target temperatures, at least about 99 target temperatures, at least about 100 target temperatures, or more than 100 target temperatures. In some cases, the plurality of target temperatures can comprise at most about 100 target temperatures, at most about 99 target temperatures, at most about 98 target temperatures, at most about 97 target temperatures, at most about 96 target temperatures, at most about 95 target temperatures, at most about 94 target temperatures, at most about 93 target temperatures, at most about 92 target temperatures, at most about 91 target temperatures, at most about 90 target temperatures, at most about 89 target temperatures, at most about 88 target temperatures, at most about 87 target temperatures, at most about 86 target temperatures, at most about 85 target temperatures, at most about 84 target temperatures, at most about 83 target temperatures, at most about 82 target temperatures, at most about 81 target temperatures, at most about 80 target temperatures, at most about 79 target temperatures, at most about 78 target temperatures, at most about 77 target temperatures, at most about 76 target temperatures, at most about 75 target temperatures, at most about 74 target temperatures, at most about 73 target temperatures, at most about 72 target temperatures, at most about 71 target temperatures, at most about 70 target temperatures, at most about 69 target temperatures, at most about 68 target temperatures, at most about 67 target temperatures, at most about 66 target temperatures, at most about 65 target temperatures, at most about 64 target temperatures, at most about 63 target temperatures, at most about 62 target temperatures, at most about 61 target temperatures, at most about 60 target temperatures, at most about 59 target temperatures, at most about 58 target temperatures, at most about 57 target temperatures, at most about 56 target temperatures, at most about 55 target temperatures, at most about 54 target temperatures, at most about 53 target temperatures, at most about 52 target temperatures, at most about 51 target temperatures, at most about 50 target temperatures, at most about 49 target temperatures, at most about 48 target temperatures, at most about 47 target temperatures, at most about 46 target temperatures, at most about 45 target temperatures, at most about 44 target temperatures, at most about 43 target temperatures, at most about 42 target temperatures, at most about 41 target temperatures, at most about 40 target temperatures, at most about 39 target temperatures, at most about 38 target temperatures, at most about 37 target temperatures, at most about 36 target temperatures, at most about 35 target temperatures, at most about 34 target temperatures, at most about 33 target temperatures, at most about 32 target temperatures, at most about 31 target temperatures, at most about 30 target temperatures, at most about 29 target temperatures, at most about 28 target temperatures, at most about 27 target temperatures, at most about 26 target temperatures, at most about 25 target temperatures, at most about 24 target temperatures, at most about 23 target temperatures, at most about 22 target temperatures, at most about 21 target temperatures, at most about 20 target temperatures, at most about 19 target temperatures, at most about 18 target temperatures, at most about 17 target temperatures, at most about 16 target temperatures, at most about 15 target temperatures, at most about 14 target temperatures, at most about 13 target temperatures, at most about 12 target temperatures, at most about 11 target temperatures, at most about 10 target temperatures, at most about nine target temperatures, at most about eight target temperatures, at most about seven target temperatures, at most about six target temperatures, at most about five target temperatures, at most about four target temperatures, at most about three target temperatures, at most about two target temperatures, at most about one target temperature, or less than one target temperature. In some cases, the plurality of target temperatures can comprise about one target temperature, about two target temperatures, about three target temperatures, about four target temperatures, about five target temperatures, about six target temperatures, about seven target temperatures, about eight target temperatures, about nine target temperatures, about ten target temperatures, about 11 target temperatures, about 12 target temperatures, about 13 target temperatures, about 14 target temperatures, about 15 target temperatures, about 16 target temperatures, about 17 target temperatures, about 18 target temperatures, about 19 target temperatures, about 20 target temperatures, about 21 target temperatures, about 22 target temperatures, about 23 target temperatures, about 24 target temperatures, about 25 target temperatures, about 26 target temperatures, about 27 target temperatures, about 28 target temperatures, about 29 target temperatures, about 30 target temperatures, about 31 target temperatures, about 32 target temperatures, about 33 target temperatures, about 34 target temperatures, about 35 target temperatures, about 36 target temperatures, about 37 target temperatures, about 38 target temperatures, about 39 target temperatures, about 40 target temperatures, about 41 target temperatures, about 42 target temperatures, about 43 target temperatures, about 44 target temperatures, about 45 target temperatures, about 46 target temperatures, about 47 target temperatures, about 48 target temperatures, about 49 target temperatures, about 50 target temperatures, about 51 target temperatures, about 52 target temperatures, about 53 target temperatures, about 54 target temperatures, about 55 target temperatures, about 56 target temperatures, about 57 target temperatures, about 58 target temperatures, about 59 target temperatures, about 60 target temperatures, about 61 target temperatures, about 62 target temperatures, about 63 target temperatures, about 64 target temperatures, about 65 target temperatures, about 66 target temperatures, about 67 target temperatures, about 68 target temperatures, about 69 target temperatures, about 70 target temperatures, about 71 target temperatures, about 72 target temperatures, about 73 target temperatures, about 74 target temperatures, about 75 target temperatures, about 76 target temperatures, about 77 target temperatures, about 78 target temperatures, about 79 target temperatures, about 80 target temperatures, about 81 target temperatures, about 82 target temperatures, about 83 target temperatures, about 84 target temperatures, about 85 target temperatures, about 86 target temperatures, about 87 target temperatures, about 88 target temperatures, about 89 target temperatures, about 90 target temperatures, about 91 target temperatures, about 92 target temperatures, about 93 target temperatures, about 94 target temperatures, about 95 target temperatures, about 96 target temperatures, about 97 target temperatures, about 98 target temperatures, about 99 target temperatures, or about 100 target temperatures.

In some embodiments, a portion of the plurality of target temperatures can be different from each other. In some cases, the portion can be between about 0% to 100% of the plurality of target temperatures can be different from each other. In some cases, between about 0% to about 10%, between about 10% to about 20%, between about 20% to about 30%, between about 30% to about 40%, between about 40% to about 50%, between about 50% to about 60%, between about 60% to about 70%, between about 70% to about 80%, between about 80% to about 90%, or between about 90% to about 100% of the plurality of target temperatures. In some cases, the portion can be at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% of the plurality of target temperatures. In some cases, the portion can be at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, or at most about 0% of the plurality of target temperatures. In some cases, the portion can be about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of target temperatures.

In some embodiments, a portion of the plurality of target temperature can be the same as each other. In some cases, the portion can be between about 0% to 100% of the plurality of target temperatures can be different from each other. In some cases, between about 0% to about 10%, between about 10% to about 20%, between about 20% to about 30%, between about 30% to about 40%, between about 40% to about 50%, between about 50% to about 60%, between about 60% to about 70%, between about 70% to about 80%, between about 80% to about 90%, or between about 90% to about 100% of the plurality of target temperatures. In some cases, the portion can be at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% of the plurality of target temperatures. In some cases, the portion can be at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, or at most about 0% of the plurality of target temperatures. In some cases, the portion can be about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of target temperatures.

In some embodiments, each incremental change (e.g., increase) in temperature is calculated by the following formula (I):

previous temperature of the bed device+(the final temperature−the initial temperature)+4 (I)

In some embodiments, there are about four incremental temperature changes (e.g., increases) between the initial temperature of the bed device and the final temperature of the bed device.

In some embodiments, the bed device is at the initial temperature for between about 10 to about 130 minutes. In some embodiments, the bed device is at the initial temperature for at least or up to about 10 minutes, at least or up to about 15 minutes, at least or up to about 20 minutes, at least or up to about 25 minutes, at least or up to about 30 minutes, at least or up to about 35 minutes, at least or up to about 40 minutes, at least or up to about 45 minutes, at least or up to about 50 minutes, at least or up to about 55 minutes, at least or up to about 60 minutes, at least or up to about 65 minutes, at least or up to about 70 minutes, at least or up to about 75 minutes, at least or up to about 80 minutes, at least or up to about 85 minutes, at least or up to about 90 minutes, at least or up to about 95 minutes, at least or up to about 100 minutes, at least or up to about 110 minutes, at least or up to about 120 minutes, at least or up to about 125 minutes, or at least or up to about 130 minutes.

In some embodiments, the bed device is at the final temperature for between about 0 minutes to 6 hours. In some embodiments, the bed device is at the final temperature for at least or up to about 0 minutes, at least or up to about 5 minutes, at least or up to about 10 minutes, at least or up to about 15 minutes, at least or up to about 20 minutes, at least or up to about 25 minutes, at least or up to about 30 minutes, at least or up to about 35 minutes, at least or up to about 40 minutes, at least or up to about 45 minutes, at least or up to about 50 minutes, at least or up to about 55 minutes, at least or up to about 60 minutes, at least or up to about 90 minutes, at least or up to about 2 hours, at least or up to about 3 hours, at least or up to about 4 hours, at least or up to about 5 hours, or at least or up to about 6 hours. In some embodiments, the bed device is at the final temperature for at least or up to about 2 hours. In some embodiments, the bed device is at the final temperature for at least or up to about 4 hours. In some embodiments, the bed device is at the final temperature for at least or up to about six hours.

In some embodiments, the subject selects a fourth temperature of the bed device. In some embodiments, the fourth temperature is a wake-up temperature. In some embodiments, the wake-up temperature occurs after the final temperature. In some embodiments, the wake-up temperature of the bed device causes the subject to awaken while on the bed device. In some embodiments, the wake-up temperature of the bed device is warmer than the final temperature of the bed device. In some embodiments, the wake-up temperature is cooler than the final temperature of the bed device. In some embodiments, the bed device is at the wake-up temperature for between about 5 minutes to about 60 minutes. In some embodiments, the bed device is at the wake-up temperature for at least or up to about 5 minutes, at least or up to about 10 minutes, at least or up to about 15 minutes, at least or up to about 20 minutes, at least or up to about 25 minutes, at least or up to about 30 minutes, at least or up to about 35 minutes, at least or up to about 40 minutes, at least or up to about 45 minutes, at least or up to about 50 minutes, at least or up to about 55 minutes, or at least or up to about 60 minutes.

In some embodiments, the subject sets the temperatures of the bed device. In some embodiments, the subject sets the temperatures of the bed device (e.g., bedtime temperature, initial temperature, final temperature, wake-up temperature) in an app. In some embodiments, the subject sets the temperatures of the bed device with a remote control. In some embodiments, the subject sets the temperatures of the bed device with physical buttons. In some embodiments, the physical buttons are located on the side of the bed device. In some embodiments, the physical buttons are located on a hub located next to the bed device. In some embodiments, the subject sets the temperature of the bed device by tapping the side of the bed device. In some embodiments, the subject sets the temperatures of the bed device with a voice command. In some embodiments, the voice command is received via Alexa. In some embodiments, the voice command is received via Google Home. In some embodiments, the bed device can be paired with a third party smart device. In some cases, the temperatures of the bed device can be controlled with the third party smart device. In some cases, the third party device can be a smart phone, a smart watch, Google House, Alexa, a tablet, a smart speaker, or any combination thereof.

In some embodiments, the temperatures of the bed device correspond to dial values. In some embodiments, the dial values for the bed device temperatures are about −10 to about +10. In some embodiments, the temperature of the bed device is between about 12° C. to about 43° C. In some embodiments, the dial values for the bed device temperatures are at least or up to about −10, at least or up to about −9.5, at least or up to about −9, at least or up to about −8.5, at least or up to about −8, at least or up to about −7.5, at least or up to about −7, at least or up to about −6.5, at least or up to about −6, at least or up to about −5.5, at least or up to about −5, at least or up to about −4.5, at least or up to about −4, at least or up to about −3.5, at least or up to about −3, at least or up to about −2.5, at least or up to about −2, at least or up to about −1.5, at least or up to about −1, at least or up to about −0.5, at least or up to about 0, at least or up to about +0.5, at least or up to about +1, at least or up to about +1.5, at least or up to about +2, at least or up to about +2.5, at least or up to about +3, at least or up to about +3.5, at least or up to about +4, at least or up to about +4.5, at least or up to about +5, at least or up to about +5.5, at least or up to about +6, at least or up to about +6.5, at least or up to about +7, at least or up to about +7.5, at least or up to about +8, at least or up to about +8.5, at least or up to about +9, at least or up to about +9.5, and at least or up to about +10. In some embodiments, the −10 dial value corresponds to about 12° C. In some embodiments, the +10 dial value corresponds to about 43° C.

In some embodiments, the dial value for the bedtime temperature of the bed device is between about −10 to about +10 In some embodiments, the dial value for the bedtime temperature of the bed device is at least or up to about −10, at least or up to about −9.5, at least or up to about −9, at least or up to about −8.5, at least or up to about −8, at least or up to about −7.5, at least or up to about −7, at least or up to about −6.5, at least or up to about −6, at least or up to about −5.5, at least or up to about −5, at least or up to about −4.5, at least or up to about −4, at least or up to about −3.5, at least or up to about −3, at least or up to about −2.5, at least or up to about −2, at least or up to about −1.5, at least or up to about −0.5, at least or up to about 0, at least or up to about +0.5, at least or up to about +1, at least or up to about +1.5, at least or up to about +2, at least or up to about +2.5, at least or up to about +3, at least or up to about +3.5, at least or up to about +4, at least or up to about +4.5, at least or up to about +5, at least or up to about +5.5, at least or up to about +6, at least or up to about +6.5, at least or up to about +7, at least or up to about +7.5, at least or up to about +8, at least or up to about +8.5, at least or up to about +9, at least or up to about +9.5, or at least or up to about +10. In some embodiments, the dial value for the bedtime temperature of the bed device is about 0. In some embodiments, the bedtime temperature of the bed device is between about 12° C. to about 43° C. In some embodiments, the bedtime temperature of the bed device is at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., at least or up to about 31° C., at least or up to about 31.5° C., at least or up to about 32° C., at least or up to about 32.5° C., at least or up to about 33° C., at least or up to about 33.5° C., at least or up to about 34° C., at least or up to about 34.5° C., at least or up to about 35° C., at least or up to about 35.5° C., at least or up to about 36° C., at least or up to about 36.5° C., at least or up to about 37° C., at least or up to about 37.5° C., at least or up to about 38° C., at least or up to about 38.5° C., at least or up to about 39° C., at least or up to about 39.5° C., at least or up to about 40° C., at least or up to about 40.5° C., at least or up to about 41° C., at least or up to about 41.5° C., at least or up to about 42° C., at least or up to about 42.5° C., or at least or up to about 43° C. In some embodiments, the bedtime temperature of the bed device is at least or up to about 28° C.

In some embodiments, the bedtime temperature of bed device can fluctuate over the duration the bed device is directed or programmed to be at the bedtime temperature (e.g., via the temperature control device as provided herein). In some embodiments, the bedtime temperature fluctuates so the subject does not feel cold. Subjects who are on a bed device that maintains the same temperature for more than about 3 hours may feel cold and wake up. In some embodiments, the bedtime temperature fluctuates between about 0° C. to about 5° C. In some embodiments, the bedtime temperature fluctuates at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., or at least or up to about 5° C.

In some embodiments, the bedtime temperature of the bed device fluctuates between every about 0 to about 60 minutes. In some embodiments, the bedtime temperature of the bed device fluctuates every at least or up to about 0 minutes, at least or up to about 5 minutes, at least or up to about 10 minutes, at least or up to about 15 minutes, at least or up to about 20 minutes, at least or up to about 25 minutes, at least or up to about 30 minutes, at least or up to about 35 minutes, at least or up to about 40 minutes, at least or up to about 45 minutes, at least or up to about 50 minutes, at least or up to about 55 minutes, or at least or up to about 60 minutes.

In some embodiments, the dial value for the initial temperature of the bed device is between about −10 to about +10. In some embodiments, the dial value for the initial temperature of the bed device is at least or up to about −10, at least or up to about −9.5, at least or up to about −9, at least or up to about −8.5, at least or up to about −8, at least or up to about −7.5, at least or up to about −7, at least or up to about −6.5, at least or up to about −6, at least or up to about −5.5, at least or up to about −5, at least or up to about −4.5, at least or up to about −4, at least or up to about −3.5, at least or up to about −3, at least or up to about −2.5, at least or up to about −2, at least or up to about −1.5, at least or up to about −1, at least or up to about −0.5, at least or up to about 0, at least or up to about +0.5, at least or up to about +1, at least or up to about +1.5, at least or up to about +2, at least or up to about +2.5, at least or up to about +3, at least or up to about +3.5, at least or up to about +4, at least or up to about +4.5, at least or up to about +5, at least or up to about +5.5, at least or up to about +6, at least or up to about +6.5, at least or up to about +7, at least or up to about +7.5, at least or up to about +8, at least or up to about +8.5, at least or up to about +9, at least or up to about +9.5, or at least or up to about +10. In some embodiments, the initial temperature of the bed device is between about 12° C. to about 43° C. In some embodiments, the initial temperature of the bed device is at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., at least or up to about 31° C., at least or up to about 31.5° C., at least or up to about 32° C., at least or up to about 32.5° C., at least or up to about 33° C., at least or up to about 33.5° C., at least or up to about 34° C., at least or up to about 34.5° C., at least or up to about 35° C., at least or up to about 35.5° C., at least or up to about 36° C., at least or up to about 36.5° C., at least or up to about 37° C., at least or up to about 37.5° C., at least or up to about 38° C., at least or up to about 38.5° C., at least or up to about 39° C., at least or up to about 39.5° C., at least or up to about 40° C., at least or up to about 40.5° C., at least or up to about 41° C., at least or up to about 41.5° C., at least or up to about 42° C., at least or up to about 42.5° C., or at least or up to about 43° C.

In some embodiments, the initial temperature of bed device can fluctuate over the duration the bed device is directed or programmed to be at the initial temperature (e.g., via the temperature control device as provided herein). In some embodiments, the initial temperature fluctuates so the subject does not feel cold. Subjects who are on a bed device that maintains the same temperature for more than about 3 hours may feel cold and wake up. In some embodiments, the initial temperature fluctuates between about 0° C. to about 5° C. In some embodiments, the initial temperature fluctuates at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., or at least or up to about 5° C.

In some embodiments, the initial temperature of the bed device fluctuates between every about 0 to about 60 minutes. In some embodiments, the initial temperature of the bed device fluctuates every at least or up to about 0 minutes, at least or up to about 5 minutes, at least or up to about 10 minutes, at least or up to about 15 minutes, at least or up to about 20 minutes, at least or up to about 25 minutes, at least or up to about 30 minutes, at least or up to about 35 minutes, at least or up to about 40 minutes, at least or up to about 45 minutes, at least or up to about 50 minutes, at least or up to about 55 minutes, or at least or up to about 60 minutes.

In some embodiments, the dial value for the final temperature of the bed device is between about −10 to about +10. In some embodiments, the dial value for the final temperature of the bed device is at least or up to about −10, at least or up to about −9.5, at least or up to about −9, at least or up to about −8.5, at least or up to about −8, at least or up to about −7.5, at least or up to about −7, at least or up to about −6.5, at least or up to about −6, at least or up to about −5.5, at least or up to about −5, at least or up to about −4.5, at least or up to about −4, at least or up to about −3.5, at least or up to about −3, at least or up to about −2.5, at least or up to about −2, at least or up to about −1.5, at least or up to about −1, at least or up to about −0.5, at least or up to about 0, at least or up to about +0.5, at least or up to about +1, at least or up to about +1.5, at least or up to about +2, at least or up to about +2.5, at least or up to about +3, at least or up to about +3.5, at least or up to about +4, at least or up to about +4.5, at least or up to about +5, at least or up to about +5.5, at least or up to about +6, at least or up to about +6.5, at least or up to about +7, at least or up to about +7.5, at least or up to about +8, at least or up to about +8.5, at least or up to about +9, at least or up to about +9.5, or at least or up to about +10. In some embodiments, the final temperature of the bed device is between about 12° C. to about 43° C. In some embodiments, the final temperature of the bed device is at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., at least or up to about 31° C., at least or up to about 31.5° C., at least or up to about 32° C., at least or up to about 32.5° C., at least or up to about 33° C., at least or up to about 33.5° C., at least or up to about 34° C., at least or up to about 34.5° C., at least or up to about 35° C., at least or up to about 35.5° C., at least or up to about 36° C., at least or up to about 36.5° C., at least or up to about 37° C., at least or up to about 37.5° C., at least or up to about 38° C., at least or up to about 38.5° C., at least or up to about 39° C., at least or up to about 39.5° C., at least or up to about 40° C., at least or up to about 40.5° C., at least or up to about 41° C., at least or up to about 41.5° C., at least or up to about 42° C., at least or up to about 42.5° C., or at least or up to about 43° C.

In some embodiments, a temperature of the bed device can fluctuate over a duration that the bed device is set to be at a target temperature (e.g., via the temperature control device as provided herein). Accordingly, the temperature of the bed over the duration may not be constant and may not be identical to the target temperature throughout the entire duration. In some cases, when the bed device is set to be at a target temperature over a given time, the temperature control device can be configured to subject the bed device to a plurality of fluctuation cycles, each fluctuation cycle comprising increasing and decreasing (or vice versa) the temperature of the bed device (or the temperature of a fluid flowing within the bed device) about the target temperature. For example, a fluctuation cycle can comprise a combination of (i) increasing the temperature of the bed device to a high fluctuation temperature that is greater than the target temperature and (ii) decreasing the temperature of the bed device to a low fluctuation temperature that is lower than the target temperature. In the fluctuation cycle, the step of increasing the temperature to the high fluctuation temperature can occur prior to or subsequent to the step of decreasing the temperature of the low fluctuation temperature. Alternatively, when the bed device is supposed to remain constant at a target temperature, the temperature control device may not implement the plurality of fluctuation cycles. In some embodiments, the plurality of fluctuation cycles during use of the bed device can result in decreased number of awakenings of the user. In some embodiments, the plurality of fluctuation cycles during use of the bed device can result in an increase in deep sleep of the user.

In some embodiments, the plurality of fluctuation cycles during use of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% in the number of awakenings of the user during use of the bed device.

In some embodiments, the plurality of fluctuation cycles during use of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% decrease in percent change of total awake time of the user.

In some embodiments, the plurality of fluctuation cycles during use of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in light sleep of the user.

In some embodiments, the plurality of fluctuation cycles during use of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in deep sleep of the user.

In some embodiments, the plurality of fluctuation cycles during use of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in REM sleep of the user.

In some embodiments, the plurality of fluctuation cycles during use of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in total sleep time of the user.

In some embodiments, the plurality of fluctuation cycles during use of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in net sleep score of the user.

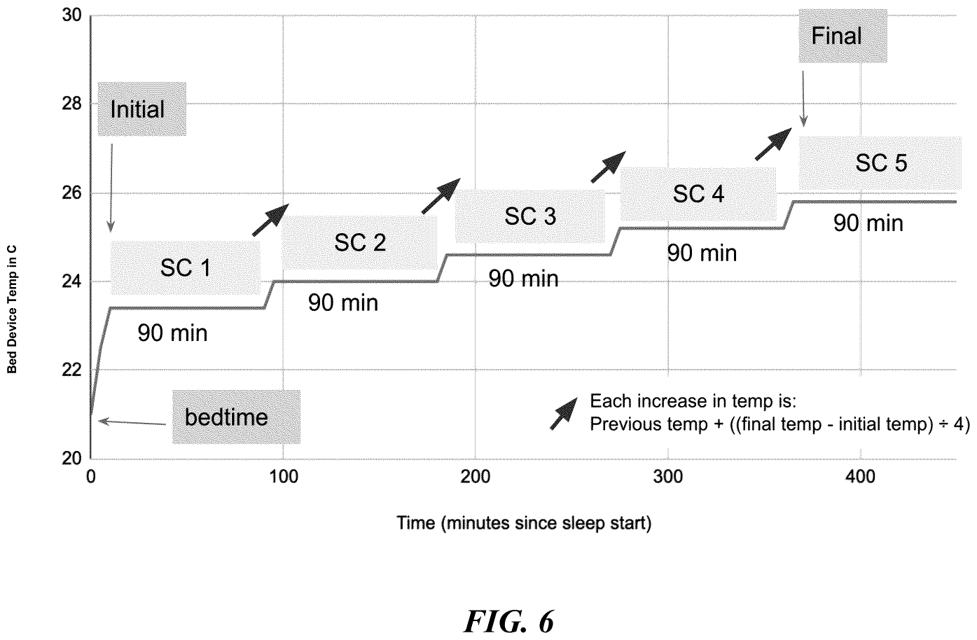

Similarly, in some embodiments, such fluctuation cycle as described herein can be implemented over a duration of time when the target temperature of the bed device is configured to change from a first target temperature to a second target temperature. Accordingly, when the bed device is set to change (e.g., increase or decrease) from a first target temperature to a second target temperature, the change of the bed device from the first target temperature to the second target temperature may not be linear (e.g., only increasing or only decreasing). Rather, the temperature control device can be configured to subject the bed device to the plurality of fluctuation cycles during the change from the first target temperature to the second target temperature, each fluctuation cycle comprising increasing and decreasing (or vice versa) the temperature of the bed device (or the temperature of a fluid flowing within the bed device). For example, when the overall temperature of the bed device is programmed to change from a first sleep cycle temperature to a second sleep cycle temperature (e.g., between SC1 and SC2, between SC2 and SC3, between SC3 and SC4, or between SC4 and SC5 in FIG. 6), the plurality of fluctuation cycles may be applied during such programmed change such that the rate of change of temperature from the first sleep cycle temperature to the second sleep cycle temperature may not be linear, but may fluctuation up and down for multiple cycles.

In some embodiments, a plurality cycle of the plurality of fluctuation cycles between two target temperatures can comprise at least or up to about 2 fluctuation cycles, at least or up to about 3 fluctuation cycles, at least or up to about 4 fluctuation cycles, at least or up to about 5 fluctuation cycles, at least or up to about 6 fluctuation cycles, at least or up to about 7 fluctuation cycles, at least or up to about 8 fluctuation cycles, at least or up to about 9 fluctuation cycles, at least or up to about 10 fluctuation cycles, at least or up to about 11 fluctuation cycles, at least or up to about 12 fluctuation cycles, at least or up to about 13 fluctuation cycles, at least or up to about 14 fluctuation cycles, at least or up to about 15 fluctuation cycles, at least or up to about 20 fluctuation cycles, at least or up to about 25 fluctuation cycles, at least or up to about 30 fluctuation cycles, at least or up to about 35 fluctuation cycles, at least or up to about 40 fluctuation cycles, at least or up to about 45 fluctuation cycles, at least or up to about 50 fluctuation cycles, at least or up to about 55 fluctuation cycles, at least or up to about 60 fluctuation cycles, at least or up to about 65 fluctuation cycles, at least or up to about 70 fluctuation cycles, at least or up to about 75 fluctuation cycles, at least or up to about 80 fluctuation cycles, at least or up to about 85 fluctuation cycles, at least or up to about 90 fluctuation cycles, at least or up to about 100 fluctuation cycles, at least or up to about 150 fluctuation cycles, at least or up to about 200 fluctuation cycles, at least or up to about 300 fluctuation cycles, at least or up to about 400 fluctuation cycles, at least or up to about 500 fluctuation cycles, at least or up to about 600 fluctuation cycles, at least or up to about 700 fluctuation cycles, at least or up to about 800 fluctuation cycles, at least or up to about 900 fluctuation cycles, or at least or up to about 1000 fluctuation cycles.

In some embodiments, the plurality of fluctuation cycles at a target temperature can comprise at least or up to about 2 fluctuation cycles, at least or up to about 3 fluctuation cycles, at least or up to about 4 fluctuation cycles, at least or up to about 5 fluctuation cycles, at least or up to about 6 fluctuation cycles, at least or up to about 7 fluctuation cycles, at least or up to about 8 fluctuation cycles, at least or up to about 9 fluctuation cycles, at least or up to about 10 fluctuation cycles, at least or up to about 11 fluctuation cycles, at least or up to about 12 fluctuation cycles, at least or up to about 13 fluctuation cycles, at least or up to about 14 fluctuation cycles, at least or up to about 15 fluctuation cycles, at least or up to about 20 fluctuation cycles, at least or up to about 25 fluctuation cycles, at least or up to about 30 fluctuation cycles, at least or up to about 35 fluctuation cycles, at least or up to about 40 fluctuation cycles, at least or up to about 45 fluctuation cycles, at least or up to about 50 fluctuation cycles, at least or up to about 55 fluctuation cycles, at least or up to about 60 fluctuation cycles, at least or up to about 65 fluctuation cycles, at least or up to about 70 fluctuation cycles, at least or up to about 75 fluctuation cycles, at least or up to about 80 fluctuation cycles, at least or up to about 85 fluctuation cycles, at least or up to about 90 fluctuation cycles, at least or up to about 100 fluctuation cycles, at least or up to about 150 fluctuation cycles, at least or up to about 200 fluctuation cycles, at least or up to about 300 fluctuation cycles, at least or up to about 400 fluctuation cycles, at least or up to about 500 fluctuation cycles, at least or up to about 600 fluctuation cycles, at least or up to about 700 fluctuation cycles, at least or up to about 800 fluctuation cycles, at least or up to about 900 fluctuation cycles, or at least or up to about 1000 fluctuation cycles.

In some embodiments, a fluctuation cycle of the plurality of fluctuation cycles at a target temperature can comprise at least or up to about 1 temperature fluctuation, at least or up to about 2 temperature fluctuations, at least or up to about 3 temperature fluctuations, at least or up to about 4 temperature fluctuations, at least or up to about 5 temperature fluctuations, at least or up to about 6 temperature fluctuations, at least or up to about 7 temperature fluctuations, at least or up to about 8 temperature fluctuations, at least or up to about 9 temperature fluctuations, at least or up to about 10 temperature fluctuations, at least or up to about 11 temperature fluctuations, at least or up to about 12 temperature fluctuations, at least or up to about 13 temperature fluctuations, at least or up to about 14 temperature fluctuations, at least or up to about 15 temperature fluctuations, at least or up to about 16 temperature fluctuations, at least or up to about 17 temperature fluctuations, at least or up to about 18 temperature fluctuations, at least or up to about 19 temperature fluctuations, at least or up to about 20 temperature fluctuations, at least or up to about 21 temperature fluctuations, at least or up to about 22 temperature fluctuations, at least or up to about 23 temperature fluctuations, at least or up to about 24 temperature fluctuations, at least or up to about 25 temperature fluctuations, at least or up to about 26 temperature fluctuations, at least or up to about 27 temperature fluctuations, at least or up to about 28 temperature fluctuations, at least or up to about 29 temperature fluctuations, at least or up to about 30 temperature fluctuations, at least or up to about 31 temperature fluctuations, at least or up to about 32 temperature fluctuations, at least or up to about 33 temperature fluctuations, at least or up to about 34 temperature fluctuations, at least or up to about 35 temperature fluctuations, at least or up to about 36 temperature fluctuations, at least or up to about 37 temperature fluctuations, at least or up to about 38 temperature fluctuations, at least or up to about 39 temperature fluctuations, at least or up to about 40 temperature fluctuations, at least or up to about 41 temperature fluctuations, at least or up to about 42 temperature fluctuations, at least or up to about 43 temperature fluctuations, at least or up to about 44 temperature fluctuations, at least or up to about 45 temperature fluctuations, at least or up to about 46 temperature fluctuations, at least or up to about 47 temperature fluctuations, at least or up to about 48 temperature fluctuations, at least or up to about 49 temperature fluctuations, at least or up to about 50 temperature fluctuations, at least or up to about 51 temperature fluctuations, at least or up to about 52 temperature fluctuations, at least or up to about 53 temperature fluctuations, at least or up to about 54 temperature fluctuations, at least or up to about 55 temperature fluctuations, at least or up to about 56 temperature fluctuations, at least or up to about 57 temperature fluctuations, at least or up to about 58 temperature fluctuations, at least or up to about 59 temperature fluctuations, or at least or up to about 60 temperature fluctuations per minute.

In some embodiments, a fluctuation cycle of the plurality of fluctuation cycles at a target temperature can comprise at least or up to about 1 temperature fluctuation, at least or up to about 2 temperature fluctuations, at least or up to about 3 temperature fluctuations, at least or up to about 4 temperature fluctuations, at least or up to about 5 temperature fluctuations, at least or up to about 6 temperature fluctuations, at least or up to about 7 temperature fluctuations, at least or up to about 8 temperature fluctuations, at least or up to about 9 temperature fluctuations, at least or up to about 10 temperature fluctuations, at least or up to about 11 temperature fluctuations, at least or up to about 12 temperature fluctuations, at least or up to about 13 temperature fluctuations, at least or up to about 14 temperature fluctuations, at least or up to about 15 temperature fluctuations, at least or up to about 16 temperature fluctuations, at least or up to about 17 temperature fluctuations, at least or up to about 18 temperature fluctuations, at least or up to about 19 temperature fluctuations, at least or up to about 20 temperature fluctuations, at least or up to about 21 temperature fluctuations, at least or up to about 22 temperature fluctuations, at least or up to about 23 temperature fluctuations, at least or up to about 24 temperature fluctuations, at least or up to about 25 temperature fluctuations, at least or up to about 26 temperature fluctuations, at least or up to about 27 temperature fluctuations, at least or up to about 28 temperature fluctuations, at least or up to about 29 temperature fluctuations, at least or up to about 30 temperature fluctuations, at least or up to about 31 temperature fluctuations, at least or up to about 32 temperature fluctuations, at least or up to about 33 temperature fluctuations, at least or up to about 34 temperature fluctuations, at least or up to about 35 temperature fluctuations, at least or up to about 36 temperature fluctuations, at least or up to about 37 temperature fluctuations, at least or up to about 38 temperature fluctuations, at least or up to about 39 temperature fluctuations, at least or up to about 40 temperature fluctuations, at least or up to about 41 temperature fluctuations, at least or up to about 42 temperature fluctuations, at least or up to about 43 temperature fluctuations, at least or up to about 44 temperature fluctuations, at least or up to about 45 temperature fluctuations, at least or up to about 46 temperature fluctuations, at least or up to about 47 temperature fluctuations, at least or up to about 48 temperature fluctuations, at least or up to about 49 temperature fluctuations, at least or up to about 50 temperature fluctuations, at least or up to about 51 temperature fluctuations, at least or up to about 52 temperature fluctuations, at least or up to about 53 temperature fluctuations, at least or up to about 54 temperature fluctuations, at least or up to about 55 temperature fluctuations, at least or up to about 56 temperature fluctuations, at least or up to about 57 temperature fluctuations, at least or up to about 58 temperature fluctuations, at least or up to about 59 temperature fluctuations, or at least or up to about 60 temperature fluctuations per hour.

In some embodiments, a fluctuation cycle of the plurality of fluctuation cycles between two target temperatures can comprise at least or up to about 1 temperature fluctuation, at least or up to about 2 temperature fluctuations, at least or up to about 3 temperature fluctuations, at least or up to about 4 temperature fluctuations, at least or up to about 5 temperature fluctuations, at least or up to about 6 temperature fluctuations, at least or up to about 7 temperature fluctuations, at least or up to about 8 temperature fluctuations, at least or up to about 9 temperature fluctuations, at least or up to about 10 temperature fluctuations, at least or up to about 11 temperature fluctuations, at least or up to about 12 temperature fluctuations, at least or up to about 13 temperature fluctuations, at least or up to about 14 temperature fluctuations, at least or up to about 15 temperature fluctuations, at least or up to about 16 temperature fluctuations, at least or up to about 17 temperature fluctuations, at least or up to about 18 temperature fluctuations, at least or up to about 19 temperature fluctuations, at least or up to about 20 temperature fluctuations, at least or up to about 21 temperature fluctuations, at least or up to about 22 temperature fluctuations, at least or up to about 23 temperature fluctuations, at least or up to about 24 temperature fluctuations, at least or up to about 25 temperature fluctuations, at least or up to about 26 temperature fluctuations, at least or up to about 27 temperature fluctuations, at least or up to about 28 temperature fluctuations, at least or up to about 29 temperature fluctuations, at least or up to about 30 temperature fluctuations, at least or up to about 31 temperature fluctuations, at least or up to about 32 temperature fluctuations, at least or up to about 33 temperature fluctuations, at least or up to about 34 temperature fluctuations, at least or up to about 35 temperature fluctuations, at least or up to about 36 temperature fluctuations, at least or up to about 37 temperature fluctuations, at least or up to about 38 temperature fluctuations, at least or up to about 39 temperature fluctuations, at least or up to about 40 temperature fluctuations, at least or up to about 41 temperature fluctuations, at least or up to about 42 temperature fluctuations, at least or up to about 43 temperature fluctuations, at least or up to about 44 temperature fluctuations, at least or up to about 45 temperature fluctuations, at least or up to about 46 temperature fluctuations, at least or up to about 47 temperature fluctuations, at least or up to about 48 temperature fluctuations, at least or up to about 49 temperature fluctuations, at least or up to about 50 temperature fluctuations, at least or up to about 51 temperature fluctuations, at least or up to about 52 temperature fluctuations, at least or up to about 53 temperature fluctuations, at least or up to about 54 temperature fluctuations, at least or up to about 55 temperature fluctuations, at least or up to about 56 temperature fluctuations, at least or up to about 57 temperature fluctuations, at least or up to about 58 temperature fluctuations, at least or up to about 59 temperature fluctuations, or at least or up to about 60 temperature fluctuations per minute.

In some embodiments, a fluctuation cycle of the plurality of fluctuation cycles between two target temperatures can comprise at least or up to about 1 temperature fluctuation, at least or up to about 2 temperature fluctuations, at least or up to about 3 temperature fluctuations, at least or up to about 4 temperature fluctuations, at least or up to about 5 temperature fluctuations, at least or up to about 6 temperature fluctuations, at least or up to about 7 temperature fluctuations, at least or up to about 8 temperature fluctuations, at least or up to about 9 temperature fluctuations, at least or up to about 10 temperature fluctuations, at least or up to about 11 temperature fluctuations, at least or up to about 12 temperature fluctuations, at least or up to about 13 temperature fluctuations, at least or up to about 14 temperature fluctuations, at least or up to about 15 temperature fluctuations, at least or up to about 16 temperature fluctuations, at least or up to about 17 temperature fluctuations, at least or up to about 18 temperature fluctuations, at least or up to about 19 temperature fluctuations, at least or up to about 20 temperature fluctuations, at least or up to about 21 temperature fluctuations, at least or up to about 22 temperature fluctuations, at least or up to about 23 temperature fluctuations, at least or up to about 24 temperature fluctuations, at least or up to about 25 temperature fluctuations, at least or up to about 26 temperature fluctuations, at least or up to about 27 temperature fluctuations, at least or up to about 28 temperature fluctuations, at least or up to about 29 temperature fluctuations, at least or up to about 30 temperature fluctuations, at least or up to about 31 temperature fluctuations, at least or up to about 32 temperature fluctuations, at least or up to about 33 temperature fluctuations, at least or up to about 34 temperature fluctuations, at least or up to about 35 temperature fluctuations, at least or up to about 36 temperature fluctuations, at least or up to about 37 temperature fluctuations, at least or up to about 38 temperature fluctuations, at least or up to about 39 temperature fluctuations, at least or up to about 40 temperature fluctuations, at least or up to about 41 temperature fluctuations, at least or up to about 42 temperature fluctuations, at least or up to about 43 temperature fluctuations, at least or up to about 44 temperature fluctuations, at least or up to about 45 temperature fluctuations, at least or up to about 46 temperature fluctuations, at least or up to about 47 temperature fluctuations, at least or up to about 48 temperature fluctuations, at least or up to about 49 temperature fluctuations, at least or up to about 50 temperature fluctuations, at least or up to about 51 temperature fluctuations, at least or up to about 52 temperature fluctuations, at least or up to about 53 temperature fluctuations, at least or up to about 54 temperature fluctuations, at least or up to about 55 temperature fluctuations, at least or up to about 56 temperature fluctuations, at least or up to about 57 temperature fluctuations, at least or up to about 58 temperature fluctuations, at least or up to about 59 temperature fluctuations, or at least or up to about 60 temperature fluctuations per hour. In some embodiments, the plurality of fluctuation cycles may or may not be substantially the same to one another. In some cases, (i) a net change in the temperature of the bed device (or the fluid controlled by the temperature control device) from a fluctuation cycle may or may not be substantially the same as (ii) an additional net change in the temperature of the bed device (or the fluid controlled by the temperature control device) from an additional fluctuation cycle. In some cases, (i) a duration (e.g., a length of time) of a fluctuation cycle may or may not be substantially the same as (ii) a duration of an additional fluctuation cycle.

In some embodiments, for a given fluctuation cycle comprising at least an increase in the temperature of the bed device (or the fluid controlled by the temperature control device) and a decrease in the temperature of the bed device (or the fluid), an absolute value of the increase in the temperature and an absolute value of the decrease in the temperature may or may not be substantially the same. For example, the increase in the temperature may be substantially the same as the decrease in the temperature, such that a net change in the temperature is substantially zero. In another example, the increase in the temperature may be greater than the decrease in the temperature, such that a net change in the temperature is greater than zero (e.g., during application of the plurality of fluctuation cycles between two target temperature of the bed device as abovementioned). In a different example, the decrease in the temperature may be greater than the increase in the temperature, such that a net change in the temperature is less than zero (e.g., during application of the plurality of fluctuation cycles between two target temperature of the bed device as abovementioned).

In some embodiments, a total (or sum of) duration of the plurality of fluctuation cycles can be at least or up to about 1 minute, at least or up to about 2 minutes, at least or up to about 5 minutes, at least or up to about 10 minutes, at least or up to about 15 minutes, at least or up to about 20 minutes, at least or up to about 25 minutes, at least or up to about 30 minutes, at least or up to about 35 minutes, at least or up to about 40 minutes, at least or up to about 45 minutes, at least or up to about 50 minutes, at least or up to about 55 minutes, at least or up to about 60 minutes, at least or up to about 70 minutes, at least or up to about 80 minutes, at least or up to about 90 minutes, at least or up to about 100 minutes, at least or up to about 110 minutes, at least or up to about 120 minutes, at least or up to about 130 minutes, at least or up to about 140 minutes, at least or up to about 150 minutes, at least or up to about 160 minutes, at least or up to about 170 minutes, at least or up to about 180 minutes, at least or up to about 190 minutes, at least or up to about 200 minutes, at least or up to about 210 minutes, at least or up to about 220 minutes, at least or up to about 230 minutes, at least or up to about 240 minutes, at least or up to about 300 minutes, at least or up to about 360 minutes, at least or up to about 420 minutes, at least or up to about 480 minutes, at least or up to about 540 minutes, or at least or up to about 600 minutes.

In some embodiments, a duration of a fluctuation cycle of the plurality of fluctuation cycles can be at least or up to about 5 seconds, at least or up to about 10 seconds, at least or up to about 15 seconds, at least or up to about 20 seconds, at least or up to about 25 seconds, at least or up to about 30 seconds, at least or up to about 40 seconds, at least or up to about 50 seconds, at least or up to about 60 seconds, at least or up to about 70 seconds, at least or up to about 80 seconds, at least or up to about 90 seconds, at least or up to about 100 seconds, at least or up to about 110 seconds, at least or up to about 120 seconds, at least or up to about 3 minutes, at least or up to about 4 minutes, at least or up to about 5 minutes, at least or up to about 6 minutes, at least or up to about 7 minutes, at least or up to about 8 minutes, at least or up to about 9 minutes, at least or up to about 10 minutes, at least or up to about 11 minutes, at least or up to about 12 minutes, at least or up to about 13 minutes, at least or up to about 14 minutes, at least or up to about 15 minutes, at least or up to about 16 minutes, at least or up to about 17 minutes, at least or up to about 18 minutes, at least or up to about 19 minutes, at least or up to about 20 minutes, at least or up to about 21 minutes, at least or up to about 22 minutes, at least or up to about 23 minutes, at least or up to about 24 minutes, at least or up to about 25 minutes, at least or up to about 26 minutes, at least or up to about 27 minutes, at least or up to about 28 minutes, at least or up to about 29 minutes, at least or up to about 30 minutes, at least or up to about 31 minutes, at least or up to about 32 minutes, at least or up to about 33 minutes, at least or up to about 34 minutes, at least or up to about 35 minutes, at least or up to about 36 minutes, at least or up to about 37 minutes, at least or up to about 38 minutes, at least or up to about 39 minutes, at least or up to about 40 minutes, at least or up to about 41 minutes, at least or up to about 42 minutes, at least or up to about 43 minutes, at least or up to about 44 minutes, at least or up to about 45 minutes, at least or up to about 46 minutes, at least or up to about 47 minutes, at least or up to about 48 minutes, at least or up to about 49 minutes, at least or up to about 50 minutes, at least or up to about 51 minutes, at least or up to about 52 minutes, at least or up to about 53 minutes, at least or up to about 54 minutes, at least or up to about 55 minutes, at least or up to about 56 minutes, at least or up to about 57 minutes, at least or up to about 58 minutes, at least or up to about 59 minutes, at least or up to about 60 minutes, at least or up to about 61 minutes, at least or up to about 62 minutes, at least or up to about 63 minutes, at least or up to about 64 minutes, at least or up to about 65 minutes, at least or up to about 66 minutes, at least or up to about 67 minutes, at least or up to about 68 minutes, at least or up to about 69 minutes, at least or up to about 70 minutes, at least or up to about 71 minutes, at least or up to about 72 minutes, at least or up to about 73 minutes, at least or up to about 74 minutes, at least or up to about 75 minutes, at least or up to about 76 minutes, at least or up to about 77 minutes, at least or up to about 78 minutes, at least or up to about 79 minutes, at least or up to about 80 minutes, at least or up to about 81 minutes, at least or up to about 82 minutes, at least or up to about 83 minutes, at least or up to about 84 minutes, at least or up to about 85 minutes, at least or up to about 86 minutes, at least or up to about 87 minutes, at least or up to about 88 minutes, at least or up to about 89 minutes, at least or up to about 90 minutes, at least or up to about 91 minutes, at least or up to about 92 minutes, at least or up to about 93 minutes, at least or up to about 94 minutes, at least or up to about 95 minutes, at least or up to about 96 minutes, at least or up to about 97 minutes, at least or up to about 98 minutes, at least or up to about 99 minutes, at least or up to about 100 minutes, at least or up to about 101 minutes, at least or up to about 102 minutes, at least or up to about 103 minutes, at least or up to about 104 minutes, at least or up to about 105 minutes, at least or up to about 106 minutes, at least or up to about 107 minutes, at least or up to about 108 minutes, at least or up to about 109 minutes, at least or up to about 110 minutes, at least or up to about 111 minutes, at least or up to about 112 minutes, at least or up to about 113 minutes, at least or up to about 114 minutes, at least or up to about 115 minutes, at least or up to about 116 minutes, at least or up to about 117 minutes, at least or up to about 118 minutes, at least or up to about 119 minutes, or at least or up to about 120 minutes.

In some embodiments, a duration of an increasing temperature step and/or a decreasing temperature step of a fluctuation cycle of the plurality of fluctuation cycles can be at least or up to about 5 seconds, at least or up to about 10 seconds, at least or up to about 15 seconds, at least or up to about 20 seconds, at least or up to about 25 seconds, at least or up to about 30 seconds, at least or up to about 40 seconds, at least or up to about 50 seconds, at least or up to about 60 seconds, at least or up to about 70 seconds, at least or up to about 80 seconds, at least or up to about 90 seconds, at least or up to about 100 seconds, at least or up to about 110 seconds, at least or up to about 120 seconds, at least or up to about 3 minutes, at least or up to about 4 minutes, at least or up to about 5 minutes, at least or up to about 6 minutes, at least or up to about 7 minutes, at least or up to about 8 minutes, at least or up to about 9 minutes, at least or up to about 10 minutes, at least or up to about 11 minutes, at least or up to about 12 minutes, at least or up to about 13 minutes, at least or up to about 14 minutes, at least or up to about 15 minutes, at least or up to about 16 minutes, at least or up to about 17 minutes, at least or up to about 18 minutes, at least or up to about 19 minutes, at least or up to about 20 minutes, at least or up to about 21 minutes, at least or up to about 22 minutes, at least or up to about 23 minutes, at least or up to about 24 minutes, at least or up to about 25 minutes, at least or up to about 26 minutes, at least or up to about 27 minutes, at least or up to about 28 minutes, at least or up to about 29 minutes, at least or up to about 30 minutes, at least or up to about 31 minutes, at least or up to about 32 minutes, at least or up to about 33 minutes, at least or up to about 34 minutes, at least or up to about 35 minutes, at least or up to about 36 minutes, at least or up to about 37 minutes, at least or up to about 38 minutes, at least or up to about 39 minutes, at least or up to about 40 minutes, at least or up to about 41 minutes, at least or up to about 42 minutes, at least or up to about 43 minutes, at least or up to about 44 minutes, at least or up to about 45 minutes, at least or up to about 46 minutes, at least or up to about 47 minutes, at least or up to about 48 minutes, at least or up to about 49 minutes, at least or up to about 50 minutes, at least or up to about 51 minutes, at least or up to about 52 minutes, at least or up to about 53 minutes, at least or up to about 54 minutes, at least or up to about 55 minutes, at least or up to about 56 minutes, at least or up to about 57 minutes, at least or up to about 58 minutes, at least or up to about 59 minutes, at least or up to about 60 minutes, at least or up to about 61 minutes, at least or up to about 62 minutes, at least or up to about 63 minutes, at least or up to about 64 minutes, at least or up to about 65 minutes, at least or up to about 66 minutes, at least or up to about 67 minutes, at least or up to about 68 minutes, at least or up to about 69 minutes, at least or up to about 70 minutes, at least or up to about 71 minutes, at least or up to about 72 minutes, at least or up to about 73 minutes, at least or up to about 74 minutes, at least or up to about 75 minutes, at least or up to about 76 minutes, at least or up to about 77 minutes, at least or up to about 78 minutes, at least or up to about 79 minutes, at least or up to about 80 minutes, at least or up to about 81 minutes, at least or up to about 82 minutes, at least or up to about 83 minutes, at least or up to about 84 minutes, at least or up to about 85 minutes, at least or up to about 86 minutes, at least or up to about 87 minutes, at least or up to about 88 minutes, at least or up to about 89 minutes, at least or up to about 90 minutes, at least or up to about 91 minutes, at least or up to about 92 minutes, at least or up to about 93 minutes, at least or up to about 94 minutes, at least or up to about 95 minutes, at least or up to about 96 minutes, at least or up to about 97 minutes, at least or up to about 98 minutes, at least or up to about 99 minutes, at least or up to about 100 minutes, at least or up to about 101 minutes, at least or up to about 102 minutes, at least or up to about 103 minutes, at least or up to about 104 minutes, at least or up to about 105 minutes, at least or up to about 106 minutes, at least or up to about 107 minutes, at least or up to about 108 minutes, at least or up to about 109 minutes, at least or up to about 110 minutes, at least or up to about 111 minutes, at least or up to about 112 minutes, at least or up to about 113 minutes, at least or up to about 114 minutes, at least or up to about 115 minutes, at least or up to about 116 minutes, at least or up to about 117 minutes, at least or up to about 118 minutes, at least or up to about 119 minutes, or at least or up to about 120 minutes.

In some embodiments, a degree (e.g., an absolute value) of change of the temperature during an increasing temperature step and/or a decreasing temperature step of a fluctuation cycle of the plurality of fluctuation cycles can be at least or up to about 0.001 degrees Celsius, at least or up to about 0.002 degrees Celsius, at least or up to about 0.003 degrees Celsius, at least or up to about 0.004 degrees Celsius, at least or up to about 0.005 degrees Celsius, at least or up to about 0.006 degrees Celsius, at least or up to about 0.007 degrees Celsius, at least or up to about 0.008 degrees Celsius, at least or up to about 0.009 degrees Celsius, at least or up to about 0.01 degrees Celsius, at least or up to about 0.02 degrees Celsius, at least or up to about 0.03 degrees Celsius, at least or up to about 0.04 degrees Celsius, at least or up to about 0.05 degrees Celsius, at least or up to about 0.06 degrees Celsius, at least or up to about 0.07 degrees Celsius, at least or up to about 0.08 degrees Celsius, at least or up to about 0.09 degrees Celsius, at least or up to about 0.1 degrees Celsius, at least or up to about 0.15 degrees Celsius, at least or up to about 0.2 degrees Celsius, at least or up to about 0.25 degrees Celsius, at least or up to about 0.3 degrees Celsius, at least or up to about 0.35 degrees Celsius, at least or up to about 0.4 degrees Celsius, at least or up to about 0.45 Celsius, at least or up to about 0.5 degrees Celsius, at least or up to about 0.55 degrees Celsius, at least or up to about 0.6 degrees Celsius, at least or up to about 0.65 at least or up to about 0.7 degrees Celsius, at least or up to about 0.75 degrees Celsius, at least or up to about 0.8 degrees Celsius, at least or up to about 0.85 degrees Celsius, at least or up to about 0.9 degrees Celsius, at least or up to about 0.95 degrees Celsius, at least or up to about 1 degree Celsius, at least or up to about 1.05 degrees Celsius, at least or up to about 1.10 degrees Celsius, at least or up to about 1.15 degrees Celsius, at least or up to about 1.2 degrees Celsius, at least or up to about 1.25 degrees Celsius, at least or up to about 1.3 degrees Celsius, at least or up to about 1.35 degrees Celsius, at least or up to about 1.4 degrees Celsius, at least or up to about 1.45 degrees Celsius, at least or up to about 1.5 degrees Celsius, at least or up to about 1.55 degrees Celsius, at least or up to about 1.6 degrees Celsius, at least or up to about 1.65 degrees Celsius, at least or up to about 1.7 degrees Celsius, at least or up to about 1.75 degrees Celsius, at least or up to about 1.8 degrees Celsius, at least or up to about 1.85 degrees Celsius, at least or up to about 1.9 degrees Celsius, at least or up to about 1.95 degrees Celsius, at least or up to about 2.0 degrees Celsius, at least or up to about 2.05 degrees Celsius, at least or up to about 2.1 degrees Celsius, at least or up to about 2.15 degrees Celsius, at least or up to about 2.2 degrees Celsius, at least or up to about 2.25 degrees Celsius, at least or up to about 2.3 degrees Celsius, at least or up to about 2.35 degrees Celsius, at least or up to about 2.4 degrees Celsius, at least or up to about 2.45 degrees Celsius, at least or up to about 2.5 degrees Celsius, at least or up to about 2.55 degrees Celsius, at least or up to about 2.6 degrees Celsius, at least or up to about 2.65 degrees Celsius, at least or up to about 2.7 degrees Celsius, at least or up to about 2.75 degrees Celsius, at least or up to about 2.8 degrees Celsius, at least or up to about 2.85 degrees Celsius, at least or up to about 2.9 degrees Celsius, at least or up to about 2.95 degrees Celsius, at least or up to about 3.0 degrees Celsius, at least or up to about 3.05 degrees Celsius, at least or up to about 3.1 degrees Celsius, at least or up to about 3.15 degrees Celsius, at least or up to about 3.2 degrees Celsius, at least or up to about 3.25 degrees Celsius, at least or up to about 3.3 degrees Celsius, at least or up to about 3.35 degrees Celsius, at least or up to about 3.4 degrees Celsius, at least or up to about 3.45 degrees Celsius, at least or up to about 3.5 degrees Celsius, at least or up to about 3.55 degrees Celsius, at least or up to about 3.6 degrees Celsius, at least or up to about 3.65 degrees Celsius, at least or up to about 3.7 degrees Celsius, at least or up to about 3.75 degrees Celsius, at least or up to about 3.8 degrees Celsius, at least or up to about 3.85 degrees Celsius, at least or up to about 3.9 degrees Celsius, at least or up to about 3.95 degrees Celsius, at least or up to about 4.0 degrees Celsius, at least or up to about 4.05 degrees Celsius, at least or up to about 4.1 degrees Celsius, at least or up to about 4.15 degrees Celsius, at least or up to about 4.2 degrees Celsius, at least or up to about 4.25 degrees Celsius, at least or up to about 4.3 degrees Celsius, at least or up to about 4.35 degrees Celsius, at least or up to about 4.4 degrees Celsius, at least or up to about 4.45 degrees Celsius, at least or up to about 4.5 degrees Celsius, at least or up to about 4.55 degrees Celsius, at least or up to about 4.6 degrees Celsius, at least or up to about 4.65 degrees Celsius, at least or up to about 4.7 degrees Celsius, at least or up to about 4.75 degrees Celsius, at least or up to about 4.8 degrees Celsius, at least or up to about 4.85 degrees Celsius, at least or up to about 4.9 degrees Celsius, at least or up to about 4.95 degrees Celsius, at least or up to about 5.0 degrees Celsius, at least or up to about 5.05 degrees Celsius, at least or up to about 5.1 degrees Celsius, at least or up to about 5.15 degrees Celsius, at least or up to about 5.2 degrees Celsius, at least or up to about 5.25 degrees Celsius, at least or up to about 5.3 degrees Celsius, at least or up to about 5.35 degrees Celsius, at least or up to about 5.4 degrees Celsius, at least or up to about 5.45 degrees Celsius, at least or up to about 5.5 degrees Celsius, at least or up to about 5.55 degrees Celsius, at least or up to about 5.6 degrees Celsius, at least or up to about 5.65 degrees Celsius, at least or up to about 5.7 degrees Celsius, at least or up to about 5.75 degrees Celsius, at least or up to about 5.8 degrees Celsius, at least or up to about 5.85 degrees Celsius, at least or up to about 5.9 degrees Celsius, at least or up to about 6.0 degrees Celsius, at least or up to about 6.05 degrees Celsius, at least or up to about 6.1 degrees Celsius, at least or up to about 6.15 degrees Celsius, at least or up to about 6.2 degrees Celsius, at least or up to about 6.25 degrees Celsius, at least or up to about 6.3 degrees Celsius, at least or up to about 6.35 degrees Celsius, at least or up to about 6.4 degrees Celsius, at least or up to about 6.45 degrees Celsius, at least or up to about 6.5 degrees Celsius, at least or up to about 6.55 degrees Celsius, at least or up to about 6.6 degrees Celsius, at least or up to about 6.65 degrees Celsius, at least or up to about 6.7 degrees Celsius, at least or up to about 6.75 degrees Celsius, at least or up to about 6.8 degrees Celsius, at least or up to about 6.85 degrees Celsius, at least or up to about 6.9 degrees Celsius, at least or up to about 6.95 degrees Celsius, at least or up to about 7.0 degrees Celsius, at least or up to about 7.05 degrees Celsius, at least or up to about 7.1 degrees Celsius, at least or up to about 7.15 degrees Celsius, at least or up to about 7.2 degrees Celsius, at least or up to about 7.25 degrees Celsius, at least or up to about 7.3 degrees Celsius, at least or up to about 7.35 degrees Celsius, at least or up to about 7.4 degrees Celsius, at least or up to about 7.45 degrees Celsius, at least or up to about 7.5 degrees Celsius, at least or up to about 7.55 degrees Celsius, at least or up to about 7.6 degrees Celsius, at least or up to about 7.65 degrees Celsius, at least or up to about 7.7 degrees Celsius, at least or up to about 7.75 degrees Celsius, at least or up to about 7.8 degrees Celsius, at least or up to about 7.85 degrees Celsius, at least or up to about 7.9 degrees Celsius, at least or up to about 7.95 degrees Celsius, at least or up to about 8.0 degrees Celsius, at least or up to about 8.05 degrees Celsius, at least or up to about 8.1 degrees Celsius, at least or up to about 8.15 degrees Celsius, at least or up to about 8.2 degrees Celsius, at least or up to about 8.25 degrees Celsius, at least or up to about 8.3 degrees Celsius, at least or up to about 8.35 degrees Celsius, at least or up to about 8.4 degrees Celsius, at least or up to about 8.45 degrees Celsius, at least or up to about 8.5 degrees Celsius, at least or up to about 8.55 degrees Celsius, at least or up to about 8.6 degrees Celsius, at least or up to about 8.65 degrees Celsius, at least or up to about 8.7 degrees Celsius, at least or up to about 8.75 degrees Celsius, at least or up to about 8.8 degrees Celsius, at least or up to about 8.85 degrees Celsius, at least or up to about 8.9 degrees Celsius, at least or up to about 8.95 degrees Celsius, at least or up to about 9.0 degrees Celsius, at least or up to about 9.05 degrees Celsius, at least or up to about 9.1 degrees Celsius, at least or up to about 9.15 degrees Celsius, at least or up to about 9.2 degrees Celsius, at least or up to about 9.25 degrees Celsius, at least or up to about 9.3 degrees Celsius, at least or up to about 9.35 degrees Celsius, at least or up to about 9.4 degrees Celsius, at least or up to about 9.45 degrees Celsius, at least or up to about 9.5 degrees Celsius, at least or up to about 9.55 degrees Celsius, at least or up to about 9.6 degrees Celsius, at least or up to about 9.65 degrees Celsius, at least or up to about 9.7 degrees Celsius, at least or up to about 9.75 degrees Celsius, at least or up to about 9.8 degrees Celsius, at least or up to about 9.85 degrees Celsius, at least or up to about 9.9 degrees Celsius, at least or up to about 9.95 degrees Celsius, or at least or up to about 10.0 degrees Celsius. Such degree of change of the temperature during the increasing temperature step and/or the decreasing temperature step of the fluctuation cycle can be instant or, alternatively, can occur over the period of at least or up to about 0.01 seconds, at least or up to about 0.02 seconds, at least or up to about 0.03 seconds, at least or up to about 0.04 seconds, at least or up to about 0.05 seconds, at least or up to about 0.06 seconds, at least or up to about 0.07 seconds, at least or up to about 0.08 seconds, at least or up to about 0.09 seconds, at least or up to about 0.1 seconds, at least or up to about 0.2 seconds, at least or up to about 0.3 seconds, at least or up to about 0.4 seconds, at least or up to about 0.5 seconds, at least or up to about 0.6 seconds, at least or up to about 0.7 seconds, at least or up to about 0.8 seconds, at least or up to about 0.9 seconds, at least or up to about 1 second, at least or up to about 2 seconds, at least or up to about 3 seconds, at least or up to about 4 seconds, at least or up to about 5 seconds, at least or up to about 6 seconds, at least or up to about 7 seconds, at least or up to about 8 seconds, at least or up to about 9 seconds, at least or up to about 10 seconds, at least or up to about 15 seconds, at least or up to about 20 seconds, at least or up to about 25 seconds, at least or up to about 30 seconds, at least or up to about 40 seconds, at least or up to about 50 seconds, at least or up to about 60 seconds, at least or up to about 70 seconds, at least or up to about 80 seconds, at least or up to about 90 seconds, or at least or up to about 100 seconds.

In some embodiments, a degree (e.g., an absolute value) of change of the temperature during an increasing temperature step and/or a decreasing temperature step of a fluctuation cycle of the plurality of fluctuation cycles can be a relative percentage of the first target temperature and/or the second target temperature. In some embodiments, the degree of change of the temperature during the increasing temperature step and/or the decreasing temperature step of the fluctuation cycle of the plurality of fluctuation cycles can be at least or up to about 0.0%, at least or up to about 0.5%, at least or up to about 1.0%, at least or up to about 1.5%, at least or up to about 2.0%, at least or up to about 2.5%, at least or up to about 3.0%, at least or up to about 3.5%, at least or up to about 4.0%, at least or up to about 4.5%, at least or up to about 5.0%, at least or up to about 5.5%, at least or up to about 6.0%, at least or up to about 6.5%, at least or up to about 7.0%, at least or up to about 7.5%, at least or up to about 8.0%, at least or up to about 8.5%, at least or up to about 9.0%, at least or up to about 9.5%, at least or up to about 10.0%, at least or up to about 10.5%, at least or up to about 11%, at least or up to about 11.5%, at least or up to about 12.0%, at least or up to about 12.5%, at least or up to about 13.0%, at least or up to about 13.5%, at least or up to about 14.0%, at least or up to about 14.5%, at least or up to about 15.0%, at least or up to about 15.5%, at least or up to about 16.0%, at least or up to about 16.5%, at least or up to about 17.0%, at least or up to about 17.5%, at least or up to about 18%, at least or up to about 18.5%, at least or up to about 19.0%, at least or up to about 19.5%, at least or up to about 20.0%, at least or up to about 20.5%, at least or up to about 21.0%, at least or up to about 21.5%, at least or up to about 22.0%, at least or up to about 22.5%, at least or up to about 23.0% at least or up to about, at least or up to about 23.5%, at least or up to about 24.0%, at least or up to about 24.5%, at least or up to about 25%, at least or up to about 25.5%, at least or up to about 26.0%, at least or up to about 26.5%, at least or up to about 27.0%, at least or up to about 27.5%, at least or up to about 28.0%, at least or up to about 28.5%, at least or up to about 29.0%, at least or up to about 29.5%, at least or up to about 30.0%, at least or up to about 30.5%, at least or up to about 31.0%, at least or up to about 31.5%, at least or up to about 32.0%, at least or up to about 32.5%, at least or up to about 33.0%, at least or up to about 33.5%, at least or up to about 34.0%, at least or up to about 34.5%, at least or up to about 35.0%, at least or up to about 35.5%, at least or up to about 36.0%, at least or up to about 36.5%, at least or up to about 37.0%, at least or up to about 37.5%, at least or up to about 38.0%, at least or up to about 38.5%, at least or up to about 39.0%, at least or up to about 39.5%, at least or up to about 40.0%, at least or up to about 40.5%, at least or up to about 41.0%, at least or up to about 41.5%, at least or up to about 42.0%, at least or up to about 42.5%, at least or up to about 43.0%, at least or up to about 43.5%, at least or up to about 44.0%, at least or up to about 44.5%, at least or up to about 45.0%, at least or up to about 45.5%, at least or up to about 46.0%, at least or up to about 46.5%, at least or up to about 47.0%, at least or up to about 47.5%, at least or up to about 48.0%, at least or up to about 48.5%, at least or up to about 49.0%, at least or up to about 49.5%, at least or up to about 50.0%, at least or up to about 50.5%, at least or up to about 51.0%, at least or up to about 51.5%, at least or up to about 52.0%, at least or up to about 52.5%, at least or up to about 53.0%, at least or up to about 53.5%, at least or up to about 54.0%, at least or up to about 54.5%, at least or up to about 55.0%, at least or up to about 55.5%, at least or up to about 56.0%, at least or up to about 56.5%, at least or up to about 57.0%, at least or up to about 57.5%, at least or up to about 58.0%, at least or up to about 58.5%, at least or up to about 59.0%, at least or up to about 59.5%, at least or up to about 60.0%, at least or up to about 60.5%, at least or up to about 61.0%, at least or up to about 61.5%, at least or up to about 62.0%, at least or up to about 62.5%, at least or up to about 63.0%, at least or up to about 63.5%, at least or up to about 64.0%, at least or up to about 64.5%, at least or up to about 65.0%, at least or up to about 65.5%, at least or up to about 66.0%, at least or up to about 66.5%, at least or up to about 67.0%, at least or up to about 67.5%, at least or up to about 68.0%, at least or up to about 68.5%, at least or up to about 69.0%, at least or up to about 69.5%, at least or up to about 70.0%, at least or up to about 70.5%, at least or up to about 71.0%, at least or up to about 71.5%, at least or up to about 72.0%, at least or up to about 72.5%, at least or up to about 73.0%, at least or up to about 73.5%, at least or up to about 74.0%, at least or up to about 74.5%, at least or up to about 75.0%, at least or up to about 75.5%, at least or up to about 76.0%, at least or up to about 76.5%, at least or up to about 77.0%, at least or up to about 77.5%, at least or up to about 78.0%, at least or up to about 78.5%, at least or up to about 79.0%, at least or up to about 79.5%, at least or up to about 80.0%, at least or up to about 80.5%, at least or up to about 81.0%, at least or up to about 81.5%, at least or up to about 82.0%, at least or up to about 82.5%, at least or up to about 83.0%, at least or up to about 83.5%, at least or up to about 84.0%, at least or up to about 84.5%, at least or up to about 85.0%, at least or up to about 85.5%, at least or up to about 86.0%, at least or up to about 86.5%, at least or up to about 87.0%, at least or up to about 87.5%, at least or up to about 88.0%, at least or up to about 88.5%, at least or up to about 89.0%, at least or up to about 89.5%, at least or up to about 90.0%, at least or up to about 90.5%, at least or up to about 91.0%, at least or up to about 91.5%, at least or up to about 92.0%, at least or up to about 92.5%, at least or up to about 93.0%, at least or up to about 93.5%, at least or up to about 94.0%, at least or up to about 94.5%, at least or up to about 95.0%, at least or up to about 95.5%, at least or up to about 96.0%, at least or up to about 96.5%, at least or up to about 97.0%, at least or up to about 97.5%, at least or up to about 98.0%, at least or up to about 98.5%, at least or up to about 99.0%, at least or up to about 99.5%, at least or up to about 100%, at least or up to about 101%, at least or up to about 102%, at least or up to about 103%, at least or up to about 104%, at least or up to about 105%, at least or up to about 106%, at least or up to about 107%, at least or up to about 108%, at least or up to about 109%, at least or up to about 110%, at least or up to about 111%, at least or up to about 112%, at least or up to about 113%, at least or up to about 114%, at least or up to about 115%, at least or up to about 116%, at least or up to about 117%, at least or up to about 118%, at least or up to about 119%, at least or up to about 120%, at least or up to about 121%, at least or up to about 122%, at least or up to about 123%, at least or up to about 124%, at least or up to about 125%, at least or up to about 126%, at least or up to about 127%, at least or up to about 128%, at least or up to about 129%, at least or up to about 130%, at least or up to about 131%, at least or up to about 132%, at least or up to about 133%, at least or up to about 134%, at least or up to about 135%, at least or up to about 136%, at least or up to about 137%, at least or up to about 138%, at least or up to about 139%, at least or up to about 140%, at least or up to about 141%, at least or up to about 142%, at least or up to about 143%, at least or up to about 144%, at least or up to about 145%, at least or up to about 146%, at least or up to about 147%, at least or up to about 148%, at least or up to about 149%, at least or up to about 150%, at least or up to about 151%, at least or up to about 152%, at least or up to about 153%, at least or up to about 154%, at least or up to about 155%, at least or up to about 156%, at least or up to about 157%, at least or up to about 158%, at least or up to about 159%, at least or up to about 160%, at least or up to about 161%, at least or up to about 162%, at least or up to about 163%, at least or up to about 164%, at least or up to about 165%, at least or up to about 166%, at least or up to about 167%, at least or up to about 168%, at least or up to about 169%, at least or up to about 170%, at least or up to about 171%, at least or up to about 172%, at least or up to about 173%, at least or up to about 174%, at least or up to about 175%, at least or up to about 176%, at least or up to about 177%, at least or up to about 178%, at least or up to about 179%, at least or up to about 180%, at least or up to about 181%, at least or up to about 182%, at least or up to about 183%, at least or up to about 184%, at least or up to about 185%, at least or up to about 186%, at least or up to about 187%, at least or up to about 188%, at least or up to about 189%, at least or up to about 190%, at least or up to about 191%, at least or up to about 192%, at least or up to about 193%, at least or up to about 194%, at least or up to about 195%, at least or up to about 196%, at least or up to about 197%, at least or up to about 198%, at least or up to about 199%, at least or up to about 200%, at least or up to about 201%, at least or up to about 202%, at least or up to about 203%, at least or up to about 204%, at least or up to about 205%, at least or up to about 206%, at least or up to about 207%, at least or up to about 208%, at least or up to about 209%, at least or up to about 210%, at least or up to about 211%, at least or up to about 212%, at least or up to about 213%, at least or up to about 214%, at least or up to about 215%, at least or up to about 216%, at least or up to about 217%, at least or up to about 218%, at least or up to about 219%, at least or up to about 220%, at least or up to about 221%, at least or up to about 222%, at least or up to about 223%, at least or up to about 224%, at least or up to about 225%, at least or up to about 226%, at least or up to about 227%, at least or up to about 228%, at least or up to about 229%, at least or up to about 230%, at least or up to about 231%, at least or up to about 232%, at least or up to about 233%, at least or up to about 234%, at least or up to about 235%, at least or up to about 236%, at least or up to about 237%, at least or up to about 238%, at least or up to about 239%, at least or up to about 240%, at least or up to about 241%, at least or up to about 242%, at least or up to about 243%, at least or up to about 244%, at least or up to about 245%, at least or up to about 246%, at least or up to about 247%, at least or up to about 248%, at least or up to about 249%, at least or up to about 250%, at least or up to about 251%, at least or up to about 252%, at least or up to about 253%, at least or up to about 254%, at least or up to about 255%, at least or up to about 256%, at least or up to about 257%, at least or up to about 258%, at least or up to about 259%, at least or up to about 260%, at least or up to about 261%, at least or up to about 262%, at least or up to about 263%, at least or up to about 264%, at least or up to about 265%, at least or up to about 266%, at least or up to about 267%, at least or up to about 268%, at least or up to about 269%, at least or up to about 270%, at least or up to about 271%, at least or up to about 272%, at least or up to about 273%, at least or up to about 274%, at least or up to about 275%, at least or up to about 276%, at least or up to about 277%, at least or up to about 278%, at least or up to about 279%, at least or up to about 280%, at least or up to about 281%, at least or up to about 282%, at least or up to about 283%, at least or up to about 284%, at least or up to about 285%, at least or up to about 286%, at least or up to about 287%, at least or up to about 288%, at least or up to about 289%, at least or up to about 290%, at least or up to about 291%, at least or up to about 292%, at least or up to about 293%, at least or up to about 294%, at least or up to about 295%, at least or up to about 296%, at least or up to about 297%, at least or up to about 298%, at least or up to about 299%, at least or up to about 300%, at least or up to about 301%, at least or up to about 302%, at least or up to about 303%, at least or up to about 304%, at least or up to about 305%, at least or up to about 306%, at least or up to about 307%, at least or up to about 308%, at least or up to about 309%, at least or up to about 310%, at least or up to about 311%, at least or up to about 312%, at least or up to about 313%, at least or up to about 314%, at least or up to about 315%, at least or up to about 316%, at least or up to about 317%, at least or up to about 318%, at least or up to about 319%, at least or up to about 320%, at least or up to about 321%, at least or up to about 322%, at least or up to about 323%, at least or up to about 324%, at least or up to about 325%, at least or up to about 326%, at least or up to about 327%, at least or up to about 328%, at least or up to about 329%, at least or up to about 330%, at least or up to about 331%, at least or up to about 332%, at least or up to about 333%, at least or up to about 334%, at least or up to about 335%, at least or up to about 336%, at least or up to about 337%, at least or up to about 338%, at least or up to about 339%, at least or up to about 340%, at least or up to about 341%, at least or up to about 342%, at least or up to about 343%, at least or up to about 344%, at least or up to about 345%, at least or up to about 346%, at least or up to about 347%, at least or up to about 348%, at least or up to about 349%, at least or up to about 350%, at least or up to about 351%, at least or up to about 352%, at least or up to about 353%, at least or up to about 354%, at least or up to about 355%, at least or up to about 356%, at least or up to about 357%, at least or up to about 358%, at least or up to about 359%, at least or up to about 360%, at least or up to about 361%, at least or up to about 362%, at least or up to about 363%, at least or up to about 364%, at least or up to about 365%, at least or up to about 366%, at least or up to about 367%, at least or up to about 368%, at least or up to about 369%, at least or up to about 370%, at least or up to about 371%, at least or up to about 372%, at least or up to about 373%, at least or up to about 374%, at least or up to about 375%, at least or up to about 376%, at least or up to about 377%, at least or up to about 378%, at least or up to about 379%, at least or up to about 380%, at least or up to about 381%, at least or up to about 382%, at least or up to about 383%, at least or up to about 384%, at least or up to about 385%, at least or up to about 386%, at least or up to about 387%, at least or up to about 388%, at least or up to about 389%, at least or up to about 390%, at least or up to about 391%, at least or up to about 392%, at least or up to about 393%, at least or up to about 394%, at least or up to about 395%, at least or up to about 396%, at least or up to about 397%, at least or up to about 398%, at least or up to about 399%, at least or up to about 400%, at least or up to about 401%, at least or up to about 402%, at least or up to about 403%, at least or up to about 404%, at least or up to about 405%, at least or up to about 406%, at least or up to about 407%, at least or up to about 408%, at least or up to about 409%, at least or up to about 410%, at least or up to about 411%, at least or up to about 412%, at least or up to about 413%, at least or up to about 414%, at least or up to about 415%, at least or up to about 416%, at least or up to about 417%, at least or up to about 418%, at least or up to about 419%, at least or up to about 420%, at least or up to about 421%, at least or up to about 422%, at least or up to about 423%, at least or up to about 424%, at least or up to about 425%, at least or up to about 426%, at least or up to about 427%, at least or up to about 428%, at least or up to about 429%, at least or up to about 430%, at least or up to about 431%, at least or up to about 432%, at least or up to about 433%, at least or up to about 434%, at least or up to about 435%, at least or up to about 436%, at least or up to about 437%, at least or up to about 438%, at least or up to about 439%, at least or up to about 440%, at least or up to about 441%, at least or up to about 442%, at least or up to about 443%, at least or up to about 444%, at least or up to about 445%, at least or up to about 446%, at least or up to about 447%, at least or up to about 448%, at least or up to about 449%, at least or up to about 450%, at least or up to about 451%, at least or up to about 452%, at least or up to about 453%, at least or up to about 454%, at least or up to about 455%, at least or up to about 456%, at least or up to about 457%, at least or up to about 458%, at least or up to about 459%, at least or up to about 460%, at least or up to about 461%, at least or up to about 462%, at least or up to about 463%, at least or up to about 464%, at least or up to about 465%, at least or up to about 466%, at least or up to about 467%, at least or up to about 468%, at least or up to about 469%, at least or up to about 470%, at least or up to about 471%, at least or up to about 472%, at least or up to about 473%, at least or up to about 474%, at least or up to about 475%, at least or up to about 476%, at least or up to about 477%, at least or up to about 478%, at least or up to about 479%, at least or up to about 480%, at least or up to about 481%, at least or up to about 482%, at least or up to about 483%, at least or up to about 484%, at least or up to about 485%, at least or up to about 486%, at least or up to about 487%, at least or up to about 488%, at least or up to about 489%, at least or up to about 490%, at least or up to about 491%, at least or up to about 492%, at least or up to about 493%, at least or up to about 494%, at least or up to about 495%, at least or up to about 496%, at least or up to about 497%, at least or up to about 498%, at least or up to about 499%, or at least or up to about 500% of the first target temperature and/or the second target temperature.

In some embodiments, a degree (e.g., an absolute value) of change of the temperature during an increasing temperature step and/or a decreasing temperature step of a fluctuation cycle of the plurality of fluctuation cycles can be a percent difference between the first target temperature and the second target temperature. In some embodiments, the percent difference of the degree of the change of the temperature during the increasing temperature step and/or the decreasing temperature step of the fluctuation cycle of the plurality of fluctuation cycles between the first target temperature and the second target temperature can be at least or up to about 0.0%, at least or up to about 0.5%, at least or up to about 1.0%, at least or up to about 1.5%, at least or up to about 2.0%, at least or up to about 2.5%, at least or up to about 3.0%, at least or up to about 3.5%, at least or up to about 4.0%, at least or up to about 4.5%, at least or up to about 5.0%, at least or up to about 5.5%, at least or up to about 6.0%, at least or up to about 6.5%, at least or up to about 7.0%, at least or up to about 7.5%, at least or up to about 8.0%, at least or up to about 8.5%, at least or up to about 9.0%, at least or up to about 9.5%, at least or up to about 10.0%, at least or up to about 10.5%, at least or up to about 11%, at least or up to about 11.5%, at least or up to about 12.0%, at least or up to about 12.5%, at least or up to about 13.0%, at least or up to about 13.5%, at least or up to about 14.0%, at least or up to about 14.5%, at least or up to about 15.0%, at least or up to about 15.5%, at least or up to about 16.0%, at least or up to about 16.5%, at least or up to about 17.0%, at least or up to about 17.5%, at least or up to about 18%, at least or up to about 18.5%, at least or up to about 19.0%, at least or up to about 19.5%, at least or up to about 20.0%, at least or up to about 20.5%, at least or up to about 21.0%, at least or up to about 21.5%, at least or up to about 22.0%, at least or up to about 22.5%, at least or up to about 23.0% at least or up to about, at least or up to about 23.5%, at least or up to about 24.0%, at least or up to about 24.5%, at least or up to about 25%, at least or up to about 25.5%, at least or up to about 26.0%, at least or up to about 26.5%, at least or up to about 27.0%, at least or up to about 27.5%, at least or up to about 28.0%, at least or up to about 28.5%, at least or up to about 29.0%, at least or up to about 29.5%, at least or up to about 30.0%, at least or up to about 30.5%, at least or up to about 31.0%, at least or up to about 31.5%, at least or up to about 32.0%, at least or up to about 32.5%, at least or up to about 33.0%, at least or up to about 33.5%, at least or up to about 34.0%, at least or up to about 34.5%, at least or up to about 35.0%, at least or up to about 35.5%, at least or up to about 36.0%, at least or up to about 36.5%, at least or up to about 37.0%, at least or up to about 37.5%, at least or up to about 38.0%, at least or up to about 38.5%, at least or up to about 39.0%, at least or up to about 39.5%, at least or up to about 40.0%, at least or up to about 40.5%, at least or up to about 41.0%, at least or up to about 41.5%, at least or up to about 42.0%, at least or up to about 42.5%, at least or up to about 43.0%, at least or up to about 43.5%, at least or up to about 44.0%, at least or up to about 44.5%, at least or up to about 45.0%, at least or up to about 45.5%, at least or up to about 46.0%, at least or up to about 46.5%, at least or up to about 47.0%, at least or up to about 47.5%, at least or up to about 48.0%, at least or up to about 48.5%, at least or up to about 49.0%, at least or up to about 49.5%, at least or up to about 50.0%, at least or up to about 50.5%, at least or up to about 51.0%, at least or up to about 51.5%, at least or up to about 52.0%, at least or up to about 52.5%, at least or up to about 53.0%, at least or up to about 53.5%, at least or up to about 54.0%, at least or up to about 54.5%, at least or up to about 55.0%, at least or up to about 55.5%, at least or up to about 56.0%, at least or up to about 56.5%, at least or up to about 57.0%, at least or up to about 57.5%, at least or up to about 58.0%, at least or up to about 58.5%, at least or up to about 59.0%, at least or up to about 59.5%, at least or up to about 60.0%, at least or up to about 60.5%, at least or up to about 61.0%, at least or up to about 61.5%, at least or up to about 62.0%, at least or up to about 62.5%, at least or up to about 63.0%, at least or up to about 63.5%, at least or up to about 64.0%, at least or up to about 64.5%, at least or up to about 65.0%, at least or up to about 65.5%, at least or up to about 66.0%, at least or up to about 66.5%, at least or up to about 67.0%, at least or up to about 67.5%, at least or up to about 68.0%, at least or up to about 68.5%, at least or up to about 69.0%, at least or up to about 69.5%, at least or up to about 70.0%, at least or up to about 70.5%, at least or up to about 71.0%, at least or up to about 71.5%, at least or up to about 72.0%, at least or up to about 72.5%, at least or up to about 73.0%, at least or up to about 73.5%, at least or up to about 74.0%, at least or up to about 74.5%, at least or up to about 75.0%, at least or up to about 75.5%, at least or up to about 76.0%, at least or up to about 76.5%, at least or up to about 77.0%, at least or up to about 77.5%, at least or up to about 78.0%, at least or up to about 78.5%, at least or up to about 79.0%, at least or up to about 79.5%, at least or up to about 80.0%, at least or up to about 80.5%, at least or up to about 81.0%, at least or up to about 81.5%, at least or up to about 82.0%, at least or up to about 82.5%, at least or up to about 83.0%, at least or up to about 83.5%, at least or up to about 84.0%, at least or up to about 84.5%, at least or up to about 85.0%, at least or up to about 85.5%, at least or up to about 86.0%, at least or up to about 86.5%, at least or up to about 87.0%, at least or up to about 87.5%, at least or up to about 88.0%, at least or up to about 88.5%, at least or up to about 89.0%, at least or up to about 89.5%, at least or up to about 90.0%, at least or up to about 90.5%, at least or up to about 91.0%, at least or up to about 91.5%, at least or up to about 92.0%, at least or up to about 92.5%, at least or up to about 93.0%, at least or up to about 93.5%, at least or up to about 94.0%, at least or up to about 94.5%, at least or up to about 95.0%, at least or up to about 95.5%, at least or up to about 96.0%, at least or up to about 96.5%, at least or up to about 97.0%, at least or up to about 97.5%, at least or up to about 98.0%, at least or up to about 98.5%, at least or up to about 99.0%, at least or up to about 99.5%, at least or up to about 100%, at least or up to about 101%, at least or up to about 102%, at least or up to about 103%, at least or up to about 104%, at least or up to about 105%, at least or up to about 106%, at least or up to about 107%, at least or up to about 108%, at least or up to about 109%, at least or up to about 110%, at least or up to about 111%, at least or up to about 112%, at least or up to about 113%, at least or up to about 114%, at least or up to about 115%, at least or up to about 116%, at least or up to about 117%, at least or up to about 118%, at least or up to about 119%, at least or up to about 120%, at least or up to about 121%, at least or up to about 122%, at least or up to about 123%, at least or up to about 124%, at least or up to about 125%, at least or up to about 126%, at least or up to about 127%, at least or up to about 128%, at least or up to about 129%, at least or up to about 130%, at least or up to about 131%, at least or up to about 132%, at least or up to about 133%, at least or up to about 134%, at least or up to about 135%, at least or up to about 136%, at least or up to about 137%, at least or up to about 138%, at least or up to about 139%, at least or up to about 140%, at least or up to about 141%, at least or up to about 142%, at least or up to about 143%, at least or up to about 144%, at least or up to about 145%, at least or up to about 146%, at least or up to about 147%, at least or up to about 148%, at least or up to about 149%, at least or up to about 150%, at least or up to about 151%, at least or up to about 152%, at least or up to about 153%, at least or up to about 154%, at least or up to about 155%, at least or up to about 156%, at least or up to about 157%, at least or up to about 158%, at least or up to about 159%, at least or up to about 160%, at least or up to about 161%, at least or up to about 162%, at least or up to about 163%, at least or up to about 164%, at least or up to about 165%, at least or up to about 166%, at least or up to about 167%, at least or up to about 168%, at least or up to about 169%, at least or up to about 170%, at least or up to about 171%, at least or up to about 172%, at least or up to about 173%, at least or up to about 174%, at least or up to about 175%, at least or up to about 176%, at least or up to about 177%, at least or up to about 178%, at least or up to about 179%, at least or up to about 180%, at least or up to about 181%, at least or up to about 182%, at least or up to about 183%, at least or up to about 184%, at least or up to about 185%, at least or up to about 186%, at least or up to about 187%, at least or up to about 188%, at least or up to about 189%, at least or up to about 190%, at least or up to about 191%, at least or up to about 192%, at least or up to about 193%, at least or up to about 194%, at least or up to about 195%, at least or up to about 196%, at least or up to about 197%, at least or up to about 198%, at least or up to about 199%, at least or up to about 200%, at least or up to about 201%, at least or up to about 202%, at least or up to about 203%, at least or up to about 204%, at least or up to about 205%, at least or up to about 206%, at least or up to about 207%, at least or up to about 208%, at least or up to about 209%, at least or up to about 210%, at least or up to about 211%, at least or up to about 212%, at least or up to about 213%, at least or up to about 214%, at least or up to about 215%, at least or up to about 216%, at least or up to about 217%, at least or up to about 218%, at least or up to about 219%, at least or up to about 220%, at least or up to about 221%, at least or up to about 222%, at least or up to about 223%, at least or up to about 224%, at least or up to about 225%, at least or up to about 226%, at least or up to about 227%, at least or up to about 228%, at least or up to about 229%, at least or up to about 230%, at least or up to about 231%, at least or up to about 232%, at least or up to about 233%, at least or up to about 234%, at least or up to about 235%, at least or up to about 236%, at least or up to about 237%, at least or up to about 238%, at least or up to about 239%, at least or up to about 240%, at least or up to about 241%, at least or up to about 242%, at least or up to about 243%, at least or up to about 244%, at least or up to about 245%, at least or up to about 246%, at least or up to about 247%, at least or up to about 248%, at least or up to about 249%, at least or up to about 250%, at least or up to about 251%, at least or up to about 252%, at least or up to about 253%, at least or up to about 254%, at least or up to about 255%, at least or up to about 256%, at least or up to about 257%, at least or up to about 258%, at least or up to about 259%, at least or up to about 260%, at least or up to about 261%, at least or up to about 262%, at least or up to about 263%, at least or up to about 264%, at least or up to about 265%, at least or up to about 266%, at least or up to about 267%, at least or up to about 268%, at least or up to about 269%, at least or up to about 270%, at least or up to about 271%, at least or up to about 272%, at least or up to about 273%, at least or up to about 274%, at least or up to about 275%, at least or up to about 276%, at least or up to about 277%, at least or up to about 278%, at least or up to about 279%, at least or up to about 280%, at least or up to about 281%, at least or up to about 282%, at least or up to about 283%, at least or up to about 284%, at least or up to about 285%, at least or up to about 286%, at least or up to about 287%, at least or up to about 288%, at least or up to about 289%, at least or up to about 290%, at least or up to about 291%, at least or up to about 292%, at least or up to about 293%, at least or up to about 294%, at least or up to about 295%, at least or up to about 296%, at least or up to about 297%, at least or up to about 298%, at least or up to about 299%, at least or up to about 300%, at least or up to about 301%, at least or up to about 302%, at least or up to about 303%, at least or up to about 304%, at least or up to about 305%, at least or up to about 306%, at least or up to about 307%, at least or up to about 308%, at least or up to about 309%, at least or up to about 310%, at least or up to about 311%, at least or up to about 312%, at least or up to about 313%, at least or up to about 314%, at least or up to about 315%, at least or up to about 316%, at least or up to about 317%, at least or up to about 318%, at least or up to about 319%, at least or up to about 320%, at least or up to about 321%, at least or up to about 322%, at least or up to about 323%, at least or up to about 324%, at least or up to about 325%, at least or up to about 326%, at least or up to about 327%, at least or up to about 328%, at least or up to about 329%, at least or up to about 330%, at least or up to about 331%, at least or up to about 332%, at least or up to about 333%, at least or up to about 334%, at least or up to about 335%, at least or up to about 336%, at least or up to about 337%, at least or up to about 338%, at least or up to about 339%, at least or up to about 340%, at least or up to about 341%, at least or up to about 342%, at least or up to about 343%, at least or up to about 344%, at least or up to about 345%, at least or up to about 346%, at least or up to about 347%, at least or up to about 348%, at least or up to about 349%, at least or up to about 350%, at least or up to about 351%, at least or up to about 352%, at least or up to about 353%, at least or up to about 354%, at least or up to about 355%, at least or up to about 356%, at least or up to about 357%, at least or up to about 358%, at least or up to about 359%, at least or up to about 360%, at least or up to about 361%, at least or up to about 362%, at least or up to about 363%, at least or up to about 364%, at least or up to about 365%, at least or up to about 366%, at least or up to about 367%, at least or up to about 368%, at least or up to about 369%, at least or up to about 370%, at least or up to about 371%, at least or up to about 372%, at least or up to about 373%, at least or up to about 374%, at least or up to about 375%, at least or up to about 376%, at least or up to about 377%, at least or up to about 378%, at least or up to about 379%, at least or up to about 380%, at least or up to about 381%, at least or up to about 382%, at least or up to about 383%, at least or up to about 384%, at least or up to about 385%, at least or up to about 386%, at least or up to about 387%, at least or up to about 388%, at least or up to about 389%, at least or up to about 390%, at least or up to about 391%, at least or up to about 392%, at least or up to about 393%, at least or up to about 394%, at least or up to about 395%, at least or up to about 396%, at least or up to about 397%, at least or up to about 398%, at least or up to about 399%, at least or up to about 400%, at least or up to about 401%, at least or up to about 402%, at least or up to about 403%, at least or up to about 404%, at least or up to about 405%, at least or up to about 406%, at least or up to about 407%, at least or up to about 408%, at least or up to about 409%, at least or up to about 410%, at least or up to about 411%, at least or up to about 412%, at least or up to about 413%, at least or up to about 414%, at least or up to about 415%, at least or up to about 416%, at least or up to about 417%, at least or up to about 418%, at least or up to about 419%, at least or up to about 420%, at least or up to about 421%, at least or up to about 422%, at least or up to about 423%, at least or up to about 424%, at least or up to about 425%, at least or up to about 426%, at least or up to about 427%, at least or up to about 428%, at least or up to about 429%, at least or up to about 430%, at least or up to about 431%, at least or up to about 432%, at least or up to about 433%, at least or up to about 434%, at least or up to about 435%, at least or up to about 436%, at least or up to about 437%, at least or up to about 438%, at least or up to about 439%, at least or up to about 440%, at least or up to about 441%, at least or up to about 442%, at least or up to about 443%, at least or up to about 444%, at least or up to about 445%, at least or up to about 446%, at least or up to about 447%, at least or up to about 448%, at least or up to about 449%, at least or up to about 450%, at least or up to about 451%, at least or up to about 452%, at least or up to about 453%, at least or up to about 454%, at least or up to about 455%, at least or up to about 456%, at least or up to about 457%, at least or up to about 458%, at least or up to about 459%, at least or up to about 460%, at least or up to about 461%, at least or up to about 462%, at least or up to about 463%, at least or up to about 464%, at least or up to about 465%, at least or up to about 466%, at least or up to about 467%, at least or up to about 468%, at least or up to about 469%, at least or up to about 470%, at least or up to about 471%, at least or up to about 472%, at least or up to about 473%, at least or up to about 474%, at least or up to about 475%, at least or up to about 476%, at least or up to about 477%, at least or up to about 478%, at least or up to about 479%, at least or up to about 480%, at least or up to about 481%, at least or up to about 482%, at least or up to about 483%, at least or up to about 484%, at least or up to about 485%, at least or up to about 486%, at least or up to about 487%, at least or up to about 488%, at least or up to about 489%, at least or up to about 490%, at least or up to about 491%, at least or up to about 492%, at least or up to about 493%, at least or up to about 494%, at least or up to about 495%, at least or up to about 496%, at least or up to about 497%, at least or up to about 498%, at least or up to about 499%, or at least or up to about 500%.

In some embodiments, a degree (e.g., an absolute value) of change of the temperature during an increasing temperature step and/or a decreasing temperature step of a fluctuation cycle of the plurality of fluctuation cycles can be a percent difference between the second target temperature and the first target temperature. In some embodiments, the percent difference of the degree of the change of the temperature during the increasing temperature step and/or the decreasing temperature step of the fluctuation cycle of the plurality of fluctuation cycles between the second target temperature and the first target temperature can be at least or up to about 0.0%, at least or up to about 0.5%, at least or up to about 1.0%, at least or up to about 1.5%, at least or up to about 2.0%, at least or up to about 2.5%, at least or up to about 3.0%, at least or up to about 3.5%, at least or up to about 4.0%, at least or up to about 4.5%, at least or up to about 5.0%, at least or up to about 5.5%, at least or up to about 6.0%, at least or up to about 6.5%, at least or up to about 7.0%, at least or up to about 7.5%, at least or up to about 8.0%, at least or up to about 8.5%, at least or up to about 9.0%, at least or up to about 9.5%, at least or up to about 10.0%, at least or up to about 10.5%, at least or up to about 11%, at least or up to about 11.5%, at least or up to about 12.0%, at least or up to about 12.5%, at least or up to about 13.0%, at least or up to about 13.5%, at least or up to about 14.0%, at least or up to about 14.5%, at least or up to about 15.0%, at least or up to about 15.5%, at least or up to about 16.0%, at least or up to about 16.5%, at least or up to about 17.0%, at least or up to about 17.5%, at least or up to about 18%, at least or up to about 18.5%, at least or up to about 19.0%, at least or up to about 19.5%, at least or up to about 20.0%, at least or up to about 20.5%, at least or up to about 21.0%, at least or up to about 21.5%, at least or up to about 22.0%, at least or up to about 22.5%, at least or up to about 23.0% at least or up to about, at least or up to about 23.5%, at least or up to about 24.0%, at least or up to about 24.5%, at least or up to about 25%, at least or up to about 25.5%, at least or up to about 26.0%, at least or up to about 26.5%, at least or up to about 27.0%, at least or up to about 27.5%, at least or up to about 28.0%, at least or up to about 28.5%, at least or up to about 29.0%, at least or up to about 29.5%, at least or up to about 30.0%, at least or up to about 30.5%, at least or up to about 31.0%, at least or up to about 31.5%, at least or up to about 32.0%, at least or up to about 32.5%, at least or up to about 33.0%, at least or up to about 33.5%, at least or up to about 34.0%, at least or up to about 34.5%, at least or up to about 35.0%, at least or up to about 35.5%, at least or up to about 36.0%, at least or up to about 36.5%, at least or up to about 37.0%, at least or up to about 37.5%, at least or up to about 38.0%, at least or up to about 38.5%, at least or up to about 39.0%, at least or up to about 39.5%, at least or up to about 40.0%, at least or up to about 40.5%, at least or up to about 41.0%, at least or up to about 41.5%, at least or up to about 42.0%, at least or up to about 42.5%, at least or up to about 43.0%, at least or up to about 43.5%, at least or up to about 44.0%, at least or up to about 44.5%, at least or up to about 45.0%, at least or up to about 45.5%, at least or up to about 46.0%, at least or up to about 46.5%, at least or up to about 47.0%, at least or up to about 47.5%, at least or up to about 48.0%, at least or up to about 48.5%, at least or up to about 49.0%, at least or up to about 49.5%, at least or up to about 50.0%, at least or up to about 50.5%, at least or up to about 51.0%, at least or up to about 51.5%, at least or up to about 52.0%, at least or up to about 52.5%, at least or up to about 53.0%, at least or up to about 53.5%, at least or up to about 54.0%, at least or up to about 54.5%, at least or up to about 55.0%, at least or up to about 55.5%, at least or up to about 56.0%, at least or up to about 56.5%, at least or up to about 57.0%, at least or up to about 57.5%, at least or up to about 58.0%, at least or up to about 58.5%, at least or up to about 59.0%, at least or up to about 59.5%, at least or up to about 60.0%, at least or up to about 60.5%, at least or up to about 61.0%, at least or up to about 61.5%, at least or up to about 62.0%, at least or up to about 62.5%, at least or up to about 63.0%, at least or up to about 63.5%, at least or up to about 64.0%, at least or up to about 64.5%, at least or up to about 65.0%, at least or up to about 65.5%, at least or up to about 66.0%, at least or up to about 66.5%, at least or up to about 67.0%, at least or up to about 67.5%, at least or up to about 68.0%, at least or up to about 68.5%, at least or up to about 69.0%, at least or up to about 69.5%, at least or up to about 70.0%, at least or up to about 70.5%, at least or up to about 71.0%, at least or up to about 71.5%, at least or up to about 72.0%, at least or up to about 72.5%, at least or up to about 73.0%, at least or up to about 73.5%, at least or up to about 74.0%, at least or up to about 74.5%, at least or up to about 75.0%, at least or up to about 75.5%, at least or up to about 76.0%, at least or up to about 76.5%, at least or up to about 77.0%, at least or up to about 77.5%, at least or up to about 78.0%, at least or up to about 78.5%, at least or up to about 79.0%, at least or up to about 79.5%, at least or up to about 80.0%, at least or up to about 80.5%, at least or up to about 81.0%, at least or up to about 81.5%, at least or up to about 82.0%, at least or up to about 82.5%, at least or up to about 83.0%, at least or up to about 83.5%, at least or up to about 84.0%, at least or up to about 84.5%, at least or up to about 85.0%, at least or up to about 85.5%, at least or up to about 86.0%, at least or up to about 86.5%, at least or up to about 87.0%, at least or up to about 87.5%, at least or up to about 88.0%, at least or up to about 88.5%, at least or up to about 89.0%, at least or up to about 89.5%, at least or up to about 90.0%, at least or up to about 90.5%, at least or up to about 91.0%, at least or up to about 91.5%, at least or up to about 92.0%, at least or up to about 92.5%, at least or up to about 93.0%, at least or up to about 93.5%, at least or up to about 94.0%, at least or up to about 94.5%, at least or up to about 95.0%, at least or up to about 95.5%, at least or up to about 96.0%, at least or up to about 96.5%, at least or up to about 97.0%, at least or up to about 97.5%, at least or up to about 98.0%, at least or up to about 98.5%, at least or up to about 99.0%, at least or up to about 99.5%, at least or up to about 100%, at least or up to about 101%, at least or up to about 102%, at least or up to about 103%, at least or up to about 104%, at least or up to about 105%, at least or up to about 106%, at least or up to about 107%, at least or up to about 108%, at least or up to about 109%, at least or up to about 110%, at least or up to about 111%, at least or up to about 112%, at least or up to about 113%, at least or up to about 114%, at least or up to about 115%, at least or up to about 116%, at least or up to about 117%, at least or up to about 118%, at least or up to about 119%, at least or up to about 120%, at least or up to about 121%, at least or up to about 122%, at least or up to about 123%, at least or up to about 124%, at least or up to about 125%, at least or up to about 126%, at least or up to about 127%, at least or up to about 128%, at least or up to about 129%, at least or up to about 130%, at least or up to about 131%, at least or up to about 132%, at least or up to about 133%, at least or up to about 134%, at least or up to about 135%, at least or up to about 136%, at least or up to about 137%, at least or up to about 138%, at least or up to about 139%, at least or up to about 140%, at least or up to about 141%, at least or up to about 142%, at least or up to about 143%, at least or up to about 144%, at least or up to about 145%, at least or up to about 146%, at least or up to about 147%, at least or up to about 148%, at least or up to about 149%, at least or up to about 150%, at least or up to about 151%, at least or up to about 152%, at least or up to about 153%, at least or up to about 154%, at least or up to about 155%, at least or up to about 156%, at least or up to about 157%, at least or up to about 158%, at least or up to about 159%, at least or up to about 160%, at least or up to about 161%, at least or up to about 162%, at least or up to about 163%, at least or up to about 164%, at least or up to about 165%, at least or up to about 166%, at least or up to about 167%, at least or up to about 168%, at least or up to about 169%, at least or up to about 170%, at least or up to about 171%, at least or up to about 172%, at least or up to about 173%, at least or up to about 174%, at least or up to about 175%, at least or up to about 176%, at least or up to about 177%, at least or up to about 178%, at least or up to about 179%, at least or up to about 180%, at least or up to about 181%, at least or up to about 182%, at least or up to about 183%, at least or up to about 184%, at least or up to about 185%, at least or up to about 186%, at least or up to about 187%, at least or up to about 188%, at least or up to about 189%, at least or up to about 190%, at least or up to about 191%, at least or up to about 192%, at least or up to about 193%, at least or up to about 194%, at least or up to about 195%, at least or up to about 196%, at least or up to about 197%, at least or up to about 198%, at least or up to about 199%, at least or up to about 200%, at least or up to about 201%, at least or up to about 202%, at least or up to about 203%, at least or up to about 204%, at least or up to about 205%, at least or up to about 206%, at least or up to about 207%, at least or up to about 208%, at least or up to about 209%, at least or up to about 210%, at least or up to about 211%, at least or up to about 212%, at least or up to about 213%, at least or up to about 214%, at least or up to about 215%, at least or up to about 216%, at least or up to about 217%, at least or up to about 218%, at least or up to about 219%, at least or up to about 220%, at least or up to about 221%, at least or up to about 222%, at least or up to about 223%, at least or up to about 224%, at least or up to about 225%, at least or up to about 226%, at least or up to about 227%, at least or up to about 228%, at least or up to about 229%, at least or up to about 230%, at least or up to about 231%, at least or up to about 232%, at least or up to about 233%, at least or up to about 234%, at least or up to about 235%, at least or up to about 236%, at least or up to about 237%, at least or up to about 238%, at least or up to about 239%, at least or up to about 240%, at least or up to about 241%, at least or up to about 242%, at least or up to about 243%, at least or up to about 244%, at least or up to about 245%, at least or up to about 246%, at least or up to about 247%, at least or up to about 248%, at least or up to about 249%, at least or up to about 250%, at least or up to about 251%, at least or up to about 252%, at least or up to about 253%, at least or up to about 254%, at least or up to about 255%, at least or up to about 256%, at least or up to about 257%, at least or up to about 258%, at least or up to about 259%, at least or up to about 260%, at least or up to about 261%, at least or up to about 262%, at least or up to about 263%, at least or up to about 264%, at least or up to about 265%, at least or up to about 266%, at least or up to about 267%, at least or up to about 268%, at least or up to about 269%, at least or up to about 270%, at least or up to about 271%, at least or up to about 272%, at least or up to about 273%, at least or up to about 274%, at least or up to about 275%, at least or up to about 276%, at least or up to about 277%, at least or up to about 278%, at least or up to about 279%, at least or up to about 280%, at least or up to about 281%, at least or up to about 282%, at least or up to about 283%, at least or up to about 284%, at least or up to about 285%, at least or up to about 286%, at least or up to about 287%, at least or up to about 288%, at least or up to about 289%, at least or up to about 290%, at least or up to about 291%, at least or up to about 292%, at least or up to about 293%, at least or up to about 294%, at least or up to about 295%, at least or up to about 296%, at least or up to about 297%, at least or up to about 298%, at least or up to about 299%, at least or up to about 300%, at least or up to about 301%, at least or up to about 302%, at least or up to about 303%, at least or up to about 304%, at least or up to about 305%, at least or up to about 306%, at least or up to about 307%, at least or up to about 308%, at least or up to about 309%, at least or up to about 310%, at least or up to about 311%, at least or up to about 312%, at least or up to about 313%, at least or up to about 314%, at least or up to about 315%, at least or up to about 316%, at least or up to about 317%, at least or up to about 318%, at least or up to about 319%, at least or up to about 320%, at least or up to about 321%, at least or up to about 322%, at least or up to about 323%, at least or up to about 324%, at least or up to about 325%, at least or up to about 326%, at least or up to about 327%, at least or up to about 328%, at least or up to about 329%, at least or up to about 330%, at least or up to about 331%, at least or up to about 332%, at least or up to about 333%, at least or up to about 334%, at least or up to about 335%, at least or up to about 336%, at least or up to about 337%, at least or up to about 338%, at least or up to about 339%, at least or up to about 340%, at least or up to about 341%, at least or up to about 342%, at least or up to about 343%, at least or up to about 344%, at least or up to about 345%, at least or up to about 346%, at least or up to about 347%, at least or up to about 348%, at least or up to about 349%, at least or up to about 350%, at least or up to about 351%, at least or up to about 352%, at least or up to about 353%, at least or up to about 354%, at least or up to about 355%, at least or up to about 356%, at least or up to about 357%, at least or up to about 358%, at least or up to about 359%, at least or up to about 360%, at least or up to about 361%, at least or up to about 362%, at least or up to about 363%, at least or up to about 364%, at least or up to about 365%, at least or up to about 366%, at least or up to about 367%, at least or up to about 368%, at least or up to about 369%, at least or up to about 370%, at least or up to about 371%, at least or up to about 372%, at least or up to about 373%, at least or up to about 374%, at least or up to about 375%, at least or up to about 376%, at least or up to about 377%, at least or up to about 378%, at least or up to about 379%, at least or up to about 380%, at least or up to about 381%, at least or up to about 382%, at least or up to about 383%, at least or up to about 384%, at least or up to about 385%, at least or up to about 386%, at least or up to about 387%, at least or up to about 388%, at least or up to about 389%, at least or up to about 390%, at least or up to about 391%, at least or up to about 392%, at least or up to about 393%, at least or up to about 394%, at least or up to about 395%, at least or up to about 396%, at least or up to about 397%, at least or up to about 398%, at least or up to about 399%, at least or up to about 400%, at least or up to about 401%, at least or up to about 402%, at least or up to about 403%, at least or up to about 404%, at least or up to about 405%, at least or up to about 406%, at least or up to about 407%, at least or up to about 408%, at least or up to about 409%, at least or up to about 410%, at least or up to about 411%, at least or up to about 412%, at least or up to about 413%, at least or up to about 414%, at least or up to about 415%, at least or up to about 416%, at least or up to about 417%, at least or up to about 418%, at least or up to about 419%, at least or up to about 420%, at least or up to about 421%, at least or up to about 422%, at least or up to about 423%, at least or up to about 424%, at least or up to about 425%, at least or up to about 426%, at least or up to about 427%, at least or up to about 428%, at least or up to about 429%, at least or up to about 430%, at least or up to about 431%, at least or up to about 432%, at least or up to about 433%, at least or up to about 434%, at least or up to about 435%, at least or up to about 436%, at least or up to about 437%, at least or up to about 438%, at least or up to about 439%, at least or up to about 440%, at least or up to about 441%, at least or up to about 442%, at least or up to about 443%, at least or up to about 444%, at least or up to about 445%, at least or up to about 446%, at least or up to about 447%, at least or up to about 448%, at least or up to about 449%, at least or up to about 450%, at least or up to about 451%, at least or up to about 452%, at least or up to about 453%, at least or up to about 454%, at least or up to about 455%, at least or up to about 456%, at least or up to about 457%, at least or up to about 458%, at least or up to about 459%, at least or up to about 460%, at least or up to about 461%, at least or up to about 462%, at least or up to about 463%, at least or up to about 464%, at least or up to about 465%, at least or up to about 466%, at least or up to about 467%, at least or up to about 468%, at least or up to about 469%, at least or up to about 470%, at least or up to about 471%, at least or up to about 472%, at least or up to about 473%, at least or up to about 474%, at least or up to about 475%, at least or up to about 476%, at least or up to about 477%, at least or up to about 478%, at least or up to about 479%, at least or up to about 480%, at least or up to about 481%, at least or up to about 482%, at least or up to about 483%, at least or up to about 484%, at least or up to about 485%, at least or up to about 486%, at least or up to about 487%, at least or up to about 488%, at least or up to about 489%, at least or up to about 490%, at least or up to about 491%, at least or up to about 492%, at least or up to about 493%, at least or up to about 494%, at least or up to about 495%, at least or up to about 496%, at least or up to about 497%, at least or up to about 498%, at least or up to about 499%, or at least or up to about 500%.

In some embodiments, the temperature of the bed device during a fluctuation cycle may increase and/or decrease (e.g., by the temperature regulator) at a rate ranging between about 0.01° C. per minute (° C./min) to about 5° C./min. In some cases, the temperature of the bed device during a fluctuation cycle may increase and/or decrease at a rate of at least or up to about 0.01° C./min, at least or up to about 0.02° C./min, at least or up to about 0.03° C./min, at least or up to about 0.04° C./min, at least or up to about 0.05° C./min, at least or up to about 0.06° C./min, at least or up to about 0.07° C./min, at least or up to about 0.08° C./min, at least or up to about 0.09° C./min, at least or up to about 0.1° C./min, at least or up to about 0.11° C./min, at least or up to about 0.12° C./min, at least or up to about 0.13° C./min, at least or up to about 0.14° C./min, at least or up to about 0.15° C./min, at least or up to about 0.16° C./min, at least or up to about 0.17° C./min, at least or up to about 0.18° C./min, at least or up to about 0.19° C./min, at least or up to about 0.2° C./min, at least or up to about 0.21° C./min, at least or up to about 0.22° C./min, at least or up to about 0.23° C./min, at least or up to about 0.24° C./min, at least or up to about 0.25° C./min, at least or up to about 0.26° C./min, at least or up to about 0.27° C./min, at least or up to about 0.28° C./min, at least or up to about 0.29° C./min, at least or up to about 0.3° C./min, at least or up to about 0.31° C./min, at least or up to about 0.32° C./min, at least or up to about 0.33° C./min, at least or up to about 0.34° C./min, at least or up to about 0.35° C./min, at least or up to about 0.36° C./min, at least or up to about 0.37° C./min, at least or up to about 0.38° C./min, at least or up to about 0.39° C./min, at least or up to about 0.4° C./min, at least or up to about 0.41° C./min, at least or up to about 0.42° C./min, at least or up to about 0.43° C./min, at least or up to about 0.44° C./min, at least or up to about 0.45° C./min, at least or up to about 0.46° C./min, at least or up to about 0.47° C./min, at least or up to about 0.48° C./min, at least or up to about 0.49° C./min, at least or up to about 0.5° C./min, at least or up to about 0.51° C./min, at least or up to about 0.52° C./min, at least or up to about 0.53° C./min, at least or up to about 0.54° C./min, at least or up to about 0.55° C./min, at least or up to about 0.56° C./min, at least or up to about 0.57° C./min, at least or up to about 0.58° C./min, at least or up to about 0.59° C./min, at least or up to about 0.6° C./min, at least or up to about 0.61° C./min, at least or up to about 0.62° C./min, at least or up to about 0.63° C./min, at least or up to about 0.64° C./min, at least or up to about 0.65° C./min, at least or up to about 0.66° C./min, at least or up to about 0.67° C./min, at least or up to about 0.68° C./min, at least or up to about 0.69° C./min, at least or up to about 0.7° C./min, at least or up to about 0.71° C./min, at least or up to about 0.72° C./min, at least or up to about 0.73° C./min, at least or up to about 0.74° C./min, at least or up to about 0.75° C./min, at least or up to about 0.76° C./min, at least or up to about 0.77° C./min, at least or up to about 0.78° C./min, at least or up to about 0.79° C./min, at least or up to about 0.8° C./min, at least or up to about 0.81° C./min, at least or up to about 0.82° C./min, at least or up to about 0.83° C./min, at least or up to about 0.84° C./min, at least or up to about 0.85° C./min, at least or up to about 0.86° C./min, at least or up to about 0.87° C./min, at least or up to about 0.88° C./min, at least or up to about 0.89° C./min, at least or up to about 0.9° C./min, at least or up to about 0.91° C./min, at least or up to about 0.92° C./min, at least or up to about 0.93° C./min, at least or up to about 0.94° C./min, at least or up to about 0.95° C./min, at least or up to about 0.96° C./min, at least or up to about 0.97° C./min, at least or up to about 0.98° C./min, at least or up to about 0.99° C./min, at least or up to about 1.0° C./min, at least or up to about 1.01° C./min, at least or up to about 1.02° C./min, at least or up to about 1.03° C./min, at least or up to about 1.04° C./min, at least or up to about 1.05° C./min, at least or up to about 1.06° C./min, at least or up to about 1.07° C./min, at least or up to about 1.08° C./min, at least or up to about 1.09° C./min, at least or up to about 1.1° C./min, at least or up to about 1.11° C./min, at least or up to about 1.12° C./min, at least or up to about 1.13° C./min, at least or up to about 1.14° C./min, at least or up to about 1.15° C./min, at least or up to about 1.16° C./min, at least or up to about 1.17° C./min, at least or up to about 1.18° C./min, at least or up to about 1.19° C./min, at least or up to about 1.2° C./min, at least or up to about 1.21° C./min, at least or up to about 1.22° C./min, at least or up to about 1.23° C./min, at least or up to about 1.24° C./min, at least or up to about 1.25° C./min, at least or up to about 1.26° C./min, at least or up to about 1.27° C./min, at least or up to about 1.28° C./min, at least or up to about 1.29° C./min, at least or up to about 1.3° C./min, at least or up to about 1.31° C./min, at least or up to about 1.32° C./min, at least or up to about 1.33° C./min, at least or up to about 1.34° C./min, at least or up to about 1.35° C./min, at least or up to about 1.36° C./min, at least or up to about 1.37° C./min, at least or up to about 1.38° C./min, at least or up to about 1.39° C./min, at least or up to about 1.4° C./min, at least or up to about 1.41° C./min, at least or up to about 1.42° C./min, at least or up to about 1.43° C./min, at least or up to about 1.44° C./min, at least or up to about 1.45° C./min, at least or up to about 1.46° C./min, at least or up to about 1.47° C./min, at least or up to about 1.48° C./min, at least or up to about 1.49° C./min, at least or up to about 1.5° C./min, at least or up to about 1.51° C./min, at least or up to about 1.52° C./min, at least or up to about 1.53° C./min, at least or up to about 1.54° C./min, at least or up to about 1.55° C./min, at least or up to about 1.56° C./min, at least or up to about 1.57° C./min, at least or up to about 1.58° C./min, at least or up to about 1.59° C./min, at least or up to about 1.6° C./min, at least or up to about 1.61° C./min, at least or up to about 1.62° C./min, at least or up to about 1.63° C./min, at least or up to about 1.64° C./min, at least or up to about 1.65° C./min, at least or up to about 1.66° C./min, at least or up to about 1.67° C./min, at least or up to about 1.68° C./min, at least or up to about 1.69° C./min, at least or up to about 1.7° C./min, at least or up to about 1.71° C./min, at least or up to about 1.72° C./min, at least or up to about 1.73° C./min, at least or up to about 1.74° C./min, at least or up to about 1.75° C./min, at least or up to about 1.76° C./min, at least or up to about 1.77° C./min, at least or up to about 1.78° C./min, at least or up to about 1.79° C./min, at least or up to about 1.8° C./min, at least or up to about 1.81° C./min, at least or up to about 1.82° C./min, at least or up to about 1.83° C./min, at least or up to about 1.84° C./min, at least or up to about 1.85° C./min, at least or up to about 1.86° C./min, at least or up to about 1.87° C./min, at least or up to about 1.88° C./min, at least or up to about 1.89° C./min, at least or up to about 1.9° C./min, at least or up to about 1.91° C./min, at least or up to about 1.92° C./min, at least or up to about 1.93° C./min, at least or up to about 1.94° C./min, at least or up to about 1.95° C./min, at least or up to about 1.96° C./min, at least or up to about 1.97° C./min, at least or up to about 1.98° C./min, at least or up to about 1.99° C./min, at least or up to about 2.0° C./min, at least or up to about 2.01° C./min, at least or up to about 2.02° C./min, at least or up to about 2.03° C./min, at least or up to about 2.04° C./min, at least or up to about 2.05° C./min, at least or up to about 2.06° C./min, at least or up to about 2.07° C./min, at least or up to about 2.08° C./min, at least or up to about 2.09° C./min, at least or up to about 2.1° C./min, at least or up to about 2.11° C./min, at least or up to about 2.12° C./min, at least or up to about 2.13° C./min, at least or up to about 2.14° C./min, at least or up to about 2.15° C./min, at least or up to about 2.16° C./min, at least or up to about 2.17° C./min, at least or up to about 2.18° C./min, at least or up to about 2.19° C./min, at least or up to about 2.2° C./min, at least or up to about 2.21° C./min, at least or up to about 2.22° C./min, at least or up to about 2.23° C./min, at least or up to about 2.24° C./min, at least or up to about 2.25° C./min, at least or up to about 2.26° C./min, at least or up to about 2.27° C./min, at least or up to about 2.28° C./min, at least or up to about 2.29° C./min, at least or up to about 2.3° C./min, at least or up to about 2.31° C./min, at least or up to about 2.32° C./min, at least or up to about 2.33° C./min, at least or up to about 2.34° C./min, at least or up to about 2.35° C./min, at least or up to about 2.36° C./min, at least or up to about 2.37° C./min, at least or up to about 2.38° C./min, at least or up to about 2.39° C./min, at least or up to about 2.4° C./min, at least or up to about 2.41° C./min, at least or up to about 2.42° C./min, at least or up to about 2.43° C./min, at least or up to about 2.44° C./min, at least or up to about 2.45° C./min, at least or up to about 2.46° C./min, at least or up to about 2.47° C./min, at least or up to about 2.48° C./min, at least or up to about 2.49° C./min, at least or up to about 2.5° C./min, at least or up to about 2.51° C./min, at least or up to about 2.52° C./min, at least or up to about 2.53° C./min, at least or up to about 2.54° C./min, at least or up to about 2.55° C./min, at least or up to about 2.56° C./min, at least or up to about 2.57° C./min, at least or up to about 2.58° C./min, at least or up to about 2.59° C./min, at least or up to about 2.6° C./min, at least or up to about 2.61° C./min, at least or up to about 2.62° C./min, at least or up to about 2.63° C./min, at least or up to about 2.64° C./min, at least or up to about 2.65° C./min, at least or up to about 2.66° C./min, at least or up to about 2.67° C./min, at least or up to about 2.68° C./min, at least or up to about 2.69° C./min, at least or up to about 2.7° C./min, at least or up to about 2.71° C./min, at least or up to about 2.72° C./min, at least or up to about 2.73° C./min, at least or up to about 2.74° C./min, at least or up to about 2.75° C./min, at least or up to about 2.76° C./min, at least or up to about 2.77° C./min, at least or up to about 2.78° C./min, at least or up to about 2.79° C./min, at least or up to about 2.8° C./min, at least or up to about 2.81° C./min, at least or up to about 2.82° C./min, at least or up to about 2.83° C./min, at least or up to about 2.84° C./min, at least or up to about 2.85° C./min, at least or up to about 2.86° C./min, at least or up to about 2.87° C./min, at least or up to about 2.88° C./min, at least or up to about 2.89° C./min, at least or up to about 2.9° C./min, at least or up to about 2.91° C./min, at least or up to about 2.92° C./min, at least or up to about 2.93° C./min, at least or up to about 2.94° C./min, at least or up to about 2.95° C./min, at least or up to about 2.96° C./min, at least or up to about 2.97° C./min, at least or up to about 2.98° C./min, at least or up to about 2.99° C./min, at least or up to about 3.0° C./min, at least or up to about 3.01° C./min, at least or up to about 3.02° C./min, at least or up to about 3.03° C./min, at least or up to about 3.04° C./min, at least or up to about 3.05° C./min, at least or up to about 3.06° C./min, at least or up to about 3.07° C./min, at least or up to about 3.08° C./min, at least or up to about 3.09° C./min, at least or up to about 3.1° C./min, at least or up to about 3.11° C./min, at least or up to about 3.12° C./min, at least or up to about 3.13° C./min, at least or up to about 3.14° C./min, at least or up to about 3.15° C./min, at least or up to about 3.16° C./min, at least or up to about 3.17° C./min, at least or up to about 3.18° C./min, at least or up to about 3.19° C./min, at least or up to about 3.2° C./min, at least or up to about 3.21° C./min, at least or up to about 3.22° C./min, at least or up to about 3.23° C./min, at least or up to about 3.24° C./min, at least or up to about 3.25° C./min, at least or up to about 3.26° C./min, at least or up to about 3.27° C./min, at least or up to about 3.28° C./min, at least or up to about 3.29° C./min, at least or up to about 3.3° C./min, at least or up to about 3.31° C./min, at least or up to about 3.32° C./min, at least or up to about 3.33° C./min, at least or up to about 3.34° C./min, at least or up to about 3.35° C./min, at least or up to about 3.36° C./min, at least or up to about 3.37° C./min, at least or up to about 3.38° C./min, at least or up to about 3.39° C./min, at least or up to about 3.4° C./min, at least or up to about 3.41° C./min, at least or up to about 3.42° C./min, at least or up to about 3.43° C./min, at least or up to about 3.44° C./min, at least or up to about 3.45° C./min, at least or up to about 3.46° C./min, at least or up to about 3.47° C./min, at least or up to about 3.48° C./min, at least or up to about 3.49° C./min, at least or up to about 3.5° C./min, at least or up to about 3.51° C./min, at least or up to about 3.52° C./min, at least or up to about 3.53° C./min, at least or up to about 3.54° C./min, at least or up to about 3.55° C./min, at least or up to about 3.56° C./min, at least or up to about 3.57° C./min, at least or up to about 3.58° C./min, at least or up to about 3.59° C./min, at least or up to about 3.6° C./min, at least or up to about 3.61° C./min, at least or up to about 3.62° C./min, at least or up to about 3.63° C./min, at least or up to about 3.64° C./min, at least or up to about 3.65° C./min, at least or up to about 3.66° C./min, at least or up to about 3.67° C./min, at least or up to about 3.68° C./min, at least or up to about 3.69° C./min, at least or up to about 3.7° C./min, at least or up to about 3.71° C./min, at least or up to about 3.72° C./min, at least or up to about 3.73° C./min, at least or up to about 3.74° C./min, at least or up to about 3.75° C./min, at least or up to about 3.76° C./min, at least or up to about 3.77° C./min, at least or up to about 3.78° C./min, at least or up to about 3.79° C./min, at least or up to about 3.8° C./min, at least or up to about 3.81° C./min, at least or up to about 3.82° C./min, at least or up to about 3.83° C./min, at least or up to about 3.84° C./min, at least or up to about 3.85° C./min, at least or up to about 3.86° C./min, at least or up to about 3.87° C./min, at least or up to about 3.88° C./min, at least or up to about 3.89° C./min, at least or up to about 3.9° C./min, at least or up to about 3.91° C./min, at least or up to about 3.92° C./min, at least or up to about 3.93° C./min, at least or up to about 3.94° C./min, at least or up to about 3.95° C./min, at least or up to about 3.96° C./min, at least or up to about 3.97° C./min, at least or up to about 3.98° C./min, at least or up to about 3.99° C./min, at least or up to about 4.0° C./min, at least or up to about 4.01° C./min, at least or up to about 4.02° C./min, at least or up to about 4.03° C./min, at least or up to about 4.04° C./min, at least or up to about 4.05° C./min, at least or up to about 4.06° C./min, at least or up to about 4.07° C./min, at least or up to about 4.08° C./min, at least or up to about 4.09° C./min, at least or up to about 4.1° C./min, at least or up to about 4.11° C./min, at least or up to about 4.12° C./min, at least or up to about 4.13° C./min, at least or up to about 4.14° C./min, at least or up to about 4.15° C./min, at least or up to about 4.16° C./min, at least or up to about 4.17° C./min, at least or up to about 4.18° C./min, at least or up to about 4.19° C./min, at least or up to about 4.2° C./min, at least or up to about 4.21° C./min, at least or up to about 4.22° C./min, at least or up to about 4.23° C./min, at least or up to about 4.24° C./min, at least or up to about 4.25° C./min, at least or up to about 4.26° C./min, at least or up to about 4.27° C./min, at least or up to about 4.28° C./min, at least or up to about 4.29° C./min, at least or up to about 4.3° C./min, at least or up to about 4.31° C./min, at least or up to about 4.32° C./min, at least or up to about 4.33° C./min, at least or up to about 4.34° C./min, at least or up to about 4.35° C./min, at least or up to about 4.36° C./min, at least or up to about 4.37° C./min, at least or up to about 4.38° C./min, at least or up to about 4.39° C./min, at least or up to about 4.4° C./min, at least or up to about 4.41° C./min, at least or up to about 4.42° C./min, at least or up to about 4.43° C./min, at least or up to about 4.44° C./min, at least or up to about 4.45° C./min, at least or up to about 4.46° C./min, at least or up to about 4.47° C./min, at least or up to about 4.48° C./min, at least or up to about 4.49° C./min, at least or up to about 4.5° C./min, at least or up to about 4.51° C./min, at least or up to about 4.52° C./min, at least or up to about 4.53° C./min, at least or up to about 4.54° C./min, at least or up to about 4.55° C./min, at least or up to about 4.56° C./min, at least or up to about 4.57° C./min, at least or up to about 4.58° C./min, at least or up to about 4.59° C./min, at least or up to about 4.6° C./min, at least or up to about 4.61° C./min, at least or up to about 4.62° C./min, at least or up to about 4.63° C./min, at least or up to about 4.64° C./min, at least or up to about 4.65° C./min, at least or up to about 4.66° C./min, at least or up to about 4.67° C./min, at least or up to about 4.68° C./min, at least or up to about 4.69° C./min, at least or up to about 4.7° C./min, at least or up to about 4.71° C./min, at least or up to about 4.72° C./min, at least or up to about 4.73° C./min, at least or up to about 4.74° C./min, at least or up to about 4.75° C./min, at least or up to about 4.76° C./min, at least or up to about 4.77° C./min, at least or up to about 4.78° C./min, at least or up to about 4.79° C./min, at least or up to about 4.8° C./min, at least or up to about 4.81° C./min, at least or up to about 4.82° C./min, at least or up to about 4.83° C./min, at least or up to about 4.84° C./min, at least or up to about 4.85° C./min, at least or up to about 4.86° C./min, at least or up to about 4.87° C./min, at least or up to about 4.88° C./min, at least or up to about 4.89° C./min, at least or up to about 4.9° C./min, at least or up to about 4.91° C./min, at least or up to about 4.92° C./min, at least or up to about 4.93° C./min, at least or up to about 4.94° C./min, at least or up to about 4.95° C./min, at least or up to about 4.96° C./min, at least or up to about 4.97° C./min, at least or up to about 4.98° C./min, at least or up to about 4.99° C./min, or at least or up to about 5.0° C./min.

In some embodiments, the bed device may comprise a plurality of zones (e.g., in accordance with a plurality of regions of a body of user of the bed device, such as a zone corresponding to a proximal/chest region of the user and an additional zone corresponding to a distal/limb region of the user), controlling the temperatures of the plurality of zones may utilize the same profile of the plurality of fluctuation cycles or different profiles of the plurality of fluctuation cycles. In some embodiments, the profile of the plurality of fluctuation cycles of the region of bed device corresponding to the proximal region of the user is the same as the profile of the plurality of fluctuation cycles of the region of the bed device corresponding to the distal region of the user. In some embodiments, the profile of the plurality of temperature settings of the region of bed device corresponding to the proximal region of the user is different from the profile of the plurality of temperature settings of the region of the bed device corresponding to the distal region of the user. In some embodiments, the profile of the plurality of temperature settings of the region of bed device corresponding to the proximal region of the user is the same as the profile of the plurality of temperature settings of the region of the bed device corresponding to the distal region of the user. In some embodiments, the profile of the plurality of temperature settings of the region of bed device corresponding to the proximal region of the user is different from the profile of the plurality of temperature settings of the region of the bed device corresponding to the distal region of the user.

In some embodiments, the region of the bed device that corresponds to the distal region of the user is a region of the bed device associated with the legs of the user. In some embodiments, the region of the bed device that corresponds to the distal region of the user is a region of the bed device associated with the feet of the user. In some embodiments, the region of the bed device that corresponds to the distal region of the user is a region of the bed device associated with the arms of the user. In some embodiments, the region of the bed device that corresponds to the distal region of the user is a region of the bed device associated with the hands of the user. In some embodiments, the region of the bed device that corresponds to the distal region of the user is a region of the bed device associated with the head of the user.

In some embodiments, the region of bed device that corresponds to the proximal region of the user is a region of the bed device associated with the chest of the user. In some embodiments, the region of the bed device that corresponds to the proximal region of the user is a region of the bed device associated with the hips of the user.

In some embodiments, the final temperature of bed device can fluctuate over the duration the bed device is directed or programmed to be at the final temperature (e.g., via the temperature control device as provided herein). In some embodiments, the final temperature fluctuates so the subject does not feel cold. Subjects who are on a bed device that maintains the same temperature for more than about 3 hours may feel cold and wake up. In some embodiments, the final temperature fluctuates between about 0° C. to about 5° C. In some embodiments, the final temperature fluctuates at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., or at least or up to about 5° C.

In some embodiments, the final temperature of the bed device fluctuates between every about 0 to about 60 minutes. In some embodiments, the final temperature of the bed device fluctuates every at least or up to about 0 minutes, at least or up to about 5 minutes, at least or up to about 10 minutes, at least or up to about 15 minutes, at least or up to about 20 minutes, at least or up to about 25 minutes, at least or up to about 30 minutes, at least or up to about 35 minutes, at least or up to about 40 minutes, at least or up to about 45 minutes, at least or up to about 50 minutes, at least or up to about 55 minutes, or at least or up to about 60 minutes.

In some embodiments, the dial value for the wake-up temperature of the bed device is between about −10 to about +10. In some embodiments, the dial value for the wake-up temperature of the bed device is at least or up to about 10, at least or up to about −9.5, at least or up to about −9, at least or up to about −8.5, at least or up to about −8, at least or up to about −7.5, at least or up to about −7, at least or up to about −6.5, at least or up to about −6, at least or up to about −5.5, at least or up to about −5, at least or up to about −4.5, at least or up to about −4, at least or up to about −3.5, at least or up to about −3, at least or up to about −2.5, at least or up to about −2, at least or up to about −1.5, at least or up to about −1, at least or up to about −0.5, at least or up to about 0, at least or up to about +0.5, at least or up to about +1, at least or up to about +1.5, at least or up to about +2, at least or up to about +2.5, at least or up to about +3, at least or up to about +3.5, at least or up to about +4, at least or up to about +4.5, at least or up to about +5, at least or up to about +5.5, at least or up to about +6, at least or up to about +6.5, at least or up to about +7, at least or up to about +7.5, at least or up to about +8, at least or up to about +8.5, at least or up to about +9, at least or up to about +9.5, or at least or up to about +10. In some embodiments, the wake-up temperature of the bed device is between about 12° C. to about 43° C. In some embodiments, the wake-up temperature of the bed device is at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., at least or up to about 31° C., at least or up to about 31.5° C., at least or up to about 32° C., at least or up to about 32.5° C., at least or up to about 33° C., at least or up to about 33.5° C., at least or up to about 34° C., at least or up to about 34.5° C., at least or up to about 35° C., at least or up to about 35.5° C., at least or up to about 36° C., at least or up to about 36.5° C., at least or up to about 37° C., at least or up to about 37.5° C., at least or up to about 38° C., at least or up to about 38.5° C., at least or up to about 39° C., at least or up to about 39.5° C., at least or up to about 40° C., at least or up to about 40.5° C., at least or up to about 41° C., at least or up to about 41.5° C., at least or up to about 42° C., at least or up to about 42.5° C., or at least or up to about 43° C.

In some embodiments, the wake-up temperature of bed device can fluctuate over the duration the bed device is directed or programmed to be at the wake-up temperature (e.g., via the temperature control device as provided herein). In some embodiments, the wake-up temperature fluctuates so the subject does not feel cold. Subjects who are on a bed device that maintains the same temperature for more than about 3 hours may feel cold and wake up. In some embodiments, the wake-up temperature fluctuates between about 0° C. to about 5° C. In some embodiments, the wake-up temperature fluctuates at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., or at least or up to about 5° C.

In some embodiments, the wake-up temperature of the bed device fluctuates between every about 0 to about 60 minutes. In some embodiments, the wake-up temperature of the bed device fluctuates every at least or up to about 0 minutes, at least or up to about 5 minutes, at least or up to about 10 minutes, at least or up to about 15 minutes, at least or up to about 20 minutes, at least or up to about 25 minutes, at least or up to about 30 minutes, at least or up to about 35 minutes, at least or up to about 40 minutes, at least or up to about 45 minutes, at least or up to about 50 minutes, at least or up to about 55 minutes, or at least or up to about 60 minutes.

In some embodiments, the rate of the temperature change of the bed device is between about 0.1° C. per minute (° C./min) to about 10° C./min. In some embodiments, the rate of the temperature change of the bed device is at least about 0.1° C./min, at least about 0.2° C./min, at least about 0.3° C./min, at least about 0.4° C./min, at least about 0.5° C./min, at least about 0.6° C./min, at least about 0.7° C./min, at least about 0.8° C./min, at least about 0.9° C./min, at least about 1° C./min, at least about 1.1° C./min, at least about 1.2° C./min, at least about 1.3° C./min, at least about 1.4° C./min, at least about 1.5° C./min, at least about 2° C./min, at least about 2.5° C./min, at least about 3° C./min, at least about 3.5° C./min, at least about 4° C./min, at least about 4.5° C./min, at least about 5° C./min, at least about 5.5° C./min, at least about 6° C./min, at least about 6.5° C./min, at least about 7° C./min, at least about 7.5° C./min, at least about 8° C./min, at least about 8.5° C./min, at least about 9° C./min, at least about 9.5° C./min, at least about 10° C./min, or more.

In some embodiments, the rate of the temperature change of the bed device is at most about 10° C./min, at most about 9.5° C./min, at most about 9° C./min, at most about 8.5° C./min, at most about 8° C./min, at most about 7.5° C./min, at most about 7° C./min, at most about 6.5° C./min, at most about 6° C./min, at most about 5.5° C./min, at most about 5° C./min, at most about 4.5° C./min, at most about 4° C./min, at most about 3.5° C./min, at most about 3° C./min, at most about 2.5° C./min at most about 2° C./min, at most about 1.5° C./min, at most about 1.4° C./min, at most abut 1.3° C./min, at most about 1.2° C./min, at most about 1.1° C./min, at most about 1° C./min, at most about 0.9° C./min, at most about 0.8° C./min, at most about 0.7° C./min, at most about 0.6° C./min, at most about 0.5° C./min, at most about 0.4° C./min, at most about 0.3° C./min, at most about 0.2° C./min, at most about 0.1° C./min, or less.

In some embodiments, the rate of the temperature change of the bed device is about 0.1° C./min, about 0.2° C./min, about 0.3° C./min, about 0.4° C./min, about 0.5° C./min, about 0.6° C./min, about 0.7° C./min, about 0.8° C./min, about 0.9° C./min, about 1° C./min, about 1.1° C./min, about 1.2° C./min, about 1.3° C./min, about 1.4° C./min, about 1.5° C./min, about 2° C./min, about 2.5° C./min, about 3° C./min, about 3.5° C./min, about 4° C./min, about 4.5° C./min, about 5° C./min, about 5.5° C./min, about 6° C./min, about 6.5° C./min, about 7° C./min, about 7.5° C./min, about 8° C./min, about 8.5° C./min, about 9° C./min, about 9.5° C./min, or about 10° C./min.

In some embodiments, the subject can update the dial values during the time the subject is on the bed device. In some embodiments, the incremental changes (e.g., increases) of the temperature of the bed device while the subject is on the bed device decreases the percent of dial value updates throughout the time while the subject is on the bed device. In some embodiments, the incremental changes (e.g., increases) of the temperature of the bed device decreases dial value updates between about 0% to about 500%. In some embodiments, the incremental changes (e.g., increases) of the temperature of the bed device decreases dial value updates by at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500%.

In some embodiments, the incremental changes (e.g., increases) of the temperature of the bed device decreases dial value updates by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less. In some embodiments, the incremental changes (e.g., increases) of the temperature of the bed device decreases dial value updates by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the incremental changes in temperature of the bed device enhances users experience of the bed device. In some embodiments, the incremental changes in temperature of the bed device enhances the user experience of the bed device by improving sleep quality of the user. In some embodiments, the incremental changes in temperature of the bed device improves the sleep quality values of the subject. Non-limiting examples of sleep quality values include percent light sleep, percent deep sleep, percent REM sleep, total sleep time, total awake time (e.g., number of awakenings per use of bed device), and net sleep score.

In some embodiments, the incremental changes (e.g., increases) of the temperature of the bed device results in an increase in percent change of light sleep. In some embodiments, the incremental changes (e.g., increases) of the temperature of the bed device results in between 0%-300% increase in percent change of light sleep. In some embodiments, the incremental changes (e.g., increases) of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change of light sleep.

In some embodiments, the incremental changes (e.g., increases) of the temperature of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of light sleep. In some embodiments, the incremental changes (e.g., increases) of the temperature of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change of light sleep.

In some embodiments, the incremental changes (e.g., increases) of the temperature of the bed device results in an increase in percent change of deep sleep. In some embodiments, the incremental changes (e.g., increases) of the temperature of the bed device results in between 0%-300% increase in percent change of deep sleep. In some embodiments, the incremental changes (e.g., increases) of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change of deep sleep.

In some embodiments, the incremental changes (e.g., increases) of the temperature of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of deep sleep. In some embodiments, the incremental changes (e.g., increases) of the temperature of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change of deep sleep.

In some embodiments, the incremental changes (e.g., increases) of the temperature of the bed device results in an increase in percent change of REM sleep. In some embodiments, the incremental changes of the temperature of the bed device results in between 0%-300% increase in percent change of REM sleep. In some embodiments, the incremental changes (e.g., increases) of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change of REM sleep.

In some embodiments, the incremental changes (e.g., increases) of the temperature of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of REM sleep. In some embodiments, the incremental changes of the temperature of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change of REM sleep.

In some embodiments, the incremental changes (e.g., increases) of the temperature of the bed device results in an increase in percent change of total sleep time. Total sleep time can correspond to the total amount of time the subject is asleep on the bed device. In some embodiments, the incremental changes (e.g., increases) of the temperature of the bed device results in between 0%-500% increase in percent change of total sleep time. In some embodiments, the incremental changes (e.g., increases) of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% increase in percent change of total sleep time.

In some embodiments, the incremental changes (e.g., increases) of the temperature of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of total sleep time. In some embodiments, the incremental changes (e.g., increases) of the temperature of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in percent change of total sleep time.

In some embodiments, the incremental changes (e.g., increases) of the temperature of the bed device results in an increase in percent change of a net sleep score. The net sleep score can be calculated by determining the percent change in how many subjects experienced a positive percent change in light, deep, REM, or total sleep time compared to a group that did not experience the incremental temperature increases (e.g., a control group). In some embodiments, the incremental changes (e.g., increases) of the temperature of the bed device results in between 0%-500% increase in percent change of net sleep score. In some embodiments, the incremental changes (e.g., increases) of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% increase in percent change of net sleep score.

In some embodiments, the incremental changes (e.g., increases) of the temperature of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of net sleep score. In some embodiments, the incremental changes (e.g., increases) of the temperature of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in percent change of net sleep score.

In some embodiments, the incremental changes (e.g., increases) of the temperature of the bed device results in a decrease in percent change of total awake time. The total awake time can be calculated by determining the length of time the user is awake during use of the bed device. In some cases, the total awake time can be the amount of time that the user is awake while on top of the bed device. In some embodiments, the incremental changes (e.g., increases) of the temperature of the bed device results in between 0%-500% decrease in percent change of total awake time. In some embodiments, the incremental changes (e.g., increases) of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% decrease in percent change of total awake time.

In some embodiments, the incremental changes (e.g., increases) of the temperature of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less decrease in percent change of total awake time. In some embodiments, the incremental changes (e.g., increases) of the temperature of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% decrease in percent change of total awake time.

In some embodiments, the incremental changes (e.g., increases) of the temperature of the bed device results in a decrease in percent change of the number of awakenings per use of the bed device. The number of awakenings can be calculated by determining the number of times the user awaken during use of the bed device. In some embodiments, the incremental changes (e.g., increases) of the temperature of the bed device results in between 0%-500% decrease in percent change of number of awakenings. In some embodiments, the incremental changes (e.g., increases) of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% decrease in percent change of numbers of awakenings.

In some embodiments, the incremental changes (e.g., increases) of the temperature of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less decrease in percent change of number of awakenings. In some embodiments, the incremental changes (e.g., increases) of the temperature of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% decrease in percent change of number of awakenings.

In some embodiments, the incremental changes (e.g., increases) of the temperature of the bed device results in improvements in light sleep, deep sleep, REM sleep, total sleep time, net sleep score, or any combination thereof for poor sleepers. Poor sleepers can be characterized as subjects who experience less than about 20% REM sleep, less than about 15% deep sleep, or less than about 7 hours of total sleep time. In some embodiments, the incremental changes (e.g., increases) of the temperature of the bed device results in improvements light sleep, deep sleep, REM sleep, total sleep time, net sleep score, or any combination thereof for subjects who experience at least or up to about 20% REM sleep. In some embodiments, the incremental changes (e.g., increases) of the temperature of the bed device results in improvements light sleep, deep sleep, REM sleep, total sleep time, net sleep score, or any combination thereof for subjects who experience at least or up to about 15% deep sleep. In some embodiments, the incremental changes (e.g., increases) of the temperature of the bed device results in improvements light sleep, deep sleep, REM sleep, total sleep time, net sleep score, or any combination thereof for subjects who experience less than about 7 hours of total sleep time.

In some embodiments, the incremental changes (e.g., increases) of the temperature of the bed device results in improvements in light sleep, deep sleep, REM sleep, total sleep time, net sleep score, or any combination thereof for regular sleepers. Regular sleepers can be characterized as subjects who experience at least or up to about 20% REM sleep, at least or up to about 15% deep sleep, or at least or up to about 7 hours of total sleep time. In some embodiments, the incremental changes (e.g., increases) of the temperature of the bed device results in improvements light sleep, deep sleep, REM sleep, total sleep time, net sleep score, or any combination thereof for subjects who experience at least or up to about 20% REM sleep. In some embodiments, the incremental changes (e.g., increases) of the temperature of the bed device results in improvements light sleep, deep sleep, REM sleep, total sleep time, net sleep score, or any combination thereof for subjects who experience at least or up to about 15% deep sleep. In some embodiments, the incremental changes (e.g., increases) of the temperature of the bed device results in improvements light sleep, deep sleep, REM sleep, total sleep time, net sleep score, or any combination thereof for subjects who experience at least or up to about 7 hours of total sleep time.

In some embodiments, the method (e.g., the computer-implemented method) further comprises automating updating the bedtime temperature, the initial temperature, or the final temperature based on dial value updates performed by the subject while on the surface of the bed device. In some embodiments, the bedtime temperature, the initial temperature, or the final temperature is automatically updated in the following use of bed device following the dial value update. In some embodiments, the following use of the bed device is the following night.

In some embodiments, the method (e.g., the computer-implemented method) comprises incrementally changing (e.g., increasing) the temperature of the bed device based on the lengths of the subject's sleep cycles. In some embodiments, the length the subject's sleep cycle can be determined by measuring the subject's HR, HRV, or respiration rate. In some embodiments, the subject's HR, HRV, or respiration rate decreases at the beginning of a sleep cycle. In some embodiments, the subject's HR, HRV, or respiration rate increases during REM cycle. In some embodiments, the length of subject's sleep cycle is measured as the length of time between decreases in the subject's HR, HRV, or respiration rate. In some embodiments, the length of the subject's sleep cycles is between about 70 to about 130 minutes. In some embodiments, the length of the subject's sleep cycles is at least or up to about 70 minutes, at least or up to about 75 minutes, at least or up to about 80 minutes, at least or up to about 85 minutes, at least or up to about 90 minutes, at least or up to about 95 minutes, at least or up to about 100 minutes, at least or up to about 105 minutes, at least or up to about 110 minutes, at least or up to about 115 minutes, at least or up to about 120 minutes, at least or up to about 125 minutes, or at least or up to about 130 minutes. In some embodiments, the method (e.g., the computer-implemented method) automatically adjusts the length of time between the incremental temperature changes (e.g., increases) based on the length of the subject's sleep cycles using the subject is using the bed device.

One aspect of the present disclosure provides a method (e.g., a computer-implemented method) for regulating the temperature of the bed device based on a subject's circadian rhythm. A subject's circadian rhythm may be the 24-hour internal clock in the subject's brain that regulates cycles of alertness and sleepiness in the subject by responding to light changes in our environment. While a subject is asleep, the circadian rhythm can be manifested in the occurrence of sleep cycles. A subject can experience multiple sleep cycles while the subject is asleep. Each sleep cycle of the multiple sleep cycles can be made up of several sleep stages. In some embodiments, each sleep cycle of the multiple sleep cycles can comprise a wake sleep stage, a light sleep stage, a deep sleep stage, a non-REM sleep stage, and a REM sleep stage. In some embodiments, the length of each sleep cycle of the multiple sleep cycles can increase while the subject is asleep.

In some embodiments, the bed device can comprise a profile of temperatures based on the sleep cycles of the user. In some cases, the profile of temperatures based on the sleep cycles of the user can comprise a plurality of different temperatures for a plurality of sleep cycles that the user experiences during a use of the bed device. In some cases, the user can experience between about 0 sleep cycles to about 20 sleep cycles during a use of the bed device. In some cases, the user can experience at least or up to about 0 sleep cycles, at least or up to about 1 sleep cycle, at least or up to about 2 sleep cycles, at least or up to about 3 sleep cycles, at least or up to about 4 sleep cycles, at least or up to about 5 sleep cycles, at least or up to about 6 sleep cycles, at least or up to about 7 sleep cycles, at least or up to about 8 sleep cycles, at least or up to about 9 sleep cycles, at least or up to about 10 sleep cycles, at least or up to about 11 sleep cycles, at least or up to about 12 sleep cycles, at least or up to about 13 sleep cycles, at least or up to about 14 sleep cycles, at least or up to about 15 sleep cycles, at least or up to about 16 sleep cycles, at least or up to about 17 sleep cycles, at least or up to about 18 sleep cycles, at least or up to about 19 sleep cycles, or at least or up to about 20 sleep cycles during a use of the bed device. In some cases, each sleep cycle of the plurality of sleep cycles can comprise a plurality of sleep stages. The plurality of sleep stages can include a wake stage, a light sleep stage, a deep sleep stage, and a REM sleep stage. In some cases, each sleep stage of the plurality of sleep stages can be associated with a temperature that enhances that sleep stage. In some examples, each sleep stage of the plurality of sleep stages can be associated with a different temperature that enhances that sleep stage.

In some embodiments, the bed device is able to detect which sleep stage the user is in during the entirety of the user's use of the bed device. In some cases, the bed device is able to detect which sleep stage the user is in every minute during the user's use of the bed device. In some cases, the sleep stage the user is in (e.g., experiencing) is determined by based the one or more biological signals detected by the bed device. In some cases, the one or more biological signals are provided to an algorithm to determine which sleep stage the user is in. In some embodiments, the bed device can adjust the temperature of the bed device automatically based on which sleep stage the user is in at the current moment time.

In some embodiments, the temperature that enhances deep sleep is lower than the temperature that enhances light sleep. In some cases, the temperature that enhances deep sleep is between about 0° C. to about 30° C. lower than the temperature that enhances light sleep. In some cases, the temperature that enhances deep sleep is at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., or at least or up to about 30° lower than the temperature that enhances light sleep.

In some embodiments, the temperature that enhances deep sleep is higher than the temperature that enhances light sleep. In some cases, the temperature that enhances deep sleep is between about 0° C. to about 30° C. higher than the temperature that enhances light sleep. In some cases, the temperature that enhances deep sleep is at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., or at least or up to about 30° higher than the temperature that enhances light sleep.

In some embodiments, the temperature that enhances deep sleep is lower than the temperature that enhances REM sleep. In some cases, the temperature that enhances deep sleep is between about 0° to about 30° lower than the temperature that enhances REM sleep. In some cases, the temperature that enhances deep sleep is at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., or at least or up to about 30° lower than the temperature that enhances REM sleep.

In some embodiments, the temperature that enhances deep sleep is higher than the temperature that enhances REM sleep. In some cases, the temperature that enhances deep sleep is between about 0° to about 30° higher than the temperature that enhances REM sleep. In some cases, the temperature that enhances deep sleep is at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., or at least or up to about 30° higher than the temperature that enhances REM sleep.

In some embodiments, the temperature that enhances light sleep is lower than the temperature that enhances REM sleep. In some cases, the temperature that enhances light sleep is between about 0° to about 30° lower than the temperature that enhances REM sleep. In some cases, the temperature that enhances light sleep is at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., or at least or up to about 30° lower than the temperature that enhances REM sleep.

In some embodiments, the temperature that enhances light sleep is higher than the temperature that enhances REM sleep. In some cases, the temperature that enhances light sleep is between about 0° to about 30° higher than the temperature that enhances REM sleep. In some cases, the temperature that enhances light sleep is at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., or at least or up to about 30° higher than the temperature that enhances REM sleep.

In some embodiments, the temperature that enhances light sleep is lower than the temperature that enhances wake sleep. In some cases, the temperature that enhances light sleep is between about 0° to about 30° lower than the temperature that enhances wake sleep. In some cases, the temperature that enhances light sleep is at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., or at least or up to about 30° lower than the temperature that enhances wake sleep.

In some embodiments, the temperature that enhances light sleep is higher than the temperature that enhances wake sleep. In some cases, the temperature that enhances light sleep is between about 0° to about 30° higher than the temperature that enhances wake sleep. In some cases, the temperature that enhances light sleep is at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., or at least or up to about 30° higher than the temperature that enhances wake sleep.

In some embodiments, the temperature that enhances deep sleep is lower than the temperature that enhances wake sleep. In some cases, the temperature that enhances deep sleep is between about 0° to about 30° lower than the temperature that enhances wake sleep. In some cases, the temperature that enhances deep sleep is at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., or at least or up to about 30° lower than the temperature that enhances wake sleep.

In some embodiments, the temperature that enhances deep sleep is higher than the temperature that enhances wake sleep. In some cases, the temperature that enhances deep sleep is between about 0° to about 30° higher than the temperature that enhances wake sleep. In some cases, the temperature that enhances deep sleep is at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., or at least or up to about 30° higher than the temperature that enhances wake sleep.

In some embodiments, the temperature that enhances REM sleep is lower than the temperature that enhances wake sleep. In some cases, the temperature that enhances REM sleep is between about 0° to about 30° lower than the temperature that enhances wake sleep. In some cases, the temperature that enhances REM sleep is at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., or at least or up to about 30° lower than the temperature that enhances wake sleep.

In some embodiments, the temperature that enhances REM sleep is higher than the temperature that enhances wake sleep. In some cases, the temperature that enhances REM sleep is between about 0° to about 30° higher than the temperature that enhances wake sleep. In some cases, the temperature that enhances REM sleep is at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., or at least or up to about 30° higher than the temperature that enhances wake sleep.

In some embodiments, the plurality of different temperatures can comprise between about a first temperature to about a 20th temperature. In some embodiments, the plurality of different temperatures can comprise a first temperature, a second temperature, and a third temperature. In some embodiments, the plurality of different temperatures can comprise a first temperature, a second temperature, a third temperature, and a fourth temperature. In some embodiments, the plurality of different temperatures can comprise a first temperature, a second temperature, a third temperature, a fourth temperature, and a fifth temperature. In some embodiments, the plurality of different temperatures can comprise a first temperature, a second temperature, a third temperature, a fourth temperature, a fifth temperature, and a sixth temperature. In some embodiments, the plurality of different temperatures can comprise a first temperature, a second temperature, a third temperature, a fourth temperature, a fifth temperature, a sixth temperature, and a seventh temperature. In some embodiments, the plurality of different temperatures can comprise a first temperature, a second temperature, a third temperature, a fourth temperature, a fifth temperature, a sixth temperature, a seventh temperature, and an eighth temperature. In some embodiments, the plurality of different temperatures can comprise a first temperature, a second temperature, a third temperature, a fourth temperature, a fifth temperature, a sixth temperature, a seventh temperature, an eight temperature, and a ninth temperature. In some embodiments, the plurality of different temperatures can comprise a first temperature, a second temperature, a third temperature, a fourth temperature, a fifth temperature, a sixth temperature, a seventh temperature, an eighth temperature, a ninth temperature, and a 10th temperature.

Alternatively, in some examples, each sleep stage of the plurality of sleep stages can be associates with the same temperature the enhances that sleep stage.

In some embodiments, the plurality of different temperatures of the plurality of sleep cycles can increase during a user of the bed device. In some cases, a first temperature of the plurality of different temperatures can be lower than a second temperature of the plurality of different temperatures. In some cases, the first temperature of the plurality of different temperatures can be between about 0° C. to about 10° C. lower than the second temperature of the plurality of different temperatures. In some cases, the first temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the second temperature of the plurality of different temperatures. In some cases, the second temperature of the plurality of different temperatures can be higher than the first temperature of the plurality of different temperatures. In some cases, the second temperature of the plurality of different temperatures can be between about 0° C. to about 10° C. higher than the first temperature of the plurality of different temperatures. In some cases, the second temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the first temperature of the plurality of different temperatures. In some cases, the second temperature of the plurality of different temperatures can be immediately preceded by the first temperature of the plurality of different temperatures.

In some cases, the second temperature of the plurality of different temperatures can be lower than a third temperature of the plurality of different temperatures. In some cases, the second temperature of the plurality of different temperatures can be between about 0° C. to about 10° C. lower than the third temperature of the plurality of different temperatures. In some cases, the second temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the third temperature of the plurality of different temperatures. In some cases, the third temperature of the plurality of different temperatures can be higher than the second temperature of the plurality of different temperatures. In some cases, the third temperature of the plurality of different temperatures can be between about 0° C. to about 10° C. higher than the second temperature of the plurality of different temperatures. In some cases, the third temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the second temperature of the plurality of different temperatures. In some cases, the third temperature of the plurality of different temperatures can be immediately preceded by the second temperature of the plurality of different temperatures.

In some cases, the third temperature of the plurality of different temperatures can be lower than a fourth temperature of the plurality of different temperatures. In some cases, the third temperature of the plurality of different temperatures can be between about 0° C. to about 10° C. lower than the fourth temperature of the plurality of different temperatures. In some cases, the third temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the fourth temperature of the plurality of different temperatures. In some cases, the fourth temperature of the plurality of different temperatures can be higher than the third temperature of the plurality of different temperatures. In some cases, the fourth temperature of the plurality of different temperatures can be between about 0° C. to about 10° C. higher than the third temperature of the plurality of different temperatures. In some cases, the fourth temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the third temperature of the plurality of different temperatures. In some cases, the fourth temperature of the plurality of different temperatures can be immediately preceded by the third temperature of the plurality of different temperatures.

In some cases, the fourth temperature of the plurality of different temperatures can be lower than a fifth temperature of the plurality of different temperatures. In some cases, the fourth temperature of the plurality of different temperatures can be between about 0° C. to about 10° C. lower than the fifth temperature of the plurality of different temperatures. In some cases, the fourth temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the fifth temperature of the plurality of different temperatures. In some cases, the fifth temperature of the plurality of different temperatures can be higher than the fourth temperature of the plurality of different temperatures. In some cases, the fifth temperature of the plurality of different temperatures can be between about 0° C. to about 10° C. higher than the fourth temperature of the plurality of different temperatures. In some cases, the fifth temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the fourth temperature of the plurality of different temperatures. In some cases, the fifth temperature of the plurality of different temperatures can be immediately preceded by the fourth temperature of the plurality of different temperatures.

In some cases, the fifth temperature of the plurality of different temperatures can be lower than a sixth temperature of the plurality of different temperatures. In some cases, the fifth temperature of the plurality of different temperatures can be between about 0° C. to about 10° C. lower than the sixth temperature of the plurality of different temperatures. In some cases, the fifth temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the sixth temperature of the plurality of different temperatures. In some cases, the sixth temperature of the plurality of different temperatures can be higher than the fifth temperature of the plurality of different temperatures. In some cases, the sixth temperature of the plurality of different temperatures can be between about 0° C. to about 10° C. higher than the fifth temperature of the plurality of different temperatures. In some cases, the sixth temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the fifth temperature of the plurality of different temperatures. In some cases, the sixth temperature of the plurality of different temperatures can be immediately preceded by the fifth temperature of the plurality of different temperatures.

In some cases, a sixth temperature of the plurality of different temperatures can be lower than a seventh temperature of the plurality of different temperatures. In some cases, the sixth temperature of the plurality of different temperatures can be between about 0° C. to about 10° C. lower than the seventh temperature of the plurality of different temperatures. In some cases, the sixth temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the seventh temperature of the plurality of different temperatures. In some cases, the seventh temperature of the plurality of different temperatures can be higher than the sixth temperature of the plurality of different temperatures. In some cases, the seventh temperature of the plurality of different temperatures can be between about 0° C. to about 10° C. higher than the sixth temperature of the plurality of different temperatures. In some cases, the seventh temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the sixth temperature of the plurality of different temperatures. In some cases, the seventh temperature of the plurality of different temperatures can be immediately preceded by the sixth temperature of the plurality of different temperatures.

In some cases, the seventh temperature of the plurality of different temperatures can be lower than an eighth temperature of the plurality of different temperatures. In some cases, the seventh temperature of the plurality of different temperatures can be between about 0° C. to about 10° C. lower than the eighth temperature of the plurality of different temperatures. In some cases, the seventh temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the eighth temperature of the plurality of different temperatures. In some cases, the eighth temperature of the plurality of different temperatures can be higher than the seventh temperature of the plurality of different temperatures. In some cases, the eighth temperature of the plurality of different temperatures can be between about 0° C. to about 10° C. higher than the seventh temperature of the plurality of different temperatures. In some cases, the eighth temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the seventh temperature of the plurality of different temperatures. In some cases, the eighth temperature of the plurality of different temperatures can be immediately preceded by the seventh temperature of the plurality of different temperatures.

In some cases, the eighth temperature of the plurality of different temperatures can be lower than a ninth temperature of the plurality of different temperatures. In some cases, the first eighth of the plurality of different temperatures can be between about 0° C. to about 10° C. lower than the ninth temperature of the plurality of different temperatures. In some cases, the eighth temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the eighth temperature of the plurality of different temperatures. In some cases, the ninth temperature of the plurality of different temperatures can be higher than the eighth temperature of the plurality of different temperatures. In some cases, the ninth temperature of the plurality of different temperatures can be between about 0° C. to about 10° C. higher than the eighth temperature of the plurality of different temperatures. In some cases, the ninth temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the eighth temperature of the plurality of different temperatures. In some cases, the ninth temperature of the plurality of different temperatures can be immediately preceded by the eighth temperature of the plurality of different temperatures.

In some cases, the ninth temperature of the plurality of different temperatures can be lower than a 10th temperature of the plurality of different temperatures. In some cases, the ninth temperature of the plurality of different temperatures can be between about 0° C. to about 10° C. lower than the 10th temperature of the plurality of different temperatures. In some cases, the ninth temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the 10th temperature of the plurality of different temperatures. In some cases, the 10th temperature of the plurality of different temperatures can be higher than the ninth temperature of the plurality of different temperatures. In some cases, the 10th temperature of the plurality of different temperatures can be between about 0° C. to about 10° C. higher than the ninth temperature of the plurality of different temperatures. In some cases, the 10th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the ninth temperature of the plurality of different temperatures. In some cases, the 10th temperature of the plurality of different temperatures can be immediately preceded by the ninth temperature of the plurality of different temperatures.

In some cases, the 10th temperature of the plurality of different temperatures can be lower than a 11th temperature of the plurality of different temperatures. In some cases, the 10th temperature of the plurality of different temperatures can be between about 0° C. to about 10° C. lower than the 11th temperature of the plurality of different temperatures. In some cases, the 10th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the 11th temperature of the plurality of different temperatures. In some cases, the 11th temperature of the plurality of different temperatures can be higher than the 10th temperature of the plurality of different temperatures. In some cases, the 11th temperature of the plurality of different temperatures can be between about 0° C. to about 10° C. higher than the 10th temperature of the plurality of different temperatures. In some cases, the 11th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the 10th temperature of the plurality of different temperatures. In some cases, the 11th temperature of the plurality of different temperatures can be immediately preceded by the 10th temperature of the plurality of different temperatures.

In some cases, the 11th temperature of the plurality of different temperatures can be lower than a 12th temperature of the plurality of different temperatures. In some cases, the 11th temperature of the plurality of different temperatures can be between about 0° C. to about 10° C. lower than the 12th temperature of the plurality of different temperatures. In some cases, the 11th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the 12th temperature of the plurality of different temperatures. In some cases, the 12th temperature of the plurality of different temperatures can be higher than the 11th temperature of the plurality of different temperatures. In some cases, the 12th temperature of the plurality of different temperatures can be between about 0° C. to about 10° C. higher than the 11th temperature of the plurality of different temperatures. In some cases, the 12th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the 11th temperature of the plurality of different temperatures. In some cases, the 12th temperature of the plurality of different temperatures can be immediately preceded by the 11th temperature of the plurality of different temperatures.

In some cases, the 12th temperature of the plurality of different temperatures can be lower than a 13th temperature of the plurality of different temperatures. In some cases, the 12th temperature of the plurality of different temperatures can be between about 0° C. to about 10° C. lower than the 13th temperature of the plurality of different temperatures. In some cases, the 12th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the 13th temperature of the plurality of different temperatures. In some cases, the 13th temperature of the plurality of different temperatures can be higher than the 12th temperature of the plurality of different temperatures. In some cases, the 13th temperature of the plurality of different temperatures can be between about 0° C. to about 10° C. higher than the 12th temperature of the plurality of different temperatures. In some cases, the 13th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the 12th temperature of the plurality of different temperatures. In some cases, the 13th temperature of the plurality of different temperatures can be immediately preceded by the 12th temperature of the plurality of different temperatures.

In some cases, the 13th temperature of the plurality of different temperatures can be lower than a 14th temperature of the plurality of different temperatures. In some cases, the 13th temperature of the plurality of different temperatures can be between about 0° C. to about 10° C. lower than the 14th temperature of the plurality of different temperatures. In some cases, the 13th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the 14th temperature of the plurality of different temperatures. In some cases, the 14th temperature of the plurality of different temperatures can be higher than the 13th temperature of the plurality of different temperatures. In some cases, the 14th temperature of the plurality of different temperatures can be between about 0° C. to about 10° C. higher than the 13th temperature of the plurality of different temperatures. In some cases, the 14th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the 13th temperature of the plurality of different temperatures. In some cases, the 14th temperature of the plurality of different temperatures can be immediately preceded by the 13th temperature of the plurality of different temperatures.

In some cases, the 14th temperature of the plurality of different temperatures can be lower than a 15th temperature of the plurality of different temperatures. In some cases, the 14th temperature of the plurality of different temperatures can be between about 0° C. to about 10° C. lower than the 15th temperature of the plurality of different temperatures. In some cases, the 14th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the 15th temperature of the plurality of different temperatures. In some cases, the 15th temperature of the plurality of different temperatures can be higher than the 14th temperature of the plurality of different temperatures. In some cases, the 15th temperature of the plurality of different temperatures can be between about 0° C. to about 10° C. higher than the 14th temperature of the plurality of different temperatures. In some cases, the 15th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the 14th temperature of the plurality of different temperatures. In some cases, the 15th temperature of the plurality of different temperatures can be immediately preceded by the 14th temperature of the plurality of different temperatures.

In some cases, the 15th temperature of the plurality of different temperatures can be lower than a 16th temperature of the plurality of different temperatures. In some cases, the 15th temperature of the plurality of different temperatures can be between about 0° C. to about 10° C. lower than the 16th temperature of the plurality of different temperatures. In some cases, the 15th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the 16th temperature of the plurality of different temperatures. In some cases, the 16th temperature of the plurality of different temperatures can be higher than the 15th temperature of the plurality of different temperatures. In some cases, the 16th temperature of the plurality of different temperatures can be between about 0° C. to about 10° C. higher than the 15th temperature of the plurality of different temperatures. In some cases, the 16th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the 15th temperature of the plurality of different temperatures. In some cases, the 16th temperature of the plurality of different temperatures can be immediately preceded by the 15th temperature of the plurality of different temperatures.

In some cases, the 16th temperature of the plurality of different temperatures can be lower than a 17th temperature of the plurality of different temperatures. In some cases, the 16th temperature of the plurality of different temperatures can be between about 0° C. to about 10° C. lower than the 17th temperature of the plurality of different temperatures. In some cases, the 16th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the 17th temperature of the plurality of different temperatures. In some cases, the 17th temperature of the plurality of different temperatures can be higher than the 16th temperature of the plurality of different temperatures. In some cases, the 17th temperature of the plurality of different temperatures can be between about 0° C. to about 10° C. higher than the 16th temperature of the plurality of different temperatures. In some cases, the 17th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the 16th temperature of the plurality of different temperatures. In some cases, the 17th temperature of the plurality of different temperatures can be immediately preceded by the 16th temperature of the plurality of different temperatures.

In some cases, the 17th temperature of the plurality of different temperatures can be lower than a 18th temperature of the plurality of different temperatures. In some cases, the 17th temperature of the plurality of different temperatures can be between about 0° C. to about 10° C. lower than the 18th temperature of the plurality of different temperatures. In some cases, the 17th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the 18th temperature of the plurality of different temperatures. In some cases, the 18th temperature of the plurality of different temperatures can be higher than the 17th temperature of the plurality of different temperatures. In some cases, the 18th temperature of the plurality of different temperatures can be between about 0° C. to about 10° C. higher than the 17th temperature of the plurality of different temperatures. In some cases, the 18th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the 17th temperature of the plurality of different temperatures. In some cases, the 18th temperature of the plurality of different temperatures can be immediately preceded by the 17th temperature of the plurality of different temperatures.

In some cases, the 18th temperature of the plurality of different temperatures can be lower than a 19th temperature of the plurality of different temperatures. In some cases, the 18th temperature of the plurality of different temperatures can be between about 0° C. to about 10° C. lower than the 19th temperature of the plurality of different temperatures. In some cases, the 18th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the 19th temperature of the plurality of different temperatures. In some cases, the 19th temperature of the plurality of different temperatures can be higher than the 18th temperature of the plurality of different temperatures. In some cases, the 19th temperature of the plurality of different temperatures can be between about 0° C. to about 10° C. higher than the 18th temperature of the plurality of different temperatures. In some cases, the 19th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the 18th temperature of the plurality of different temperatures. In some cases, the 19th temperature of the plurality of different temperatures can be immediately preceded by the 18th temperature of the plurality of different temperatures.

In some cases, the 19th temperature of the plurality of different temperatures can be lower than a 20th temperature of the plurality of different temperatures. In some cases, the 19th temperature of the plurality of different temperatures can be between about 0° C. to about 10° C. lower than the 20th temperature of the plurality of different temperatures. In some cases, the 19th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the 20th temperature of the plurality of different temperatures. In some cases, the 20th temperature of the plurality of different temperatures can be higher than the 19th temperature of the plurality of different temperatures. In some cases, the 20th temperature of the plurality of different temperatures can be between about 0° C. to about 10° C. higher than the 19th temperature of the plurality of different temperatures. In some cases, the 20th temperature of the plurality temperatures can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° higher than the 19th temperature of the plurality of different temperatures. In some cases, the 20th temperature of the plurality of different temperatures can be immediately preceded by the 19th temperature of the plurality of different temperatures.

In some embodiments, the first temperature of the plurality of different temperatures can comprise four or more different temperatures. In some embodiments, the second temperature of the plurality of different temperatures can comprise four or more different temperatures. In some embodiments, the third temperature of the plurality of different temperatures can comprise four or more different temperatures. In some embodiments, the fourth temperature of the plurality of different temperatures can comprise four or more different temperatures. In some embodiments, the fifth temperature of the plurality of different temperatures can comprise four or more different temperatures. In some embodiments, the sixth temperature of the plurality of different temperatures can comprise four or more different temperatures. In some embodiments, the seventh temperature of the plurality of different temperatures can comprise four or more different temperatures. In some embodiments, the eighth temperature of the plurality of different temperatures can comprise four or more different temperatures. In some embodiments, the ninth temperature of the plurality of different temperatures can comprise four or more different temperatures. In some embodiments, the 10th temperature of the plurality of different temperatures can comprise four or more different temperatures. In some embodiments, the 11^{th} temperature of the plurality of different temperatures can comprise four or more different temperatures. In some embodiments, the 12th temperature of the plurality of different temperatures can comprise four or more different temperatures. In some embodiments, the 13th temperature of the plurality of different temperatures can comprise four or more different temperatures. In some embodiments, the 14th temperature of the plurality of different temperatures can comprise four or more different temperatures. In some embodiments, the 15th temperature of the plurality of different temperatures can comprise four or more different temperatures. In some embodiments, the 16th temperature of the plurality of different temperatures can comprise four or more different temperatures. In some embodiments, the 17th temperature of the plurality of different temperatures can comprise four or more different temperatures. In some embodiments, the 18th temperature of the plurality of different temperatures can comprise four or more different temperatures. In some embodiments, the 19th temperature of the plurality of different temperatures can comprise four or more different temperatures. In some embodiments, the 20th temperature of the plurality of different temperatures can comprise four or more different temperatures. In some cases, the four or more different temperatures correspond to four or more different sleep stages of a sleep cycle.

In some embodiments, the four or more different temperatures can comprise a temperature for enhancing light sleep. In some cases, the four or more different temperatures can comprise a temperature for enhancing deep sleep. In some cases, the four or more different temperatures can comprise a temperature for enhancing REM sleep. In some cases, the four or more different temperatures can comprise a temperature for enhancing wake sleep. In some cases, the four or more different temperatures can comprise a temperature for enhancing light sleep and a temperature for enhancing deep sleep. In some cases, the four or more different temperatures can comprise a temperature for enhancing light sleep and a temperature of enhancing REM sleep. In some cases, the four or more different temperatures can comprise a temperature for enhancing deep sleep and a temperature for enhancing REM sleep. In some cases, the four or more different temperatures can comprise a temperature for enhancing wake sleep and a temperature for enhancing light sleep. In some cases, the four or more different temperatures can comprise a temperature for enhancing wake sleep and a temperature for enhancing REM sleep. In some cases, the four or more different temperatures can comprise a temperature for enhancing wake sleep and a temperature for enhancing deep sleep. In some cases, the four or more different temperatures can comprise a temperature for enhancing light sleep, a temperature for enhancing deep sleep, and a temperature for enhancing REM sleep. In some cases, the four or more different temperatures can comprise a temperature for enhancing wake sleep, a temperature for enhancing light sleep, and a temperature for enhancing REM sleep. In some cases, the four or more different temperatures can comprise a temperature for enhancing wake sleep, a temperature for enhancing light sleep, and a temperature for enhancing deep sleep. In some cases, the four or more different temperatures can comprise a temperature for enhancing wake sleep, a temperature for enhancing REM sleep, and a temperature for enhancing deep sleep. In some cases, the four or more different temperatures can comprise a temperature for enhancing wake sleep, a temperature for enhancing light sleep, a temperature for enhancing deep sleep, and a temperature for enhancing REM sleep. In some cases, the four or more different temperatures are different from each other.

In some cases, each sleep stage of a sleep cycle can be enhanced based on the temperature of the subject while sleeping. In some cases, an amount of sleep in a sleep stage of the sleep cycle can be increased based on the temperature of the subject while sleeping in that sleep stage.

In some embodiments, the temperature for enhancing wake sleep of the user can be between about 12° C. to about 43° C. In some embodiments, the temperature for enhancing wake sleep of the user can be at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., at least or up to about 31° C., at least or up to about 31.5° C., at least or up to about 32° C., at least or up to about 32.5° C., at least or up to about 33° C., at least or up to about 33.5° C., at least or up to about 34° C., at least or up to about 34.5° C., at least or up to about 35° C., at least or up to about 35.5° C., at least or up to about 36° C., at least or up to about 36.5° C., at least or up to about 37° C., at least or up to about 37.5° C., at least or up to about 38° C., at least or up to about 38.5° C., at least or up to about 39° C., at least or up to about 39.5° C., at least or up to about 40° C., at least or up to about 40.5° C., at least or up to about 41° C., at least or up to about 41.5° C., at least or up to about 42° C., at least or up to about 42.5° C., or at least or up to about 43° C. In some cases, a user sleeping at the temperature for enhancing wake sleep can result in an increase of the amount of wake sleep the user experiences while sleeping. In some embodiments, the amount of wake sleep the user experiences while sleeping at the temperature for enhancing wake sleep can be increased by between about 0% to about 500% compared to a user not sleeping at the temperature to enhance wake sleep. In some cases, the user not sleeping at the temperature to enhance wake sleep can be a separate user or the same user prior to using the temperature to enhance wake sleep. In some cases, the amount of wake sleep the user experiences while sleeping at the temperature for enhancing wake sleep can be increased by at least or up to about 0%, at least or up to about 5%, at least or up to about 10%, at least or up to about 15%, at least or up to about 20%, at least or up to about 25%, at least or up to about 30%, at least or up to about 35%, at least or up to about 40%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 95%, at least or up to about 98%, at least or up to about 99%, at least or up to about 100%, at least or up to about 110%, at least or up to about 120%, at least or up to about 130%, at least or up to about 140%, at least or up to about 150%, at least or up to about 160%, at least or up to about 170%, at least or up to about 180%, at least or up to about 190%, at least or up to about 200%, at least or up to about 210%, at least or up to about 220%, at least or up to about 230%, at least or up to about 240%, at least or up to about 250%, at least or up to about 260%, at least or up to about 270%, at least or up to about 280%, at least or up to about 290%, at least or up to about 300%, at least or up to about 310%, at least or up to about 320%, at least or up to about 330%, at least or up to about 340%, at least or up to about 350%, at least or up to about 360%, at least or up to about 370%, at least or up to about 380%, at least or up to about 390%, at least or up to about 400%, at least or up to about 410%, at least or up to about 420%, at least or up to about 430%, at least or up to about 440%, at least or up to about 450%, at least or up to about 460%, at least or up to about 470%, at least or up to about 480%, at least or up to about 490%, or at least or up to about 500% compared to a user not sleeping at the temperature to enhance wake sleep.

In some embodiments, the temperature for enhancing light sleep of the user can be between about 12° C. to about 43° C. In some embodiments, the temperature for enhancing light sleep of the user can be at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., at least or up to about 31° C., at least or up to about 31.5° C., at least or up to about 32° C., at least or up to about 32.5° C., at least or up to about 33° C., at least or up to about 33.5° C., at least or up to about 34° C., at least or up to about 34.5° C., at least or up to about 35° C., at least or up to about 35.5° C., at least or up to about 36° C., at least or up to about 36.5° C., at least or up to about 37° C., at least or up to about 37.5° C., at least or up to about 38° C., at least or up to about 38.5° C., at least or up to about 39° C., at least or up to about 39.5° C., at least or up to about 40° C., at least or up to about 40.5° C., at least or up to about 41° C., at least or up to about 41.5° C., at least or up to about 42° C., at least or up to about 42.5° C., or at least or up to about 43° C. In some cases, a user sleeping at the temperature for enhancing light sleep can result in an increase of the amount of light sleep the user experiences while sleeping. In some embodiments, the amount of light sleep the user experiences while sleeping at the temperature for enhancing light sleep can be increased by between about 0% to about 500% compared to a user not sleeping at the temperature to enhance light sleep. In some cases, the user not sleeping at the temperature to enhance light sleep can be a separate user or the same user prior to using the temperature to enhance light sleep. In some cases, the amount of light sleep the user experiences while sleeping at the temperature for enhancing light sleep can be increased by at least or up to about 0%, at least or up to about 5%, at least or up to about 10%, at least or up to about 15%, at least or up to about 20%, at least or up to about 25%, at least or up to about 30%, at least or up to about 35%, at least or up to about 40%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 95%, at least or up to about 98%, at least or up to about 99%, at least or up to about 100%, at least or up to about 110%, at least or up to about 120%, at least or up to about 130%, at least or up to about 140%, at least or up to about 150%, at least or up to about 160%, at least or up to about 170%, at least or up to about 180%, at least or up to about 190%, at least or up to about 200%, at least or up to about 210%, at least or up to about 220%, at least or up to about 230%, at least or up to about 240%, at least or up to about 250%, at least or up to about 260%, at least or up to about 270%, at least or up to about 280%, at least or up to about 290%, at least or up to about 300%, at least or up to about 310%, at least or up to about 320%, at least or up to about 330%, at least or up to about 340%, at least or up to about 350%, at least or up to about 360%, at least or up to about 370%, at least or up to about 380%, at least or up to about 390%, at least or up to about 400%, at least or up to about 410%, at least or up to about 420%, at least or up to about 430%, at least or up to about 440%, at least or up to about 450%, at least or up to about 460%, at least or up to about 470%, at least or up to about 480%, at least or up to about 490%, or at least or up to about 500% compared to a user not sleeping at the temperature to enhance light sleep.

In some embodiments, the temperature for enhancing deep sleep of the user can be between about 12° C. to about 43° C. In some embodiments, the temperature for enhancing deep sleep of the user can be at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., at least or up to about 31° C., at least or up to about 31.5° C., at least or up to about 32° C., at least or up to about 32.5° C., at least or up to about 33° C., at least or up to about 33.5° C., at least or up to about 34° C., at least or up to about 34.5° C., at least or up to about 35° C., at least or up to about 35.5° C., at least or up to about 36° C., at least or up to about 36.5° C., at least or up to about 37° C., at least or up to about 37.5° C., at least or up to about 38° C., at least or up to about 38.5° C., at least or up to about 39° C., at least or up to about 39.5° C., at least or up to about 40° C., at least or up to about 40.5° C., at least or up to about 41° C., at least or up to about 41.5° C., at least or up to about 42° C., at least or up to about 42.5° C., or at least or up to about 43° C. In some cases, a user sleeping at the temperature for enhancing deep sleep can result in an increase of the amount of deep sleep the user experiences while sleeping. In some embodiments, the amount of deep sleep the user experiences while sleeping at the temperature for enhancing deep sleep can be increased by between about 0% to about 500% compared to a user not sleeping at the temperature to enhance deep sleep. In some cases, the user not sleeping at the temperature to enhance deep sleep can be a separate user or the same user prior to using the temperature to enhance deep sleep. In some cases, the amount of deep sleep the user experiences while sleeping at the temperature for enhancing deep sleep can be increased by at least or up to about 0%, at least or up to about 5%, at least or up to about 10%, at least or up to about 15%, at least or up to about 20%, at least or up to about 25%, at least or up to about 30%, at least or up to about 35%, at least or up to about 40%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 95%, at least or up to about 98%, at least or up to about 99%, at least or up to about 100%, at least or up to about 110%, at least or up to about 120%, at least or up to about 130%, at least or up to about 140%, at least or up to about 150%, at least or up to about 160%, at least or up to about 170%, at least or up to about 180%, at least or up to about 190%, at least or up to about 200%, at least or up to about 210%, at least or up to about 220%, at least or up to about 230%, at least or up to about 240%, at least or up to about 250%, at least or up to about 260%, at least or up to about 270%, at least or up to about 280%, at least or up to about 290%, at least or up to about 300%, at least or up to about 310%, at least or up to about 320%, at least or up to about 330%, at least or up to about 340%, at least or up to about 350%, at least or up to about 360%, at least or up to about 370%, at least or up to about 380%, at least or up to about 390%, at least or up to about 400%, at least or up to about 410%, at least or up to about 420%, at least or up to about 430%, at least or up to about 440%, at least or up to about 450%, at least or up to about 460%, at least or up to about 470%, at least or up to about 480%, at least or up to about 490%, or at least or up to about 500% compared to a user not sleeping at the temperature to enhance deep sleep.

In some embodiments, the temperature for enhancing REM sleep of the user can be between about 12° C. to about 43° C. In some embodiments, the temperature for enhancing REM sleep of the user can be at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., at least or up to about 31° C., at least or up to about 31.5° C., at least or up to about 32° C., at least or up to about 32.5° C., at least or up to about 33° C., at least or up to about 33.5° C., at least or up to about 34° C., at least or up to about 34.5° C., at least or up to about 35° C., at least or up to about 35.5° C., at least or up to about 36° C., at least or up to about 36.5° C., at least or up to about 37° C., at least or up to about 37.5° C., at least or up to about 38° C., at least or up to about 38.5° C., at least or up to about 39° C., at least or up to about 39.5° C., at least or up to about 40° C., at least or up to about 40.5° C., at least or up to about 41° C., at least or up to about 41.5° C., at least or up to about 42° C., at least or up to about 42.5° C., or at least or up to about 43° C. In some cases, a user sleeping at the temperature for enhancing REM sleep can result in an increase of the amount of REM sleep the user experiences while sleeping. In some embodiments, the amount of REM sleep the user experiences while sleeping at the temperature for enhancing REM sleep can be increased by between about 0% to about 500% compared to a user not sleeping at the temperature to enhance REM sleep. In some cases, the user not sleeping at the temperature to enhance REM sleep can be a separate user or the same user prior to using the temperature to enhance REM sleep. In some cases, the amount of REM sleep the user experiences while sleeping at the temperature for enhancing REM sleep can be increased by at least or up to about 0%, at least or up to about 5%, at least or up to about 10%, at least or up to about 15%, at least or up to about 20%, at least or up to about 25%, at least or up to about 30%, at least or up to about 35%, at least or up to about 40%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 95%, at least or up to about 98%, at least or up to about 99%, at least or up to about 100%, at least or up to about 110%, at least or up to about 120%, at least or up to about 130%, at least or up to about 140%, at least or up to about 150%, at least or up to about 160%, at least or up to about 170%, at least or up to about 180%, at least or up to about 190%, at least or up to about 200%, at least or up to about 210%, at least or up to about 220%, at least or up to about 230%, at least or up to about 240%, at least or up to about 250%, at least or up to about 260%, at least or up to about 270%, at least or up to about 280%, at least or up to about 290%, at least or up to about 300%, at least or up to about 310%, at least or up to about 320%, at least or up to about 330%, at least or up to about 340%, at least or up to about 350%, at least or up to about 360%, at least or up to about 370%, at least or up to about 380%, at least or up to about 390%, at least or up to about 400%, at least or up to about 410%, at least or up to about 420%, at least or up to about 430%, at least or up to about 440%, at least or up to about 450%, at least or up to about 460%, at least or up to about 470%, at least or up to about 480%, at least or up to about 490%, or at least or up to about 500% compared to a user not sleeping at the temperature to enhance REM sleep.

In some embodiments, the bed device can optimize the temperature for enhancing wake sleep, temperature for enhancing light sleep, the temperature for enhancing deep sleep, and/or the temperature for enhancing REM sleep for the user based on the adjustments to the user makes in the bed device. In some cases, if the user wakes up during wake sleep and decreases the temperature to enhance wake sleep, then the bed device can adjust the temperature of the temperature to enhance wake sleep to decrease during the next subsequent use of the bed device. In some cases, if the user wakes up during wake sleep and increases the temperature of the temperature to enhance wake sleep, then the bed device can adjust the temperature of the temperature to enhance wake sleep to increase during the next subsequent use of the bed device. In some cases, if the user wakes up during light sleep and decreases the temperature to enhance light sleep, then the bed device can adjust the temperature of the temperature to enhance light sleep to decrease during the next subsequent use of the bed device. In some cases, if the user wakes up during light sleep and increases the temperature of the temperature to enhance light sleep, then the bed device can adjust the temperature of the temperature to enhance light sleep to increase during the next subsequent use of the bed device. In some cases, if the user wakes up during deep sleep and decreases the temperature of the temperature to enhance deep sleep, then the bed device can adjust the temperature of the temperature to enhance deep sleep to decrease during the next subsequent use of the bed device. In some cases, if the user wakes up during deep sleep and increases the temperature of the temperature to enhance deep sleep, then the bed device can adjust the temperature of the temperature to enhance deep sleep to increase during the next subsequent use of the bed device. In some cases, if the user wakes up during REM sleep and decreases the temperature of the temperature to enhance REM sleep, then the bed device can adjust the temperature of the temperature to enhance REM sleep to decrease during the next subsequent use of the bed device. In some cases, if the user wakes up during REM sleep and increases the temperature of the temperature to enhance REM sleep, then the bed device can adjust the temperature of the temperature to enhance REM sleep to increase during the next subsequent use of the bed device.

In some embodiments, the bed device can adjust the temperature of the bed device in real time to a temperature to enhance a specific sleep stage of the sleep cycle upon detection that the user is experiencing that specific sleep stage. In some cases, the detection of the specific sleep stage can be based on the one or more biological signals detected from the user. In some cases, upon detection that the user is in wake sleep, the bed device can adjust the temperature of the bed device to the temperature that enhances wake sleep. In some cases, upon detection that the user is in light sleep, the bed device can adjust the temperature of the bed device to the temperature that enhances light sleep. In some cases, upon detection that the user is in deep sleep, the bed device can adjust the temperature of the bed device to the temperature that enhances deep sleep. In some cases, upon detection that the user is in REM sleep, the bed device can adjust the temperature of the bed device to the temperature that enhances REM sleep.

In some embodiments, the temperature of the bed device adjusts throughout the night based on the subject's core temperature. In some embodiments, the one or more sensors in the bed device measures the body temperature (e.g., skin temperature) of the subject throughout the use of the bed device. In some embodiments, the body temperature data measured through the use of the bed device by the subject determines when the subject has the lowest body temperature during use of the bed device (e.g., the core temperature nadir).

In some embodiments, the core temperature nadir can be a time after a subject has fallen asleep where the body temperature of the subject stops decreasing and begins to increase. In some cases, prior to the core temperature nadir, the sign of the rate of change of the temperature can be negative (e.g., the temperature of the subject is decreasing). In some cases, subsequent to the core temperature nadir, the sign of the rate of change of the temperature can be positive (e.g., the temperature of the subject is increasing).

Alternatively, the core temperature can be a time after a subject has fallen asleep wherein the body temperature of the subject stops increasing and begins to decrease. In some cases, prior to the core temperature, nadir the sign of the rate of change of the temperature can be positive (e.g., the temperature of the subject is increasing). In some cases, the subsequent to the core temperature nadir, the sign of the rate of change of the temperature can be negative (e.g., the temperature of the subject is decreasing).

In some embodiments, the core temperature nadir can be the lowest body temperature of a user between the two highest body temperatures of the user during use of the bed device. In some cases, the core temperature nadir can be lower than a first highest body temperature of the user during use of the bed device. In some examples, the core temperature nadir can be between about 0° to about 10° lower than the first highest body temperature of the user during use of the bed device. In some examples, the core temperature nadir can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the first highest body temperature of the user during use of the bed device. In some cases, the first highest body temperature can occur around the time the user falls asleep during use of the bed device. In some cases, the core temperature nadir can be lower than a second highest body temperature of the user during use of the bed device. In some cases, the second highest body temperature can occur around the time when the user wake up during use of the bed device. In some examples, the core temperature nadir can be between about 0° to about 10° lower than the second highest body temperature of the user during use of the bed device. In some examples, the core temperature nadir can be at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1.0° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2.0° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., at least or up to about 3.0° C., at least or up to about 3.1° C., at least or up to about 3.2° C., at least or up to about 3.3° C., at least or up to about 3.4° C., at least or up to about 3.5° C., at least or up to about 3.6° C., at least or up to about 3.7° C., at least or up to about 3.8° C., at least or up to about 3.9° C., at least or up to about 4.0° C., at least or up to about 4.1° C., at least or up to about 4.2° C., at least or up to about 4.3° C., at least or up to about 4.4° C., at least or up to about 4.5° C., at least or up to about 4.6° C., at least or up to about 4.7° C., at least or up to about 4.8° C., at least or up to about 4.9° C., at least or up to about 5.0° C., at least or up to about 5.1° C., at least or up to about 5.2° C., at least or up to about 5.3° C., at least or up to about 5.4° C., at least or up to about 5.5° C., at least or up to about 5.6° C., at least or up to about 5.7° C., at least or up to about 5.8° C., at least or up to about 5.9° C., at least or up to about 6.0° C., at least or up to about 6.1° C., at least or up to about 6.2° C., at least or up to about 6.3° C., at least or up to about 6.4° C., at least or up to about 6.5° C., at least or up to about 6.6° C., at least or up to about 6.7° C., at least or up to about 6.8° C., at least or up to about 6.9° C., at least or up to about 7.0° C., at least or up to about 7.1° C., at least or up to about 7.2° C., at least or up to about 7.3° C., at least or up to about 7.4° C., at least or up to about 7.5° C., at least or up to about 7.6° C., at least or up to about 7.7° C., at least or up to about 7.8° C., at least or up to about 7.9° C., at least or up to about 8.0° C., at least or up to about 8.1° C., at least or up to about 8.2° C., at least or up to about 8.3° C., at least or up to about 8.4° C., at least or up to about 8.5° C., at least or up to about 8.6° C., at least or up to about 8.7° C., at least or up to about 8.8° C., at least or up to about 8.9° C., at least or up to about 9.0° C., at least or up to about 9.1° C., at least or up to about 9.2° C., at least or up to about 9.3° C., at least or up to about 9.4° C., at least or up to about 9.5° C., at least or up to about 9.6° C., at least or up to about 9.7° C., at least or up to about 9.8° C., at least or up to about 9.8° C., or at least or up to about 10° lower than the second highest body temperature of the user during use of the bed device.

In some embodiments, the core temperature nadir can be the lowest body temperature of the user measured during a use of the bed device.

In some embodiments, the core temperature nadir of the subject may be detectable by detecting the body temperature of the subject over time. In some embodiments, the bed device can determine a time or an estimated time when the core temperature nadir occurs. In some embodiments, the bed device determines the time or the estimated time that the core temperature nadir occurs by analyzing a plurality of data points. In some embodiments, analyzing the plurality of data points to determine when a sign of a rate of change of the temperature of the subject becomes different. In some embodiments, the sign of the rate of change of the temperature can change from negative to positive. In some embodiments, the sign of the rate of change of the temperature can change from positive to negative.

Alternatively, in some embodiments, the core temperature nadir of the subject may be detectable by detecting one or more biological signals that is not body temperature. In some embodiments, the analyzing the plurality of data points to determine when a sign of a rate of change of the one or more biological signals of the subject becomes different. In some embodiments, the sign of the rate of change of the one or more biological signals can change from negative to positive. In some embodiments, the sign of the rate of change of the one or more biological signals can change from positive to negative.

In some embodiments, the core temperature nadir of the subject can be determined when a subject is on top of a portion of the bed device. In some cases, the core temperature nadir of the subject can be determined when the bed device detects a presence of the subject on top of a portion of the bed device.

In some embodiments, the core temperature nadir of the subject can be determined by collecting data from the one or more biological signals for a period of time. In some cases, the period of time that data is collected for can be between about 0 minutes to about 10 years. In some cases, the period of time that data is collected for can be at least or up to about 0 minutes, at least or up to about 1 hour, at least or up to about 4 hours, at least or up to about 8 hours, at least or up to about 12 hours, at least or up to about 1 day, at least or up to about 2 days, at least or up to about 3 days, at least or up to about 4 days, at least or up to about 5 days, at least or up to about 6 days, at least or up to about 1 week, at least or up to about 2 weeks, at least or up to about 3 weeks, at least or up to about 1 month, at least or up to about 2 months, at least or up to about 3 months, at least or up to about 4 months, at least or up to about 5 months, at least or up to about 6 months, at least or up to about 7 months, at least or up to about 8 months, at least or up to about 9 months, at least or up to about 10 months, at least or up to about 11 months, at least or up to about 1 year, at least or up to about 2 years, at least or up to about 3 years, at least or up to about 4 years, at least or up to about 5 years, at least or up to about 6 years, at least or up to about 7 years, at least or up to about 8 years, at least or up to about 9 years, or at least or up to about 10 years.

In some embodiments, the sign of the rate of change of the temperature can be determined by collected data from the one or more biological signal for a period of time. In some cases, the period of time that data is collected for can be between about 0 minutes to about 10 years. In some cases, the period of time that data is collected for can be at least or up to about 0 minutes, at least or up to about 1 hour, at least or up to about 4 hours, at least or up to about 8 hours, at least or up to about 12 hours, at least or up to about 1 day, at least or up to about 2 days, at least or up to about 3 days, at least or up to about 4 days, at least or up to about 5 days, at least or up to about 6 days, at least or up to about 1 week, at least or up to about 2 weeks, at least or up to about 3 weeks, at least or up to about 1 month, at least or up to about 2 months, at least or up to about 3 months, at least or up to about 4 months, at least or up to about 5 months, at least or up to about 6 months, at least or up to about 7 months, at least or up to about 8 months, at least or up to about 9 months, at least or up to about 10 months, at least or up to about 11 months, at least or up to about 1 year, at least or up to about 2 years, at least or up to about 3 years, at least or up to about 4 years, at least or up to about 5 years, at least or up to about 6 years, at least or up to about 7 years, at least or up to about 8 years, at least or up to about 9 years, or at least or up to about 10 years.

In some embodiments, the sign of the rate of change can change from negative to positive between about 0 minutes to about 300 minutes of the subject getting on top a portion of the bed device. In some cases, the sign of the rate of change can change from negative to positive at least or up to about 0 minutes, at least or up to about 10 minutes, at least or up to about 20 minutes, at least or up to about 30 minutes, at least or up to about 40 minutes, at least or up to about 50 minutes, at least or up to about 60 minutes, at least or up to about 70 minutes, at least or up to about 80 minutes, at least or up to about 90 minutes, at least or up to about 100 minutes, at least or up to about 110 minutes, at least or up to about 120 minutes, at least or up to about 130 minutes, at least or up to about 140 minutes, at least or up to about 150 minutes, at least or up to about 160 minutes, at least or up to about 170 minutes, at least or up to about 180 minutes, at least or up to about 190 minutes, at least or up to about 200 minutes, at least or up to about 210 minutes, at least or up to about 220 minutes, at least or up to about 230 minutes, at least or up to about 240 minutes, at least or up to about 250 minutes, at least or up to about 260 minutes, at least or up to about 270 minutes, at least or up to about 280 minutes, at least or up to about 290 minutes, or at least or up to about 300 minutes of the subject getting on top a portion of the bed device.

In some embodiments, the sign of the rate of change can change from positive to negative between about 0 minutes to about 300 minutes of the subject getting on top a portion of the bed device. In some cases, the sign of the rate of change can change from positive to negative at least or up to about 0 minutes, at least or up to about 10 minutes, at least or up to about 20 minutes, at least or up to about 30 minutes, at least or up to about 40 minutes, at least or up to about 50 minutes, at least or up to about 60 minutes, at least or up to about 70 minutes, at least or up to about 80 minutes, at least or up to about 90 minutes, at least or up to about 100 minutes, at least or up to about 110 minutes, at least or up to about 120 minutes, at least or up to about 130 minutes, at least or up to about 140 minutes, at least or up to about 150 minutes, at least or up to about 160 minutes, at least or up to about 170 minutes, at least or up to about 180 minutes, at least or up to about 190 minutes, at least or up to about 200 minutes, at least or up to about 210 minutes, at least or up to about 220 minutes, at least or up to about 230 minutes, at least or up to about 240 minutes, at least or up to about 250 minutes, at least or up to about 260 minutes, at least or up to about 270 minutes, at least or up to about 280 minutes, at least or up to about 290 minutes, or at least or up to about 300 minutes of the subject getting on top a portion of the bed device.

In some embodiments, the sign of the rate of change can change from negative to positive between about 0 minutes to about 300 minutes of the subject falling asleep on top of a portion of the bed device. In some cases, the sign of the rate of change can change from negative to positive at least or up to about 0 minutes, at least or up to about 10 minutes, at least or up to about 20 minutes, at least or up to about 30 minutes, at least or up to about 40 minutes, at least or up to about 50 minutes, at least or up to about 60 minutes, at least or up to about 70 minutes, at least or up to about 80 minutes, at least or up to about 90 minutes, at least or up to about 100 minutes, at least or up to about 110 minutes, at least or up to about 120 minutes, at least or up to about 130 minutes, at least or up to about 140 minutes, at least or up to about 150 minutes, at least or up to about 160 minutes, at least or up to about 170 minutes, at least or up to about 180 minutes, at least or up to about 190 minutes, at least or up to about 200 minutes, at least or up to about 210 minutes, at least or up to about 220 minutes, at least or up to about 230 minutes, at least or up to about 240 minutes, at least or up to about 250 minutes, at least or up to about 260 minutes, at least or up to about 270 minutes, at least or up to about 280 minutes, at least or up to about 290 minutes, or at least or up to about 300 minutes of the subject falling asleep on top of a portion of the bed device.

In some embodiments, the sign of the rate of change can change from positive to negative between about 0 minutes to about 300 minutes of the subject falling asleep on top of a portion of the bed device. In some cases, the sign of the rate of change can change from positive to negative at least or up to about 0 minutes, at least or up to about 10 minutes, at least or up to about 20 minutes, at least or up to about 30 minutes, at least or up to about 40 minutes, at least or up to about 50 minutes, at least or up to about 60 minutes, at least or up to about 70 minutes, at least or up to about 80 minutes, at least or up to about 90 minutes, at least or up to about 100 minutes, at least or up to about 110 minutes, at least or up to about 120 minutes, at least or up to about 130 minutes, at least or up to about 140 minutes, at least or up to about 150 minutes, at least or up to about 160 minutes, at least or up to about 170 minutes, at least or up to about 180 minutes, at least or up to about 190 minutes, at least or up to about 200 minutes, at least or up to about 210 minutes, at least or up to about 220 minutes, at least or up to about 230 minutes, at least or up to about 240 minutes, at least or up to about 250 minutes, at least or up to about 260 minutes, at least or up to about 270 minutes, at least or up to about 280 minutes, at least or up to about 290 minutes, or at least or up to about 300 minutes of the subject falling asleep on top of a portion of the bed device.

In some embodiments, the one or more biological signals that is not body temperature is HR, HRV, and/or skin temperature. In some embodiments, the one or more biological signals that is not body temperature is HR. In some embodiments, the core temperature nadir of the subject may be detectable by detecting HR. For example, prior to the core temperature nadir, the HR of the subject can be decreasing, and subsequent to the core temperature nadir, the HR of the subject can be increasing. In some examples, the core temperature nadir can be determined by detecting the point of time when the HR of the subject starts to increase. In some examples, the bed device detects a pattern of data points of a heart signal (e.g., HR). In some examples, the pattern of data points detected by the bed device can be a change from a decreasing HR to an increasing HR. In some examples, the pattern of data points detected by the bed device can be a change of a rate of the heart signal. In some examples, the change of the rate of the heart signal can be a change from a negative rate of the heart signal to a position rate of the heart signal.

In some embodiments, the time or estimated time of the core temperature nadir is when a negative rate of change of the body temperature of the subject becomes a positive rate of change of the body temperature of the subject. In some embodiments, the time or estimated time of the core temperature nadir is when a positive rate of change of the body temperature of the subject becomes a negative rate of change of the body temperature of the subject.

In some embodiments, the time or estimated time of the core temperature nadir is when a positive rate of change of the body temperature of the subject becomes substantially zero change of the body temperature of the subject. In some embodiments, the time or estimated time of the core temperature nadir is when a substantially zero rate of change of the body temperature of the subject becomes a positive rate of the body temperature. In some embodiments, the time or estimated time of the core temperature nadir is when a negative rate of change of the body temperature of the subject becomes substantially zero change of the body temperature of the subject. In some embodiments, the time or estimated time of the core temperature nadir is when a substantially zero rate of change of the body temperature of the subject becomes a negative rate of the body temperature.

As used herein, the lowest body temperature (e.g., the core temperature nadir) may be a local minimum of the body temperature (e.g., not an absolute minimum of the body temperature).

Without wishing to be bound by theory, the local minimum of the body temperature may be indicative of a core temperature nadir or circadian rhythm temperature of the user. The local minimum of the body temperature may occur between about 12:00 ante meridiem (a.m.) and about 6:30 a.m., between about 12:30 a.m. and about 6:30 a.m., between about 1:00 a.m. and about 6:30 a.m., between about 1:30 a.m. and about 6:30 a.m., between about 2:00 a.m. and about 6:30 a.m., between about 2:30 a.m. and about 6:30 a.m., between about 3:00 a.m. and about 6:30 a.m., between about 12:00 a.m. and about 6:00 a.m., between about 12:30 a.m. and about 6:00 a.m., between about 1:00 a.m. and about 6:00 a.m., between about 1:30 a.m. and about 6:00 a.m., between about 2:00 a.m. and about 6:00 a.m., between about 2:30 a.m. and about 6:00 a.m., between about 3:00 a.m. and about 6:00 a.m., between about 3:30 a.m. and about 5:30 a.m., between about 3:00 a.m. and about 5:30 a.m., between about 2:30 a.m. and about 5:30 a.m., between about 2:00 a.m. and about 5:30 a.m., between about 1:00 a.m. and about 5:30 a.m., between about 1:00 a.m. and about 5:00 a.m., or between about 5:00 a.m. and about 6:30 a.m.

In some embodiments, the lowest body temperature of the subject is measured from the length of time from when the subject falls asleep. In some embodiments, the lowest body temperature of the subject occurs between about one to seven hours from when the subject falls asleep. In some embodiments, the lowest body temperature of the subject occurs at least about one hour, at least about two hours, at least about three hours, at least about four hours, at least about five hours, at least about six hours, at least about seven hours, or more after the subject falls asleep. In some embodiments, the lowest body temperature of the subject occurs at most about seven hours, at most about six hours, at most about five hours, at most about four hours, at most about three hours, at most about two hours, at most about one hours, or less after the subject falls asleep. In some embodiments, the lowest body temperature of the subject occurs about one hour, about two hours, about three hours, about four hours, about five hours, about six hours, or about seven hours after the subject falls asleep.

In some embodiments, the lowest body temperature of the subject is measured from the length of time from when the subject gets on the bed device. In some embodiments, the lowest body temperature of the subject occurs at least about one hour, at least about two hours, at least about three hours, at least about four hours, at least about five hours, at least about six hours, at least about seven hours, or more after the subject gets on the bed device. In some embodiments, the lowest body temperature of the subject occurs at most about seven hours, at most about six hours, at most about five hours, at most about four hours, at most about three hours, at most about two hours, at most about one hour, or less after the subject gets on the bed device. In some embodiments, the lowest body temperature of the subject occurs about one hour, about two hours, about three hours, about four hours, about five hours, about six hours, or about seven hours after the subject gets on the bed device.

In some embodiments, the temperature of the bed device prior to the lowest body temperature of the subject is lower than the temperature of the bed device after the lowest body temperature of the subject. In some embodiments, the temperature of the bed device subsequent to the lowest body temperature of the subject is higher than the temperature of the bed device prior to the lowest body temperature of the subject. Changing (e.g., increasing) the temperature of the bed device subsequent to when the lowest body temperature occurs can promote more deep sleep while the subject sleeps. In some embodiments, the temperature of the bed device subsequent to the lowest body temperature changes (e.g., increases) in one or more increments to the final temperature. In some embodiments, the one or more increments is between about one increment to about six increments. In some embodiments, the one or more increments is at least or up to about one increment, at least or up to about two increments, at least or up to about three increments, at least or up to about four increments, at least or up to about five increments, or at least or up to about six increments. In some embodiments, the one or more increments is at least or up to about four increments.

In some embodiments, the lowest body temperature of the subject is measured by the skin temperature of the subject. In some embodiments, the lowest body temperature of the subject is measured by the HR of the subject. The core temperature of the subject can follow the same trend of the HR of the subject.

In some embodiments, the bed device automatically changes (e.g., increases) the temperature of the bed device upon detection of the subject's lowest body temperature. In some embodiments, the method (e.g., the computer-implemented method) further comprises collecting data over a period of time to determine when the lowest body temperature of the subject occurs. In some embodiments, the method collects data for between 24 hours to about 7 days to determine when the lowest body temperature of the subject occurs. In some embodiments, the method collects data for at least about 24 hours, at least about two days, at least about three days, at least about four days, at least about five days, at least about six days, at least about seven days, or more to determine when the lowest body temperature of the subject occurs. In some embodiments, the method collects data for at most about seven days, at most about two six, at most about five days, at most about four days, at most about three days, at most about two days, at most about 24 hours, or less to determine when the lowest body temperature of the subject occurs. In some embodiments, the method (e.g., computer-implemented method) automatically updates when the lowest body temperature of the subject occurs following the collection of the data.

In some embodiments, the temperature of the bed device adjusts throughout the night based on the subject's heart signal. In some embodiments, the one or more sensors in the bed device measures the subject's heart signal (e.g., heart rate, HRV) of the subject throughout the use of the bed device. In some embodiments, the heart signal data measured through the use of the bed device by the subject determines when the subject has the lowest heart signal during use of the bed device (e.g., the heart signal nadir).

In some embodiments, the heart signal nadir can be a time after a subject has fallen asleep where the heart signal of the subject stops decreasing and begins to increase. In some cases, prior to the heart signal nadir, the sign of the rate of change of the heart signal can be negative (e.g., the heart signal of the subject is decreasing). In some cases, subsequent to the heart signal nadir, the sign of the rate of change of the heart signal can be positive (e.g., the heart signal of the subject is increasing).

Alternatively, the heart signal can be a time after a subject has fallen asleep wherein the heart signal of the subject stops increasing and begins to decrease. In some cases, prior to the heart signal, nadir the sign of the rate of change of the heart signal can be positive (e.g., the heart signal of the subject is increasing). In some cases, the subsequent to the heart signal nadir, the sign of the rate of change of the heart signal can be negative (e.g., the heart signal of the subject is decreasing).

In some embodiments, the heart signal nadir can be the lowest heart signal of a user between the two highest heart signal of the user during use of the bed device. In some cases, the heart signal nadir can be lower than a first highest heart signal of the user during use of the bed device. In some cases, the first highest heart signal can occur around the time the user falls asleep during use of the bed device. In some cases, the heart signal nadir can be lower than a second highest heart signal of the user during use of the bed device.

In some embodiments, the heart signal nadir can be the lowest heart signal of the user measured during a use of the bed device.

In some embodiments, the heart signal nadir of the subject may be detectable by detecting the heart signal of the subject over time. In some embodiments, the bed device can determine a time or an estimated time when the heart signal nadir occurs. In some embodiments, the bed device determines the time or the estimated time that the heart signal nadir occurs by analyzing a plurality of data points. In some embodiments, analyzing the plurality of data points to determine when a sign of a rate of change of the heart signal of the subject becomes different. In some embodiments, the sign of the rate of change of the heart signal can change from negative to positive. In some embodiments, the sign of the rate of change of the heart signal can change from positive to negative.

In some embodiments, the heart signal nadir of the subject can be determined when a subject is on top of a portion of the bed device. In some cases, the heart signal nadir of the subject can be determined when the bed device detects a presence of the subject on top of a portion of the bed device.

In some embodiments, the heart signal nadir of the subject can be determined by collecting data from the one or more biological signals for a period of time. In some cases, the period of time that data is collected for can be between about 0 minutes to about 10 years. In some cases, the period of time that data is collected for can be at least or up to about 0 minutes, at least or up to about 1 hour, at least or up to about 4 hours, at least or up to about 8 hours, at least or up to about 12 hours, at least or up to about 1 day, at least or up to about 2 days, at least or up to about 3 days, at least or up to about 4 days, at least or up to about 5 days, at least or up to about 6 days, at least or up to about 1 week, at least or up to about 2 weeks, at least or up to about 3 weeks, at least or up to about 1 month, at least or up to about 2 months, at least or up to about 3 months, at least or up to about 4 months, at least or up to about 5 months, at least or up to about 6 months, at least or up to about 7 months, at least or up to about 8 months, at least or up to about 9 months, at least or up to about 10 months, at least or up to about 11 months, at least or up to about 1 year, at least or up to about 2 years, at least or up to about 3 years, at least or up to about 4 years, at least or up to about 5 years, at least or up to about 6 years, at least or up to about 7 years, at least or up to about 8 years, at least or up to about 9 years, or at least or up to about 10 years.

In some embodiments, the sign of the rate of change of the heart signal can be determined by collected data from the one or more biological signal for a period of time. In some cases, the period of time that data is collected for can be between about 0 minutes to about 10 years. In some cases, the period of time that data is collected for can be at least or up to about 0 minutes, at least or up to about 1 hour, at least or up to about 4 hours, at least or up to about 8 hours, at least or up to about 12 hours, at least or up to about 1 day, at least or up to about 2 days, at least or up to about 3 days, at least or up to about 4 days, at least or up to about 5 days, at least or up to about 6 days, at least or up to about 1 week, at least or up to about 2 weeks, at least or up to about 3 weeks, at least or up to about 1 month, at least or up to about 2 months, at least or up to about 3 months, at least or up to about 4 months, at least or up to about 5 months, at least or up to about 6 months, at least or up to about 7 months, at least or up to about 8 months, at least or up to about 9 months, at least or up to about 10 months, at least or up to about 11 months, at least or up to about 1 year, at least or up to about 2 years, at least or up to about 3 years, at least or up to about 4 years, at least or up to about 5 years, at least or up to about 6 years, at least or up to about 7 years, at least or up to about 8 years, at least or up to about 9 years, or at least or up to about 10 years.

In some embodiments, the sign of the rate of change can change from negative to positive between about 0 minutes to about 300 minutes of the subject getting on top a portion of the bed device. In some cases, the sign of the rate of change can change from negative to positive at least or up to about 0 minutes, at least or up to about 10 minutes, at least or up to about 20 minutes, at least or up to about 30 minutes, at least or up to about 40 minutes, at least or up to about 50 minutes, at least or up to about 60 minutes, at least or up to about 70 minutes, at least or up to about 80 minutes, at least or up to about 90 minutes, at least or up to about 100 minutes, at least or up to about 110 minutes, at least or up to about 120 minutes, at least or up to about 130 minutes, at least or up to about 140 minutes, at least or up to about 150 minutes, at least or up to about 160 minutes, at least or up to about 170 minutes, at least or up to about 180 minutes, at least or up to about 190 minutes, at least or up to about 200 minutes, at least or up to about 210 minutes, at least or up to about 220 minutes, at least or up to about 230 minutes, at least or up to about 240 minutes, at least or up to about 250 minutes, at least or up to about 260 minutes, at least or up to about 270 minutes, at least or up to about 280 minutes, at least or up to about 290 minutes, or at least or up to about 300 minutes of the subject getting on top a portion of the bed device.

In some embodiments, the sign of the rate of change can change from positive to negative between about 0 minutes to about 300 minutes of the subject getting on top a portion of the bed device. In some cases, the sign of the rate of change can change from positive to negative at least or up to about 0 minutes, at least or up to about 10 minutes, at least or up to about 20 minutes, at least or up to about 30 minutes, at least or up to about 40 minutes, at least or up to about 50 minutes, at least or up to about 60 minutes, at least or up to about 70 minutes, at least or up to about 80 minutes, at least or up to about 90 minutes, at least or up to about 100 minutes, at least or up to about 110 minutes, at least or up to about 120 minutes, at least or up to about 130 minutes, at least or up to about 140 minutes, at least or up to about 150 minutes, at least or up to about 160 minutes, at least or up to about 170 minutes, at least or up to about 180 minutes, at least or up to about 190 minutes, at least or up to about 200 minutes, at least or up to about 210 minutes, at least or up to about 220 minutes, at least or up to about 230 minutes, at least or up to about 240 minutes, at least or up to about 250 minutes, at least or up to about 260 minutes, at least or up to about 270 minutes, at least or up to about 280 minutes, at least or up to about 290 minutes, or at least or up to about 300 minutes of the subject getting on top a portion of the bed device.

In some embodiments, the sign of the rate of change can change from negative to positive between about 0 minutes to about 300 minutes of the subject falling asleep on top of a portion of the bed device. In some cases, the sign of the rate of change can change from negative to positive at least or up to about 0 minutes, at least or up to about 10 minutes, at least or up to about 20 minutes, at least or up to about 30 minutes, at least or up to about 40 minutes, at least or up to about 50 minutes, at least or up to about 60 minutes, at least or up to about 70 minutes, at least or up to about 80 minutes, at least or up to about 90 minutes, at least or up to about 100 minutes, at least or up to about 110 minutes, at least or up to about 120 minutes, at least or up to about 130 minutes, at least or up to about 140 minutes, at least or up to about 150 minutes, at least or up to about 160 minutes, at least or up to about 170 minutes, at least or up to about 180 minutes, at least or up to about 190 minutes, at least or up to about 200 minutes, at least or up to about 210 minutes, at least or up to about 220 minutes, at least or up to about 230 minutes, at least or up to about 240 minutes, at least or up to about 250 minutes, at least or up to about 260 minutes, at least or up to about 270 minutes, at least or up to about 280 minutes, at least or up to about 290 minutes, or at least or up to about 300 minutes of the subject falling asleep on top of a portion of the bed device.

In some embodiments, the sign of the rate of change can change from positive to negative between about 0 minutes to about 300 minutes of the subject falling asleep on top of a portion of the bed device. In some cases, the sign of the rate of change can change from positive to negative at least or up to about 0 minutes, at least or up to about 10 minutes, at least or up to about 20 minutes, at least or up to about 30 minutes, at least or up to about 40 minutes, at least or up to about 50 minutes, at least or up to about 60 minutes, at least or up to about 70 minutes, at least or up to about 80 minutes, at least or up to about 90 minutes, at least or up to about 100 minutes, at least or up to about 110 minutes, at least or up to about 120 minutes, at least or up to about 130 minutes, at least or up to about 140 minutes, at least or up to about 150 minutes, at least or up to about 160 minutes, at least or up to about 170 minutes, at least or up to about 180 minutes, at least or up to about 190 minutes, at least or up to about 200 minutes, at least or up to about 210 minutes, at least or up to about 220 minutes, at least or up to about 230 minutes, at least or up to about 240 minutes, at least or up to about 250 minutes, at least or up to about 260 minutes, at least or up to about 270 minutes, at least or up to about 280 minutes, at least or up to about 290 minutes, or at least or up to about 300 minutes of the subject falling asleep on top of a portion of the bed device.

In some embodiments, the time or estimated time of the heart signal nadir is when a negative rate of change of the heart signal of the subject becomes a positive rate of change of the heart signal of the subject. In some embodiments, the time or estimated time of the heart signal nadir is when a positive rate of change of the heart signal of the subject becomes a negative rate of change of the heart signal of the subject.

In some embodiments, the time or estimated time of the heart signal nadir is when a positive rate of change of the heart signal of the subject becomes substantially zero change of the heart signal of the subject. In some embodiments, the time or estimated time of the heart signal nadir is when a substantially zero rate of change of the heart signal of the subject becomes a positive rate of the heart signal. In some embodiments, the time or estimated time of the heart signal nadir is when a negative rate of change of the heart signal of the subject becomes substantially zero change of the heart signal of the subject. In some embodiments, the time or estimated time of the heart signal nadir is when a substantially zero rate of change of the heart signal of the subject becomes a negative rate of the heart signal.

As used herein, the lowest heart signal (e.g., the heart signal nadir) may be a local minimum of the heart signal (e.g., not an absolute minimum of the heart signal). Without wishing to be bound by theory, the local minimum of the heart signal may be indicative of a heart signal nadir or circadian rhythm heart signal of the user. The local minimum of the heart signal may occur between about 12:00 ante meridiem (a.m.) and about 6:30 a.m., between about 12:30 a.m. and about 6:30 a.m., between about 1:00 a.m. and about 6:30 a.m., between about 1:30 a.m. and about 6:30 a.m., between about 2:00 a.m. and about 6:30 a.m., between about 2:30 a.m. and about 6:30 a.m., between about 3:00 a.m. and about 6:30 a.m., between about 12:00 a.m. and about 6:00 a.m., between about 12:30 a.m. and about 6:00 a.m., between about 1:00 a.m. and about 6:00 a.m., between about 1:30 a.m. and about 6:00 a.m., between about 2:00 a.m. and about 6:00 a.m., between about 2:30 a.m. and about 6:00 a.m., between about 3:00 a.m. and about 6:00 a.m., between about 3:30 a.m. and about 5:30 a.m., between about 3:00 a.m. and about 5:30 a.m., between about 2:30 a.m. and about 5:30 a.m., between about 2:00 a.m. and about 5:30 a.m., between about 1:00 a.m. and about 5:30 a.m., between about 1:00 a.m. and about 5:00 a.m., or between about 5:00 a.m. and about 6:30 a.m.

In some embodiments, the lowest heart signal of the subject is measured from the length of time from when the subject falls asleep. In some embodiments, the lowest heart signal of the subject occurs between about one to seven hours from when the subject falls asleep. In some embodiments, the lowest heart signal of the subject occurs at least about one hour, at least about two hours, at least about three hours, at least about four hours, at least about five hours, at least about six hours, at least about seven hours, or more after the subject falls asleep. In some embodiments, the lowest heart signal of the subject occurs at most about seven hours, at most about six hours, at most about five hours, at most about four hours, at most about three hours, at most about two hours, at most about one hours, or less after the subject falls asleep. In some embodiments, the lowest heart signal of the subject occurs about one hour, about two hours, about three hours, about four hours, about five hours, about six hours, or about seven hours after the subject falls asleep.

In some embodiments, the lowest heart signal of the subject is measured from the length of time from when the subject gets on the bed device. In some embodiments, the lowest heart signal of the subject occurs at least about one hour, at least about two hours, at least about three hours, at least about four hours, at least about five hours, at least about six hours, at least about seven hours, or more after the subject gets on the bed device. In some embodiments, the lowest heart signal of the subject occurs at most about seven hours, at most about six hours, at most about five hours, at most about four hours, at most about three hours, at most about two hours, at most about one hour, or less after the subject gets on the bed device. In some embodiments, the lowest heart signal of the subject occurs about one hour, about two hours, about three hours, about four hours, about five hours, about six hours, or about seven hours after the subject gets on the bed device.

In some embodiments, the heart signal of the bed device prior to the lowest heart signal of the subject is lower than the heart signal of the bed device after the lowest heart signal of the subject. In some embodiments, the heart signal of the bed device subsequent to the lowest heart signal of the subject is higher than the heart signal of the bed device prior to the lowest heart signal of the subject. Changing (e.g., increasing) the heart signal of the bed device subsequent to when the lowest heart signal occurs can promote more deep sleep while the subject sleeps. In some embodiments, the heart signal of the bed device subsequent to the lowest heart signal changes (e.g., increases) in one or more increments to the final heart signal. In some embodiments, the one or more increments is between about one increment to about six increments. In some embodiments, the one or more increments is at least or up to about one increment, at least or up to about two increments, at least or up to about three increments, at least or up to about four increments, at least or up to about five increments, or at least or up to about six increments. In some embodiments, the one or more increments is at least or up to about four increments.

In some embodiments, the bed device automatically changes (e.g., increases) the temperature of the bed device upon detection of the subject's lowest heart signal. In some embodiments, the method (e.g., the computer-implemented method) further comprises collecting data over a period of time to determine when the lowest heart signal of the subject occurs. In some embodiments, the method collects data for between 24 hours to about 7 days to determine when the lowest heart signal of the subject occurs. In some embodiments, the method collects data for at least about 24 hours, at least about two days, at least about three days, at least about four days, at least about five days, at least about six days, at least about seven days, or more to determine when the lowest heart signal of the subject occurs. In some embodiments, the method collects data for at most about seven days, at most about two six, at most about five days, at most about four days, at most about three days, at most about two days, at most about 24 hours, or less to determine when the lowest heart signal of the subject occurs. In some embodiments, the method (e.g., computer-implemented method) automatically updates when the lowest heart signal of the subject occurs following the collection of the data.

In some embodiments, the bed device utilizes real-time data to determine what the temperature of the bed device should be for a given time of night or sleep cycle.

In some embodiments, the bed device optimizes the bed device temperature during the night to promote more time in a specific sleep stage (e.g., more time in deep or REM sleep) depending on what type of sleep the subject has been recently lacking. In some embodiments, optimizing the bed device temperature to promote more time in a specific sleep stage is accomplished by modifying the temperature schedule and/or modifying the temperature itself for each sleep stage. For example, increasing temperature (e.g., cooler to warmer temp) midway through the night can promote more deep sleep, while decreasing temperature midway through the night can promote more REM sleep. In some embodiments, modifying the temperature of the bed device to correspond with the subject's circadian rhythm for certain sleep stages can improve overall user experience, sleep quality, and health.

In some embodiments, the bed device can automatically modify the bed device temperature to align with the subject's sleep cycles e.g. non-REM temperatures would be slightly cooler than REM temperatures). In some embodiments, increasing the temperatures of the bed device after the core temperature nadir occurs can improve sleep quality (e.g., optimize bed device for specific sleep stage).

In some embodiments, the method (e.g., the computer-implemented method) comprises inputting information into an app. In some embodiments, the information inputted into the app includes: if the subject has been away from the bed device, if the subject has traveled, where did the subject travel to, heat acclimatization, or exercise information. In some embodiments, the exercise information is regarding regular exercise practices. In some embodiments, the exercise information is collected from a wearable device the subject wears while exercising. In some embodiments, the information inputted into the app adjusts the temperature of the bed device when the subject uses the bed device next. In some embodiments, the information inputted into the app adjusts the length of time between temperature changes (e.g., increases).

### Recommended Temperatures for an Initial Number of Days (e.g., 7 Days)

One aspect of the present disclosure provides a method (e.g., a computer implemented method) of recommending first temperatures (e.g., first bedtime temperature, first initial temperature, first final temperature) of the bed device for the subject. In some embodiments, the bedtime temperature, the initial temperature, and/or the final temperature are provided by the user of the article of furniture (e.g., bed device). In some embodiments, the bedtime temperature, the initial temperature, and/or the final temperature are automatically generated by a computer processer. In some embodiments, the subject has never used the bed device. In some embodiments, the first temperatures includes the temperature for a first use of the bed device by the subject. In some embodiments, the combination of first temperatures (e.g., target temperatures) is provided when an identity of the user is undetermined.

In some embodiments, the bed device further comprises a processor. In some embodiments, the processor analyzes the data collected by the one or more sensors. In some embodiments, the bed device further comprises a temperature regulator. In some embodiments, the temperature regulator is operatively connected to the processor. In some embodiments, the processor can provide a control signal to the temperature regulator. In some embodiments, the control signal can be an instruction for the temperature regulator to change a temperature of the article of furniture. In some embodiments, the control signal can be an instruction for the temperature regulator to change a temperature of a portion of the article of furniture (e.g., bed device). In some embodiments, the control signal can be an instruction to change the temperature of the bed device based on the subject's age. In some embodiments, the control signal can be an instruction to change the temperature of the bed device based on the subject's biological sex. In some embodiments, the control signal can be an instruction to change the temperature of the bed device based on the subject's geolocation. In some embodiments, the control signal can be an instruction to change the temperature of the bed device based on the season of the subject's geolocation.

In some embodiments, the first temperature provided herein can be recommended by the bed device (e.g., by a computer processor operatively coupled to the bed device). Such recommendation can be based on information from other users and/or sensing data collected from the user while using the bed device.

In some embodiments, the first temperatures are determined by geolocation of the subject, biological sex of the subject, age of the subject, or the season of the geolocation the subject is experiencing. Other criteria for determining the first temperatures can include one or more of the following: weight of the user, body mass index of the subject, height of the subject, preferred sleep posture of the subject, body shape of the subject, waistline of the subject, ethnic background of the subject, or any combination thereof. In some embodiments, the first temperatures are determined by geolocation of the subject. In some embodiments, the first temperatures are determined by biological sex of the subject. In some embodiments, the first temperatures are determined by age of the subject. In some embodiments, the first temperatures are determined by the season the geolocation of the subject is experiencing. In some embodiments, the first temperatures are determined by the geolocation of the subject, the biological sex of the subject, the age of the subject, and the season the geolocation of the subject is experiencing. In some cases, a geolocation of the subject can refer to actual geolocation of the subject (e.g., location within the globe), or any condition of the geolocation such as season, weather, temperature, humidity, etc.

In some embodiments, the first temperatures (e.g., combination of predetermined temperatures) are automatically set in the bed device prior to the subject's first use of the bed device. In some embodiments, the first temperatures are determined by inputting subject information into the bed device app. In some embodiments, the subject inputs the subject's biological sex into the app. In some embodiments, the subject inputs the subject's age into the app. In some embodiments, the subject inputs the subject's geolocation into the app. In some embodiments, the app automatically determines the season of the geolocation once the subject inputs the geolocation information into the app.

In some embodiments, the first temperatures (e.g., combination of predetermined temperatures) are automatically set in the bed device prior to a single user of the bed device by the user. In some embodiments, the first temperatures are determined prior to the single user of the bed device by the user. In some embodiments, the first temperatures are determined by inputting subject information into the bed device app. In some embodiments, the subject inputs the subject's biological sex into the app. In some embodiments, the subject inputs the subject's age into the app. In some embodiments, the subject inputs the subject's geolocation into the app. In some embodiments, the app automatically determines the season of the geolocation once the subject inputs the geolocation information into the app. In some embodiments, the subject inputs the subject's biological sex and the subject's geolocation into the app. In some embodiments, the subject inputs the subject's biological sex and the subject's age into the app. In some embodiments, the subject inputs the subject's age and the subject's geolocation into the app. In some embodiments, the subject inputs the subject's biological sex, the subject's age, and the subject's geolocation into the app.

In some embodiments, the user may not be permitted to change one or more temperature of the first temperatures until the user uses the bed device at the first temperatures for at least one use (e.g., at least one day of use). At least one use can comprise a single use, at least or up to about 2 uses, at least or up to about 3 uses, at least or up to about 4 uses, at least or up to about 5 uses, at least or up to about 6 uses, at least or up to about 7 uses, at least or up to about 8 uses, at least or up to about 9 uses, at least or up to about 10 uses, at least or up to about 12 uses, at least or up to about 15 uses, at least or up to about 20 uses, at least or up to about 25 uses, or at least or up to about 30 uses. Alternatively, the user may be permitted to change the first temperatures without any use of the bed device at the first temperatures. Any change of the first temperatures can be done, for example, via a graphical user interface (GUI) operatively coupled to the user, such as GUI of a mobile device (e.g., user device such as a mobile phone, or a remote of the bed device).

In some embodiments, it is recommended that the user not change one or more temperature of the first temperatures until the user uses the bed device at the first temperatures for at least one use (e.g., at least one day of use). At least one use can comprise a single use, at least or up to about 2 uses, at least or up to about 3 uses, at least or up to about 4 uses, at least or up to about 5 uses, at least or up to about 6 uses, at least or up to about 7 uses, at least or up to about 8 uses, at least or up to about 9 uses, at least or up to about 10 uses, at least or up to about 12 uses, at least or up to about 15 uses, at least or up to about 20 uses, at least or up to about 25 uses, or at least or up to about 30 uses. In some embodiments, the bed device app can send a notification to the user via the GUI. In some cases, the notification can recommend that the user not change the one or more temperature of the first temperatures for at least one use. Alternatively, in some embodiments, the bed device app can be preprogrammed to prohibit the user from changing the one or more temperature of the first temperatures for at least about one day. At least one day can comprise at least about one day, at least or up to about two days, at least or up to about three days, at least or up to about four days, at least or up to about five days, at least or up to about six days, at least or up to about seven days, at least or up to about eight days, at least or up to about nine days, at least or up to about ten days, at least or up to about 11 days, at least or up to about 12 days, at least or up to about 13 days, at least or up to about 14 days, at least or up to about 15 days, at least or up to about 16 days, at least or up to about 17 days, at least or up to about 18 days, at least or up to about 19 days, at least or up to about 20 days, at least or up to about 21 days, at least or up to about 22 days, at least or up to about 23 days, at least or up to about 24 days, at least or up to about 25 days, at least or up to about 26 days, at least or up to about 27 days, at least or up to about 28 days, at least or up to about 29 days, or at least or up to about 30 days.

In some embodiments, the subject cannot adjust the first temperature automatically set in the bed device prior to the subject's first use of the bed device. In some embodiments, the subject can adjust the bedtime temperature, the initial temperature, or the final temperature subsequent to the subject's first use of the bed device. In some embodiments, the subject adjusts the bedtime temperature, the initial temperature, or the final temperature in the bed device app.

Alternatively, in some embodiments, the subject can adjust the first temperatures automatically set in the bed device prior to the subject's first use of the bed device. In some embodiments, the subject can adjust the bedtime temperature, the initial temperature, or the final temperature prior to the subject's first use of the bed device. In some embodiments, the subject adjusts the bedtime temperature, the initial temperature, or the final temperature in the bed device app.

In some embodiments, the first temperatures may continue to be modified following the period of time where the user is not permitted or not recommended to change the first temperatures. In some cases, the first temperatures may continue to be modified based on the geolocation of the user at the current time of the use of the bed device. In some cases, the first temperatures can be modified for every use of the bed device based on the geolocation of the user at the current time of the use of the bed device.

In some embodiments, the bedtime temperature of the first temperatures is between about 12° C. to about 43° C. In some embodiments, the bedtime temperature of the first temperatures is at least or up to about 12° C., at least or up to about 13%, at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., at least or up to about 31° C., at least or up to about 32° C., at least or up to about 33° C., at least or up to about 34° C., at least or up to about 35° C., at least or up to about 36° C., at least or up to about 37° C., at least or up to about 38° C., at least or up to about 39° C., at least or up to about 40° C., at least or up to about 41° C., at least or up to about 42° C., or at least or up to about 43° C. In some embodiments, the bedtime temperature of the first temperatures is between the dial value of about −10 dial values to about +10 dial values of the bed device. In some embodiments, the bedtime temperature of the first temperatures is the dial value of at least or up to about −10, at least or up to about −9.5, at least or up to about −9, at least or up to about −8.5, at least or up to about −8, at least or up to about −7.5, at least or up to about −7, at least or up to about −6.5, at least or up to about −6, at least or up to about −5.5, at least or up to about −5, at least or up to about −4.5, at least or up to about −4, at least or up to about −3.5, at least or up to about −3, at least or up to about −2.5, at least or up to about −2, at least or up to about −1.5, at least or up to about −1, at least or up to about −0.5, at least or up to about 0, at least or up to about +0.5, at least or up to about +1, at least or up to about +1.5, at least or up to about +2, at least or up to about +2.5, at least or up to about +3, at least or up to about +3.5, at least or up to about +4, at least or up to about +4.5, at least or up to about +5, at least or up to about +5.5, at least or up to about +6, at least or up to about +6.5, at least or up to about +7, at least or up to about +7.5, at least or up to about +8, at least or up to about +8.5, at least or up to about +9, at least or up to about +9.5, or at least or up to about +10 dial values of the bed device.

In some embodiments, the initial temperature of the first temperatures is between about 12° C. to about 43° C. In some embodiments, the initial temperature of the first temperatures is at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., at least or up to about 31° C., at least or up to about 32° C., at least or up to about 33° C., at least or up to about 34° C., at least or up to about 35° C., at least or up to about 36° C., at least or up to about 37° C., at least or up to about 38° C., at least or up to about 39° C., at least or up to about 40° C., at least or up to about 41° C., at least or up to about 42° C., or at least or up to about 43° C. In some embodiments, the initial temperature of the first temperatures is between the dial value of about −10 to about +10. In some embodiments, the initial temperature of the first temperatures is the dial value of at least or up to about −10, at least or up to about −9.5, at least or up to about −9, at least or up to about −8.5, at least or up to about −8, at least or up to about −7.5, at least or up to about −7, at least or up to about −6.5, at least or up to about −6, at least or up to about −5.5, at least or up to about −5, at least or up to about −4.5, at least or up to about −4, at least or up to about −3.5, at least or up to about −3, at least or up to about −2.5, at least or up to about −2, at least or up to about −1.5, at least or up to about −1, at least or up to about −0.5, at least or up to about 0, at least or up to about +0.5, at least or up to about +1, at least or up to about +1.5, at least or up to about +2, at least or up to about +2.5, at least or up to about +3, at least or up to about +3.5, at least or up to about +4, at least or up to about +4.5, at least or up to about +5, at least or up to about +5.5, at least or up to about +6, at least or up to about +6.5, at least or up to about +7, at least or up to about +7.5, at least or up to about +8, at least or up to about +8.5, at least or up to about +9, at least or up to about +9.5, or at least or up to about +10.

In some embodiments, the final temperature of the first temperatures is between about 12° C. to about 43° C. In some embodiments, the final temperature of the first temperatures is at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., at least or up to about 31° C., at least or up to about 32° C., at least or up to about 33° C., at least or up to about 34° C., at least or up to about 35° C., at least or up to about 36° C., at least or up to about 37° C., at least or up to about 38° C., at least or up to about 39° C., at least or up to about 40° C., at least or up to about 41° C., at least or up to about 42° C., or at least or up to about 43° C. In some embodiments, the final temperature of the first temperatures is between the dial value of about −9 to about +10. In some embodiments, the initial temperature of the first temperatures is the dial value of at least or up to about −10, at least or up to about −9.5, at least or up to about −9, at least or up to about −8.5, at least or up to about −8, at least or up to about −7.5, at least or up to about −7, at least or up to about −6.5, at least or up to about −6, at least or up to about −5.5, at least or up to about −5, at least or up to about −4.5, at least or up to about −4, at least or up to about −3.5, at least or up to about −3, at least or up to about −2.5, at least or up to about −2, at least or up to about −1.5, at least or up to about −1, at least or up to about −0.5, at least or up to about 0, at least or up to about +0.5, at least or up to about +1, at least or up to about +1.5, at least or up to about +2, at least or up to about +2.5, at least or up to about +3, at least or up to about +3.5, at least or up to about +4, at least or up to about +4.5, at least or up to about +5, at least or up to about +5.5, at least or up to about +6, at least or up to about +6.5, at least or up to about +7, at least or up to about +7.5, at least or up to about +8, at least or up to about +8.5, at least or up to about +9, at least or up to about +9.5, or at least or up to about +10.

In some embodiments, the bed device app collects data of the temperatures of the bed device over one or more days, e.g., at least or up to about 1 day, at least or up to about 2 days, at least or up to about 3 days, at least or up to about 4 days, at least or up to about 5 days, at least or up to about 6 days, at least or up to about 7 days, at least or up to about 8 days, at least or up to about 9 days, at least or up to about 10 days, at least or up to about 11 days, at least or up to about 12 days, at least or up to about 13 days, at least or up to about 14 days, at least or up to about 15 days, or at least or up to about 20 days. In some embodiments, the bed device app collects data of the temperatures of the bed device between about 24 hours to about 7 days after the subject's first use of the bed device. In some embodiments, the bed device app collects data of the dial value updates between about 24 hours to about 7 days after the subject's first use of the bed device. A dial value update of the bed device can be when temperature that is automatically set by the bed device is changed by the subject in the bed device app. In some embodiments, the bed device app collects data for at least or up to about 24 hours, at least or up to about 48 hours, at least or up to about 3 days, at least or up to about 4 days, at least or up to about 5 days, at least or up to about 6 days, or at least or up to about 7 days after the subject's first use of the bed device. In some embodiments, the bed device app uses the collected data to automatically adjust the temperature of the bed device for the subject's next use of the bed device. In some embodiments, the bed device app uses the collected data to automatically adjust the dial values of the bed device or the subject's next use of the bed device.

In some embodiments, the subject has lower percentage of dial value updates after about the first seven days of use of the bed device using the first temperatures compared to a subject that did not use the first temperature during the about first seven days of use of the bed device. In some embodiments, the subject who uses the first temperatures has between about 0% to about 500% decrease in percent change of dial value updates compared to a subject that did not use the first temperatures. In some embodiments, the subject who uses the first temperatures has at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% decrease in percent change of dial value updates compared to a subject that did not use the first temperatures. In some embodiments, the subject who uses the first temperatures has about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% decrease in percent change of dial value updates compared to a subject that did not use the first temperatures.

In some embodiments, the first temperatures of the bed device enhance user experience of the bed device. In some embodiments, the first temperatures of the bed device enhance the user experience of the bed device by improving sleep quality of the user. In some embodiments, the first temperatures of the bed device improve the sleep quality values of the subject. Non-limiting examples of sleep quality values include percent light sleep, percent deep sleep, percent REM sleep, total sleep time, total awake time, and net sleep score.

In some embodiments, the first temperatures of the bed device result in an increase in percent change of light sleep. In some embodiments, the first temperatures of the bed device result in between 0%-300% increase in percent change of light sleep. In some embodiments, the first temperatures of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change of light sleep.

In some embodiments, the first temperatures of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of light sleep. In some embodiments, the first temperatures of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change of light sleep.

In some embodiments, the first temperatures of the bed device result in an increase in percent change of deep sleep. In some embodiments, the first temperatures of the bed device result in between 0%-300% increase in percent change of deep sleep. In some embodiments, the first temperatures of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change of deep sleep.

In some embodiments, the first temperatures of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of deep sleep. In some embodiments, the first temperatures of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change of deep sleep.

In some embodiments, the first temperatures of the bed device result in an increase in percent change of REM sleep. In some embodiments, the first temperatures of the bed device result in between 0%-300% increase in REM sleep. In some embodiments, the first temperatures of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change of REM sleep.

In some embodiments, the first temperatures of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of REM sleep. In some embodiments, the first temperatures of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change of REM sleep.

In some embodiments, the first temperatures of the bed device result in an increase in percent change of total sleep time. Total sleep time can correspond to the total amount of time the subject is asleep on the bed device. In some embodiments, the first temperatures of the bed device result in between 0%-500% increase in percent change of total sleep time. In some embodiments, the first temperatures results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% increase in percent change of total sleep time.

In some embodiments, the first temperatures of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of total sleep time. In some embodiments, the first temperatures of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in percent change of total sleep time.

In some embodiments, the first temperatures of the bed device result in an increase in a net sleep score. The net sleep score can be calculated by determining the percent change in how many subjects experienced a positive percent change in light, deep, REM, or total sleep time compared to a group that did not experience the first temperatures (e.g., a control group). In some embodiments, the first temperatures of the bed device result in between 0%-500% increase in percent change of net sleep score. In some embodiments, the first temperatures of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% increase in percent change of net sleep score.

In some embodiments, the first temperatures of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of net sleep score. In some embodiments, the first temperatures of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in percent change of net sleep score.

In some embodiments, the first temperatures of the bed device result in a decrease in percent change of total awake time. The total awake time can be calculated by determining the length of time the user is awake during use of the bed device. In some embodiments, the first temperatures of the bed device result in between 0%-500% decrease in percent change of total awake time. In some embodiments, the first temperatures of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% decrease in percent change of total awake time.

In some embodiments, the first temperatures of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less decrease in percent change of total awake time. In some embodiments, the first temperatures of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% decrease in percent change of total awake time.

In some embodiments, the first temperatures of the bed device result in a decrease in percent change of the number of awakenings per use of the bed device. The number of awakenings can be calculated by determining the number of times the user awaken during use of the bed device. In some embodiments, the first temperatures of the bed device result in between 0%-500% decrease in percent change of number of awakenings. In some embodiments, the first temperatures of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% decrease in percent change of numbers of awakenings.

In some embodiments, the first temperatures of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less decrease in percent change of number of awakenings. In some embodiments, the first temperatures of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% decrease in percent change of number of awakenings.

### Geolocation

In some embodiments, the first temperature can be determined by geolocation of the subject. In some cases, the geolocation can include the outdoor temperature. In some cases, the geolocation can include the outdoor humidity. In some cases, the geolocation can include the indoor temperature. In some cases, the geolocation can include the indoor humidity. In some cases, the geolocation can include the outdoor temperature and the outdoor humidity. In some cases, the geolocation can include the indoor temperature and the indoor humidity. In some cases, the geolocation can include the outdoor temperature, the outdoor humidity, the indoor temperature, and the indoor humidity. In some cases, the geolocation of the subject can be a cold climate. In some cases, the geolocation of the subject can be an alpine climate. An alpine climate can be a climate where no month has a mean temperature higher than about 10° C. In some cases, the geolocation of the subject can be an ice cap climate. An ice cap climate can be a climate where there can be no months above about 0° C. In some cases, the geolocation of the subject can be a subarctic climate. A subarctic climate can be a climate with a short summer (e.g., no more than about three months of the year) and winters where the temperature can drop below about −50° C. In some cases, the geolocation of the subject can be a tundra climate. A tundra climate can be a climate where at least about one month has an average temperature of about 0° C. but no month has an average temperature greater than about 10° C. In some cases, the geolocation of the subject can be a polar climate. A polar climate can be a climate where every month has an average temperature less than about 0° C. In some embodiments, the geolocation of the subject can be a dry climate. In some cases, the geolocation of the subject can be a desert climate. A desert climate can be climate where there can be greater evaporation of moisture over precipitation. In some cases, the geolocation of the subject can be a semi-arid climate. A semi-arid climate can be a climate that can receive precipitation below potential evapotranspiration. In some embodiments, the geolocation of the subject can be a humid climate. In some cases, the geolocation of the subject can be a humid continental climate. A humid continental climate can be a climate where the temperature of the coldest month is below about 0° C. and there can be at least about four months where the mean temperatures are at or above about 10° C. In some cases, the geolocation of the subject can be a humid subtropical climate. A humid subtropical climate can be a climate where at least about 8 months can have temperatures above about 10° C. In some cases, the geolocation of the subject can be a Mediterranean climate. A Mediterranean climate can be a climate with dry summers and wet winters. In some cases, the geolocation of the subject can be an oceanic climate. An oceanic climate can be a climate with a mean temperature of about 0° C. or higher in the coldest month. In some cases, the geolocation of the subject can be a tropical climate. In some instances, a tropical climate can be a topical monsoon climate. A tropical monsoon climate can be a climate with monthly mean temperature above about 18° C. and a dry season. In some instances, the tropical climate can be a tropical rainforest climate. A tropical rainforest climate can be a tropical climate that has no dry season. In some instances, the tropical climate can be a tropical savanna climate. A tropical savanna climate can be a climate that has a dry summer and a dry winter.

In some embodiments, the first temperatures can be determined by the geolocation of the subject. In some cases, the first temperatures of the pod for a subject in a cold climate can be warmer than the first temperatures of the pod for a subject in a dry climate. In some cases, the first temperatures of the pod for a subject in a cold climate can be warmer than the first temperatures of the pod for a subject in a humid climate. In some cases, the first temperatures of the pod for a subject in a cold climate can be warmer than the first temperatures of the pod for a subject in a Mediterranean climate. In some cases, the first temperatures of the pod for a subject in a cold climate can be warmer than the first temperatures of the pod for a subject in an oceanic climate. In some cases, the first temperatures of the pod for a subject in a cold climate can be warmer than the first temperatures of the pod for a subject in a tropical climate.

In some cases, the first temperatures of the pod for a subject in a dry climate can be colder than the first temperatures of the pod for a subject in a cold climate. In some cases, the first temperatures of the pod for a subject in a dry climate can be colder than the first temperatures of the pod for a subject in a humid climate. In some cases, the first temperatures of the pod for a subject in a dry climate can be warmer than the first temperatures of the pod for a subject in a humid climate. In some cases, the first temperatures of the pod for a subject in a dry climate can be colder than the first temperatures of the pod for a subject in a Mediterranean climate. In some cases, the first temperatures of the pod for a subject in a dry climate can be warmer than the first temperatures of the pod for a subject in a Mediterranean climate. In some cases, the first temperatures of the pod for a subject in a dry climate can be colder than the first temperatures of the pod for a subject in an oceanic climate. In some cases, the first temperatures of the pod for a subject in a dry climate can be warmer than the first temperatures of the pod for a subject in an oceanic climate. In some cases, the first temperatures of the pod for a subject in a dry climate can be colder than the first temperatures of the pod for a subject in a tropical climate. In some cases, the first temperatures of the pod for a subject in a dry climate can be warmer than the first temperatures of the pod for a subject in a tropical climate.

In some cases, the first temperatures of the pod for a subject in a humid climate can be colder than the first temperatures of the pod for a subject in a cold climate. In some cases, the first temperatures of the pod for a subject in a humid climate can be colder than the first temperatures of the pod for a subject in a dry climate. In some cases, the first temperatures of the pod for a subject in a humid climate can be warmer than the first temperatures of the pod for a subject in a dry climate. In some cases, the first temperatures of the pod for a subject in a humid climate can be colder than the first temperatures of the pod for a subject in a Mediterranean climate. In some cases, the first temperatures of the pod for a subject in a humid climate can be warmer than the first temperatures of the pod for a subject in a Mediterranean climate. In some cases, the first temperatures of the pod for a subject in a humid climate can be colder than the first temperatures of the pod for a subject in an oceanic climate. In some cases, the first temperatures of the pod for a subject in a humid climate can be warmer than the first temperatures of the pod for a subject in an oceanic climate. In some cases, the first temperatures of the pod for a subject in a humid climate can be colder than the first temperatures of the pod for a subject in a tropical climate. In some cases, the first temperatures of the pod for a subject in a humid climate can be warmer than the first temperatures of the pod for a subject in a tropical climate.

In some cases, the first temperatures of the pod for a subject in a Mediterranean climate can be colder than the first temperatures of the pod for a subject in a cold climate. In some cases, the first temperatures of the pod for a subject in a Mediterranean climate can be colder than the first temperatures of the pod for a subject in a dry climate. In some cases, the first temperatures of the pod for a subject in a Mediterranean climate can be warmer than the first temperatures of the pod for a subject in a dry climate. In some cases, the first temperatures of the pod for a subject in a Mediterranean climate can be colder than the first temperatures of the pod for a subject in a dry climate. In some cases, the first temperatures of the pod for a subject in a Mediterranean climate can be warmer than the first temperatures of the pod for a subject in a dry climate. In some cases, the first temperatures of the pod for a subject in a Mediterranean climate can be colder than the first temperatures of the pod for a subject in an oceanic climate. In some cases, the first temperatures of the pod for a subject in a Mediterranean climate can be warmer than the first temperatures of the pod for a subject in an oceanic climate. In some cases, the first temperatures of the pod for a subject in a Mediterranean climate can be colder than the first temperatures of the pod for a subject in a tropical climate. In some cases, the first temperatures of the pod for a subject in a Mediterranean climate can be warmer than the first temperatures of the pod for a subject in a tropical climate.

In some cases, the first temperatures of the pod for a subject in an oceanic climate can be colder than the first temperatures of the pod for a subject in a cold climate. In some cases, the first temperatures of the pod for a subject in an oceanic climate can be colder than the first temperatures of the pod for a subject in a dry climate. In some cases, the first temperatures of the pod for a subject in an oceanic climate can be warmer than the first temperatures of the pod for a subject in a dry climate. In some cases, the first temperatures of the pod for a subject in an oceanic climate can be colder than the first temperatures of the pod for a subject in a dry climate. In some cases, the first temperatures of the pod for a subject in an oceanic climate can be warmer than the first temperatures of the pod for a subject in a dry climate. In some cases, the first temperatures of the pod for a subject in an oceanic climate can be colder than the first temperatures of the pod for a subject in a Mediterranean climate. In some cases, the first temperatures of the pod for a subject in an oceanic climate can be warmer than the first temperatures of the pod for a subject in a Mediterranean climate. In some cases, the first temperatures of the pod for a subject in an oceanic climate can be colder than the first temperatures of the pod for a subject in a tropical climate. In some cases, the first temperatures of the pod for a subject in an oceanic climate can be warmer than the first temperatures of the pod for a subject in a tropical climate.

In some cases, the first temperatures of the pod for a subject in a tropical climate can be colder than the first temperatures of the pod for a subject in a cold climate. In some cases, the first temperatures of the pod for a subject in a tropical climate can be colder than the first temperatures of the pod for a subject in a dry climate. In some cases, the first temperatures of the pod for a subject in a tropical climate can be warmer than the first temperatures of the pod for a subject in a dry climate. In some cases, the first temperatures of the pod for a subject in a tropical climate can be colder than the first temperatures of the pod for a subject in a dry climate. In some cases, the first temperatures of the pod for a subject in a tropical climate can be warmer than the first temperatures of the pod for a subject in a dry climate. In some cases, the first temperatures of the pod for a subject in a tropical climate can be colder than the first temperatures of the pod for a subject in a Mediterranean climate. In some cases, the first temperatures of the pod for a subject in a tropical climate can be warmer than the first temperatures of the pod for a subject in a Mediterranean climate. In some cases, the first temperatures of the pod for a subject in a tropical climate can be colder than the first temperatures of the pod for a subject in an oceanic climate. In some cases, the first temperatures of the pod for a subject in a tropical climate can be warmer than the first temperatures of the pod for a subject in an oceanic climate.

In some embodiments, the first temperatures are determined by geolocation of the subject. In some embodiments, the geolocation of the subject is in a tropic region. A tropic region can be located between the Tropic of Cancer and the Tropic of Capricorn. In some embodiments, the geolocation of the subject is in a temperate region. A temperate region can be located between the Tropic of Cancer and the Artic circle or between the Tropic of Capricorn and the Antarctic circle. In some embodiments, the geolocation of the subject is in a Frigid region. A frigid region can be located above the Arctic circle or between the Antarctic circle.

In some embodiments, the first temperatures of the pod for a subject in a tropic region are cooler than the first temperatures of the pod for a subject in a temperate region. In some embodiments, the first temperatures of the pod for a subject in a tropic region are cooler than the first temperatures of the pod for a subject in a frigid region. In some embodiments, the first temperatures of the pod for a subject in a temperate region are cooler than the first temperatures of the pod for a subject in a tropic region. In some embodiments, the first temperatures of the pod for a subject in a temperate region are warmer than the first temperatures of the pod for a subject in a frigid region. In some embodiments, the first temperatures of the pod for a subject in a frigid region are warmer than the first temperatures of the pod for a subject in a temperate region. In some embodiments, the first temperatures of the pod for a subject in a frigid region is warmer than the first temperatures of the pod for a subject in a tropic region.

### Biological Sex

In some embodiments, the first temperatures of the bed device are determined by the biological sex of the subject. In some embodiments, the biological sex of the subject is male. In some embodiments, the biological sex of the subject is female. In some embodiments, the biological sex of the subject is unidentified. In some embodiments, the first temperatures of the bed device for male subjects are cooler than the first temperature of the bed device for female or unidentified subjects. In some embodiments, the first temperatures of the bed device for female or unidentified subjects are warmer than the first temperature of the bed device for male subjects. In some embodiments, the first temperatures of the bed device for female or unidentified subjects are between about 1° C. to about 5° C. warmer than the first temperature of the bed device for male subjects. In some embodiments, the first temperatures of the bed device for female or unidentified subjects are at least or up to about 1° C., at least or up to about 2° C., at least or up to about 3° C., at least or up to about 4° C., or at least or up to about 5° C. warmer than the first temperatures for the bed device for male subjects. In some embodiments, the first temperatures of the bed device for male subjects are between about 1° C. to about 5° C. cooler than the first temperatures of the bed device for female or unidentified subjects. In some embodiments, the first temperatures of the bed device for male subjects are at least or up to about 1° C., at least or up to about 2° C., at least or up to about 3° C., at least or up to about 4° C., or at least or up to about 5° C. cooler than the first temperatures of the bed device for female or unidentified subjects. In some embodiments, the first temperatures for the female or unidentified subjects are higher than the first temperature for male subject regardless of the age of the subject, the geolocation of the subject, and/or the season of the geolocation of the subject.

In some embodiments, the first temperatures of the bed device for male subjects are between about 1 to about 8 dial values lower than the dial values for female or unidentified subjects. In some embodiments, the first temperatures of the bed device for male subjects are at least or up to about 1 dial value, at least or up to about 2 dial values, at least or up to about 3 dial values, at least or up to about 4 dial values, at least or up to about 5 dial values, at least or up to about 6 dial values, at least or up to about 7 dial values, or at least or up to about 8 vial values lower than the dial values for female or unidentified subjects. In some embodiments, the first temperatures of the bed device for female or unidentified subjects are between about 1 to about 8 dial values higher than the dial values for male subjects. In some embodiments, the first temperatures of the bed device for female or unidentified subjects are at least or up to about 1 dial value, at least or up to about 2 dial values, at least or up to about 3 dial values, at least or up to about 4 dial values, at least or up to about 5 dial values, at least or up to about 6 dial values, at least or up to about 7 dial values, or at least or up to about 8 vial values higher than the dial values for male subjects.

In some embodiments, the first temperatures of the bed device can be higher for female subjects or unidentified subjects compared to male subjects. In some cases, the first temperatures of the female subjects or the unidentified subjects can be compared to the first temperatures of the male subjects. In some cases, the first temperature of the female subjects or the unidentified subjects can be compared to the first temperatures of the male subjects for a specific sleep stage of the user.

In some examples, the first temperatures of the female subject or unidentified subject during and the first temperatures of the male subject can be compared when the female or unidentified subject and the male subject are in light sleep. In some cases, the first temperatures of the female subject or the unidentified subject can be higher than the first temperatures of the male subject when the female or unidentified subject and the male subject are in light sleep.

Alternatively, in some examples, the first temperatures of the female subject or the unidentified subject can be lower than the first temperatures of the male subject when the female or unidentified subject and the male subject are in light sleep. In some examples, the first temperatures of the female subject or the unidentified subject can be the same as the first temperatures of the male subject when the female or unidentified subject and the male subject are in light sleep.

In some examples, the first temperatures of the female subject or unidentified subject during and the first temperatures of the male subject can be compared when the female or unidentified subject and the male subject are in deep sleep. In some cases, the first temperatures of the female subject or the unidentified subject can be higher than the first temperatures of the male subject when the female or the unidentified subject and the male subject are in deep sleep.

Alternatively, in some examples, the first temperatures of the female subject or the unidentified subject can be lower than the first temperatures of the male subject when the female or unidentified subject and the male subject are in deep sleep. In some examples, the first temperatures of the female subject or the unidentified subject can be the same as the first temperatures of the male subject when the female or unidentified subject and the male subject are in deep sleep.

In some examples, the first temperatures of the female subject or unidentified subject during and the first temperatures of the male subject can be compared when the female or unidentified subject and the male subject are in REM sleep. In some cases, the first temperatures of the female or unidentified subject can be higher than the first temperatures of the male subject when the female or unidentified subject and the male subject are in REM sleep.

Alternatively, in some examples, the first temperatures of the female subject or the unidentified subject can be lower than the first temperatures of the male subject when the female or unidentified subject and the male subject are in REM sleep. In some examples, the first temperatures of the female subject or the unidentified subject can be the same as the first temperatures of the male subject when the female or unidentified subject and the male subject are in REM sleep.

In some cases, the term “biological sex” as provided herein may be a biological sex of the subject (e.g., based on anatomical feature of the subject at birth) or a biological sex determined by the subject.

### Age

In some embodiments, the first temperatures of the bed device are determined by the age of the subject. In some embodiments, the subject enters the subject's birthday into the bed device app and the bed device app automatically calculates the age of the subject. In some embodiments, the subject enters the subject's age into the bed device app. In some embodiments, the age of the subject is less than about 35 years old. In some embodiments, the age of the subject is between about 35 years old to about 51 years old. In some embodiments, the age of the subject is greater than about 51 years old.

In some embodiments, the first temperatures of the bed device for a subject less than about 35 years old are cooler than the first temperatures of the bed device for a subject between about 35 years old to about 51 years old. In some embodiments, the first temperatures of the bed device for a subject less than about 35 years old are between about 0 to about 4 dial values lower than the dial values for a subject between the age of about 35 years old to about 51 years old. In some embodiments, the first temperatures of the bed device for a subject less than about 35 years old are at least or up to about 1 dial value, at least or up to about 2 dial values, at least or up to about 3 dial values, or at least or up to about 4 dial values lower than the dial values for a subject between about 35 years old to about 51 years old.

In some embodiments, the first temperatures of the bed device for a subject between the age of about 35 years to about 51 years old are warmer than the first temperatures of the bed device for a subject less than about 35 years old. In some embodiments, the first temperatures of the bed device for a subject between the age of about 35 years old to about 51 years old are between about 0 to about 4 dial values higher than the dial values for a subject less than 35 years old. In some embodiments, the first temperatures of the bed device for a subject between the age of about 35 years old to about 51 years old are at least or up to about 1 dial value, at least or up to about 2 dial values, at least or up to about 3 dial values, or at least or up to about 4 dial values higher than the dial values for a subject less than about 35 years old.

In some embodiments, the first temperatures of the bed device for a subject less than about 35 years old are between about 0° C. to about 7° C. cooler than the first temperatures of the bed device for a subject between the age of about 35 years old to about 51 years old. In some embodiments, the first temperatures of the bed device for a subject less than about 35 years old are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., or at least or up to about 7° C., cooler than the temperatures for a subject between about 35 years old to about 51 years old.

In some embodiments, the first temperatures of the bed device for a subject between the age of about 35 years old to about 51 years old are between about 0° C. to about 7° C. warmer than the first temperatures of the bed device for a subject less than 35 years old. In some embodiments, the first temperatures of the bed device for a subject between the age of about 35 years old to about 51 years old are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., or at least or up to about 7° C. warmer than the first temperatures of the bed device for a subject less than about 35 years old.

In some embodiments, the first temperatures of the bed device for a subject less than about 35 years old are cooler than the first temperatures of the bed device for a subject older than about 51 years old. In some embodiments, the first temperatures of the bed device for a subject less than about 35 years old are between about 0 to about 10 dial values lower than the dial values for a subject older than about 51 years old. In some embodiments, the first temperatures of the bed device for a subject less than about 35 years old are at least or up to about 1 dial value, at least or up to about 2 dial values, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, or at least or up to about 10 dial values lower than the dial values for a subject older than about 51 years old.

In some embodiments, the first temperatures of the bed device for a subject older than about 51 years old are warmer than the first temperatures of the bed device for a subject less than about 35 years old. In some embodiments, the first temperatures of the bed device for a subject older than about 51 years old are between about 0 to about 10 dial values higher than the dial values for a subject less than about 35 years old. In some embodiments, the first temperatures of the bed device for a subject older than about 51 years old are at least or up to about 1 dial value, at least or up to about 2 dial values, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, or at least or up to about 10 dial values higher than the dial values for a subject less than about 35 years old.

In some embodiments, the first temperatures of the bed device for a subject less than about 35 years old are cooler than the first temperatures of the bed device for a subject older than about 51 years old. In some embodiments, the first temperatures of the bed device for a subject less than about 35 years old are between about 0° C. to about 13° C. cooler than the first temperatures for the bed device for a subject older than about 51 years old. In some embodiments, the first temperatures of the bed device for a subject less than about 35 years old are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., or at least or up to about 16° C. cooler than the first temperatures for the bed device for a subject older than about 51 years old.

In some embodiments, the first temperatures of the bed device for a subject older than about 51 years old are between about 0° C. to about 13° C. warmer than the first temperatures for the bed device for a subject less than about 35 years old. In some embodiments, the first temperatures of the bed device for a subject older than about 51 years old are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., or at least or up to about 16° C. warmer than the first temperatures for the bed device for a subject less than about 35 years old.

In some embodiments, the first temperatures of the bed device for a subject between the age of about 35 years old to about 51 years old are cooler than the first temperatures of the bed device for a subject older than 51 years old. In some embodiments, the first temperatures of the bed device for a subject between the age of about 35 years old to about 51 years old are between about 0 to about 8 dial values lower than the dial values for a subject older than about 51 years old. In some embodiments, the first temperatures of the bed device for a subject between the age of 35 years old to about 51 years old are at least or up to about 1 dial value, at least or up to about 2 dial values, at least or up to about 3 dial values, at least or up to about 4 dial values, at least or up to about 5 dial values, at least or up to about 6 dial values, at least or up to about 7 dial values, or at least or up to about 8 dial values lower than the dial values for a subject older than about 51 years old.

In some embodiments, the first temperatures of the bed device for a subject older than about 51 years old are between about 0 to about 8 dial values higher than the dial values for a subject between the age of about 35 years old to about 51 years old. In some embodiments, the first temperatures of the bed device for a subject older than about 51 years old are at least or up to about 0 dial values, at least or up to about 1 dial values, at least or up to about 2 dial values, at least or up to about 3 dial values, at least or up to about 4 dial values, at least or up to about 5 dial values, at least or up to about 6 dial values, at least or up to about 7 dial values, or at least or up to about 8 vial values higher than the dial values for a subject between the age of about 35 years old to about 51 years old.

In some embodiments, the first temperatures of the bed device for a subject between the age of about 35 years old to about 51 years old are between about 0° C. to about 13° C. cooler than the first temperatures of the bed device for a subject older than about 51 years old. In some embodiments, the first temperatures of the bed device for a subject between the age of 35 years old to about 51 years old are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., or at least or up to about 13° C., cooler than the first temperatures of the bed device for a subject older than about 51 years old.

In some embodiments, the first temperatures of the bed device for a subject older than about 51 years old are between about 0° C. to about 13° C. warmer than the first temperatures of the bed device a subject between the age of about 35 years old to about 51 years old. In some embodiments, the first temperatures of the bed device for a subject older than about 51 years old are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., or at least or up to about 13° C., warmer than the first temperatures of the bed device for a subject between the age of about 35 years old to about 51 years old

### Season

In some embodiments, the first temperatures of the bed device are determined by the season of the geolocation where the subject is located. In some embodiments, the season of the geolocation is spring. In some embodiments, the season of the geolocation is winter. In some embodiments, the season of the geolocation is summer. In some embodiments, the season of the geolocation is fall. In some embodiments, the season of the geolocation is a dry season. In some cases, the dry season can correspond with summer. In some cases, the dry season can correspond with spring. In some cases, the dry season can correspond with fall. In some cases, the dry season can correspond with winter. In some embodiments, the season of the geolocation is a wet season. In some cases, the wet season can correspond with spring. In some cases, the wet season can correspond with summer. In some cases, the wet season can correspond with fall. In some cases, the wet season can correspond with winter.

In some embodiments, the season of the geolocation is a transition period between winter and spring. In some cases, the first temperatures of the bed device in the transition period between winter and spring can be colder than the first temperatures of the bed device in winter. In some embodiments, the first temperatures of the bed device in the transition period between winter and spring are warmer than the first temperatures of the bed device in the spring. In some embodiments, the first temperatures of the bed device in the transition period between winter and spring are between about 0° C. to about 31° C. warmer than the first temperatures of the bed device in the spring. In some embodiments, the first temperatures of the bed device in the transition period between winter and spring are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. warmer than the first temperatures of the bed device in the spring.

In some embodiments, the first temperatures of the bed device in the transition period between winter and spring are between about 0 to about 20 dial values of the bed device higher than the dial values of the bed device in the spring. In some embodiments, the first temperatures of the bed device in the transition period between winter and spring are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates higher than the dial values of the bed device in the spring.

In some cases, the first temperatures of the bed device in the transition period between winter and spring can be warmer than the first temperatures of the bed device in spring. In some embodiments, the first temperatures of the bed device in the transition period between winter and spring are between about 0° C. to about 31° C. cooler than the first temperatures of the bed device in the winter. In some embodiments, the first temperatures of the bed device in the transition period between winter and spring are at least or up to about 0° C., at least or up to about 0.5° C., at least or up to about 1° C., at least or up to about 1.5° C., at least or up to about 2° C., at least or up to about 2.5° C., or at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. cooler than the first temperatures of the bed device in the winter.

In some embodiments, the first temperatures of the bed device in the transition period between winter and spring are between about 0 to about 20 dial values of the bed device lower than the dial values of the bed device in the winter. In some embodiments, the first temperatures of the bed device in the transition period between winter and spring are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates lower than the dial values of the bed device in the winter.

In some embodiments, the season of the geolocation is a transition period between spring and summer. In some cases, the first temperatures of the bed device in the transition period between spring and summer can be colder than the first temperatures of the bed device in spring. In some embodiments, the first temperatures of the bed device in the transition period between spring and summer are between about 0° C. to about 31° C. cooler than the first temperatures of the bed device in the spring. In some embodiments, the first temperatures of the bed device in the transition period between spring and summer are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. cooler than the first temperatures of the bed device in the spring.

In some embodiments, the first temperatures of the bed device in the transition period between spring and summer are between about 0 to about 20 dial values of the bed device lower than the dial values of the bed device in the spring. In some embodiments, the first temperatures of the bed device in the transition period between spring and summer are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates lower than the dial values of the bed device in the spring.

In some cases, the first temperatures of the bed device in the transition period between spring and summer can be warmer than the first temperatures of the bed device in summer. In some embodiments, the first temperatures of the bed device in the transition period spring and summer are between about 0° C. to about 31° C. warmer than the first temperatures of the bed device in the summer. In some embodiments, the first temperatures of the bed device in the transition period spring and summer are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. warmer than the first temperatures of the bed device in the summer.

In some embodiments, the first temperatures of the bed device in the transition period spring and summer are between about 0 to about 20 dial values of the bed device higher than the dial values of the bed device in the summer. In some embodiments, the first temperatures of the bed device in the transition period spring and summer are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates higher than the dial values of the bed device in the summer.

In some embodiments, the season of the geolocation is a transition period between summer and fall. In some cases, the first temperatures of the bed device in the transition period between summer and fall can be warmer than the first temperatures of the bed device during the summer. In some embodiments, the first temperatures of the bed device in the transition period between summer and fall are between about 0° C. to about 31° C. warmer than the first temperatures of the bed device in the summer. In some embodiments, the first temperatures of the bed device in the transition period between summer and fall are at least or up to about 0° C., at least or up to about 0.5° C., at least or up to about 1° C., at least or up to about 1.5° C., at least or up to about 2° C., at least or up to about 2.5° C., or at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. warmer than the first temperatures of the bed device in the summer.

In some embodiments, the first temperatures of the bed device in the transition period between summer and fall are between about 0 to about 20 dial values of the bed device higher than the dial values of the bed device in the summer. In some embodiments, the first temperatures of the bed device in the transition period between summer and fall are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates higher than the dial values of the bed device in the summer.

In some cases, the first temperatures in the transition period between summer and fall can be colder than the first temperatures of the bed device during the fall. In some embodiments, the first temperatures of the bed device in the transition period between summer and fall are between about 0° C. to about 31° C. cooler than the first temperatures of the bed device in the fall. In some embodiments, the first temperatures of the bed device in the transition period between summer and fall are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. cooler than the first temperatures of the bed device in the fall.

In some embodiments, the first temperatures of the bed device in the transition period between summer and fall are between about 0 to about 20 dial values of the bed device lower than the dial values of the bed device in the fall. In some embodiments, the first temperatures of the bed device in the transition period between summer and fall are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates lower than the dial values of the bed device in the fall.

In some embodiments, the season of the geolocation is a transition period between fall and winter. In some cases, the first temperatures of the bed device in the transition period between fall and winter can be warmer than the first temperatures of the bed device during the fall. In some embodiments, the first temperatures of the bed device in the transition period between fall and winter are between about 0° C. to about 31° C. warmer than the first temperatures of the bed device in the fall. In some embodiments, the first temperatures of the bed device in the transition period between fall and winter are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. warmer than the first temperatures of the bed device in the fall.

In some embodiments, the first temperatures of the bed device in the transition period between fall and winter are between about 0 to about 20 dial values of the bed device higher than the dial values of the bed device in the fall. In some embodiments, the first temperatures of the bed device in the transition period between fall and winter are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates higher than the dial values of the bed device in the fall.

In some cases, the first temperatures of the bed device in the transition period between fall and winter can be colder than the first temperatures of the bed device during winter. In some embodiments, the first temperatures of the bed device in the transition period between fall and winter are between about 0° C. to about 31° C. cooler than the first temperatures of the bed device in the winter. In some embodiments, the first temperatures of the bed device in the transition period between fall and winter are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. cooler than the first temperatures of the bed device in the winter.

In some embodiments, the first temperatures of the bed device in the transition period between fall and winter are between about 0 to about 20 dial values of the bed device lower than the dial values of the bed device in the winter. In some embodiments, the first temperatures of the bed device in the transition period between fall and winter are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates lower than the dial values of the bed device in the winter.

In some embodiments, the first temperatures of the bed device in the winter are warmer than the first temperatures of the bed device in the summer. In some embodiments, the first temperatures of the bed device in the winter are between about 0° C. to about 31° C. warmer than the first temperatures of the bed device in the summer. In some embodiments, the first temperatures of the bed device in the winter are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. warmer than the first temperatures of the bed device in the summer.

In some embodiments, the first temperatures of the bed device in the winter are between about 0 to about 20 dial values of the bed device higher than the dial values of the bed device in the summer. In some embodiments, the first temperatures of the bed device in the winter are at least or up to about 0 dial value, at least or up to about 1 dial value, at least or up to about 2 dial values, at least or up to about 3 dial values, at least or up to about 4 dial values, at least or up to about 5 dial values, at least or up to about 6 dial values, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up toa bout 16 dial updates, at least or up to about 17 dial updates, at least or up to about 18 dial updates, at least or up to about 19 dial updates, or at least or up to about 20 dial updates higher than the dial values of the bed device in the summer.

In some embodiments, the first temperatures of the bed device in the summer are cooler than the first temperatures of the bed device in the winter. In some embodiments, the first temperatures of the bed device in the summer are between about 0° C. to about 31° C. cooler than the first temperatures of the bed device in the winter. In some embodiments, the first temperatures of the bed device in the summer are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. cooler than the first temperatures of the bed device in the winter.

In some embodiments, the first temperatures of the bed device in the summer are between about 0 to about 20 dial values of the bed device lower than the dial values of the bed device in the winter. In some embodiments, the first temperatures of the bed device in the summer are at least or up to about 0 dial value, at least or up to about 1 dial value, at least or up to about 2 dial values, at least or up to about 3 dial values, at least or up to about 4 dial values, at least or up to about 5 dial values, at least or up to about 6 dial values, at least or up to about 7 dial values, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates lower than the dial values of the bed device in the winter.

In some embodiments, the first temperatures of the bed device in the fall are warmer than the first temperatures of the bed device in the summer. In some embodiments, the first temperatures of the bed device in the fall are between about 0° C. to about 31° C. warmer than the first temperatures of the bed device in the summer. In some embodiments, the first temperatures of the bed device in the winter are at least or up to about 0° C., at least or up to about 0.5° C., at least or up to about 1° C., at least or up to about 1.5° C., at least or up to about 2° C., at least or up to about 2.5° C., or at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. warmer than the first temperatures of the bed device in the summer.

In some embodiments, the first temperatures of the bed device in the fall are between about 0 to about 20 dial values of the bed device higher than the dial values of the bed device in the summer. In some embodiments, the first temperatures of the bed device in the fall are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates higher than the dial values of the bed device in the summer.

In some embodiments, the first temperatures of the bed device in the spring are warmer than the first temperatures of the bed device in the summer. In some embodiments, the first temperatures of the bed device in the spring are between about 0° C. to about 31° C. warmer than the first temperatures of the bed device in the summer. In some embodiments, the first temperatures of the bed device in the spring are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. warmer than the first temperatures of the bed device in the summer.

In some embodiments, the first temperatures of the bed device in the spring are between about 0 to about 20 dial values of the bed device higher than the dial values of the bed device in the summer. In some embodiments, the first temperatures of the bed device in the spring are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates higher than the dial values of the bed device in the summer.

In some embodiments, the first temperatures of the bed device in the fall are warmer than the first temperatures of the bed device in the spring. In some embodiments, the first temperatures of the bed device in the fall are between about 0° C. to about 31° C. warmer than the first temperatures of the bed device in the spring. In some embodiments, the first temperatures of the bed device in the fall are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. warmer than the first temperatures of the bed device in the spring.

In some embodiments, the first temperatures of the bed device in the fall are between about 0 to about 20 dial values of the bed device higher than the dial values of the bed device in the spring. In some embodiments, the first temperatures of the bed device in the fall are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates higher than the dial values of the bed device in the spring.

In some embodiments, the first temperatures of the bed device in the winter are warmer than the first temperatures of the bed device in the spring. In some embodiments, the first temperatures of the bed device in the winter are between about 0° C. to about 31° C. warmer than the first temperatures of the bed device in the spring. In some embodiments, the first temperatures of the bed device in the winter are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. warmer than the first temperatures of the bed device in the spring.

In some embodiments, the first temperatures of the bed device in the winter are between about 0 to about 20 dial values of the bed device higher than the dial values of the bed device in the spring. In some embodiments, the first temperatures of the bed device in the winter are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates higher than the dial values of the bed device in the spring.

In some embodiments, the first temperatures of the bed device in the winter are warmer than the first temperatures of the bed device in the fall. In some embodiments, the first temperatures of the bed device in the winter are between about 0° C. to about 31° C. warmer than the first temperatures of the bed device in the fall. In some embodiments, the first temperatures of the bed device in the winter are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. warmer than the first temperatures of the bed device in the fall.

In some embodiments, the first temperatures of the bed device in the winter are between about 0 to about 20 dial values of the bed device higher than the dial values of the bed device in the fall. In some embodiments, the first temperatures of the bed device in the winter are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates higher than the dial values of the bed device in the fall.

In some embodiments, the first temperatures of the bed device in the spring are cooler than the first temperatures of the bed device in the fall. In some embodiments, the first temperatures of the bed device in the spring are between about 0° C. to about 31° C. cooler than the first temperatures of the bed device in the fall. In some embodiments, the first temperatures of the bed device in the spring are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. cooler than the first temperatures of the bed device in the fall.

In some embodiments, the first temperatures of the bed device in the spring are between about 0 to about 20 dial values of the bed device lower than the dial values of the bed device in the fall. In some embodiments, the first temperatures of the bed device in the spring are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates lower than the dial values of the bed device in the fall.

In some embodiments, the first temperatures of the bed device in the summer are cooler than the first temperatures of the bed device in the spring. In some embodiments, the first temperatures of the bed device in the summer are between about 0° C. to about 31° C. cooler than the first temperatures of the bed device in the spring. In some embodiments, the first temperatures of the bed device in the summer are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. cooler than the first temperatures of the bed device in the spring.

In some embodiments, the first temperatures of the bed device in the summer are between about 0 to about 20 dial values of the bed device lower than the dial values of the bed device in the spring. In some embodiments, the first temperatures of the bed device in the summer are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates lower than the dial values of the bed device in the spring.

In some embodiments, the first temperatures of the bed device in the summer are cooler than the first temperatures of the bed device in the fall. In some embodiments, the first temperatures of the bed device in the summer are between about 0° C. to about 31° C. cooler than the first temperatures of the bed device in the fall. In some embodiments, the first temperatures of the bed device in the summer are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. cooler than the first temperatures of the bed device in the fall.

In some embodiments, the first temperatures of the bed device in the summer are between about 0 to about 20 dial values of the bed device lower than the dial values of the bed device in the fall. In some embodiments, the first temperatures of the bed device in the summer are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates lower than the dial values of the bed device in the fall.

In some embodiments, the first temperatures of the bed device in the spring are cooler than the first temperatures of the bed device in the winter. In some embodiments, the first temperatures of the bed device in the spring are between about 0° C. to about 31° C. cooler than the first temperatures of the bed device in the winter. In some embodiments, the first temperatures of the bed device in the spring are at least or up to about 0° C., at least or up to about 0.5° C., at least or up to about 1° C., at least or up to about 1.5° C., at least or up to about 2° C., at least or up to about 2.5° C., or at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. cooler than the first temperatures of the bed device in the winter.

In some embodiments, the first temperatures of the bed device in the spring are between about 0 to about 20 dial values of the bed device lower than the dial values of the bed device in the winter. In some embodiments, the first temperatures of the bed device in the spring are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates lower than the dial values of the bed device in the winter.

In some embodiments, the first temperatures of the bed device in the fall are cooler than the first temperatures of the bed device in the winter. In some embodiments, the first temperatures of the bed device in the fall are between about 0° C. to about 31° C. cooler than the first temperatures of the bed device in the winter. In some embodiments, the first temperatures of the bed device in the fall are at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., at least or up to about 30° C., at least or up to about 30.5° C., or at least or up to about 31° C. cooler than the first temperatures of the bed device in the winter.

In some embodiments, the first temperatures of the bed device in the fall are between about 0 to about 20 dial values of the bed device lower than the dial values of the bed device in the winter. In some embodiments, the first temperatures of the bed device in the fall are at least or up to about 0 dial value, at least or up to about 1 dial value, at least or up to about 2 dial values, at least or up to about 3 dial values, at least or up to about 4 dial updates, at least or up to about 5 dial updates, at least or up to about 6 dial updates, at least or up to about 7 dial updates, at least or up to about 8 dial updates, at least or up to about 9 dial updates, at least or up to about 10 dial updates, at least or up to about 11 dial updates, at least or up to about 12 dial updates, at least or up to about 13 dial updates, at least or up to about 14 dial updates, at least or up to about 15 dial updates, at least or up to about 16 dial updates, at least or up to about 17 dial updates, at least or up to about 18 dial updates, at least or up to about 19 dial updates, or at least or up to about 20 dial updates lower than the dial values of the bed device in the winter.

### Women's Health

One aspect of the present disclosure provides methods (e.g., computer-implemented methods) for regulating the temperature of the bed device based on a woman's biological state. A woman can be in one or more biological states: menstruating (e.g., premenopausal), menopausal, pregnant, perimenopausal, post-menopausal, or post-partum. In some embodiments, the subject inputs into the bed device which woman's biological state the subject is in. In some embodiments, the subject is in a menstruating biological state. In some embodiments, the subject is in a menopausal biological state. In some embodiments, the subject is in a pregnant biological state. In some embodiments, the subject is in a post-partum biological state. In some embodiments, the bed device regulates temperatures based on the biological state of the woman subject. In some embodiments, the bed device regulates temperatures based on the menstruating biological state of the woman subject. In some embodiments, the bed device regulates temperatures based on the menopausal biological state of the woman subject. In some embodiments, the bed device regulates the temperatures based on the pregnancy state of the woman subject. In some embodiments, the bed device regulates the temperatures based on the post-partum state of the woman subject.

In some embodiments, the woman subject classifies the type of biological sex the woman identifies as. In some embodiments, the woman subject inputs the subject's biological sex into the bed device app via a graphical user interface (GUI). In some cases, the user device is wirelessly connected to the bed device. In some cases, the user inputs user information in the bed device app via the GUI. In some cases, the bed device app digitally communicates the user information to the bed device. In some embodiments, the woman subject inputs the subject's biological sex as “female” into the bed device app. In some embodiments, the woman subject classifies the type of biological state that the subject experiences. Non-limiting examples of types of biological states that women subjects' experiences include regular menstrual cycle (e.g., every 28-32 days) with no birth control, regular menstrual cycle with some form of birth control, irregular menstrual cycle with some form of birth control, irregular menstrual cycle with no birth control, amenorrheic (e.g., absence of menstruation), pre-menopausal with hormone replacement, pre-menopausal without hormone replacement, pregnant, post-partum, breast-feeding, post-menopausal with hormone replacement therapy, post-menopausal without hormone replacement therapy, polycystic ovarian syndrome, and other women-specific conditions or diseases. In some embodiments, the subject selects at least one classification of the biological state that best describes the subject in the bed device app. In some embodiments, the subject selects at least two classifications of the biological state that best describes the subject in the bed device app. In some embodiments, the subject selects at least two classifications of the biological state that best describes the subject in the bed device app. In some embodiments, the bed device app recommends a mode for the subject based on the at least one classification of the biological state that the subject selects in the app. In some embodiments, the subject can accept the mode recommendation from the bed device app. In some embodiments, the subject can reject the mode recommendation from the bed device app. In some embodiments, the bed device app recommends a menstruation mode. In some embodiments, the bed device app recommends a menopausal mode. In some embodiments, the bed device app recommends a pregnancy mode.

In some embodiments, each mode of the bed device (e.g., menstruation mode, menopausal mode, pregnancy mode, etc.) comprises a predetermined profile of temperatures associated with each mode. In some cases, the menstruation mode comprises a predetermined profile of temperatures. In some examples, the menstruation mode comprises a predetermined profile of temperatures based on a schedule of menstrual cycles of the user, a time within the menstrual cycle, or lack of menstrual cycle. In some examples, the menstruation mode comprises a predetermined profile of temperatures based on use of birth control or lack thereof. In some cases, the menopausal mode comprises a predetermined profile of temperatures. In some examples, the menopausal mode comprises a predetermined profile of temperatures based whether the user has started menopause or not. In some examples, the menopausal mode comprises a predetermined profile of temperatures based on use of hormonal therapy or lack thereof. In some cases, the pregnancy mode comprises a predetermined profile of temperatures. In some examples, the pregnancy mode comprises a predetermined profile of temperatures based on whether the user is pregnant or not. In some examples, the pregnancy mode comprises a predetermined profile of temperatures based on whether the user is post-partum. In some examples, the pregnancy mode comprises a predetermined profile of temperatures based on whether the user is breast-feeding. In some cases, the bed device comprises a predetermined profile of temperatures based on whether the user has polycystic ovarian syndrome. In some cases, the bed device comprises a predetermined profile of temperatures based on whether the user experiences hot flashes. In some cases, the bed device comprises a predetermined profile of temperatures based on other women-specific conditions or diseases. In some embodiments, the predetermined profile of temperatures for each mode of the bed device is stored in a database. In some cases, the database is stored in the bed device. In some cases, the database is stored in the cloud.

In some embodiments, the one or more sensors detect one or more biometrics from the subject during use of the bed device. In some embodiments, the bed device app collects data regarding the one or more biometrics from the subject during the use of the bed device. In some embodiments, the bed device app uses the one or more biometrics collected to determine the biological sex of the subject. In some embodiments, the bed device app determines the biological sex of the subject with a probability of between about 0% to about 100%. In some embodiments, the bed device app determines the biological sex of the subject with a probability of at least or up to about 0%, at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 95%, at least or up to about 98%, at least or up to about 99%, or at least or up to about 100%. In some embodiments, the bed device app determines the biological sex of the subject with a probability of about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the one or more sensors detect one or more biometrics from the subject during use of the bed device. In some embodiments, the bed device app collects data regarding the one or more biometrics from the subject during the use of the bed device. In some embodiments, the bed device app uses the one or more biometrics collected to determine the type of biological state that the subject experiences. In some embodiments, the bed device app determines at least or up to about one type of biological state, at least or up to about two types of biological states, or at least or up to about three types of biologicals states that the subject experiences. In some embodiments, the bed device app determines the type of biological state that the subject experiences with a probability of between about 0% to about 100%. In some embodiments, the bed device app determines the type of biological state that the subject experiences with a probability of at least or up to about 0%, at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 95%, at least or up to about 98%, at least or up to about 99%, or at least or up to about 100%. In some embodiments, the bed device app determines the type of biological state of the subject experiences with a probability of about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the biological sex of the subject is a female. In some embodiments, the biological sex of the subject is unidentified.

i. Menstruation

In some embodiments, the bed device app recommends a menstruation mode to the subject. Non-limiting classification of biological states that triggers a menstruation mode recommendation includes regular menstrual cycle with no birth control, regular menstrual cycle with some form of birth control, irregular menstrual cycle with some form of birth control, irregular menstrual cycle with no birth control, amenorrheic, and polycystic ovarian syndrome. In some embodiments, the subject can accept the menstruation mode recommendation. In some embodiments, the subject can reject the menstruation mode recommendation. In some embodiments, the subject inputs additional information into the bed device if the menstruation mode recommendation is accepted.

In some embodiments, the subject inputs additional information into the bed device app if regular menstrual cycle with no birth control or regular menstrual cycle with birth control is selected. In some embodiments, the additional information is the date of the last period (e.g., start of menstruation) of the subject. In some embodiments, the additional information is the average length of menstruation (e.g., number of days between starts of period). In some embodiments, the additional information is the date of the last period and the average length of menstruation.

In some embodiments, the menstruation mode regulates the temperatures of the bed device based on the menstruation cycle of the subject. In some embodiments, the menstruation mode regulates the temperature of the bed device based on the phase of the menstrual cycle of the subject. The menstrual cycle can have two phases: luteal and follicular. Follicular phase can start on the first day of a subject's period (e.g., start of menstruation) and can last for between about 8 to about 21 days. Ovulation can occur at the end of the follicular phase. Luteal phase can after the start of the follicular phase. Luteal phase can last for between about 9 to about 27 days.

In some embodiments, the bed device app calculates the predicted start dates of the follicular phase and luteal phase for subjects with regular periods. In some embodiments, the bed device app uses the information input by the subject to calculate the predicted start dates of the subject period, the start of the follicular phase, or the start of the luteal phase.

In some embodiments, the bed device app calculates the predicted start date of the subject's period with the following formula:

$$Start date of follicular phase + average cycle length \qquad ( II )$$

In some embodiments, the predicted start date of the follicular phase is based on the average cycle length information inputted by the subject. When the average cycle length is between about 21 to about 24 days, the follicular phase length can be at least or up to about 12 days. When the average cycle length is between about 25 to about 30 days, the follicular phase length can be at least or up to about 15 days. When the average cycle length is between about 31 to about 35 days, the follicular phase length can be at least or up to about 19 days.

In some embodiments, the bed device app calculates the start of the luteal phase of the subject. In some embodiments, the bed device app calculates the start of the luteal phase of the subject using the following formula:

$$next period start date - 10 days \qquad ( III )$$

In some embodiments, the bed device app calculates the end of the luteal phase of the subject. In some embodiments, the bed device app calculates the end of the luteal phase of the subject using the following formula:

$$next period start date - 1 day \qquad ( IV )$$

In some embodiments, the bed device app uses these calculations to determine when the follicular phase and the luteal phase of the subject starts. In some embodiments, the temperature of the bed device during the luteal phase of the subject is different from the temperature of the bed device during the follicular phase of the subject.

In some embodiments, the bed device further comprises a processor. In some embodiments, the processor analyzes the data collected by the one or more sensors. In some embodiments, the bed device further comprises a temperature regulator. In some embodiments, the temperature regulator is operatively connected to the processor. In some embodiments, the processor can provide a control signal to the temperature regulator. In some embodiments, the control signal can be an instruction for the temperature regulator to change a temperature of the article of furniture. In some embodiments, the control signal can be an instruction for the temperature regulator to change a temperature of a portion of the article of furniture (e.g., bed device). In some embodiments, the control signal can be an instruction to change the temperature of the bed device to the bed device temperature associated with the follicular phase of the menstrual cycle. In some embodiments, the control signal can be an instruction to change the temperature of the bed device to the bed device temperature associated with the luteal phase of the menstrual cycle.

In some embodiments, the temperatures of the bed device during the subject's menstrual cycle provided herein can be recommended by the bed device (e.g., by a computer processor operatively coupled to the bed device). Such recommendation can be based on information from other users and/or sensing data collected from the user while using the bed device.

In some embodiments, the body temperature of the subject is higher in luteal phase compared to in follicular phase. In some embodiments, the body temperature of the subject is between about 0° C. to about 3° C. higher during luteal phase compared to follicular phase. In some embodiments, the body temperature of the subject is at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., or at least or up to about 3° C. higher during luteal phase compared to follicular phase.

In some embodiments, the temperature of the bed device is higher in luteal phase compared to in follicular phase. In some embodiments, the temperature of the bed device is between about 0° C. to about 3° C. higher during luteal phase compared to follicular phase. In some embodiments, the temperature of the bed device is at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., or at least or up to about 3° C. higher during luteal phase compared to follicular phase.

In some embodiments, the body temperature of the subject is lower in follicular phase compared to in luteal phase. In some embodiments, the body temperature of the subject is between about 0° C. to about 3° C. lower during follicular phase compared to luteal phase. In some embodiments, the body temperature of the subject is between about 0° C. to about 3° C. lower during luteal phase compared to follicular phase. In some embodiments, the body temperature of the subject is at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., or at least or up to about 3° C. lower during follicular phase compared to luteal phase.

In some embodiments, the temperature of the bed device is lower in follicular phase compared to in luteal phase. In some embodiments, the temperature of the bed device is between about 0° C. to about 3° C. lower during follicular phase compared to luteal phase. In some embodiments, the temperature of the bed device is between about 0° C. to about 3° C. lower during luteal phase compared to follicular phase. In some embodiments, the temperature of the bed device is at least or up to about 0° C., at least or up to about 0.1° C., at least or up to about 0.2° C., at least or up to about 0.3° C., at least or up to about 0.4° C., at least or up to about 0.5° C., at least or up to about 0.6° C., at least or up to about 0.7° C., at least or up to about 0.8° C., at least or up to about 0.9° C., at least or up to about 1° C., at least or up to about 1.1° C., at least or up to about 1.2° C., at least or up to about 1.3° C., at least or up to about 1.4° C., at least or up to about 1.5° C., at least or up to about 1.6° C., at least or up to about 1.7° C., at least or up to about 1.8° C., at least or up to about 1.9° C., at least or up to about 2° C., at least or up to about 2.1° C., at least or up to about 2.2° C., at least or up to about 2.3° C., at least or up to about 2.4° C., at least or up to about 2.5° C., at least or up to about 2.6° C., at least or up to about 2.7° C., at least or up to about 2.8° C., at least or up to about 2.9° C., or at least or up to about 3° C. lower during follicular phase compared to luteal phase.

In some embodiments, the bed device app calculates the recommended temperatures of the bed device for the subject for use during the subject's luteal phase. In some embodiments, the bed device app calculates the recommended bedtime temperature of the bed device during the subject's luteal phase using the following formula:

$$Follicular phase temperature at bedtime - 1 ° C . \qquad ( V )$$

In some embodiments, the follicular phase bedtime temperature used in the above formula is the most recent bedtime temperature used by the subject during follicular phase.

In some embodiments, the bed device app calculates the recommended initial temperature of the bed device during the subject's luteal phase using the following formula:

$$Follicular phase initial temperature - 1. ° C . \qquad ( VI )$$

In some embodiments, the follicular initial phase temperature used in the above formula is the most recent initial temperature used by the subject during follicular phase.

In some embodiments, the luteal phase final temperature is the same final temperature used by the subject during follicular phase. In some embodiments, the subject can update the final temperature used during luteal phase. If the subject adjusts the final temperature during luteal phase more than about 1.0° C. from the final temperature used during follicular phase, then the bed device app can automatically adjust the final temperature during the next luteal phase to be the adjusted temperature.

In some embodiments, the temperature of the bed device during the subject's follicular phase is between about 0° C. to about 31° C. higher than the temperature of the device during the subject's luteal phase. In some embodiments, the temperature of the bed device during the subject's follicular phase is at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. higher than the temperature of the bed device during the subject's luteal phase.

In some embodiments, the temperature dial values of the bed device during the subject's follicular phase are increased between about 1 to about 20 dial values compared to the temperature dial values of the bed device during the subject's luteal phase. In some embodiments, the temperature dial values of the bed device during the subject's follicular phase are increased at least or up to about 0 dial value, at least or up to about 1 dial value, at least or up to about 2 dial values, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial updates, at least or up to about 19 dial updates, or at least or up to about 20 dial values compared to the temperature dial values of the bed device during the subject's luteal phase.

In some embodiments, the temperature of the bed device during the subject's follicular phase is between about 0° C. to about 31° C. lower than the temperature of the device during the subject's luteal phase. In some embodiments, the temperature of the bed device during the subject's follicular phase is at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. lower than the temperature of the bed device during the subject's luteal phase. In some embodiments, the temperature dial values of the bed device during the subject's follicular phase are decreased between about 1 to about 20 dial values compared to the temperature dial values of the bed device during the subject's luteal phase. In some embodiments, the temperature dial values of the bed device during the subject's follicular phase are decreased at least or up to about 0 dial value, at least or up to about 1 dial value, at least or up to about 2 dial values, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial updates, at least or up to about 19 dial updates, or at least or up to about 20 dial values compared to the temperature dial values of the bed device during the subject's luteal phase.

In some embodiments, the temperature of the bed device during the subject's luteal phase is between about 0° C. to about 31° C. lower than the temperature of the device during the subject's follicular phase. In some embodiments, the temperature of the bed device during the subject's luteal phase is at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. lower than the temperature of the bed device during the subject's follicular phase.

In some embodiments, the temperature dial values of the bed device during the subject's luteal phase are decreased between about 1 to about 20 dial values compared to the temperature dial values of the bed device during the subject's follicular phase. In some embodiments, the temperature dial values of the bed device during the subject's follicular phase are decreased at least or up to about 0 dial value, at least or up to about 1 dial value, at least or up to about 2 dial values, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial updates, at least or up to about 19 dial updates, or at least or up to about 20 dial values compared to the temperature dial values of the bed device during the subject's follicular phase.

In some embodiments, the temperature of the bed device during the subject's luteal phase is between about 0° C. to about 31° C. higher than the temperature of the device during the subject's follicular phase. In some embodiments, the temperature of the bed device during the subject's luteal phase is at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. higher than the temperature of the bed device during the subject's follicular phase.

In some embodiments, the temperature dial values of the bed device during the subject's luteal phase are increased between about 1 to about 20 dial values compared to the temperature dial values of the bed device during the subject's follicular phase. In some embodiments, the temperature dial values of the bed device during the subject's follicular phase are increased at least or up to about 0 dial value, at least or up to about 1 dial value, at least or up to about 2 dial values, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial updates, at least or up to about 19 dial updates, or at least or up to about 20 dial values compared to the temperature dial values of the bed device during the subject's follicular phase.

In some embodiments, the temperatures of the bed device adjust to the follicular phase temperatures on the predicted start date of the subject's period. In some embodiments, the temperatures of the bed device adjust to the luteal phase temperatures at least or up to about 10 days before the start of the subject's period.

In some embodiments, the recommended bedtime, initial, and final temperatures of the bed device for during luteal phase and follicular phase of the subject is recalculated based on any dial updates that occurred during the previous luteal phase and follicular phase of the subject.

In some embodiments, the recommended bedtime, initial, and final temperatures of the bed device for during luteal phase and follicular phase is determined by the subject using the bed device for between about one day to about 14 days during the luteal phase and follicular phase respectively. In some embodiments, the recommended bedtime, initial, and final temperature of the bed device for during luteal phase is determined by the subject using the bed device for at least or up to about one day, at least or up to about two days, at least or up to about three days, at least or up to about four days, at least or up to about five days, at least or up to about six days, at least or up to about seven days, at least or up to about eight days, at least or up to about nine days, at least or up to about ten days, at least or up to about 11 days, at least or up to about 12 days, at least or up to about 13 days, or at least or up to about 14 days during the luteal phase of the subject. In some embodiments, the recommended bedtime, initial, and final temperature of the bed device for during follicular phase is determined by the subject using the bed device for at least or up to about one day, at least or up to about two days, at least or up to about three days, at least or up to about four days, at least or up to about five days, at least or up to about six days, at least or up to about seven days, at least or up to about eight days, at least or up to about nine days, at least or up to about ten days, at least or up to about 11 days, at least or up to about 12 days, at least or up to about 13 days, or at least or up to about 14 days during the follicular phase of the subject.

In some embodiments, the subject has irregular periods. In some embodiments, the bed device app sends the subject a notification on the predicted start date of the subject's period. In some embodiments, the notification has the subject input if the subject's period started on that date. In some embodiments, the notification has the subject to input if the subject's period has started. If the subject's period did not start on the date the notification was sent, then the bed device may continue using the luteal bedtime, initial, and final temperatures. In some embodiments, the bed device app sends the notification each subsequent day after the predicted start date of the subject's period. In some embodiments, the notifications continue to be sent each subsequent day until the subject's period starts. In some embodiments, the notifications can continue to be sent for at least or up to about one day, at least or up to about two days, at least or up to about three days, at least or up to about four days, at least or up to about five days, at least or up to about six days, at least or up to about seven days, at least or up to about eight days, at least or up to about nine days, or at least or up to about ten days after the predicted start date of the subject's period. In some embodiments, the bed device will adjust to follicular phase bedtime, initial, and final temperatures once the subject has inputted into the bed device app that the subject's period has started.

In some embodiments, the one or more sensors collect data about the one or more biometrics from the subject. In some embodiments, the one or more sensors detect (e.g., automatically detect) a change in biometrics of the subject (e.g., a female subject), e.g., while the subject is using the bed device and/or while the subject is on the bed device. The biometrics can be indicative of a particular phase of the menstrual cycle. For example, the one or more sensors detect an increase in body temperature due to the subject being in luteal phase. In some embodiments, the bed device automatically decreases the temperature of bed device upon detection of an increased body temperature of the subject. In some embodiments, the one or more sensors detects a decrease in body temperature due to the subject being in follicular phase. In some embodiments, the device automatically increases the temperature of the bed device upon detection of a decreased body temperature of the subject.

In some embodiments, the one or more sensors collect data about the one or more biometrics during the luteal phase of the subject. In some embodiments, the one or more sensors collect data about the one or more biometrics during the follicular phase of the subject. In some embodiments, the subject is sent the summary described herein. In some embodiments, the summary includes information specific to use during the luteal phase. In some embodiments, the summary includes information specific to use during the follicular phase.

In some embodiments, the menstruation mode of the bed device decreases the percent of dial value updates throughout the time while the subject is on the bed device. In some embodiments, the menstruation mode of the bed device decreases dial value updates between about 0% to about 500%. In some embodiments, the menstruation mode of the bed device decreases dial value updates by at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500%.

In some embodiments, the menstruation mode of the bed device decreases dial value updates by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less. In some embodiments, the menstruation mode of the bed device decreases dial value updates by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the menstruation mode of the bed device enhances users experience of the bed device. In some embodiments, the menstruation mode of the bed device enhances the user experience of the bed device by improving sleep quality of the user. In some embodiments, the menstruation mode of the bed device improves the sleep quality values of the subject. Non-limiting examples of sleep quality values include percent light sleep, percent deep sleep, percent REM sleep, total sleep time, wake time, number of awakenings, and net sleep score.

In some embodiments, the menstruation mode of the bed device results in an increase in percent change of light sleep. In some embodiments, the menstruation mode of the bed device results in between 0%-300% increase in percent change in light sleep. In some embodiments, the menstruation mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change of light sleep.

In some embodiments, the menstruation mode of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of light sleep. In some embodiments, the menstruation mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100% increase, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change of light sleep.

In some embodiments, the menstruation mode of the bed device results in an increase in percent change of deep sleep. In some embodiments, the menstruation mode of the bed device results in between 0%-300% increase in deep sleep. In some embodiments, the menstruation mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change of deep sleep.

In some embodiments, the menstruation mode of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of deep sleep. In some embodiments, the menstruation mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change of deep sleep.

In some embodiments, the menstruation mode of the bed device results in an increase in percent change of REM sleep. In some embodiments, the menstruation mode of the bed device results in between 0%-300% increase in percent change of REM sleep. In some embodiments, the menstruation mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change of REM sleep.

In some embodiments, the menstruation mode of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of REM sleep. In some embodiments, the menstruation mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change of REM sleep.

In some embodiments, menstruation mode of the bed device results in an increase in percent change of total sleep time. Total sleep time can correspond to the total amount of time the subject is asleep on the bed device. In some embodiments, the menstruation mode of the bed device results in between 0%-500% increase in total sleep time. In some embodiments, the menstruation mode results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% increase in percent change of total sleep time.

In some embodiments, the menstruation mode of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of total sleep time. In some embodiments, the menstruation mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in percent change in total sleep time.

In some embodiments, the menstruation mode of the bed device results in an increase in percent change of a net sleep score. The net sleep score can be calculated by determining the percent change in how many subjects experienced a positive percent change in light, deep, REM, or total sleep time compared to a group that did not experience the menstruation mode (e.g., a control group). In some embodiments, the menstruation mode of the bed device results in between 0%-500% increase in percent change of net sleep score. In some embodiments, the menstruation mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% increase in percent change of net sleep score.

In some embodiments, the menstruation mode of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of net sleep score. In some embodiments, the menstruation mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in percent change of net sleep score.

In some embodiments, the menstruation mode of the bed device results in a decrease in percent change of total awake time. The total awake time can be calculated by determining the length of time the user is awake during use of the bed device. In some embodiments, the menstruation mode of the bed device results in between 0%-500% decrease in percent change of total awake time. In some embodiments, the menstruation mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% decrease in percent change of total awake time.

In some embodiments, the menstruation mode of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less decrease in percent change of total awake time. In some embodiments, the menstruation mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% decrease in percent change of total awake time.

In some embodiments, the menstruation mode of the bed device results in a decrease in percent change of the number of awakenings per use of the bed device. The number of awakenings can be calculated by determining the number of times the user awaken during use of the bed device. In some embodiments, the menstruation mode of the bed device results in between 0%-500% decrease in percent change of number of awakenings. In some embodiments, the menstruation mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% decrease in percent change of numbers of awakenings.

In some embodiments, the menstruation mode of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less decrease in percent change of number of awakenings. In some embodiments, the menstruation mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% decrease in percent change of number of awakenings.

ii. Menopause

In some embodiments, the bed device app recommends a menopause mode to the subject. Non-limiting classification of biological states that triggers a menopause mode recommendation includes pre-menopausal with hormone replacement, pre-menopausal without hormone replacement, post-menopausal with hormone replacement therapy, and post-menopausal without hormone replacement therapy. In some embodiments, the subject can accept the menopause mode recommendation. In some embodiments, the subject can reject the menopause mode recommendation. In some embodiments, the subject provides additional information to the bed device app following accepting the menopause mode recommendation. In some embodiments, the additional information is whether the subject has hot flashes while using the bed device. In some embodiments, the additional information is whether the subject has night sweats while using the bed device. If the subject answers yes to having night sweats and/or hot flashes while using the bed device, the bed device app can require a second additional information. In some embodiments, the second additional information is whether the night sweats and/or hot flashes occur on a regular schedule.

In some embodiments, the bed device further comprises a processor. In some embodiments, the processor analyzes the data collected by the one or more sensors. In some embodiments, the bed device further comprises a temperature regulator. In some embodiments, the temperature regulator is operatively connected to the processor. In some embodiments, the processor can provide a control signal to the temperature regulator. In some embodiments, the control signal can be an instruction for the temperature regulator to change a temperature of the article of furniture. In some embodiments, the control signal can be an instruction for the temperature regulator to change a temperature of a portion of the article of furniture (e.g., bed device). In some embodiments, the control signal can be an instruction to change the temperature of the bed device to cooler (e.g., lower temperature) in response to a hot flash/night sweats. In some embodiments, the control signal can be an instruction to change the temperature of the bed device to cooler (e.g., lower temperature) in response to the subject activating the rapid cool widget. In some embodiments, the control signal can be an instruction to change the temperature of the bed device to warmer (e.g., higher temperature) in response to a chills. In some embodiments, the control signal can be an instruction to change the temperature of the bed device to warmer (e.g., higher temperature) in response to the subject activating the rapid heat widget.

In some embodiments, the temperatures of the bed device during the subject's hot flash/night sweats provided herein can be recommended by the bed device (e.g., by a computer processor operatively coupled to the bed device). Such recommendation can be based on information from other users and/or sensing data collected from the user while using the bed device.

In some embodiments, the bed device app schedules a decrease in the bed device temperature if the subject experiences regular hot flashes or night sweats. In some embodiments, the subject inputs when the hot flashes or night sweats occur while the subject is using the bed device into the app. In some embodiments, the hot flashes or night sweats occur at least one time during the use of the bed device. In some embodiments, the hot flashes or night sweats occur at least or up to about one time, at least or up to about two times, at least or up to about three times, at least or up to about four times, at least or up to about five times, at least or up to about six times, at least or up to about seven times, at least or up to about eight times, at least or up to about nine times, at least or up to about ten times, or more during the use of the bed device. In some embodiments, the hot flashes or night sweats occur between about 0 minutes to about 10 hours subsequent to the subject falling sleeping on the bed device. In some embodiments, the hot flashes or night sweats occur at least or up to about 0 minutes, at least or up to about 30 minutes, at least or up to about 60 minutes, at least or up to about 1.5 hours, at least or up to about 2 hours, at least or up to about 2.5 hours, at least or up to about 3 hours, at least or up to about 3.5 hours, at least or up to about 4 hours, at least or up to about 4.5 hours, at least or up to about 5 hours, at least or up to about 5.5 hours, at least or up to about 6 hours, at least or up to about 6.5 hours, at least or up to about 7 hours, at least or up to about 7.5 hours, at least or up to about 8 hours, at least or up to about 8.5 hours, at least or up to about 9 hours, at least or up to about 9.5 hours, or at least or up to about 10 hours subsequent to the subject falling asleep on the bed device.

In some embodiments, the subject inputs when the hot flashes and/or night sweats occur while using the bed device and the temperature the subject wants the bed device to decrease to when the hot flashes and/or night sweats occurs.

In some embodiments, the subject sets the temperature to be between about 12° C. to about 43° C. during when the hot flashes and/or night sweats occur. In some embodiments, the subject sets the temperature to be at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., at least or up to about 31° C., at least or up to about 32° C., at least or up to about 33° C., at least or up to about 34° C., at least or up to about 35° C., at least or up to about 36° C., at least or up to about 37° C., at least or up to about 38° C., at least or up to about 39° C., at least or up to about 40° C., at least or up to about 41° C., at least or up to about 42° C., or at least or up to about 43° C. during when the hot flashes and/or night sweats occur. In some embodiments, the subject sets the temperature to be between the dial values of about −10 to about +10 during when the hot flashes and/or night sweats occur. In some embodiments, the subject sets the temperature to be the dial values of at least or up to about −10, at least or up to about −9.5, at least or up to about −9, at least or up to about −8.5, at least or up to about −8, at least or up to about −7.5, at least or up to about −7, at least or up to about −6.5, at least or up to about −6, at least or up to about −5.5, at least or up to about −5, at least or up to about −4.5, at least or up to about −4, at least or up to about −3.5, at least or up to about −3, at least or up to about −2.5, at least or up to about −2, at least or up to about −1.5, at least or up to about −1, at least or up to about −0.5, at least or up to about 0, at least or up to about +0.5, at least or up to about +1, at least or up to about +1.5, at least or up to about +2, at least or up to about +2.5, at least or up to about +3, at least or up to about +3.5, at least or up to about +4, at least or up to about +4.5, at least or up to about +5, at least or up to about +5.5, at least or up to about +6, at least or up to about +6.5, at least or up to about +7, at least or up to about +7.5, at least or up to about +8, at least or up to about +8.5, at least or up to about +9, at least or up to about +9.5, or at least or up to about +10 during when the host flashes and/or night sweats occur.

In some embodiments, the bed device stays at the temperature set by the subject for between about 5 minutes to about 6 hours. In some embodiments, the bed device stays at the temperature set by the subject for at least or up to about 5 minutes, at least or up to about 10 minutes, at least or up to about 15 minutes, at least or up to about 20 minutes, at least or up to about 25 minutes, at least or up to about 30 minutes, at least or up to about 35 minutes, at least or up to about 40 minutes, at least or up to about 45 minutes, at least or up to about 50 minutes, at least or up to about 55 minutes, at least or up to about 1 hour, at least or up to about 1.5 hours, at least or up to about 2 hours, at least or up to about 2.5 hours, at least or up to about 3 hours, at least or up to about 3.5 hours, at least or up to about 4 hours, at least or up to about 4.5 hours, at least or up to about 5 hours, at least or up to about 5.5 hours, or at least or up to about 6 hours.

Alternatively, in some embodiments, the subject does not have regular hot flashes and/or night sweats.

In some embodiments, the menopause mode further comprises a rapid cool widget. In some cases, the rapid cool widget is displayed in the bed device app via a graphical user interface (GUI). In some embodiments, the rapid cool widget cools the bed device down to a preset temperature upon activation of the rapid cool widget. In some cases, the cooling of the bed device to a present temperature can be activated by the user selecting the rapid cool widget in the bed device app. In some embodiments, the subject sets the preset temperature prior to using the bed device. In some embodiments the preset temperature for the rapid cool widget is between about 12° C. to about 43° C. In some embodiments, the preset temperature for the rapid cool widget is at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., at least or up to about 31° C., at least or up to about 32° C., at least or up to about 33° C., at least or up to about 34° C., at least or up to about 35° C., at least or up to about 36° C., at least or up to about 37° C., at least or up to about 38° C., at least or up to about 39° C., at least or up to about 40° C., at least or up to about 41° C., at least or up to about 42° C., or at least or up to about 43° C. In some embodiments, the preset temperature for the rapid cool widget is between the dial values of −10 to about +10. In some embodiments, the preset temperature for the rapid cool widget is the dial value of at least or up to about −10, at least or up to about −9.5, at least or up to about −9, at least or up to about −8.5, at least or up to about −8, at least or up to about −7.5, at least or up to about −7, at least or up to about −6.5, at least or up to about −6, at least or up to about −5.5, at least or up to about −5, at least or up to about −4.5, at least or up to about −4, at least or up to about −3.5, at least or up to about −3, at least or up to about −2.5, at least or up to about −2, at least or up to about −1.5, at least or up to about −1, at least or up to about −0.5, at least or up to about 0, at least or up to about +0.5, at least or up to about +1, at least or up to about +1.5, at least or up to about +2, at least or up to about +2.5, at least or up to about +3, at least or up to about +3.5, at least or up to about +4, at least or up to about +4.5, at least or up to about +5, at least or up to about +5.5, at least or up to about +6, at least or up to about +6.5, at least or up to about +7, at least or up to about +7.5, at least or up to about +8, at least or up to about +8.5, at least or up to about +9, at least or up to about +9.5, or at least or up to about +10. In some embodiments, the bed device remains at the preset temperature until the rapid cool widget is deactivated by the subject. Alternatively, in some embodiments, the bed device remains at the preset temperature for between about 0 minutes to about 6 hours. In some embodiments, the bed device stays at the preset temperature for at least or up to about 5 minutes, at least or up to about 10 minutes, at least or up to about 15 minutes, at least or up to about 20 minutes, at least or up to about 25 minutes, at least or up to about 30 minutes, at least or up to about 35 minutes, at least or up to about 40 minutes, at least or up to about 45 minutes, at least or up to about 50 minutes, at least or up to about 55 minutes, at least or up to about 1 hour, at least or up to about 1.5 hours, at least or up to about 2 hours, at least or up to about 2.5 hours, at least or up to about 3 hours, at least or up to about 3.5 hours, at least or up to about 4 hours, at least or up to about 4.5 hours, at least or up to about 5 hours, at least or up to about 5.5 hours, or at least or up to about 6 hours.

In some embodiments, the present disclosure provides a method comprising: receiving input from a user of an article of furniture, based on the input from the user, providing a control signal for a temperature regulator that is operatively coupled to the article of furniture, wherein the control signal comprises an instruction for the temperature regulator to adjust a temperature of at least a portion of the article of furniture from a predetermined temperature profile to a preset temperature in real time to adjust the current temperature of the article of furniture. In some cases, the article of furniture is the bed device of the present disclosure. In some cases, the predetermined temperature profile can be the first temperatures of the present disclosure, the plurality of temperatures (e.g., the at least four temperature settings) of the present disclosure, the menstruation mode of the present disclosure, the pregnancy mode of the present disclosure, the menopause mode of the present disclosure, or the temperature profile based on the user's circadian rhythm as described in the present disclosure. In some cases, the preset temperature can be the temperature that the user set in the rapid cool widget in the bed device app. In some cases, the preset temperature can be the temperature that the user set in the rapid heat widget in the bed device app.

In some embodiments, the method comprises: (a) directing a temperature regulator to maintain the temperature of the at least the portion of an article of furniture substantially at a target temperature based on the predetermined temperature profile for the article of furniture, the predetermined temperature profile comprising the target temperature, (b) while the at least the portion of the article of furniture is maintained substantially at the target temperature, receiving the input from the user; and (c) based on the input, providing the control signal to the temperature regulator, the control signal comprising the instruction to change the temperature of the article of furniture to the preset temperature, wherein the target temperature and the preset temperature are different. In some cases, the receiving the input from the user can comprise the user activating the rapid cool widget in the bed device app. In some cases, the receiving the input from the user can comprise the user activating the rapid heat widget in the bed device app.

In some cases, the preset temperature and the target temperature can differ by between about 0° to about 30°. In some cases, the preset temperature and the target temperature can differ by at least or up to about 0° C., at least or up to about 0.5° C., at least or up to about 1° C., at least or up to about 1.5° C., at least or up to about 2° C., at least or up to about 2.5° C., at least or up to about 3° C., at least or up to about 3.5° C., at least or up to about 4° C., at least or up to about 4.5° C., at least or up to about 5° C., at least or up to about 5.5° C., at least or up to about 6° C., at least or up to about 6.5° C., at least or up to about 7° C., at least or up to about 7.5° C., at least or up to about 8° C., at least or up to about 8.5° C., at least or up to about 9° C., at least or up to about 9.5° C., at least or up to about 10° C., at least or up to about 10.5° C., at least or up to about 11° C., at least or up to about 11.5° C., at least or up to about 12° C., at least or up to about 12.5° C., at least or up to about 13° C., at least or up to about 13.5° C., at least or up to about 14° C., at least or up to about 14.5° C., at least or up to about 15° C., at least or up to about 15.5° C., at least or up to about 16° C., at least or up to about 16.5° C., at least or up to about 17° C., at least or up to about 17.5° C., at least or up to about 18° C., at least or up to about 18.5° C., at least or up to about 19° C., at least or up to about 19.5° C., at least or up to about 20° C., at least or up to about 20.5° C., at least or up to about 21° C., at least or up to about 21.5° C., at least or up to about 22° C., at least or up to about 22.5° C., at least or up to about 23° C., at least or up to about 23.5° C., at least or up to about 24° C., at least or up to about 24.5° C., at least or up to about 25° C., at least or up to about 25.5° C., at least or up to about 26° C., at least or up to about 26.5° C., at least or up to about 27° C., at least or up to about 27.5° C., at least or up to about 28° C., at least or up to about 28.5° C., at least or up to about 29° C., at least or up to about 29.5° C., or at least or up to about 30°.

In some embodiments, the rate of the temperature change of the bed device following the providing the control signal can be between about 0.1° C. per minute (° C./min) to about 10° C./min. In some embodiments, the rate of the temperature change of the bed device following the providing the control signal can be at least or up to about 0.1° C./min, at least or up to about 0.2° C./min, at least or up to about 0.3° C./min, at least or up to about 0.4° C./min, at least or up to about 0.5° C./min, at least or up to about 0.6° C./min, at least or up to about 0.7° C./min, at least or up to about 0.8° C./min, at least or up to about 0.9° C./min, at least or up to about 1° C./min, at least or up to about 1.1° C./min, at least or up to about 1.2° C./min, at least or up to about 1.3° C./min, at least or up to about 1.4° C./min, at least or up to about 1.5° C./min, at least or up to about 2° C./min, at least or up to about 2.5° C./min, at least or up to about 3° C./min, at least or up to about 3.5° C./min, at least or up to about 4° C./min, at least or up to about 4.5° C./min, at least or up to about 5° C./min, at least or up to about 5.5° C./min, at least or up to about 6° C./min, at least or up to about 6.5° C./min, at least or up to about 7° C./min, at least or up to about 7.5° C./min, at least or up to about 8° C./min, at least or up to about 8.5° C./min, at least or up to about 9° C./min, at least or up to about 9.5° C./min, at least or up to about 10° C./min, or more.

In some cases, the instruction is to change the temperature of the article of furniture to the preset temperature transiently for a predetermined duration of time. In some cases, the predetermined duration of time can be between about 0 seconds to about 2 hours. In some cases, the predetermined duration of time can be at least or up to about 0 seconds, at least or up to about 5 seconds, at least or up to about 10 seconds, at least or up to about 15 seconds, at least or up to about 20 seconds, at least or up to about 25 seconds, at least or up to about 30 seconds, at least or up to about 35 seconds, at least or up to about 40 seconds, at least or up to about 45 seconds, at least or up to about 50 seconds, at least or up to about 55 seconds, at least or up to about 60 seconds, at least or up to about 5 minutes, at least or up to about 10 minutes, at least or up to about 15 minutes, at least or up to about 20 minutes, at least or up to about 25 minutes, at least or up to about 30 minutes, at least or up to about 35 minutes, at least or up to about 40 minutes, at least or up to about 45 minutes, at least or up to about 50 minutes, at least or up to about 55 minutes, at least or up to about 60 minutes, at least or up to about 65 minutes, at least or up to about 70 minutes, at least or up to about 75 minutes, at least or up to about 80 minutes, at least or up to about 85 minutes, at least or up to about 90 minutes, at least or up to about 95 minutes, at least or up to about 100 minutes, at least or up to about 105 minutes, at least or up to about 110 minutes, at least or up to about 115 minutes, or at least or up to about 120 minutes.

In some cases, the method further comprises subsequent to (c), directing the temperature regulator to regulate the temperature of the at least the portion of the article of furniture based on the predetermined temperature profile. In some examples, the method further comprises, subsequent to (c), directing the temperature regulator to change the temperature of the at least the portion of the article of furniture back to the target temperature. In some examples, the method further comprises, subsequent to (c), directing the temperature regulator to change the temperature of the at least the portion of the article of furniture to an additional target temperature of the predetermined temperature profile. In some cases, the additional target temperature of the predetermined temperature profile is a temperature of the predetermined temperature profile that is different from the target temperature. In some cases, the additional target temperature of the predetermined temperature profile is a temperature of the predetermined temperature profile that is the same of the target temperature.

In some cases, the preset temperature is predetermined and stored in a database prior to (a). In some cases, the preset temperature is predetermined in the bed device app.

In some embodiments, the rate of the temperature change of the bed device after activating the rapid cool widget is between about 0.1° C. per minute (° C./min) to about 10° C./min. In some embodiments, the rate of the temperature change of the bed device after activating the rapid cool widget is at least or up to about 0.1° C./min, at least or up to about 0.2° C./min, at least or up to about 0.3° C./min, at least or up to about 0.4° C./min, at least or up to about 0.5° C./min, at least or up to about 0.6° C./min, at least or up to about 0.7° C./min, at least or up to about 0.8° C./min, at least or up to about 0.9° C./min, at least or up to about 1° C./min, at least or up to about 1.1° C./min, at least or up to about 1.2° C./min, at least or up to about 1.3° C./min, at least or up to about 1.4° C./min, at least or up to about 1.5° C./min, at least or up to about 2° C./min, at least or up to about 2.5° C./min, at least or up to about 3° C./min, at least or up to about 3.5° C./min, at least or up to about 4° C./min, at least or up to about 4.5° C./min, at least or up to about 5° C./min, at least or up to about 5.5° C./min, at least or up to about 6° C./min, at least or up to about 6.5° C./min, at least or up to about 7° C./min, at least or up to about 7.5° C./min, at least or up to about 8° C./min, at least or up to about 8.5° C./min, at least or up to about 9° C./min, at least or up to about 9.5° C./min, at least or up to about 10° C./min, or more.

In some embodiments, the rapid cool widget is used for subjects with irregular hot flashes and/or night sweats.

In some embodiments, the rapid cool widget is not limited to use in the menopause mode. In some embodiments, the rapid cool widget is used for subjects with medical conditions. Non-limiting examples of medical conditions include Lupus, cancer, viral infections, long-lasting COVID, and fever. In some embodiments, the rapid cool widget is used for subjects taking medications. Non-limiting examples of medications include chemotherapy, antibiotics, anti-depressants, immunotherapies, and anti-psychotics. In some embodiments, the rapid cool widget is used by female subject. In some embodiments, the rapid cool widget is used by male subjects. In some embodiments, the rapid cool widget is used by subjects with an unidentified biological sex.

In some embodiments, the menopause mode further comprises a rapid heat widget. In some embodiments, the rapid heat widget heats the bed device up to a second preset temperature upon activation of the rapid heat widget. In some cases, the heating of the bed device to the second preset temperature is activated by the user selecting the rapid heat widget in the bed device app. In some embodiments, the subject sets the second preset temperature prior to using the bed device. In some embodiments, the second preset temperature for the rapid heat widget is between about 12° C. to about 43° C. In some embodiments, the second present temperature for the rapid heat widget is at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., at least or up to about 31° C., at least or up to about 32° C., at least or up to about 33° C., at least or up to about 34° C., at least or up to about 35° C., at least or up to about 36° C., at least or up to about 37° C., at least or up to about 38° C., at least or up to about 39° C., at least or up to about 40° C., at least or up to about 41° C., at least or up to about 42° C., or at least or up to about 43° C. In some embodiments, the second preset temperature for the rapid heat widget is between the dial values of about −10 to about +10. In some embodiments, the second preset temperature for the rapid heat widget is the dial value of at least or up to about −10, at least or up to about −9.5, at least or up to about −9, at least or up to about −8.5, at least or up to about −8, at least or up to about −7.5, at least or up to about −7, at least or up to about −6.5, at least or up to about −6, at least or up to about −5.5, at least or up to about −5, at least or up to about −4.5, at least or up to about −4, at least or up to about −3.5, at least or up to about −3, at least or up to about −2.5, at least or up to about −2, at least or up to about −1.5, at least or up to about −1, at least or up to about −0.5, at least or up to about 0, at least or up to about +0.5, at least or up to about +1, at least or up to about +1.5, at least or up to about +2, at least or up to about +2.5, at least or up to about +3, at least or up to about +3.5, at least or up to about +4, at least or up to about +4.5, at least or up to about +5, at least or up to about +5.5, at least or up to about +6, at least or up to about +6.5, at least or up to about +7, at least or up to about +7.5, at least or up to about +8, at least or up to about +8.5, at least or up to about +9, at least or up to about +9.5, or at least or up to about +10. In some embodiments, the bed device remains at the second preset temperature until the rapid heat cool is deactivated by the subject.

In some embodiments, the rate of the temperature change of the bed device after activating the rapid heat widget is between about 0.1° C. per minute (° C./min) to about 10° C./min. In some embodiments, the rate of the temperature change of the bed device after activating the rapid heat widget is at least or up to about 0.1° C./min, at least or up to about 0.2° C./min, at least or up to about 0.3° C./min, at least or up to about 0.4° C./min, at least or up to about 0.5° C./min, at least or up to about 0.6° C./min, at least or up to about 0.7° C./min, at least or up to about 0.8° C./min, at least or up to about 0.9° C./min, at least or up to about 1° C./min, at least or up to about 1.1° C./min, at least or up to about 1.2° C./min, at least or up to about 1.3° C./min, at least or up to about 1.4° C./min, at least or up to about 1.5° C./min, at least or up to about 2° C./min, at least or up to about 2.5° C./min, at least or up to about 3° C./min, at least or up to about 3.5° C./min, at least or up to about 4° C./min, at least or up to about 4.5° C./min, at least or up to about 5° C./min, at least or up to about 5.5° C./min, at least or up to about 6° C./min, at least or up to about 6.5° C./min, at least or up to about 7° C./min, at least or up to about 7.5° C./min, at least or up to about 8° C./min, at least or up to about 8.5° C./min, at least or up to about 9° C./min, at least or up to about 9.5° C./min, at least or up to about 10° C./min, or more.

In some embodiments, the rapid heat widget is not limited to use in the menopause mode. In some embodiments, the rapid heat widget is used for subjects with medical conditions. Non-limiting examples of medical conditions include Lupus, cancer, viral infections, long-lasting COVID, and fever. In some embodiments, the rapid heat widget is used for subjects taking medications. Non-limiting examples of medications include chemotherapy, antibiotics, anti-depressants, immunotherapies, and anti-psychotics. In some embodiments, the heat cool widget is used by female subject. In some embodiments, the rapid heat widget is used by male subjects. In some embodiments, the rapid heat widget is used by subjects with an unidentified biological sex.

In some embodiments, the bed device app collects data regarding the occurrence of hot flashes and/or night sweats. In some embodiments, the one or more sensors detects an increase in skin temperature or perspiration of the subject while on the bed device. In some embodiments, the bed device app correlates an increase in skin temperature or appearance of perspiration on the one or more sensors has a night sweat and/or hot flash. In some embodiments, the bed device app tracks the occurrence of night sweats and/or hot flashes during use of the bed device by the subject. In some embodiments, the summary described herein includes when a hot flash and/or night sweat occurred during the use of the bed device by the subject.

In some embodiments, the bed device app automatically cools the bed device down to the preset temperature upon detection of increased skin temperature or perspiration by the one or more sensors. In some embodiments, the bed device app automatically adjusts the bed device to the initial temperature of the bed device prior to the preset temperature once the skin temperature of the subject decreases or perspiration is no longer detected.

In some embodiments, the menopause mode of the bed device decreases the percent of dial value updates throughout the time while the subject is on the bed device. In some embodiments, the menstruation mode of the bed device decreases dial value updates between about 0% to about 500%. In some embodiments, the menstruation mode of the bed device decreases dial value updates by at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500%.

In some embodiments, the menopause mode of the bed device decreases dial value updates by at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less. In some embodiments, the menopause mode of the bed device decreases dial value updates by about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

In some embodiments, the menopause mode of the bed device enhances users experience of the bed device. In some embodiments, the menopause mode of the bed device enhances the user experience of the bed device by improving sleep quality of the user. In some embodiments, the menopause mode of the bed device improves the sleep quality values of the subject. Non-limiting examples of sleep quality values include percent light sleep, percent deep sleep, percent REM sleep, total sleep time, total awake time, number of awakenings, and net sleep score.

In some embodiments, the menopause mode of the bed device results in an increase in percent change of light sleep. In some embodiments, the menopause mode of the bed device results in between 0%-300% increase in percent change light sleep. In some embodiments, the menopause mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change of light sleep.

In some embodiments, the menopause mode of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of light sleep. In some embodiments, the menopause mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change of light sleep.

In some embodiments, the menopause mode of the bed device results in an increase in percent change of deep sleep. In some embodiments, the menopause mode of the bed device results in between 0%-300% increase in percent change of deep sleep. In some embodiments, the menopause mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change of deep sleep.

In some embodiments, the menopause mode of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change in deep sleep. In some embodiments, the menopause mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change in deep sleep.

In some embodiments, the menopause mode of the bed device results in an increase in percent change of REM sleep. In some embodiments, the menopause mode of the bed device results in between 0%-300% increase in percent change of REM sleep. In some embodiments, the menopause mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, %, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change of REM sleep.

In some embodiments, the menopause mode of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of REM sleep. In some embodiments, the menopause mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change of REM sleep.

In some embodiments, the menopause mode of the bed device results in an increase in percent change of total sleep time. Total sleep time can correspond to the total amount of time the subject is asleep on the bed device. In some embodiments, the menopause mode of the bed device results in between 0%-500% increase in percent change of total sleep time. In some embodiments, the menopause mode results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% increase in percent change of total sleep time.

In some embodiments, the menopause mode of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of total sleep time. In some embodiments, the menopause mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in percent change of total sleep time.

In some embodiments, the menopause mode of the bed device results in an increase in percent change of a net sleep score. The net sleep score can be calculated by determining the percent change in how many subjects experienced a positive percent change in light, deep, REM, or total sleep time compared to a group that did not experience the menopause mode (e.g., a control group). In some embodiments, the menopause mode of the bed device results in between 0%-500% increase in percent change in net sleep score. In some embodiments, the menopause mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% increase in percent change of net sleep score.

In some embodiments, the menopause mode of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of net sleep score. In some embodiments, the menopause mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in percent change of net sleep score.

In some embodiments, the menopause mode of the bed device results in a decrease in percent change of total awake time. The total awake time can be calculated by determining the length of time the user is awake during use of the bed device. In some embodiments, the menopause mode of the bed device results in between 0%-500% decrease in percent change of total awake time. In some embodiments, the menopause mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% decrease in percent change of total awake time.

In some embodiments, the menopause mode of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less decrease in percent change of total awake time. In some embodiments, the menopause mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% decrease in percent change of total awake time.

In some embodiments, the menopause mode of the bed device results in a decrease in percent change of the number of awakenings per use of the bed device. The number of awakenings can be calculated by determining the number of times the user awaken during use of the bed device. In some embodiments, menopause mode of the bed device results in between 0%-500% decrease in percent change of number of awakenings. In some embodiments, the menopause mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% decrease in percent change of numbers of awakenings.

In some embodiments, the menopause mode of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less decrease in percent change of number of awakenings. In some embodiments, the menopause mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% decrease in percent change of number of awakenings.

iii. Pregnancy

In some embodiments, the bed device app recommends a pregnancy mode to the subject. Non-limiting classifications of biological states that triggers a pregnancy mode recommendation include pregnant, breastfeeding, and postpartum. In some embodiments, the subject can accept the pregnancy mode recommendation. In some embodiments, the subject can reject the pregnancy mode recommendation. In some embodiments, the subject provides additional information to the bed device app following accepting the pregnancy mode recommendation.

In some embodiments, the additional information is the subject's expected due date. In some embodiments, the bed device app automatically calculates the start date of the subject's second trimester and third trimester based on the expected due date. In some embodiments, the bed device automatically updates the bed device temperatures to be different (e.g., lower) than the bed device temperatures during the first trimester. In some cases, the automatic updates to the bed device temperatures may occur to account for changing in the mother's body temperature during that trimester of pregnancy. In some embodiments, the update to the second trimester bed device temperatures occurs automatically on the start date of the subject's second trimester. In some embodiments, the update to the third trimester bed device temperatures occurs automatically on the start date of the subject's third trimester.

In some embodiments, the bed device further comprises a processor. In some embodiments, the processor analyzes the data collected by the one or more sensors. In some embodiments, the bed device further comprises a temperature regulator. In some embodiments, the temperature regulator is operatively connected to the processor. In some embodiments, the processor can provide a control signal to the temperature regulator. In some embodiments, the control signal can be an instruction for the temperature regulator to change a temperature of the article of furniture. In some embodiments, the control signal can be an instruction for the temperature regulator to change a temperature of a portion of the article of furniture (e.g., bed device). In some embodiments, the control signal can be an instruction to change the temperature of the bed device to the bed device temperature associated with the first trimester of pregnancy. In some embodiments, the control signal can be an instruction to change the temperature of the bed device to the bed device temperature associated with the second trimester of pregnancy. In some embodiments, the control signal can be an instruction to change the temperature of the bed device to the bed device temperature associated with the third trimester of pregnancy. In some embodiments, the control signal can be an instruction to change the temperature of the bed device to the bed device temperature associated with postpartum.

In some embodiments, the temperatures of the bed device during the subject's pregnancy provided herein can be recommended by the bed device (e.g., by a computer processor operatively coupled to the bed device). Such recommendation can be based on information from other users and/or sensing data collected from the user while using the bed device.

In some embodiments, the bed device temperatures in the second trimester are between about 0° C. to about 31° C. cooler than the bed device temperatures in the first trimester. In some embodiments, the bed device temperatures in the second trimester are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. cooler than the bed device temperatures in the first trimester. In some embodiments, the bed device temperatures dial values in the second trimester are decreased between about 0 to about 20 dial values from the dial values in the first trimester. In some embodiments, the bed device temperatures dial values in the second trimester are decreased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial, at least or up to about 14 dial, at least or up to about 15 dial values, at least or up to about 16 dial values, at least or up to about 17 dial values, at least or up to about 18 dial values, at least or up to about 19 dial values, or at least or up to about 20 dial values from the dial values of the first trimester.

In some embodiments, the bed device temperatures in the second trimester are between about 0° C. to about 31° C. warmer than the bed device temperatures in the first trimester. In some embodiments, the bed device temperatures in the second trimester are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. warmer than the bed device temperatures in the first trimester. In some embodiments, the bed device temperatures dial values in the second trimester are increased between about 0 to about 20 dial values from the dial values in the first trimester. In some embodiments, the bed device temperatures dial values in the second trimester are increased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values from the dial values of the first trimester.

In some embodiments, the bed device temperatures in the first trimester are between about 0° C. to about 31° C. cooler than the bed device temperatures of subject prior to the subject becoming pregnant. In some embodiments, the bed device temperatures in the first trimester are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. cooler than the bed device temperatures of subject prior to the subject becoming pregnant. In some embodiments, the bed device temperatures dial values in the first trimester are decreased between about 0 to about 20 dial values from the dial values of bed device prior to the subject becoming pregnant. In some embodiments, the bed device temperatures dial values in the first trimester are decreased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values of the bed device prior to the subject becoming pregnant.

In some embodiments, the bed device temperatures in the first trimester are between about 0° C. to about 31° C. warmer than the bed device temperatures of subject prior to the subject becoming pregnant. In some embodiments, the bed device temperatures in the first trimester are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. warmer than the bed device temperatures of subject prior to the subject becoming pregnant. In some embodiments, the bed device temperatures dial values in the first trimester are increased between about 0 to about 20 dial values from the dial values of bed device prior to the subject becoming pregnant. In some embodiments, the bed device temperatures dial values in the first trimester are increased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values of the bed device prior to the subject becoming pregnant.

In some embodiments, the bed device temperatures of the bed device prior to the subject becoming pregnant are between about 0° C. to about 31° C. warmer than the bed device temperatures in the first trimester. In some embodiments, the bed device temperatures of the bed device prior to the subject becoming pregnant are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. warmer than the bed device temperatures in the first trimester. In some embodiments, the bed device temperatures dial values of the bed device prior to the subject becoming pregnant are increased between about 0 to about 20 dial values from the dial values in the first trimester. In some embodiments, the bed device temperatures dial values of the bed device prior to the subject becoming pregnant are increased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values from the dial values of the first trimester.

In some embodiments, the bed device temperatures of the bed device prior to the subject becoming pregnant are between about 0° C. to about 31° C. cooler than the bed device temperatures in the first trimester. In some embodiments, the bed device temperatures of the bed device prior to the subject becoming pregnant are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. cooler than the bed device temperatures in the first trimester. In some embodiments, the bed device temperatures dial values of the bed device prior to the subject becoming pregnant are decreased between about 0 to about 20 dial values from the dial values in the first trimester. In some embodiments, the bed device temperatures dial values of the bed device prior to the subject becoming pregnant are decreased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values from the dial values of the first trimester.

In some embodiments, the bed device temperatures in the first trimester are between about 0° C. to about 31° C. warmer than the bed device temperatures in the second trimester. In some embodiments, the bed device temperatures in the first trimester are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. warmer than the bed device temperatures in the second trimester. In some embodiments, the bed device temperatures dial values in the first trimester are increased between about 0 to about 20 dial values from the dial values in the second trimester. In some embodiments, the bed device temperatures dial values in the first trimester are increased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values from the dial values of the second trimester.

In some embodiments, the bed device temperatures in the first trimester are between about 0° C. to about 31° C. cooler than the bed device temperatures in the second trimester. In some embodiments, the bed device temperatures in the first trimester are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. cooler than the bed device temperatures in the second trimester. In some embodiments, the bed device temperatures dial values in the first trimester are decreased between about 0 to about 20 dial values from the dial values in the second trimester. In some embodiments, the bed device temperatures dial values in the first trimester are decreased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values from the dial values of the second trimester.

In some embodiments, the bed device temperatures in the third trimester are between about 0° C. to about 31° C. cooler than the bed device temperatures in the second trimester. In some embodiments, the bed device temperatures in the third trimester are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. cooler than the bed device temperatures in the second trimester. In some embodiments, the bed device temperatures dial values in the third trimester are decreased between about 0 to about 20 dial values from the dial values in the second trimester. In some embodiments, the bed device temperatures dial values in the third trimester are decreased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values from the dial values of the second trimester.

In some embodiments, the bed device temperatures in the third trimester are between about 0° C. to about 31° C. warmer than the bed device temperatures in the second trimester. In some embodiments, the bed device temperatures in the third trimester are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. warmer than the bed device temperatures in the second trimester. In some embodiments, the bed device temperatures dial values in the third trimester are increased between about 0 to about 20 dial values from the dial values in the second trimester. In some embodiments, the bed device temperatures dial values in the third trimester are increased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values from the dial values of the second trimester.

In some embodiments, the bed device temperatures in the second trimester are between about 0° C. to about 31° C. warmer than the bed device temperatures in the third trimester. In some embodiments, the bed device temperatures in the second trimester are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. warmer than the bed device temperatures in the third trimester. In some embodiments, the bed device temperatures dial values in the second trimester are increased between about 0 to about 20 dial values from the dial values in the third trimester. In some embodiments, the bed device temperatures dial values in the second trimester are increased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values from the dial values of the third trimester.

In some embodiments, the bed device temperatures in the second trimester are between about 0° C. to about 31° C. cooler than the bed device temperatures in the third trimester. In some embodiments, the bed device temperatures in the second trimester are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. cooler than the bed device temperatures in the third trimester. In some embodiments, the bed device temperatures dial values in the second trimester are decreased between about 0 to about 20 dial values from the dial values in the third trimester. In some embodiments, the bed device temperatures dial values in the second trimester are decreased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values from the dial values of the third trimester.

In some embodiments, the bed device temperatures in the third trimester are between about 0° C. to about 31° C. cooler than the bed device temperatures in the first trimester. In some embodiments, the bed device temperatures in the third trimester are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., or at least or up to about 30° C., at least or up to about 31° C. cooler than the bed device temperatures in the first trimester. In some embodiments, the bed device temperatures dial values in the third trimester are decreased between about 0 to about 20 dial values from the dial values in the first trimester. In some embodiments, the bed device temperatures dial values in the third trimester are decreased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values from the dial values of the first trimester.

In some embodiments, the bed device temperatures in the third trimester are between about 0° C. to about 31° C. warmer than the bed device temperatures in the first trimester. In some embodiments, the bed device temperatures in the third trimester are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. warmer than the bed device temperatures in the first trimester. In some embodiments, the bed device temperatures dial values in the third trimester are increased between about 0 to about 20 dial values from the dial values in the first trimester. In some embodiments, the bed device temperatures dial values in the third trimester are increased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values from the dial values of the first trimester.

In some embodiments, the bed device temperatures in the first trimester are between about 0° C. to about 31° C. warmer than the bed device temperatures in the third trimester. In some embodiments, the bed device temperatures in the first trimester are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. warmer than the bed device temperatures in the third trimester. In some embodiments, the bed device temperatures dial values in the first trimester are increased between about 0 to about 20 dial values from the dial values in the third trimester. In some embodiments, the bed device temperatures dial values in the second trimester are increased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values from the dial values of the third trimester.

In some embodiments, the bed device temperatures in the first trimester are between about 0° C. to about 31° C. cooler than the bed device temperatures in the third trimester. In some embodiments, the bed device temperatures in the first trimester are at least or up to about 0° C., at least or up to about 1° C., at least or up to about 2° C., at least or up to about 3° C., at least or up to about 4° C., at least or up to about 5° C., at least or up to about 6° C., at least or up to about 7° C., at least or up to about 8° C., at least or up to about 9° C., at least or up to about 10° C., at least or up to about 11° C., at least or up to about 12° C., at least or up to about 13° C., at least or up to about 14° C., at least or up to about 15° C., at least or up to about 16° C., at least or up to about 17° C., at least or up to about 18° C., at least or up to about 19° C., at least or up to about 20° C., at least or up to about 21° C., at least or up to about 22° C., at least or up to about 23° C., at least or up to about 24° C., at least or up to about 25° C., at least or up to about 26° C., at least or up to about 27° C., at least or up to about 28° C., at least or up to about 29° C., at least or up to about 30° C., or at least or up to about 31° C. cooler than the bed device temperatures in the third trimester. In some embodiments, the bed device temperatures dial values in the first trimester are decreased between about 0 to about 20 dial values from the dial values in the third trimester. In some embodiments, the bed device temperatures dial values in the second trimester are decreased at least or up to about 0 dial values, at least or up to about 1 dial values, at least or up to about 2 dial value, at least or up to about 3 dial values, at least or up to about 4 dial values, at least or up to about 5 dial values, at least or up to about 6 dial values, at least or up to about 7 dial values, at least or up to about 8 dial values, at least or up to about 9 dial values, at least or up to about 10 dial values, at least or up to about 11 dial values, at least or up to about 12 dial values, at least or up to about 13 dial values, at least or up to about 14 dial values, at least or up to about 15 dial values, at least or up to about 16 dial values, at least or up to about 17 dial values, at least or up to about 18 dial values, at least or up to about 19 dial values, or at least or up to about 20 dial values from the dial values of the third trimester.

In some embodiments, the subject selects the postpartum mode after giving birth. In some embodiments, the subject selects the postpartum mode to automatically adjust the bed device temperature to the first trimester temperatures. In some embodiments, the subject selects the postpartum mode to automatically adjust the bed device temperature to the bed device temperatures prior to the subject becoming pregnant. In some embodiments, the subject can collect the postpartum mode if the subject has a miscarriage or pregnancy loss.

In some embodiments, the one or more sensors collects data from one or more biometrics. In some embodiments, the one or more sensors detects a change in one or more biometrics in the pregnant subject. In some embodiments, the bed device automatically adjusts the temperature of the bed device upon detection of a change in the one or more biometrics in the pregnant subject.

In some embodiments, the bed device app sends a pregnancy summary. Non-limiting information included in the pregnancy summary include HR, HRV, respiration rate, and sleep quality values. In some embodiments, the pregnancy summary is sent to the subject at the end of the first trimester, the second trimester, and/or the third trimester. In some embodiments, the pregnancy summary sends the subject information regarding changes in the one or more biometrics during their pregnancy.

In some embodiments, the pregnancy mode of the bed device enhances users experience of the bed device. In some embodiments, the pregnancy mode of the bed device enhances the user experience of the bed device by improving sleep quality of the user. In some embodiments, the pregnancy mode of the bed device improves the sleep quality values of the subject. Non-limiting examples of sleep quality values include percent light sleep, percent deep sleep, percent REM sleep, total sleep time, total awake time, and net sleep score.

In some embodiments, the pregnancy mode of the bed device results in an increase in percent change of light sleep. In some embodiments, the pregnancy mode of the bed device results in between 0%-300% increase in percent change of light sleep. In some embodiments, the pregnancy mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change of light sleep.

In some embodiments, the pregnancy mode of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of light sleep. In some embodiments, the pregnancy mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change of light sleep.

In some embodiments, the pregnancy mode of the bed device results in an increase in percent change of deep sleep. In some embodiments, the pregnancy mode of the bed device results in between 0%-300% increase in percent change of deep sleep. In some embodiments, the pregnancy mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change in deep sleep.

In some embodiments, the pregnancy mode of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of deep sleep. In some embodiments, the pregnancy mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, %, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change of deep sleep.

In some embodiments, the pregnancy mode of the bed device results in an increase in percent change of REM sleep. In some embodiments, the pregnancy mode of the bed device results in between 0%-300% increase in percent change of REM sleep. In some embodiments, the pregnancy mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% increase in percent change of REM sleep.

In some embodiments, the pregnancy mode of the bed device results in at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of REM sleep. In some embodiments, the pregnancy mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% increase in percent change of REM sleep.

In some embodiments, the pregnancy mode of the bed device results in an increase in percent change of total sleep time. Total sleep time can correspond to the total amount of time the subject is asleep on the bed device. In some embodiments, the pregnancy mode of the bed device results in between 0%-500% increase in percent change of total sleep time. In some embodiments, the pregnancy mode results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% increase in percent change of total sleep time.

In some embodiments, the pregnancy mode of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of total sleep time. In some embodiments, the pregnancy mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in percent change of total sleep time.

In some embodiments, the pregnancy mode of the bed device results in an increase in percent change of a net sleep score. The net sleep score can be calculated by determining the percent change in how many subjects experienced a positive percent change in light, deep, REM, or total sleep time compared to a group that did not experience the pregnancy mode (e.g., a control group). In some embodiments, the pregnancy mode of the bed device results in between 0%-500% increase in percent change of net sleep score. In some embodiments, the pregnancy mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% increase in percent change of net sleep score.

In some embodiments, the pregnancy mode of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less increase in percent change of net sleep score. In some embodiments, the pregnancy mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% increase in percent change of net sleep score.

In some embodiments, the pregnancy mode of the bed device results in a decrease in percent change of total awake time. The total awake time can be calculated by determining the length of time the user is awake during use of the bed device. In some embodiments, the pregnancy mode of the bed device results in between 0%-500% decrease in percent change of total awake time. In some embodiments, the pregnancy mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% decrease in percent change of total awake time.

In some embodiments, the pregnancy mode of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less decrease in percent change of total awake time. In some embodiments, the pregnancy mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% decrease in percent change of total awake time.

In some embodiments, the pregnancy mode of the bed device results in a decrease in percent change of the number of awakenings per use of the bed device. The number of awakenings can be calculated by determining the number of times the user awaken during use of the bed device. In some embodiments, the pregnancy mode of the bed device results in between 0%-500% decrease in percent change of number of awakenings. In some embodiments, the pregnancy mode of the bed device results in at least about 0%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% decrease in percent change of numbers of awakenings.

In some embodiments, the pregnancy mode of the bed device results in at most about 500%, at most about 490%, at most about 480%, at most about 470%, at most about 460%, at most about 450%, at most about 440%, at most about 430%, at most about 420%, at most about 410%, at most about 400%, at most about 390%, at most about 380%, at most about 370%, at most about 360%, at most about 350%, at most about 340%, at most about 330%, at most about 320%, at most about 310%, at most about 300%, at most about 290%, at most about 280%, at most about 270%, at most about 260%, at most about 250%, at most about 240%, at most about 230%, at most about 220%, at most about 210%, at most about 200%, at most about 190%, at most about 180%, at most about 170%, at most about 160%, at most about 150%, at most about 140%, at most about 130%, at most about 120%, at most about 110%, at most about 100%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30% at most about 25%, at most about 20%, at most about 15%, at most about 10%, at most about 5%, or less decrease in percent change of number of awakenings. In some embodiments, the pregnancy mode of the bed device results in about 0%, about 5%, about 10%, about 15%, about 20%, about 25%, about 30%, about 35%, about 40%, about 50%, about 55%, about 60%, about 65%, about 70%, about 75%, about 80%, about 85%, about 90%, about 95%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% decrease in percent change of number of awakenings.

### Additional Aspects of the Article of Furniture

In some embodiments, the article of furniture can be a bed device. The bed device of the present disclosure may assist the user(s) to fall asleep (e.g., assist the user(s) to fall asleep faster) on the bed. The bed of the present disclosure may assist the user(s) to fall asleep for at least about 0.1 hour faster as compared to sleeping on a different bed. The bed of the present disclosure may assist the user(s) to fall asleep for at least about 0.1, 0.2, 0.3, 0.4, 0.5, 0.6, 0.7, 0.8, 0.9, 1, 1.5, 2, or more hours as compared to sleeping on a different bed. The bed of the present disclosure may assist the user(s) to fall asleep for at most about 2, 1.5, 1, 0.9, 0.8, 0.7, 0.6, 0.5, 0.4, 0.3, 0.2, 0.1, or less hours as compared to sleeping on a different bed. The bed of the present disclosure may assist the user(s) to stay asleep longer (e.g., for a non-determined period of time or a predetermined period of time) on the bed. The bed of the present disclosure may assistant the user(s) to stay asleep for at least about 0.5 hour as compared to sleeping on a different bed. The bed of the present disclosure may assist the user(s) to stay asleep for at least about 0.1, 0.2, 0.3, 0.4, 0.5, 1, 1.5, 2, 2.5, 3, 3.5, 4, 4.5, 5, or more hours as compared to sleeping on a different bed. The bed of the present disclosure may assist the user(s) to stay asleep for at least about 5, 4.5, 4, 3.5, 3, 2.5, 2, 1.5, 1, 0.5, 0.4, 0.3, 0.2, 0.1, or less hours as compared to sleeping on a different bed. The bed may shorten or extend a sleep phase of the user(s) while sleeping or resting on the bed. The bed may assist the user(s) to enter or exit a sleep phase while awake, sleeping, or resting on the bed. The bed may improve quality of sleep of the user(s).

The bed device of the present disclosure may assist the user to wake up from sleeping. The bed of the present disclosure may use one or more alarm mechanisms to wake up the user from sleeping. The alarm mechanism(s) may include a personal device (e.g., a mobile device, a computer, a digital alarm clock, etc.) or the bed itself (e.g., mattress, bed sheet, blanket, pillow, mattress frame, etc.). In some cases, the bed may regulate (or adjust) one or more settings of the bed. Such setting(s) of the bed may comprise temperature, position relative to a rest position of the bed, movement (e.g., vibration, translation, rotation, etc.). In an example, the bed may be capable of increasing and/or decreasing a temperature of a portion of the bed (e.g., a portion of a surface of the bed) to wake up the user that is sleeping on the portion of the bed. Such bed may be referred to as a thermal alarm. In some cases, the bed may be configured to wake up the user at a predetermined wake-up time that is input by the user prior to sleeping. In some cases, the bed may not receive data indicative of a predetermined wake-up time from the user. In some cases, the bed may be configured to automatically determine a wake-up time (e.g., an optimal wake-up time) to wake up the user based at least in part on one or more detected biological signals of the user of the bed. The bed may be able to use one or more sensors to detect a movement, presence, and/or absence of the user of the bed, thereby to determine whether the user is awake and/or out of the bed. Additionally, the bed may be configured to automatically diminish and/or turn off the alarm mechanism(s) when it is determined, at least in part by the one or more sensors, that the user is awake and/or out of the bed.

A temperature of the article of furniture (e.g., the bed, such as the mattress, the mattress pad, or the blanket) may be controlled (e.g., increasing, decreasing, or maintaining the temperature of the bed). A temperature of at least a portion of the article of furniture may be controlled. The temperature of the article of furniture may be adjustable or maintained prior to, during, or subsequent to a use (e.g., sleeping or resting for a period of time) by the user(s). In an example, the bed may be pre-warmed (e.g., automatically or per user preference) prior to the use by the user(s). In some cases, temperatures or two or more portions of the article of furniture (e.g., the bed) may be controlled separately or in sync.

The terms “biological signal” and “bio signal” can be used interchangeably. Examples of the biological signal can include a heart signal (e.g., heart rate or heart rate variability), a respiration (breathing) signal (e.g., respiration rate or sound), a motion, a temperature, a movement, perspiration, sound, neural activity, etc. The article of furniture (e.g., the bed) may be capable of detecting one or more biological signals of the user(s). The article of furniture may be capable of adjusting a property of the article of furniture (e.g., temperature or movement of the article of furniture, such as vibration, geometric configuration, etc.) to control (e.g., increase, decrease, or maintain) the biological signal(s) of the user(s) of the article of furniture.

The term “sleep phase,” as used herein, can refer to a light sleep, deep sleep, rapid eye movement (“REM”) sleep, or wake-up. There can be two major stages of sleep: a non-REM sleep and a REM sleep. A person can experience a non-REM sleep first, followed by a shorter period of REM sleep. In some cases, the person can experience a continued cycle of the non-REM sleep and the REM sleep. There may be three stages of non-REM sleep. Each stage can last from 5 to greater than 15 minutes. The person can go through all three stages before reaching REM sleep. In stage one, the person's eyes may be closed, but the person may be easily woken up. This stage may last for 5 to 10 minutes. This stage may be considered as a light sleep. In stage two, the person may be in light sleep. The person's heart rate may slow and the person's body temperature may drop. The person's body may be getting ready for deep sleep. Stage three may be a deep sleep stage. The person may be harder to rouse during this stage, and if the person was woken up, the person would feel disoriented for a few minutes. During the deep stage of the non-REM sleep, the body may repair and regrow tissues, build bone and muscle, and strengthen the immune system. The REM sleep can happen 90 minutes after a person falls asleep. In some cases, the person may have dreams during the REM sleep. An initial period of the REM sleep may typically last 10 minutes. Any latter period of the REM sleep may get longer, and the final period of the REM sleep may last up to about an hour. The person's heart rate and respiration may quicken during the REM sleep (e.g., during the final period of the REM sleep). The person may have intense dreams during the REM sleep, since the brain is more active. The REM sleep may affect learning of certain mental skills.

A “sleep pattern”, as used herein, can indicate a recurrence or change in (i) one or more biological signals and/or (i) one or more sleep phases of the user of the bed. The sleep pattern may be described over a period of time (e.g., 0.5 hour, 1 hour, 2 hour, 3 hour, 4 hour, 5 hour, 6 hour, etc.), along with a count of the biological signal(s) or the sleep phase(s). The sleep pattern may comprise a preferred setting of the biological signal(s) or sleep phase(s) of the user. The preferred setting of the biological signal(s) may comprise a type of the biological signal(s), along with a preferred value or range of values of the biological signal(s) (e.g., a preferred body temperature or range of body temperature of the user). The preferred setting of the sleep phase(s) may comprise a type of the sleep phase(s), along with a preferred value or range of values of the sleep phase(s).

The bed may identify a sleep disorder of the user(s). Examples of the sleep disorder may include dyssomnias, such as insomnia, primary hypersomnia (e.g., narcolepsy, idiopathic hypersomnia, recurrent hypersomnia, posttraumatic hypersomnia, menstrual-related hypersomnia), sleep disordered breathing (e.g., sleep apnea, snoring, upper airway resistance syndrome), circadian rhythm sleep disorders (e.g., delayed sleep phase disorder, advanced sleep phase disorder, non-24-hour sleep-wake disorder), parasomnias (e.g., bedwetting, bruxism, catathrenia, exploding head syndrome, sleep terror, REM sleep behavior disorder, sleep talking), jet lag, restless legs syndrome, etc. Methods and systems for monitoring a person's sleep patterns on a bed and detecting the person's sleep disorder (e.g., snoring, sleep apnea, etc.) are described in U.S. Patent Publication No. 2017/0135632 (“DETECTING SLEEPING DISORDERS”), which is entirely incorporated herein by reference.

The article of furniture (e.g., the bed) may use one or more sensors and/or one or more computer systems to identify the biological signal(s) and/or the sleep order of the user(s). The sensor(s) may or may not be a part of the article of furniture. The sensor(s) may be a part of a space (e.g., room) surrounding the article of furniture. The sensor(s) may be worn by the user(s). The sensor(s) may be used to detect a property (e.g., temperature, movement, etc.) of the article of furniture.

The term “module” refers broadly to software, hardware, or firmware components (or any combination thereof). Modules are typically functional components that can generate useful data or another output using specified input(s). A module may or may not be self-contained. An application program (also called an “application”) may include one or more modules, or a module may include one or more application programs.

The term “on top of” can mean that the two objects, where the first object is “on top of” the second object, can be rotated so that the first object is above the second object relative to the ground. The two objects can be in direct or indirect contact, or may not be in contact at all.

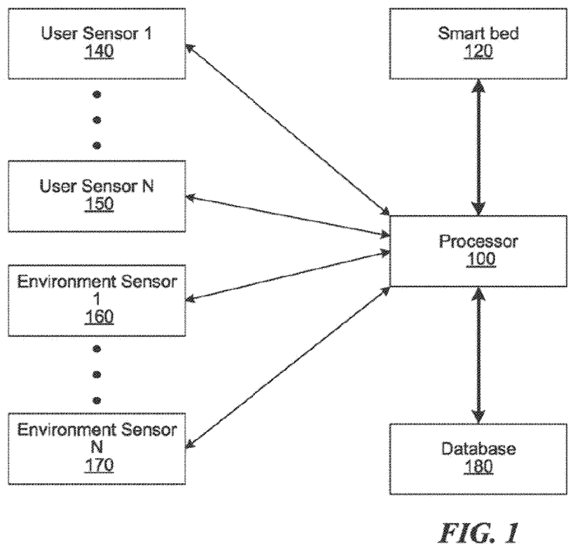

FIGS. 1-3 demonstrate a bed device, as an exemplary embodiment of an article of furniture as provided herein. Any disclosures related to the bed device can be applied to any other examples of the article of furniture.

FIG. 1 is a diagram of an example article of furniture, specifically a bed device (e.g., a mattress or a bed pad), according to one embodiment. Any number of sensors (or user sensors) 140, 150 monitor the bio signals associated with a user, such as the heart rate, the respiration rate, the temperature, motion, or presence, associated with the user. Any number of environment sensors 160, 170 monitor environment properties, such as temperature, sound, light, or humidity. The user sensors 140, 150 and the environment sensors 160, 170 communicate their measurements to the processor 100. The environment sensors 160, 170, measure the properties of the environment that the environment sensors 160, 170 are associated with. In one embodiment, the environment sensors 160, 170 are placed next to the bed. The processor 100 determines, based on the bio signals associated with the user, historical bio signals associated with the user, user-specified preferences, exercise data associated with the user, or the environment properties received, a control signal, and a time to send the control signal to a bed device 120.

According to one embodiment, the processor 100 is connected to a database 180, which stores the biological signals associated with a user or plurality of users of said article of furniture (e.g., the bed device). Additionally, the database 180 can store average biological signals associated with the user, history of biological signals associated with a user, etc. The database 180 can be associated with a user, or the database 180 can be associated with article of furniture (e.g., the bed device).

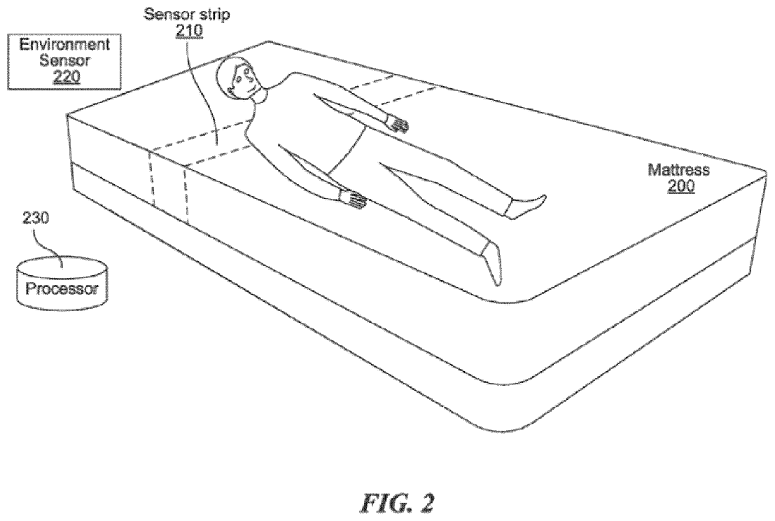

FIG. 2 illustrates an example of the article of furniture (e.g., the bed device) of FIG. 1, according to one embodiment. A sensor (e.g., a sensor strip) 210, associated with a mattress 200 of the bed device 120, monitors bio signals associated with a user sleeping on the mattress 200. The sensor strip 210 can be built into the mattress 200, or can be part of a bed pad device. Alternatively, the sensor 210 can be a part of any other piece of furniture, such as a rocking chair, a couch, an armchair etc. The sensor 210 comprises a temperature sensor, or a piezo sensor. The environment sensor 220 measures environment properties such as temperature, sound, light or humidity.

According to one embodiment, the environment sensor 220 is associated with the environment surrounding the mattress 200. The sensor 210 and the environment sensor 220 communicate the measured environment properties to the processor 230. In some embodiments, the processor 230 can be similar to the processor 100 of FIG. 1. A processor 230 can be connected to the sensor 210, or the environment sensor 220 by a computer bus, such as an I2C bus. Also, the processor 230 can be connected to the sensor 210, or the environment sensor 220 by a communication network.

By way of example, the communication network connecting the processor 230 to the sensor 210, or the environment sensor 220 includes one or more networks such as a data network, a wireless network, a telephony network, or any combination thereof. The data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (WiFi), wireless LAN (WLAN), Bluetooth®, Internet Protocol (IP) data casting, satellite, mobile ad-hoc network (MANET), and the like, or any combination thereof.

The processor 230 is any type of microcontroller, or any processor in a mobile terminal, fixed terminal, or portable terminal including a mobile handset, station, unit, device, multimedia computer, multimedia tablet, Internet node, cloud computer, communicator, desktop computer, laptop computer, notebook computer, netbook computer, tablet computer, personal communication system (PCS) device, personal navigation device, personal digital assistants (PDAs), audio/video player, digital camera/camcorder, positioning device, television receiver, radio broadcast receiver, electronic book device, game device, the accessories and peripherals of these devices, or any combination thereof.

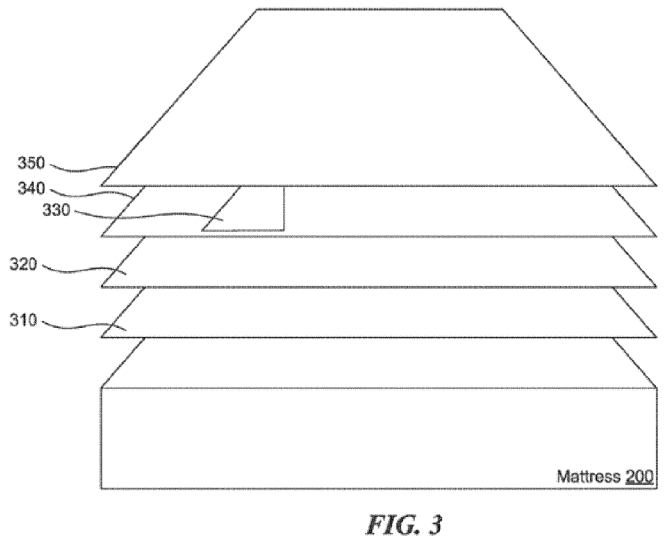

FIG. 3 illustrates an example of at least a portion of the components (e.g., layers) of the article of furniture (e.g., the bed pad device) of FIG. 1, according to one embodiment. In some embodiments, the bed pad device 120 is a pad that can be placed on top of the mattress. Bed pad device 120 comprises a plurality of portions (e.g., a plurality of layers). A top portion (e.g., a top layer) 350 comprises fabric. Another portion (e.g., another layer) 340 comprises a matrix (e.g., a batting) and a sensor (e.g., a sensor strip) 330. A different portion (e.g., a different layer) 320 may be at least a portion of a temperature control device. In an example, the layer 320 comprises coils for cooling or heating the bed device. Alternatively, the layer 320 may comprise a fluid in a fluid flow channel for cooling or heating the article of furniture. A layer 310 comprises waterproof material.

According to another embodiment, the layer 320 comprises a material (e.g., solid, semi-solid, gel, liquid, or a combination thereof) that can be heated or cooled from about 0.5 degrees Celsius (° C.) to about 50° C. In some cases, the material may be heated or cooled from about 0.5° C. to about 50° C. without changing the materials properties such as the state of matter. Alternatively, the materials properties may change during heating or cooling, and such materials properties may be reversible. In some cases, the material can be cooled from about 10° C. to about 50° C. without changing the materials properties such as the state of matter. An example of such materials can be air, water, argon, a synthetic material such as polymers, carbon nanotubes, etc. According to one embodiment, the layer 320 is connected to an external thermal regulator which heats or cools the material, based on the signal received from the processor 230. The material of the layer 320 may be heated or cooled to a temperature in a range between about 10° C. to about 50° C. A temperature of such material that may be adjusted by at least about 0.1° C., 0.2° C., 0.3° C., 0.4° C., 0.5° C., 0.6° C., 0.7° C., 0.8° C., 0.9° C., 1° C., 2° C., 3° C., 4° C., 5° C., 6° C., 7° C., 8° C., 9° C., 10° C., 11° C., 12° C., 13° C., 14° C., 15° C., 20° C., 25° C., 30° C., 35° C., 40° C., 45° C., 50° C., or more. The temperature of such material that may be adjusted by at most about 50° C., 49° C., 48° C., 47° C., 46° C., 45° C., 40° C., 35° C., 30° C., 25° C., 20° C., 15° C., 14° C., 13° C., 12° C., 11° C., 10° C., 9° C., 8° C., 7° C., 6° C., 5° C., 4° C., 3° C., 2° C., 1° C., 0.9° C., 0.8° C., 0.7° C., 0.6° C., 0.5° C., 0.4° C., 0.3° C., 0.2° C., 0.1° C., or less. The external thermal regulator may be a part of a temperature control device that is operatively coupled to the article of furniture.

According to another embodiment, the layer 320 comprising the material is integrated into the mattress, the bed sheets, the bed cover, the bed frame, etc. The layer 320 comprising the material can also be integrated with any piece of furniture.

In some embodiments, the bed device as provided herein can comprise one or more components comprising: at least a portion of the bed device configured to hold a fluid; a reservoir in fluid communication with the at least the portion of the bed device, configured to contain the fluid; a temperature regulator in fluid communication with the at least the portion of the bed device and the reservoir, configured to modulate a temperature of the fluid; and a processor operatively coupled to the temperature regulator, programmed to control the temperature regulator to modulate the temperature of the fluid, thereby to regulate the temperature of the at least the portion of the bed device.

In some embodiments, the processor can provide a control signal to the temperature regulator. In some embodiments, the control signal can be an instruction for the temperature regulator to change a temperature of the article of furniture. In some embodiments, the control signal can be an instruction for the temperature regulator to change a temperature of a portion of the article of furniture. In some embodiments, the control signal can be an instruction to change the temperature to the bedtime temperature. In some embodiments, the control signal can be an instruction to change the bedtime temperature to the initial temperature. In some embodiments, the control signal can be an instruction to change the temperature to the initial temperature. In some embodiments, the control signal can be an instruction to change the initial temperature to the final temperature. In some embodiments, the control signal can be an instruction to change the temperature to the final temperature. In some embodiments, the control signal can be an instruction to change the temperature to the wake-up temperature. In some embodiments, the control signal can be an instruction to change the final temperature to the wake-up temperature.

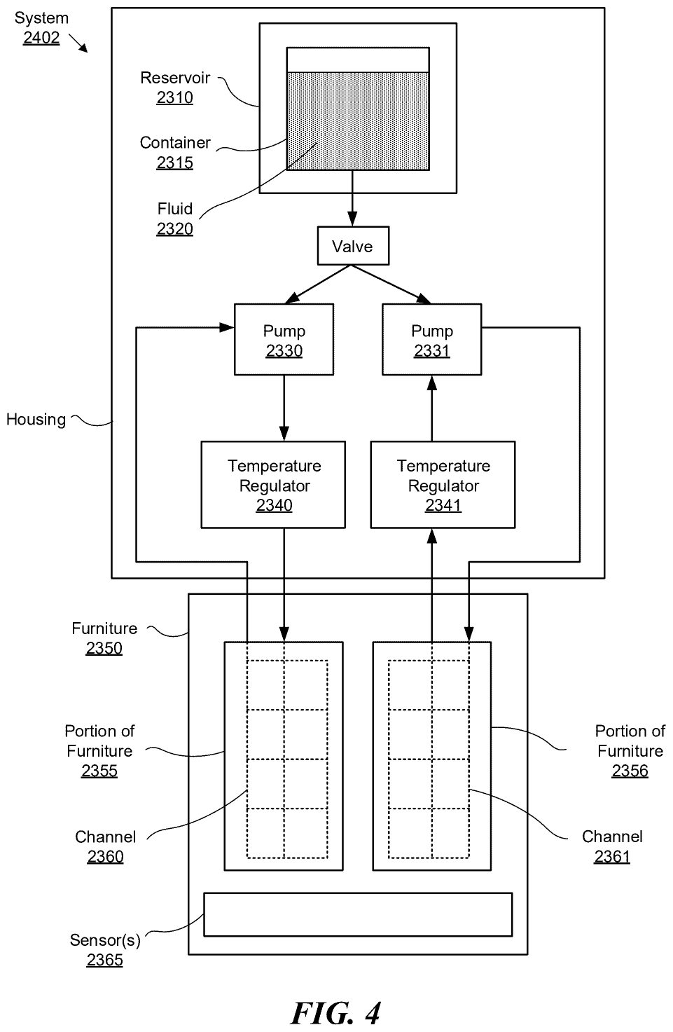

FIG. 4 schematically illustrates an example of a system for regulating temperatures of two portions of an article of furniture (e.g., a bed, mattress, or mattress pad), which system comprises two fluid loops (e.g., two water loops). The system 2402 may comprise a reservoir 2310 configured to contain the fluid 2320 (e.g., water). The reservoir may comprise a container 2315 (e.g., a removable or non-removable container) configured to contain the fluid. Neither the reservoir 2310 nor the container 2315 is configured to modulate the temperature of the fluid that is contained in the container 2315. The system 2402 may comprise two fluid loops that are in fluid communication with the container 2315 of the reservoir 2310. The two fluid loops may or may not be in fluid communication with each other. The reservoir 2310 may serve as a common reservoir for the two fluid loops of the system 2402. The first fluid loop comprises (i) the pump 2330, (ii) the temperature regulator 2340, and (iii) the channel 2360 of the portion 2355 of the article of furniture 2350. The pump 2330 may be configured to retrieve or receive the fluid 2320 from the container 2315 of the reservoir 2310. The pump 2330 (and/or a valve as provided herein) may be configured to prevent flow of the fluid 2320 away from the pump 2330 and back into the container 2315 of the reservoir 2310. The pump 2330 may be configured to direct flow of the fluid 2320 in the first fluid loop, from the pump 2330, to the temperature regulator 2340, to the channel 2360, and back to the pump 2330. The temperature regulator 2340 may be configured to modulate a temperature of the fluid 2320 in the first fluid loop. The second loop may comprise features that may or may not be identical to the first loop. The second fluid loop comprises (i) the pump 2331, (ii) the temperature regulator 2341, and (iii) the channel 2361 of the portion 2356 of the article of furniture 2350. The pump 2331 may be configured to retrieve or receive the fluid 2320 from the container 2315 of the reservoir 2310. The pump 2331 (and/or a valve as provided herein) is configured to prevent flow of the fluid 2320 away from the pump 2331 and back into the container 2315 of the reservoir 2310. The pump 2331 may be configured to direct flow of the fluid 2320 in the second fluid loop, from the pump 2331, to the channel 2361, to the temperature regulator 2341, and back to the pump 2331. The temperature regulator 2341 may be configured to modulate a temperature of the fluid 2320 in the second fluid loop. The system 2402 may further comprise one or more sensor(s) 2365 configured to detect a biological signal (e.g., a heart signal, a respiration signal, a motion, a temperature, and/or perspiration) of at least one user of the article of furniture 2350. The sensor(s) 2365 may be part of the article of furniture 2350. The sensor(s) 2365, the portion of furniture 2355, and the portion of furniture 2356 may be in different parts of the article of furniture 2350. The system 2402 may regulate the temperature of the portion of furniture 2355 and/or the temperature of the portion of furniture 2356 based at least in part on the detected biological signal of the at least one user (e.g., one or two users) of the article of furniture 2350. In some cases, as shown in FIG. 4, one or more valves can control the flow (e.g., time, direction, flow rate, etc.) of the fluid 2320 between the container 2315 and any one of the pumps 2330 and 2331. The valve(s) can be (i) disposed separately from the container, the reservoir, and the pumps, (ii) disposed as part of the container or the reservoir, or (iii) disposed as part of any one of the pumps. In some cases, as shown in FIG. 4, the bed device may comprise a housing, which housing containing therewithin one or more members of the following: the reservoir 2310, the container 2315, the pump(s) 2330/2331, the valve(s) between the container 2315 and the pump(s), the temperature regulator(s) 2340/2341, and any fluid channels connecting there between. The furniture 2350 may not be contained within the housing.

Non-limiting examples of a pump as provided herein can include positive displacement pump, gear pump, screw pump, progressing cavity pump, roots-type pump, peristaltic pump, plunger pump, compressed-air-powered double-diaphragm pump, hydraulic pump, velocity pump, radial flow pump, axial flow pump, eductor jet pump, gravity pump, steam pump, valveless pump, etc.

Non-limiting examples of a valve as provided herein can include a check valve, clack valve, non-return valve, reflux valve, retention valve or one-way valve.

A pump and/or a valve (e.g., individually or a combination thereof) can operate as a flow controller (e.g., a gate) to control flow (e.g., time, direction, flow rate, etc.) of the fluid as provided herein. The gate can function as a one-way gate or a multi-way gate, such as two-way gate, three-way gate, four-way gate, etc.

In some cases, the temperature regulator can achieve temperature gelation via thermoelectric temperature regulation. Thermoelectric temperature regulation (e.g., heating and/or cooling) can be implemented using an electric-based system (e.g., by a thermoelectric engine). The thermoelectric engine can be configured to convert electrical energy into a heat flux (or a temperature difference), or convert the heat flux into electrical energy. The thermoelectric engine can be a solid-state device. In some examples, the temperature regulator can be a thermoelectric cooler, a thermoelectric heat pump, and/or a Peltier device.

Any of the systems and methods disclosed herein can be processed or performed (e.g., automatically) in real-time. The term “real time” or “real-time,” as used interchangeably herein, generally refers to an event (e.g., an operation, a process, a method, a technique, a computation, a calculation, an analysis, an optimization, etc.) that is performed using recently obtained (e.g., collected or received) data. Examples of the event may include, but are not limited to, receiving data from a source of the data (e.g., sensor, database, etc.), analyzing data, determining a conclusion or hypothesis based on the data, sending an instruction to a device (e.g., a temperature regulator), etc. In some cases, a real time event may be performed almost immediately or within a short enough time span, such as within at least 0.0001 ms, 0.0005 ms, 0.001 ms, 0.005 ms, 0.01 ms, 0.05 ms, 0.1 ms, 0.5 ms, 1 ms, 5 ms, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 ms, 1 ms, 0.5 ms, 0.1 ms, 0.05 ms, 0.01 ms, 0.005 ms, 0.001 ms, 0.0005 ms, 0.0001 ms, or less.

Other features of the article of furniture, such as a bed device, may be described in, for example, U.S. patent application Ser. No. 14/732,608 (“Methods and systems for gathering human biological signals and controlling a bed device”), U.S. patent application Ser. No. 14/732,624 (“Methods and systems for gathering and analyzing human biological signals”), U.S. patent application Ser. No. 15/178,124 (“Bed device system and methods”), Patent Cooperation Treaty Patent Application No. PCT/US2016/031054 (“An apparatus and methods for heating or cooling a bed based on human biological signals”), Patent Cooperation Treaty Patent Application No. PCT/US2016/030594 (“Vibrating alarm system and operating methods”), Patent Cooperation Treaty Patent Application No. PCT/US2019/014217 (“Sleep pod”), and Patent Cooperation Treaty Patent Application No. PCT/US2019/064056 (“Systems and methods for regulating a temperature of an article of furniture”), each of which is entirely incorporated herein by reference.

### Computer Systems

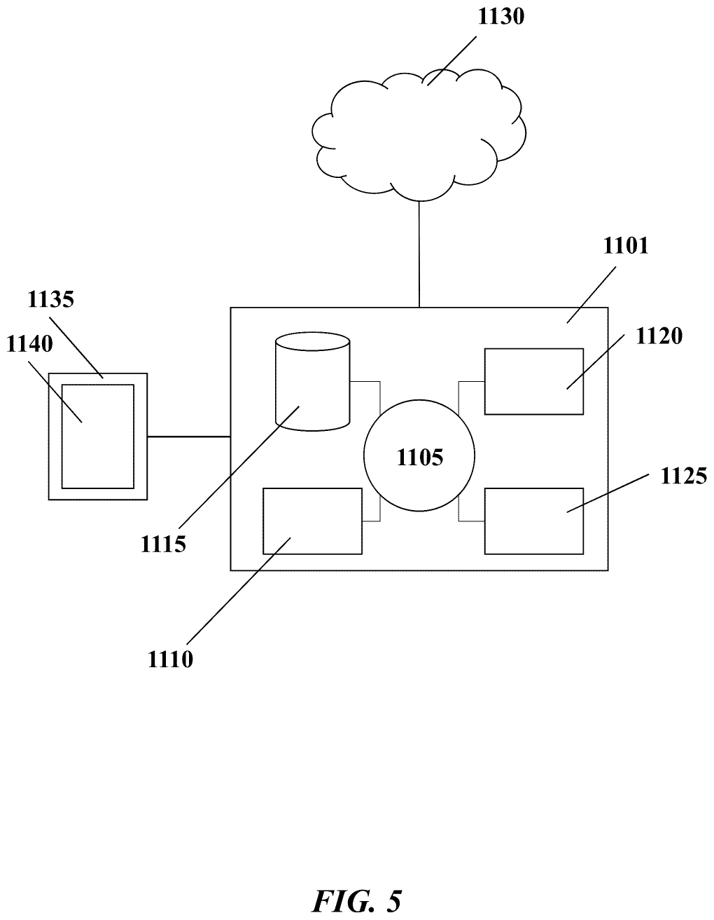

The present disclosure provides computer systems that are programmed to implement methods of the disclosure. FIG. 5 shows a computer system 1101 that is programmed or otherwise configured to adjust a setting (e.g., temperature) of an article of furniture (e.g., a bed device) in accordance with any of the methods provided herein. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user (e.g., a wearable device, a mobile device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, (i) a medium to provide feedback to the user (e.g., sleep quality score, sleep patterns, biological signal(s) of the user during use of the article of furniture, etc.) and/or (ii) receive input from the user (e.g., age, biological sex, health condition(s) such as any information about women's health as provided herein, desired temperature(s) for one or more sleep temperature settings, etc.). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, determine an optimal setting (e.g., temperature) of the article of furniture for the user. The algorithm can, in another example, generate an instruction for changing or optimizing an operation of the temperature regulator for regulating a temperature of a portion of the article of furniture (e.g., the bed device) as provided herein. The algorithm can be performed via artificial intelligence (e.g., an artificial intelligence algorithm) or machine learning (e.g., a machine learning algorithm).

The term “artificial intelligence algorithm” or “artificial intelligence” as used herein generally refers to software-based algorithms that are executable on computing hardware and are operable to adapt and adjust their operating parameters in an adaptive manner depending upon information that is presented to the software-based algorithms when executed on the computing hardware. An example of the artificial intelligence algorithm can include a machine learning algorithm.

The term “machine learning algorithm” or “machine learning” as used herein generally refers to one or more software-based algorithms that are executable upon computing hardware, such as the data processing arrangement, and are operable to adapt and adjust their operating parameters in an adaptive manner depending upon information that is presented to the software-based algorithms when executed upon the computing hardware. Optionally, the machine learning algorithms can include neural networks such as recurrent neural networks, recursive neural networks, feed-forward neural networks, convolutional neural networks, deep belief networks, and convolutional deep belief networks; self-organizing maps; deep Boltzmann machines; and stacked de-noising auto-encoders. Optionally, the machine learning algorithms can employ any one or combination of the following computational techniques: constraint program, fuzzy logic, classification, symbolic manipulation, fuzzy set theory, evolutionary computation, cybernetics, data mining, approximate reasoning, derivative-free optimization, decision trees, or soft computing.

### EXAMPLES

The application may be better understood by reference to the following non-limiting examples, which are provided as exemplary embodiments of the present disclosure. The following examples are presented in order to more fully illustrate embodiments and should in no way be construed, however, as limiting the broad scope of the present disclosure.

### Example 1: Temperature Adjustments Using Bedtime, Initial, and Final Temperatures of Bed Device

Sleep cycles can last 90-110 minutes on average; there is variability among people in sleep cycle length.

### Test Groups

The control group had a total of 566 total subjects (487 males, 64 females, 18 unidentified) and the mean age of the subjects was 39.2 years old (range 18-71 years old). The experimental group had a total of 523 subjects (438 males, 66 females, 18 unidentified) and the mean age of the subjects was 40.2 years old (range 20-78 years old). Control & test groups were matched for distribution of age, sex, temperature profiles and percent time in sleep stages.

### Experimental Design

Each subject participated for 2 weeks in the study. In both the control group and the experimental group, the subjects had the three temperatures profile. In the second week, the experimental group had the 6 temperatures profile, with the temperature incrementally increasing every 90 minutes. In the second week for the control group, the subject had the same three temperatures profiles as the first week.

FIG. 6 illustrates what the increasing temps will look like throughout the night. For example, if a subject has a warmer bedtime temperature vs. initial, the bed device temperature slowly cooled the subject to the preferred initial temp during Sleep Cycle 1 (SC1). In the experimental group, there were 4 key temperature transitions throughout the night. SC1 was the subject's initial temp and Sleep Cycle 5 (SC5) was the subject's final temp.

All data were run through the sleep algorithm (Hercules™), which has improved accuracy for deep, REM, and light sleep stages.

### Filtering Inclusion Criteria

Subjects had to be on the bed device for at least 5 out of 7 nights in Week 1 and at least 5 out of 7 nights in Week 2 for both control and experimental groups. Experimental group was filtered for nights where there was a dial update in the initial phase because the dial updated removed the subject out of the experiment; however, these subjects were included in the dial updates and settings updates summaries. Only subjects that had an increase of ≥1 on the temperature dial between initial & final temperature phases were included in the analysis.

### Results

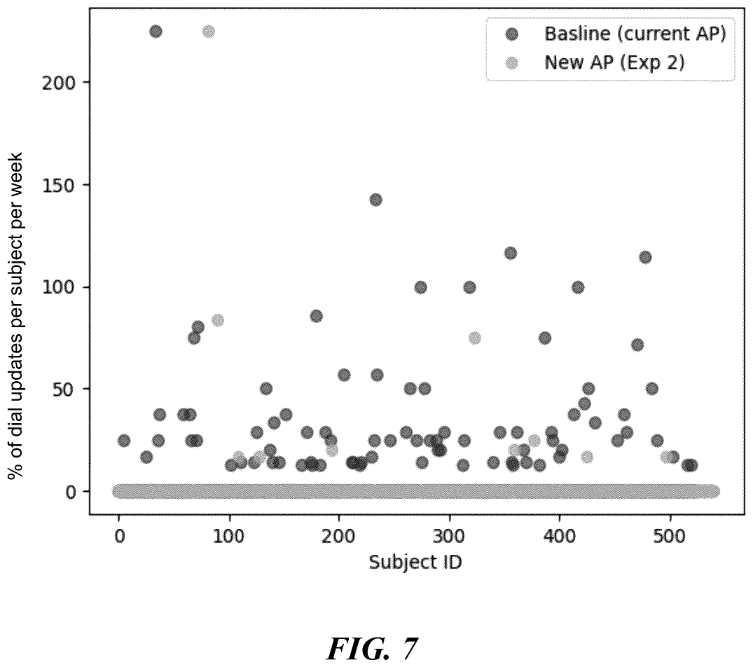

The experimental group had a reduction in dial updates by 94% during the initial temperature; this reduction was similar for both men and women. Average percent dial updates per subject per week equaled ˜6-7% for subjects in the control group. This was reduced to 1% in the experimental group, as shown in FIG. 7. There was no difference in percent dial updates for the final temperature.

In addition to reducing dial updates for both men and women, subjects in the experimental group had specifically improved deep and light sleep for women, leading to increased total sleep time overall. There was a 15% increase in the percentage of women with 25% increase in deep sleep and ˜5% increase in deep sleep on average. There was a 10% increase in the percentage of women with ≥5% increase in light sleep (˜3% increase in light sleep on average). There was a 13% increase in the percentage of women with ≥5% increase in total sleep time (˜3% increase in total sleep time on average). There was ˜85% improvement in women's overall net sleep score, where the average percentage of subjects with positive changes in sleep stages (e.g., ≥5%) increased.

The effect of the incremental increases in temperature was also evaluated in subjects classified as “Poor sleepers”. For subjects who experience low (<20%) REM sleep, there was a 25% increase in the percentage of women with 25% increase in deep sleep, a 20% increase in the percentage of women with ≥5% increase in REM sleep, a 45% increase in the percentage of women with ≥5% increase in total sleep time, a 25% increase in the percentage of women with ≥5% increase in light sleep, and a 7% improvement in total deep sleep, 13% increase in REM sleep, and 9% increase in total sleep time. Overall, the incremental increases in temperature resulted in ˜99% improvement in women's overall net sleep score, where the average percentage of subjects with positive changes in sleep stages (e.g., ≥5%) is increased.

For subjects who experience low (<7 hours) total sleep time on average, there was a 25% increase in the percentage of women with 25% increase in deep sleep, a 34% increase in the percentage of women with ≥5% increase in total sleep time, a 41% increase in the percentage of women with ≥5% increase in light sleep, a 7% improvement in total deep sleep, 5% increase in REM sleep, 8% increase in light sleep, and 9% increase in total sleep time. Overall, there was a ˜62% improvement in women's overall net sleep score, where the average percentage of subjects with positive changes in sleep stages (e.g., ≥5%) is increased.

For subjects who experience low (<15%) deep sleep on average, there was a 10% increase in the percentage of men with ≥5% increase in deep sleep.

### Conclusions

Increasing temperature in 90-minute cycles decreased dial updates during the initial temperature. Increasing temperature incrementally in-line with average sleep cycles (e.g., 90 min) improved deep sleep, light sleep, and total sleep time in women.

### Example 2: Temperature Regulation of the Bed Device and Biometrics

A decline in core temperature during sleep onset can increase slow wave sleep (SWS) and decrease heart rate (HR).

49 subjects (24 men, 25 women; mean±SD (range): age=34.4±11.5 (21.8-64.6) years) slept for 16 nights each (784 total nights) with the bed device temperature-control off (OFF) for the first week as baseline, the bed device temperatures on (ON) at the subject's chosen temperatures for the second week, and the bed device OFF for two nights. Subjects wore a home sleep test device (HST) for eight nights total: four nights during each OFF and ON. Subjects wore a Fitbit device throughout the study to collect nightly HR and heart rate variability (HRV). Paired t-tests compared each subject's sleep and HR/HRV for bed device ON vs. OFF, separating for biological sex and bed device temperature. A multiple linear regression was used to evaluate the percent change in SWS from OFF to ON.

Four factors explained 64% of the variance in the percent change in SWS from OFF to ON (r²=0.64; P<0.001): subjects' baseline SWS during OFF, age, average bed device temperature, and sex. Men sleeping at cooler bed device temperatures (<26° C.) increased SWS by 17 min (P=0.0001; cohen's−d=0.65), decreased HR 1.5 beats·minute⁻¹ (P=0.007, cohen's−d=0.20), and increased HRV 13% (P=0.045; cohen's−d=0.23). Women's sleep was unaffected by bed device ON (all P>0.05); however, sleeping at warmer temperatures (>27° C.) decreased HR 1.5 beats·minute⁻¹ (P=0.021; cohen's−d=0.18).

Sleeping on the bed device at cooler temperatures significantly improved SWS in males, especially those with below-average SWS pre-bed device and increasing age. One week of sleeping on the bed device improved HR in both sexes similar to exercise-training.

### Example 3: Initial Temperature Profiles of the Bed Device

First-time bed device users have a hard time choosing the temperatures for the first week of use on the bed device. The wrong choice of temperature leads to increased wake-ups and frustration. The goal of this experiment was to prescribe starting temperatures that are more neutral, and specific to the subject's age, sex, and geographic location (e.g., winter vs. summer temperatures). Overall, these temperature profiles were not a long-term solution, but more of a baseline to help subjects get adjusted to the bed device in a neutral way and reduce subject discomfort. Also, by prescribing a temperature profile, subjects were likely to keep the same “shape” of the temperature profile that was prescribed (e.g., banana shape or linearly increase).

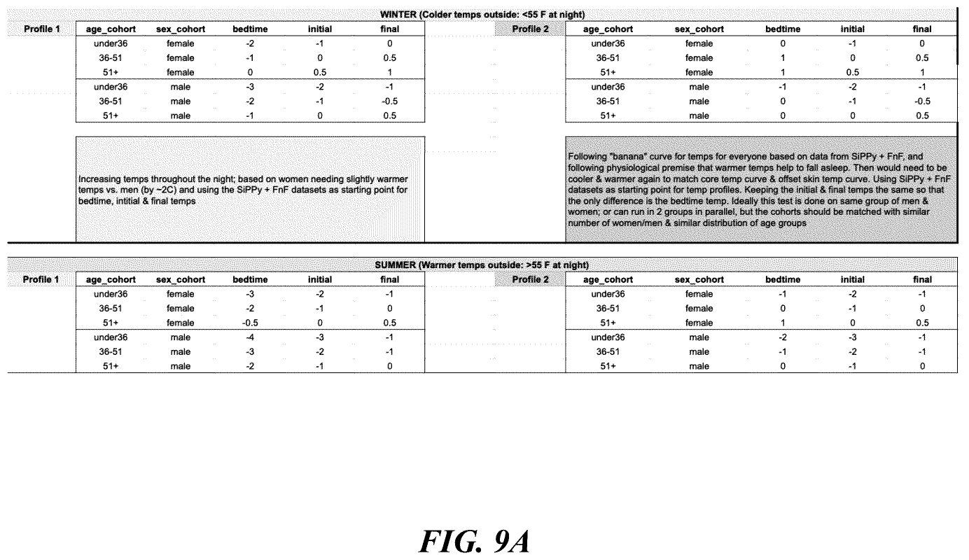

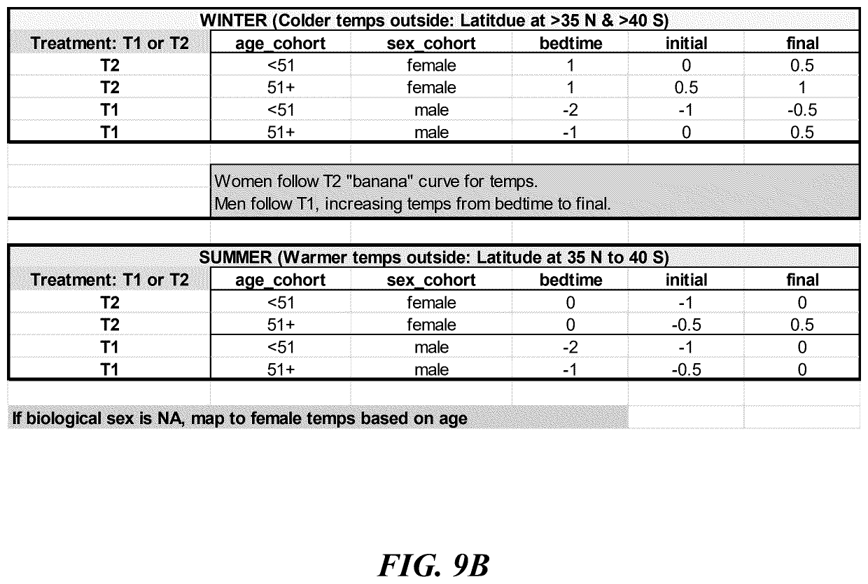

When subject's first created a profile in the app, the subjects were randomly assigned to the control, T1 (linearly increasing temperature from bedtime to initial to final), or T2 (banana shaped profile: warm bedtime temp, cool initial temp, and warm final temp) group. The initial and final temperatures for T1 and T2 were similar for age and sex; the only difference between T1 and T2 was a cooler bedtime temp for T1 compared to a warmer bedtime temp for T2. Within those three groups, subjects were assigned to the temperature profile based on their age bin, biological sex and geographic location (e.g., latitude). FIGS. 9A and 9B shows the dial values for the temperature profiles to be assigned to the subjects in this study.

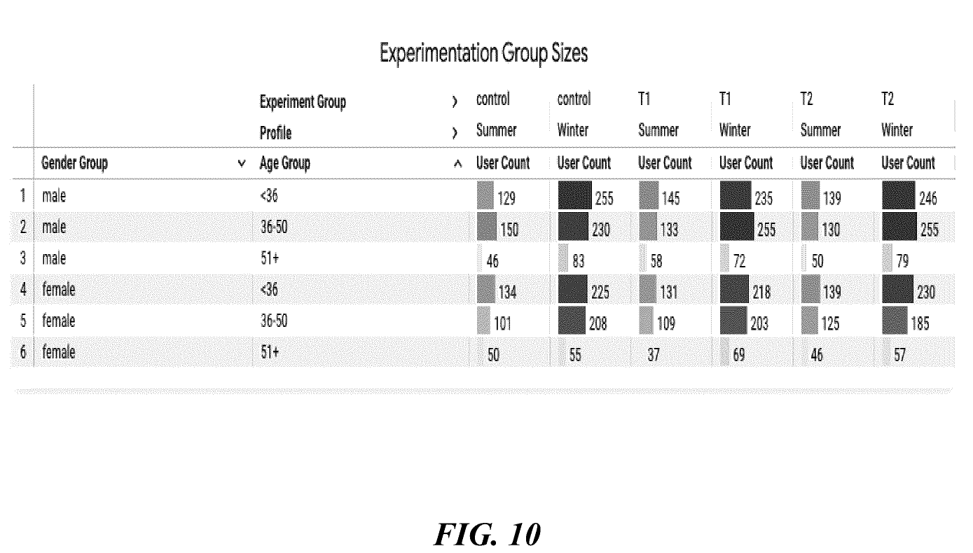

FIG. 10 shows the breakdown of the subjects included in the study based on the subject's age, biological sex, geographical location, and temperature group (e.g., control, T1, T2).

The subjects assigned to each temperature group answered a number of survey questions regarding the subject's experience with the bed device. The largest survey responses were n=20-30 responses for the 36-50 y old men and women in the Winter profiles. Since there was a similar number of responses for this age group across control, T1 and T2, the results focused primarily on the Night 1 responses from these groups.

### Men, 36-50 y, Winter Profile—1st Night Responses

T1 was the best profile (e.g., linear increase in temps) compared to T2 (banana shaped) and control (original temperature regulation).

Below for the responses of male subjects between 36 to 50 years old in a winter profile to the survey questions.

How was your first night on the Pod?
T1 had ˜46% increase in first night on the bed device rating vs. control.
T1: 3.8/5 mean rating on the bed device, with the median rating of 4.0/5.0.
Control group: 2.6/5 mean rating on the bed device, with the median rating of 3.0/5.0.

What was the main issue hurting your experience?
T1 & T2 had ˜50% reduction in Temperature being the main issue vs. control
T1: 26% said temperature was main issue hurting experience
T2: 24% said temperature was main issue hurting experience
Control: 52% said temperature was main issue hurting experience

How satisfied were you with your sleep last night? (fully+fairly; 4 & 5/5 ratings)
T1 had ˜97% increase in sleep satisfaction vs. control
T1: 73% fairly or fully satisfied
T2: 58% fairly or fully satisfied
Control: 37% fairly or fully satisfied

On average, how comfortable were you with your body temperature? (Comfortable rating only)
T1 had ˜51% increase in comfort of body temperature vs. control
T1: 65% said they were comfortable with their body temperature last night
T2: 58% said they were comfortable with their body temperature last night
Control: 43% said they were comfortable with their body temperature last night. 23% were “uncomfortable” or “very uncomfortable”.

Percentage ratings of “Still finding my temperature settings”
T1 had ˜32% reduction in the number of people that said they were still finding their temperature settings vs. control.
T1: 52% men said they were still finding their temperature settings
T2: 64% men said they were still finding their temperature settings
Control: 76% men said they were still finding their temperature settings

The bed device was too cold during the night and woke me up.
T1 had ˜40% reduction in cold rating vs. control
T1: 2.6% men said the bed vice was too cold and woke them up during the night.
T2 & control groups: 4.3% men said the bed device was too cold and woke them up during the night.

The bed device was too hot during the night and woke me up.
T1 had similar rating vs. control
1.3% of men said the bed device was too hot and woke them up during the night.

The bed device was too hot, I could not fall asleep.
T1 & T2 had NO ratings of this response vs. control
1.4% of men in control group said the bed device was too hot at bedtime and prevented them from falling asleep.

The bed device was too cold, I could not fall asleep.
T1 & T2 had NO ratings of this response vs. control.
1.4% of men in control group said the bed device was too cold at bedtime and prevented them from falling asleep.
   Women, 36-50 y, Winter Profile—1st night responses

T1 was the best profile (e.g., linear increase in temps) compared to T2 (banana shaped) and control.

Below for the responses of female subjects between 36 to 50 years old in a winter profile to the survey questions.

How was your first night on the Pod?
T1 had ˜28% increase in first night on bed device rating vs. control
T1: 2.9/5.0 mean rating on the bed device, with the median rating of 3.0/5.0 & max rating of 5.0.
T2: 3.2/5.0 mean rating on the bed device, with the median rating of 3.0/5.0 & max rating of 5.0.
Control group: 2.5/5 mean rating on the bed device, with the median rating of 3.0/5.0 & max rating of 4.0.

What was the main issue hurting your experience?
T2 had ˜35% reduction in Temperature being the main issue vs. control
T1: 53% said temperature was main issue hurting experience
T2: 35% said temperature was main issue hurting experience
Control: 54% said temperature was main issue hurting experience

How satisfied were you with your sleep last night? (fully+fairly; 4 & 5/5 ratings)
T2 had ˜150% increase in sleep satisfaction vs. control
T1: 46% were fairly or fully satisfied
T2: 45% were fairly or fully satisfied
Control: 18% were fairly satisfied; there was no one that said they were fully satisfied.

On average, how comfortable were you with your body temperature? (Comfortable rating only)
T2 had ˜122% increase in comfort of body temperature vs. control
T1: 33% said they were comfortable with their body temperature last night
T2: 40% said they were comfortable with their body temperature last night; only 20% were “uncomfortable” or “very uncomfortable”.
Control: 18% said they were comfortable with their body temperature last night 45% were “uncomfortable” or “very uncomfortable”.

Percentage ratings of “Still finding my temperature settings”
T2 had ˜25% reduction in the number of people that said they were still finding their temperature settings vs. control.
T1: 47% women said they were still finding their temperature settings
T2: 60% women said they were still finding their temperature settings
Control: 82% women said they were still finding their temperature settings

The bed device was too cold during the night and woke me up.
T2 had ˜63% reduction in cold rating vs. control
T1: 20% women said the bed device was too cold and woke them up during the night.
T2: 20% women said the bed device was too cold and woke them up during the night.
Control: 54% women said the bed device was too cold and woke them up during the night.

The bed device was too hot during the night and woke me up.
T2 had 5% higher rating of bed device being too hot vs. control
T1: 13% women said the bed device was too hot and woke them up during the night.
T2: 5% women said the bed device was too hot and woke them up during the night.
Control: 0% women said the bed device was too hot and woke them up during the night.

The bed device was too hot, I could not fall asleep.
No group had any ratings of this response
The bed device was too cold, I could not fall asleep.
T2 reduced by 44% the number of people saying Pod was too cold.
T1: 27% of women said the bed device was too cold at bedtime and prevented them from falling asleep.
T2: 5% of women said the bed device was too cold at bedtime and prevented them from falling asleep.
Control: 9% of women said the bed device was too cold at bedtime and prevented them from falling asleep.

### Example 4: Bed Device Temperature Regulation and Menstruation

Younger women (e.g., less than about 35 years old) preferred a warmer bed device temperature during the follicular phase of their menstrual cycle. This change in bed device temperature matched the body temperature difference between the follicular and luteal phases of at least about 0.3° C. Women overall had a higher percentage of dial updates than men during bedtime and initial phases, which may have been the result of women adjusting their temperatures to accommodate the menstrual cycle phase.

An automatic 0.3° C. increase in bedtime & initial temperature was applied during the follicular phase of the subject's menstrual cycle. The body temperature of a female subject is higher by at least about 0.3 to about 0.7° C. during the luteal phase compared to the follicular phase during the subject's menstrual cycle. This increase in body temperature leads to discomfort, night sweats, and poor sleep. The goal of the experiment was to determine if female subjects set their bed device to warmer temperatures during the follicular phase of the subject's menstrual cycle compared to during the subject's luteal phase.

Terra data from female subjects (n=98 female subjects) using a Garmin wearable was collected. The Garmin wearable had the female subject's menstruation data already incorporated. The data collected included temperature events for these subjects and those temperature events were filtered for female subjects with regular cycle lengths (e.g., 21-35 days) and filtered for female subjects that had temperature event data at least 3 consecutive menstrual cycles (e.g., n=34 female subjects). The filtered female subjects were then grouped into the following age bins for analysis: age less than 35 years old (e.g., 17 female subjects), age greater than or equal to 35 years old to less than or equal to 60 years old (e.g., 17 female subjects), and age greater than 60 years old (e.g., 0 female subjects). The temperature event data was further filtered to ensure that female subjects include in the analysis had at least 8 days of temperature data in each of the follicular phase and the luteal phase for at least 2 menstrual cycles.

Temperatures were found for each of the bedtime, initial and final phases by taking the last adjusted temperature for that phase (either via smart transition, dial update or settings update). To find the aggregate values: the data was grouped by user, period, and phase (follicular vs. luteal), and the median temperature values for bedtime, initial, final, and the relative % dial updates were found (#dial updates/#nights with temp data in each group).

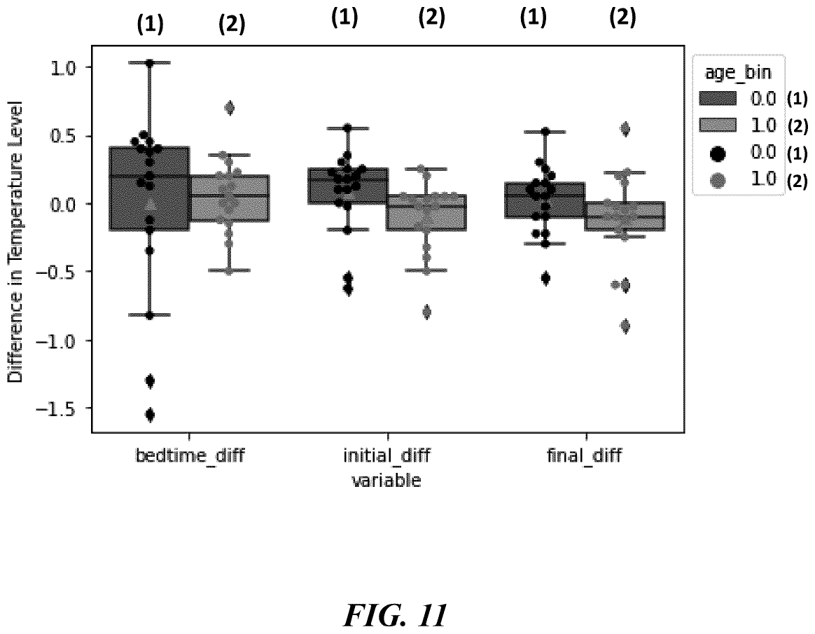

Analysis of the temperature event data found that the bed device temperatures were higher during the subject's follicular phase compared to during the subject's luteal phase. FIG. 11 shows the difference in temperature level of the bed device during the subject's follicular phase (1) and luteal phase (2) for both female subjects under 35 years old and female subjects between the age of 35 to 60 years old. However, the temperature difference between the follicular phase and the luteal phase is greater in female subjects under the age of 35.

Core body temperature tends to be at least about 0.3° C. to about 0.7° C. lower during follicular phase compared to during luteal phase. Younger female subjects (e.g., <35 y) preferred a bed device temperature 0.2 warmer (on the temperature dial) during follicular phase for both the bedtime & initial phases, but not during the final phase. This dial change corresponded to about 0.3° C. change in bed device temperature, which is the average change in core body temperature (and skin temperatures) during the luteal vs. follicular phase. This data also indicated that younger female subjects may be more sensitive to these menstrual cycle fluctuations vs. older (e.g., >35 y) female subjects.

As shown in Table 1, the median dial value difference for female subjects during the subject's menstrual cycle in the two age groups analyzed. The medial dial value difference was calculated by finding the difference between median bed device temperatures of follicular phase and luteal phase of each menstrual cycle and then taking the mean temperature difference of the bed device across menstrual cycles for each user and then calculated the median for all users in each age group.

TABLE 1
Median dial value differences for female users at
the different temperature settings and age groups.

| variable | age_bin | value |
|---|---|---|
| bedtime_diff | 0.0 | 0.200 |
| | 1.0 | 0.050 |
| final_diff | 0.0 | 0.050 |
| | 1.0 | −0.100 |
| initial_diff | 0.0 | 0.175 |
| | 1.0 | −0.025 |

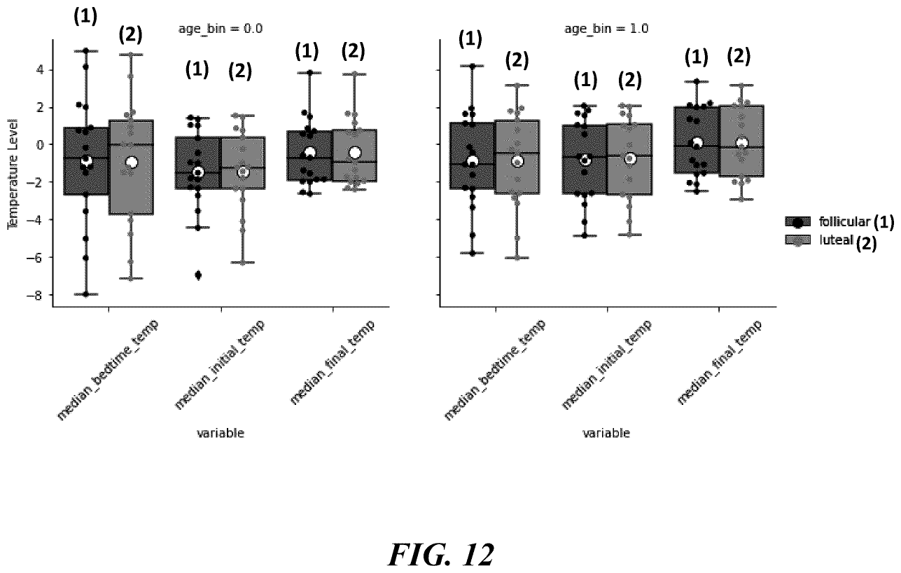

When the median temperatures for each phase across all months of data and the mean temperature across each user were analyzed, slight changes in median temperature during follicular (1) vs. luteal (2) phases were observed (FIG. 12).

At bedtime, the temperatures increased from the follicular phase to the luteal phase when looking at the median value. Overall, the median temperatures across all phases tend to be fairly neutral for women in this dataset (e.g., close to 0 on the temperature dial). In the younger group (e.g., <35 years old), the temperatures also appeared to follow a curve of having warmer temperatures at bedtime and final, and cooler temperatures at initial. In summary, it appeared that female users adjusted the bed device temperature to account for the slight change in their core & skin temperatures throughout the menstrual cycle phases. This pattern was most evident in younger (<35 y old) women for bedtime & initial temperatures, but not final temperatures.

The Percent Dial Updates were calculated by the following equation:

$$( total dial updates during the phase / total nights in that phase ) * 100$$

Following calculation, outliers that had greater than 100% were removed. The median number of dial updates in the luteal phase was higher for younger women (e.g., <35 years old) than in the follicular phase.

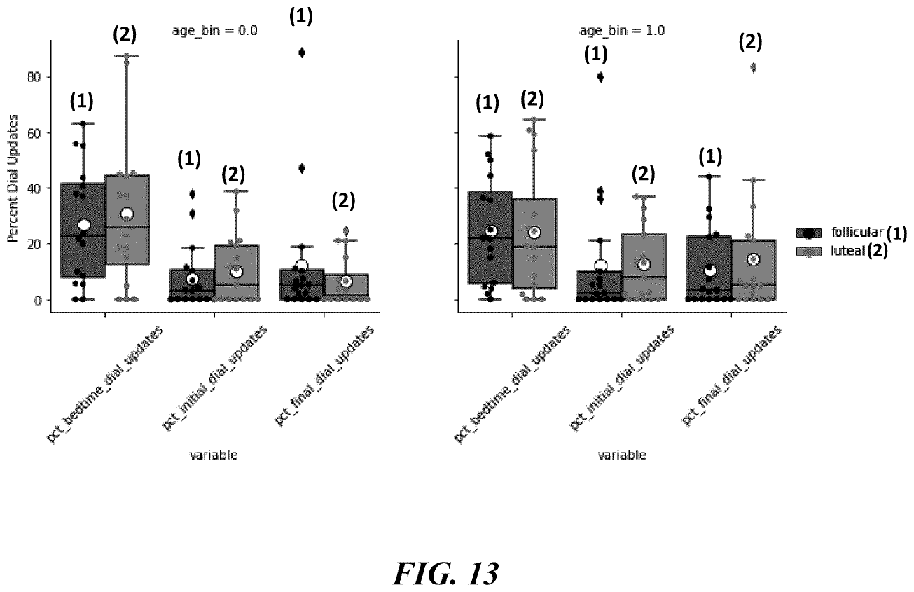

FIG. 13 shows the relative percentage of dial updates in each phase (follicular (1) vs. luteal (2)) for each age group. Younger women (e.g., <35 years old) had a 3% increase in dial updates in luteal phase vs. follicular phase at bedtime. Overall, across all female subjects had a 1% increase at bedtime, 3.75% percent increase at initial, and a 0.9% increase at final across the two phases. Overall, the dial updates did not change much across the luteal and follicular phases.

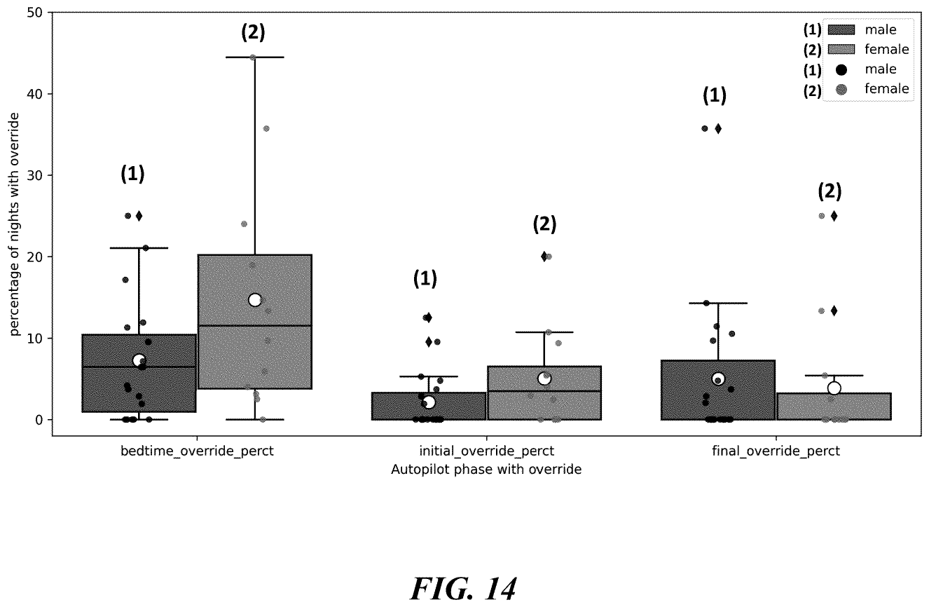

Next, the number of dial updates in male subjects (n=19 males) and female subjects (n=12 females) were analyzed. For the female subjects, 8 had a regular cycle and 3 had an irregular cycle. Females had a higher percentage of dial updates at bedtime & initial phases vs. men; whereas men (1) tend to have more overrides in the final phase vs. women (2), as shown in FIG. 14.

While the number of dial updates for women do not change much across the luteal and follicular phases, they tend to have more dial updates overall than men—in particular during the bedtime and initial phases.

### Example 5: Bed Device Temperature Regulation Affects Sleep Quality and Cardiovascular Recovery

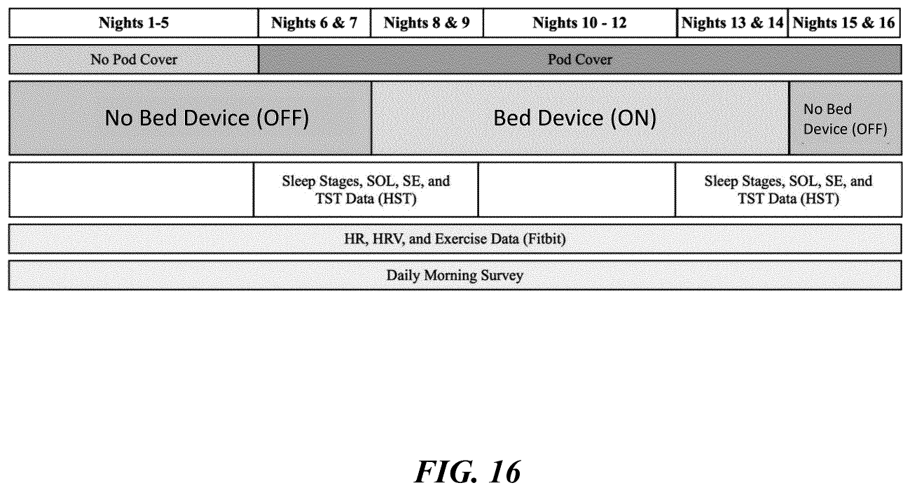

The main goal of this study was to understand how sleeping on an active temperature-controlled mattress cover (FIG. 16) affected sleep, cardiovascular, and perceptual outcomes. To answer this question, subjects were instructed to sleep on the bed device with the temperature regulation turned off (Pod OFF) for one week in order to collect baseline physiological and perceptual responses. Next, subjects slept with the bed device's temperature regulation on (Pod ON) for one week to assess the impact of temperature regulation during sleep on sleep, cardiovascular, and perceptual metrics. Cardiovascular metrics included heart rate (HR) and heart rate variability (HRV) during sleep. Sleep metrics included time spent in each sleep stage, total sleep time (TST), sleep onset latency (SOL), and sleep efficiency (SE). Perceptual metrics included thermal comfort and sensation, sleep satisfaction, calmness, refreshed, ease of falling asleep and waking up, and sleep quality. Lastly, subjects slept for two nights with bed device OFF at the end of the 16-night study to determine whether any physiological changes persisted after the one-week intervention with bed device ON. See FIG. 16 for a schematic of the experimental design. To maximize ecological validity, all subjects completed the study in their normal bedroom under free-living conditions (e.g., no experimental control over ambient temperature, lighting, or sleep/wake times).

75 subjects were recruited for this study, and 69 subjects completed the study. After filtering out nights with noisy or missing EEG data and non-temperature compliant subjects (see below for filtering criteria), 54 subjects successfully completed the 16-night study (see Results for anthropometric details). Subjects were excluded if they were unavailable to sleep on the bed device for two consecutive weeks, were under 18 years old, or had an unsupported bed size. Subjects were also excluded if they reported having any of the following criteria: a pacemaker, restless leg syndrome, an apnea hypopnea index (AHI) above 30, insomnia, or taking beta-blockers. Subjects were also excluded if they reported that they normally sleep less than 4 hours per night on more than three days per week. Seven subjects reported conditions (heart and respiratory) not listed in the exclusion criteria: six subjects reported a respiratory condition and one subject reported both heart and respiratory conditions (Table 2). Subjects' self reported ethnicities were African American (1), Latino (2), Caucasian (45), multiple ethnicities (5), and other (1). All subjects provided written consent to participate in the study, approved by Sterling Institutional Review Board (IRB identification number 10282).

TABLE 2
Population Characteristics stratified by gender.

| | Total (n = 54) | Male (n = 27) | Female (n = 27) |
|---|---|---|---|
| Variable | Mean ± SD (% of n) | Mean ± SD (% of n) | Mean ± SD (% of n) |
| Age (mean) | 36.0 ± 14.4 | [symbol] 8.3 ± 14.7 | [symbol] 3.7 ± 13.9 |
| Reported use of sleep medication | 9 (16.7%) | 2 (7.4%) | 7 (25.9%) |
| Heart conditions | 1 (1.9%) | 1 (3.7%) | 0 (0%) |
| Respiratory conditions | 7 (13.0%) | 4 (14.8%) | 3 (11.1%) |
| Global PSQI (mean) | 4.96 ± 2.33 | 4.3 [symbol] ± 2.25 | 5.22 ± 2.22 |

Note:
subjects were excluded if they were taking heart medications or had a pacemaker. Subjects with severe sleep apnea were also excluded (see Methods). Heart and respiratory conditions listed here included: chronic asthma, sleep apnea ( [symbol] 30), and arrhythmia. [symbol]
indicates data missing or illegible when filed

Before starting the study, subjects filled out a medical history survey and the Pittsburgh Sleep Quality Index (PSQI; pre-PSQI). The pre-PSQI asks subjects about their normal sleeping habits over the previous month. The PSQI generates a global PSQI score and seven sub-component scores for subjective sleep quality, sleep onset latency, sleep duration, sleep efficiency, sleep disturbances, use of sleeping medications, and daytime dysfunction.

For the first seven nights during bed device OFF, baseline physiological and perceptual data were collected. To collect sleeping HR and HRV along with exercise data, subjects were mailed a Fitbit Versa 2 (Fitbit Inc., San Francisco, CA) to start wearing on Night 1. Subjects wore their Fitbit throughout the entire study and were asked to log any exercise sessions that they completed. Subjects were also sent a daily survey each morning which included perceptual questions about thermal comfort, sleep satisfaction, and ease of falling asleep and waking up.

Subjects who did not already own a bed device (n=42) slept on their own mattress for Nights 1-5, until the Pod was installed on Night 6. Subjects who already owned a bed device (n=12) were instructed to start sleeping on the bed device with temperature OFF starting three nights before Night 1 to mitigate any potential carryover effects of sleeping with bed device ON. These subjects were required to have had at least three consecutive nights with bed device OFF before wearing Fitbit. All subjects wore the Fitbit and filled out the daily survey during Nights 1-5. For Nights 6 and 7, subjects slept on the bed device with temperature OFF. Sleep stages, SOL, SE, TST, and deep and REM sleep onset latencies were collected on these nights via a home sleep test device (HST; Zmachine Synergy, General Sleep Corp., Cleveland, OH).

For the next seven nights (Nights 8-14), subjects slept on the bed device with the temperature regulation on (ON). To assess whether there were any short term vs. longer-term changes in sleep with bed device ON, subjects wore the HST on Nights 8-9 & 13-14, which corresponded with the first two nights and last two nights of the one-week intervention of bed device ON. Subjects continued to wear the Fitbit and fill out the daily surveys for Nights 8-14. After Night 14, subjects filled out a post-bed device PSQI that asked subjects questions about their sleep habits during the week of bed device ON. The post-bed device PSQI had slightly altered questions to account for the adjusted timeline (one week vs. one month). Therefore, only a subset of PSQI subcomponents could be compared from pre- to post-PSQI.

On Nights 15 & 16, subjects slept with bed device OFF again to determine whether any long-lasting effects of sleeping with bed device ON persisted after the bed device temperature was turned OFF. Subjects wore the HST for the last two nights and continued to wear Fitbit and fill out the daily surveys. On day 17, study equipment was collected from subjects.

To ensure subjects complied with the experimental design for bed device ON and bed device OFF, their bed device temperature data were monitored daily. The temperature data were recorded in real-time and stored in a relational database (Postgres). These data were queried for each subject over the course of the study to determine their temperature values for each night. A subject was temperature compliant if they slept with bed device OFF for their first two nights on the bed device (Nights 6 & 7), slept with bed device ON for the subsequent seven nights (Nights 8-14), and then turned the bed device OFF for the final two nights (Nights 15 & 16). If subjects previously owned a Pod, then they were considered temperature compliant if they had bed device OFF at least three days before the study began and up through Night 7. Subjects were asked to repeat a night if they did not follow this schedule. If a subject had to repeat a night for any reason, they could still be considered temperature compliant if they had more than two nights at the beginning with bed device OFF, at least six nights during Week 2 with bed device ON, and two or more nights with bed device OFF at the end. Only seven subjects had to repeat a night to remain temperature compliant.

For eight nights, subjects wore the Zmachine Synergy, an HST that records the following: single-channel electroencephalogram (EEG) from three electrodes, respiratory effort via respiratory inductance plethysmography, respiratory airflow via nasal cannula, oxygen saturation via pulse oximeter, heart rate via photoplethysmography, and body position via tri-axis accelerometer (rotation and tilt in degrees). The Zmachine sleep monitoring system takes the EEG signal and generates sleep stages via an automated single-channel EEG sleep staging algorithm, ZALG, that is FDA cleared, with a Cohen's kappa agreement of 0.72. At the bed device installation (for subjects without a bed device) or via zoom (for subjects with a bed device), the subjects were provided guidance on proper HST setup. To ensure maximum adherence of the EEG electrodes throughout the night, subjects were instructed to attach the three EEG electrodes to the skin (behind the ears and back of the neck) ˜20 mins before attaching the wires. Subjects fit the HST respiratory belt over light clothing on their breastbone/nipple line and wore the nasal cannula taped to their cheeks and secured behind their ears. Subjects were additionally provided with General Sleep videos and manuals on proper HST installation, as well as Zoom call check-ins and warnings on actions that may impede data collection. If subjects reported any issues with the HST in the daily survey, the research associate would follow up with them to ask questions and help mitigate any repeated issues. All data from the HST were exported as an EDF file and preprocessed in EDF browser (version 2.0). The sleep stages are stored as annotations in the EDF file in 30 second epochs. The stages are wake (W), combined light sleep (N1 and N2), combined deep sleep (N3 and N4), Rapid Eye Movement (REM; R), and inconclusive (“?”). If there were fewer than three hours of sleep stage data, we classified the night as invalid. If a subject's night was invalid due to HST checks or temperature compliance checks (see above), then they would be asked to repeat a night. Out of our total population, nine subjects repeated a night with the HST during the study; however, this repeat night was only included if it was in proper sequence with when the bed device was ON or OFF. Furthermore, within bed device ON, the repeat HST night had to occur at the beginning or the end of the week, because there were three days without HST in the middle of the week.

The Fitbit was worn continuously throughout the study to collect sleeping HR and HRV, along with physical activity data including time spent in HR zones and total daily steps. The Fitbit Versa has been validated for HR compared to electrocardiogram (ECG) r=0.91. For each exercise session that was logged by a subject or autodetected by Fitbit, each exercise minute was categorized into a heart rate zone, based on the heart rate for that minute as a percentage of the subject's age-predicted maximum HR. Subjects were asked to enter their demographic information (age, weight, height, and sex) in the Fitbit application at the beginning of the study to ensure accurate classification of HR zones based on age-predicted maximum heart rate. Fitbit classifies fat burn, cardio, and peak heart rate zones as 40-59%, 60-84%, and >85% of maximum HR, respectively. After completion of the study, the Fitbit data was accessed through a combination of the free Fitbit data web export (via logging in to each subject's account that the research team created for them), and through Terra API (England, UK) integration in the Eight Sleep app which was then queried from Terra API. Through these two data sources, the daily information was obtained for each subject including minutes spent in each heart rate zone, total steps, minimum sleeping HR, and median sleeping HRV. Fitbit sleep data were not used for any analyses except to serve as a secondary check for HST data when there was substantial missing data due to noisy EEG (see details below).

Further processing was done with the HST data in order to calculate SOL, LPS (latency to persistent sleep), TST, REM sleep latency, deep sleep latency (Ldeep) and SE for the entire night. In order to calculate SOL, sleep onset was determined first. Sleep onset was defined as the first occurrence of sleep in any sleep stage within the first hour with at least 10 minutes of sleep in any sleep stage. Since subjects turned on the HST right before going to sleep, SOL was found using the difference in minutes between sleep onset and the start of the nightly HST recording. SOL itself is often calculated using the first epoch of stage 2 sleep 31, but Z-ALG returns stages 1 and 2 together as light sleep, so stage 1 and stage 2 could not be distinguished. To overcome this, the first hour where the individual was asleep for more than 10 minutes was found. Within this first hour of substantial sleep, the exact time when sleep started was found. The 10-minute threshold avoids counting intermittent light sleep as the start of the sleep period. This method was manually reviewed by two individuals blinded to bed device OFF vs. Pod bed device ON. LPS was defined as the start of the HST recording until the first occurrence of 10 consecutive minutes of sleep in any phase. If the HST was removed before stopping the recording, there could be upwards of 10 hours of “?” stages, so the end of the file (sleep offset) was defined as the last occurrence of REM, deep, light, or wake sleep stages. TST was found by taking the sum of all minutes spent in REM, deep, light, sleep from sleep onset until the end of the file. The number of minutes spent in REM, deep, light, Wake, and “?” stages were calculated by taking the total number of 30 second epochs in each stage and dividing that by two. REM latency was defined as the amount of time between SOL and the first occurrence of REM sleep. The number of awakenings was defined as the number of awake periods following SOL, which was calculated by finding the distinct occurrences of consecutive wake stages. SE was defined as TST divided by the duration of the HST recording up until the calculated the end of the file, excluding from both the numerator and denominator the minutes spent in “?” stage. Additionally, TST was calculated, total minutes spent in each sleep stage, the percentage of time spent in each sleep stage relative to TST, and the total number of awakenings for both initial temperature phase and final temperature phase. To calculate these metrics for each Phase, the 30 second epochs were divided into the respective Phases. From there, the same method as above was used to calculate the number of minutes spent in REM, deep, light and Wake stages respective to each Phase. The TST was found by taking the sum of all minutes spent in REM, deep, and light sleep during that Phase. Percentage of total Wake time was calculated by taking the minutes spent in the Wake stage divided by the total number of minutes in each Phase. The percentage of time spent in the rest of the sleep stages was calculated by dividing the minutes spent in each sleep stage during the initial or final Phase by TST for that Phase.

Out of the original 69 subjects that completed the study, the dataset was further filtered to only include subjects who were temperature compliant and had sufficient sleep data. If a subject was not temperature compliant, then they were not used for data analysis. A total of 56 subjects were temperature compliant: 49 subjects were temperature compliant by following the original schedule, and 7 subjects were temperature compliant by repeating a night. For both the HST and Fitbit datasets, each night was matched with the subject's temperature status (e.g., bed device ON or bed device OFF) for that night. The temperature status categories are: 1) bed device OFF Baseline (first seven nights of baseline with bed device OFF or not yet installed), 2) bed device ON beginning (first four days of the week with bed device ON), 3) bed device ON end (last three days of the week with bed device ON), and 4) bed device OFF End (final two days of the experiment with bed device OFF). Twelve subjects were previous bed device owners, and four of these subjects did not turn temperature OFF at least three days prior to Night 1. For these four individuals, up to two nights of Fitbit and survey data were removed from the beginning of their bed device OFF Baseline data to ensure they had at least three nights with bed device OFF before their data were used for analyses. Fitbit sleep sessions with a duration of less than three hours were removed from the dataset because Fitbit does not report HR, HRV, or sleep staging for sessions under 3 hours. Subjects were included in the Fitbit analysis (n=54) only if they had at least four out of seven nights for bed device OFF Baseline, five out of seven nights for bed device ON beginning and bed device ON end combined, and one out of the two nights for Post-bed device OFF. After applying these filtering criteria, HST sleep sessions with TST <3 h (˜7% of nights) were removed from the dataset to match the Fitbit cutoff of 3 hours. Since there were several HST nights with a significant portion of the night containing “?” stage (due to loose electrodes or noisy EEG data), there was further filtering to ensure that only nights with reliable sleep staging were kept. For each night, the longest time period of consecutive minutes with a “?” stage was calculated. This highest count of missing data for a given night was divided by the total duration (including sleep and wake time) of the HST recording to define a percentage of missing (“?”) data. Any night with >16% missing data was removed from the dataset. The 16% threshold was determined by visually examining the percent of time spent in deep and REM stages. In evaluating different thresholds for the missing data cutoff, it was found that there was little difference in the aggregate statistics for percentage of time spent in deep and REM stages by filtering out nights with >10% vs. >16% missing data. However, the nights with greater than 16% missing data had significant differences in time spent in deep and REM stages when compared to Fitbit's sleep staging percentages. After all these filtering steps, subjects were only included in the final analyses if they had at least one HST night in each of the four temperature status categories. After applying these filtering methods, 44 out of 56 subjects were included in the HST analysis and 54 out of 56 subjects for the Fitbit analyses (see Table 2).

To obtain each subject's temperature data for each Phase (Bedtime, initial, final) for bed device ON, a weighted mean temperature was calculated. If a subject woke up in the middle of a Phase and changed their temperature, a weighted mean better represented the overall temperature they experienced by accounting for the time spent at each temperature during that Phase. For bed device OFF nights, there were no temperatures, but the total time spent in each sleep stage was still binned into initial and final Phases by using the same time cutoffs for each Phase as when bed device ON (defined above). Cool vs. warm bed device temperatures were defined by dichotomizing bed device temperatures for men and women separately as the median for each of the three temperature Phases on the bed device (Bedtime, initial Phase, and initial Phase). For each Phase, temperatures below the median were defined as cool temperatures and those above the median were defined as warm temperatures. The median bed device temperatures during bed device ON for women vs. men at Bedtime were 25.8° C. vs. 23.4° C., respectively; for initial Phase were 26.0° C. vs. 25.4° C., respectively; and for final Phase were 28.6° C. vs. 26.8° C., respectively. Linear mixed models were used to evaluate differences in the physiological data (HR and HRV), along with time spent in each sleep stage for bed device OFF vs. ON. All models met the assumption of linearity, normality of errors and homoscedasticity. This was evaluated visually using qq plots, histograms of residuals, and plots of residuals against the outcome. bed device temperature was analyzed in three separate models as 1) a binary variable (ON vs. OFF), 2) as a categorical variable where bed device OFF Baseline was the reference compared to sleeping with bed device ON or Post-bed device OFF, and 3) a second categorical variable, where all bed device OFF days were the reference compared to sleeping at cool vs. warm bed device temperatures when bed device ON. Models evaluating cool vs. warm temperatures were stratified by sex. Sleep-stage specific analyses were restricted to the subset of subjects who had at least one night of HST data in bed device OFF Baseline and Post-bed device OFF, and at least two nights of HST data during bed device ON (n=44; see detailed explanation above). Cumulative link mixed models (CLMM) were used to estimate odds ratios (OR) and 95% confidence intervals (CI) for the relationship between bed device temperature, as well as the ordinal daily perceptual questions and the six PSQI components. The proportional odds assumption for these models was evaluated by comparing CLMM models to multinomial models which have no assumption of proportional odds. Models were compared using Akaike's information criteria (AIC), a model goodness of fit measurement. The CLMM models produced lower AICs compared to the multinomial regression indicating the CLMM model is better fit for the data. The PSQI analysis evaluated changes in six out of the seven PSQI sleep components: sleep quality, sleep onset latency, sleep duration, sleep efficiency, sleep medication use, and daytime dysfunction. Changes in the sleep disturbances component and global PSQI score were not evaluated because the validated scoring for these metrics uses responses that require an assessment over one month, which was not possible to assess after one week of bed device ON. bed device temperature was analyzed in separate models as both a binary variable (bed device ON vs. OFF) and a categorical variable. In the case of the bed device temperature as a categorical variable, bed device OFF Baseline was the reference compared to sleeping at a cool vs. warm bed device temperature. Each model was fit with a random intercept to account for differences in responses for both within—and between—subject correlations. For each of the sleep components in the PSQI, an increased score is considered a worse sleep outcome. Therefore, an OR<1 for any PSQI component indicated a sleep improvement compared to bed device OFF Baseline. Conversely, a higher score on the daily perceptual questions, except for the thermal sensation question, indicated better sleep outcomes. In this case, an OR>1 indicated improvements in sleep compared to bed device OFF Baseline. With respect to the daily survey question on thermal sensation, an OR<1 indicated that, on average, subjects felt warmer during sleep, whereas an OR>1 indicated subjects felt cooler during sleep compared to bed device OFF. Sensitivity analyses were conducted where the 12 subjects who were previous bed device owners were excluded from the daily survey perceptual analyses.

This was to account for the fact that current bed device owners may be accustomed to the bed device and feel differently during bed device OFF vs. those who have never slept on the bed device. To evaluate the mind-body connection, how changes in the nightly sleep metrics from bed device OFF to ON impacted perceptual responses the following day were examined. In the subset of individuals with HST data (n=44), CLMM were used to evaluate the relationship between the daily perceptual questions and changes in HST-measured sleep metrics. Changes in sleep metrics were calculated for each individual within each temperature Phase by subtracting the average time spent in each sleep stage during bed device OFF from each night's sleep stage duration with bed device ON. These values were dichotomized, where a change score>0 indicated an improvement in sleep with bed device ON, and a change score<0 indicated a negative change in sleep with bed device ON. For the change in wake time and number of awakenings, values were inverted so that negative change scores were considered an improvement in sleep. The goal of these analyses was to demonstrate whether improvements in the sleep metrics (measured via HST) actually resulted in perceived sleep improvements. For all models, alpha was set 0.05. All models were evaluated for effect modification by sex using a multiplicative interaction term. All results were evaluated and visualized using R statistical software (version 4.2.2) 37 and all preprocessing of the data was done with Python (version 3.7).

A total of 54 subjects completed the study (27 females and 27 males, mean±SD age=36.0±14.4 y; range 21-74 y) (Table 2). Subjects completed 160 nights of HST and 466 nights of Fitbit with bed device OFF, and 158 nights of HST and 367 nights of Fitbit with bed device ON. The population was predominantly healthy with 1.9% and 13.0% reporting heart conditions and respiratory conditions, respectively. Approximately 17% of subjects reported using sleep medication at baseline; however, the study population comprised normal (healthy) sleepers according to a similar global PSQI score to the normal population 38 (Table 2).

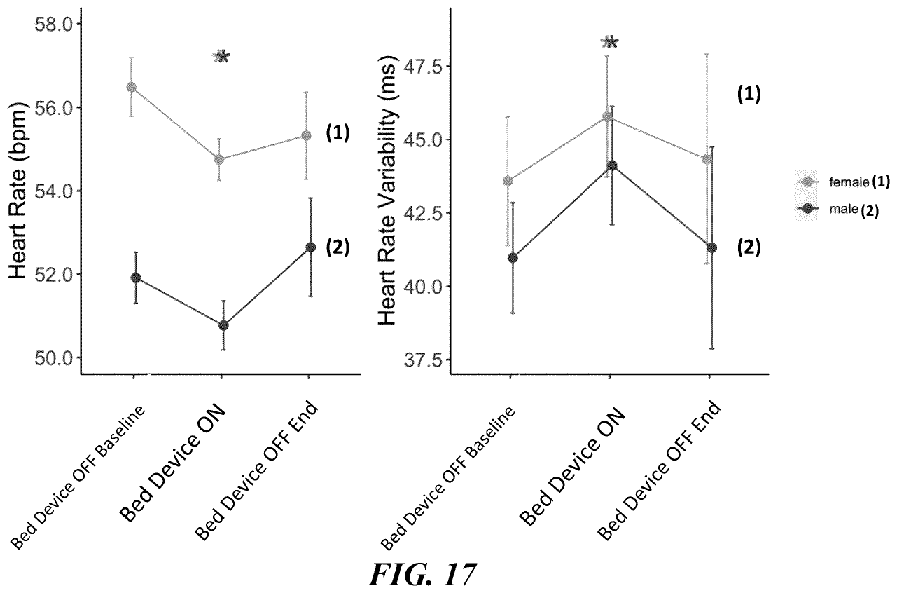

Comparing bed device ON to bed device OFF Baseline, minimum sleeping HR was significantly lower on average by 1.2 bpm (p<0.001), and median sleeping HRV significantly higher on average by 2.0 ms (p=0.009) (FIG. 17, female (1), male (2)). The median and mean percent changes in HR from bed device OFF to ON were −2.4% and −1.9%, respectively, with a maximum percent change of −13.2%. The median and mean percent changes in HRV from bed device OFF to ON were 5.2% and 7.1%, respectively, with a maximum percent change of 16.5%.

TABLE 3
Least Squared Means and Standard Errors for each Sleep Stage by sex and temperature for
bed device OFF as a reference compared to bed device ON at cool or warm temperatures.

| | Men | | | | | Women | | | | |
|---|---|---|---|---|---|---|---|---|---|---|
| | | Cool | | Warm | | OFF | Cool | | Warm | |
| | OFF | lsmean ± | | lsmean ± | | (ref) | lsmean ± | | lsmean ± | |
| Variable | (ref) | SE | p-value | SE | p-value | lsmean ± SE | SE | p-value | SE | p-value |

Bedtime

| | | | | | | | | | | |
|---|---|---|---|---|---|---|---|---|---|---|
| SOL | 28.5 ± 4.1 | 28.7 ± 5.1 | 0.965 | 26.0 ± 4.6 | 0.452 | 31.5 ± 3.9 | 33.0 ± 5.3 | 0.756 | 24.2 ± 4.5 | 0.079 |
| Deep | 16.8 ± 2.2 | 10.0 ± 3.2 | 0.045 | 11.5 ± 2.7 | 0.066 | 18.4 ± 4.3 | 8.5 ± 6.2 | 0.124 | 23.1 ± 5.0 | 0.377 |
| SOL | | | | | | | | | | |

Early Phase

| | | | | | | | | | | |
|---|---|---|---|---|---|---|---|---|---|---|
| Deep | 64.3 ± 4.9 | 78.6 ± 5.7 | 0.003 | 70.9 ± 6.3 | 0.205 | 68.5 ± 6.5 | 63.6 ± 7.1 | 0.305 | 61.8 ± 7.3 | 0.194 |
| (min) | | | | | | | | | | |
| Deep | 17.3 ± 1.5 | 20.1 ± 1 .7 | 0.051 | 18.7 ± 1.9 | 0.389 | 17.4 ± 1.7 | 16.8 ± 1.9 | 0.669 | 15.9 ± 1.9 | 0.292 |
| (%) | | | | | | | | | | |
| REM | 37.2 ± 3.34 | 30.5 ± 4.0 | 0.072 | 35.3 ± 4.5 | 0.628 | 35.0 ± 4.2 | 44.3 ± 4.9 | 0.033 | 35.5 ± 5.1 | 0.924 |
| (min) | | | | | | | | | | |
| REM | 10.0 ± 0.9 | 7.8 ± 1.1 | 0.061 | 9.1 ± 1.3 | 0.476 | 9.3 ± 1.1 | 11.7 ± 1.3 | 0.028 | 8.8 ± 1.4 | 0.686 |
| (%) | | | | | | | | | | |
| Light | 113.0 ± 5.9 | 105.0 ± 6.6 | 0.075 | 108.0 ± 7.0 | 0.341 | 115.0 ± 6.7 | 108.0 ± 7.4 | 0.169 | 109.0 ± 7.6 | 0.301 |
| (min) | | | | | | | | | | |
| Light | 30.5 ± 1.9 | 27.0 ± 2.2 | 0.047 | 28.4 ± 2.4 | 0.291 | 29.8 ± 1.9 | 28.4 ± 2.1 | 0.326 | 28.8 ± 2.2 | 0.527 |
| (%) | | | | | | | | | | |
| Wake | 22.8 ± 2.2 | 22.0 ± 2.8 | 0.754 | 21.7 ± 3.1 | 0.713 | 20.6 ± 3.1 | 23.5 ± 3.6 | 0.375 | 24.7 ± 3.8 | 0.236 |
| (min) | | | | | | | | | | |
| REM | 111.0 ± 45.9 | 116.6 ± 51.2 | 0.694 | 132.6 ± 81.1 | 0.443 | 122.4 ± 64.5 | 101.8 ± 75.2 | 0.181 | 144.6 ± 52.4 | 0.202 |
| SOL | | | | | | | | | | |

Late Phase

| | | | | | | | | | | |
|---|---|---|---|---|---|---|---|---|---|---|
| Deep | 7.3 ± 1.4 | 7.3 ± 1.6 | 0.993 | 4.6 ± 2.0 | 0.181 | 9.2 ± 2.1 | 10.4 ± 2.7 | 0.654 | 7.1 ± 2.7 | 0.460 |
| (min) | | | | | | | | | | |
| Deep | 1.8 ± 0.3 | 1.8 ± 0.4 | 0.957 | 1.1 ± 0.5 | 0.188 | 2.3 ± 0.5 | 2.6 ± 0.6 | 0.622 | 1.8 ± 0.6 | 0.499 |
| (%) | | | | | | | | | | |
| REM | 55.6 ± 5.6 | 48.7 ± 6.5 | 0.257 | 66.5 ± 7.9 | 0.142 | 45.6 ± 4.3 | 48.0 ± 5.5 | 0.678 | 47.8 ± 5.5 | 0.706 |
| (min) | | | | | | | | | | |
| REM | 13.4 ± 1.2 | 11.6 ± 1.4 | 0.159 | 15.2 ± 1.7 | 0.252 | 11.4 ± 0.9 | 11.8 ± 1.2 | 0.792 | 11.4 ± 1.2 | 0.990 |
| Light | 94.4 ± 6.9 | 105.0 ± 8.2 | 0.204 | 117.8 ± 10.2 | 0.023 | 105.0 ± 7.1 | 106.0 ± 8.7 | 0.967 | 113.0 ± 8.9 | 0.405 |
| (min) | | | | | | | | | | |
| Light | 23.1 ± 1.3 | 25.5 ± 1.6 | 0.166 | 28.2 ± 2.0 | 0.016 | 26.1 ± 1.4 | 26.1 ± 1.7 | 0.983 | 27.5 ± 1.8 | 0.403 |
| (%) | | | | | | | | | | |
| Wake | 21.0 ± 2.4 | 18.3 ± 2.8 | 0.216 | 24.2 ± 3.4 | 0.046 | 18.7 ± 3.2 | 24.0 ± 4.0 | 0.186 | 26.9 ± 4.0 | 0.044 |
| (min) | | | | | | | | | | |

Note:
OFF indicates all nights of Pod OFF for that subject as a reference for comparing with Pod ON at Cool or Warm temperatures for Bedtime, Early, and Late Phases for Women and Men. See methods for description of Cool vs. Warm bins. p < 0.05 defined significance. lsmeans: least squared means, SE: standard error.

TABLE 4
Odds ratios and 95% confidence intervals evaluating changes in
responses to PSQI from bed device OFF to bed device ON overall.

| Variable | Odds Ratio | Std Err | 95% CI | z value | p-value |
|---|---|---|---|---|---|
| Component 1 (Duration) | 0.3 [symbol] 2 | 0.336 | (0.187, 0.700) | −3.023 | 0.003 |
| Component 3 (Latency) | 0.232 | 0.229 | (0. [symbol] , 0.363) | −6.378 | <0.001 |
| Component 4 (Dysfunction | 0.2 [symbol] | 0.341 | (0.104, 0.397) | −4.573 | <0.001 |
| During Day) | | | | | |
| Component 5 (Efficiency) | 0.976 | 0.224 | (0.629, [symbol] ) | [symbol] | [symbol] |
| Component 6 (Quality) | 0.120 | 0.277 | (0.057, 0.251) | [symbol] | <0.001 |
| Component 7 (Medication) | 0.224 | 0. [symbol] | (0.124, 0.402) | −4.987 | <0.00 [symbol] |

Note:
P < 0.05 defined significance. [symbol]
indicates data missing or illegible when filed

HR and HRV were similar between bed device OFF Baseline vs. Post-bed device OFF (all p>0.05), indicating changes with bed device ON were acute responses. There were no significant differences between men and women with regard to changes in minimum HR and median HRV with bed device ON (all p>0.05). Notably, these changes in HR and HRV were not a result of changes in exercise habits from bed device OFF Baseline to ON, as there was no significant difference in the time subjects spent exercising in Fat Burn Zone, Cardio Zone, or number of steps taken each day (all p>0.05).

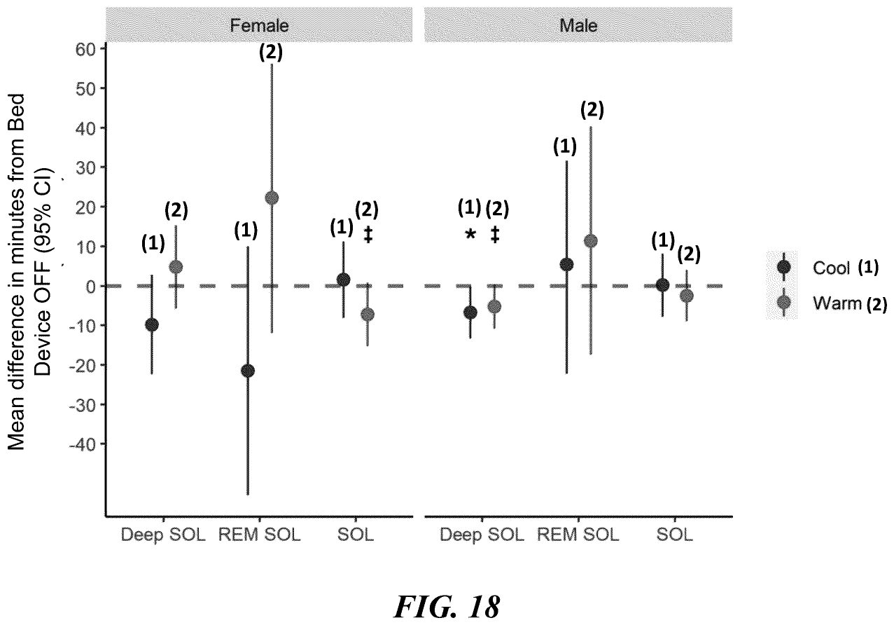

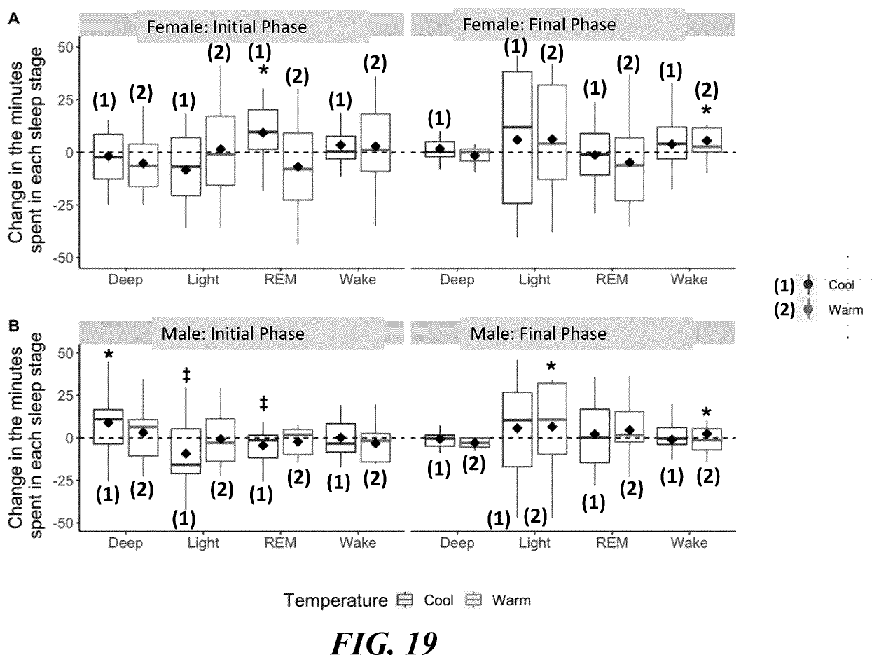

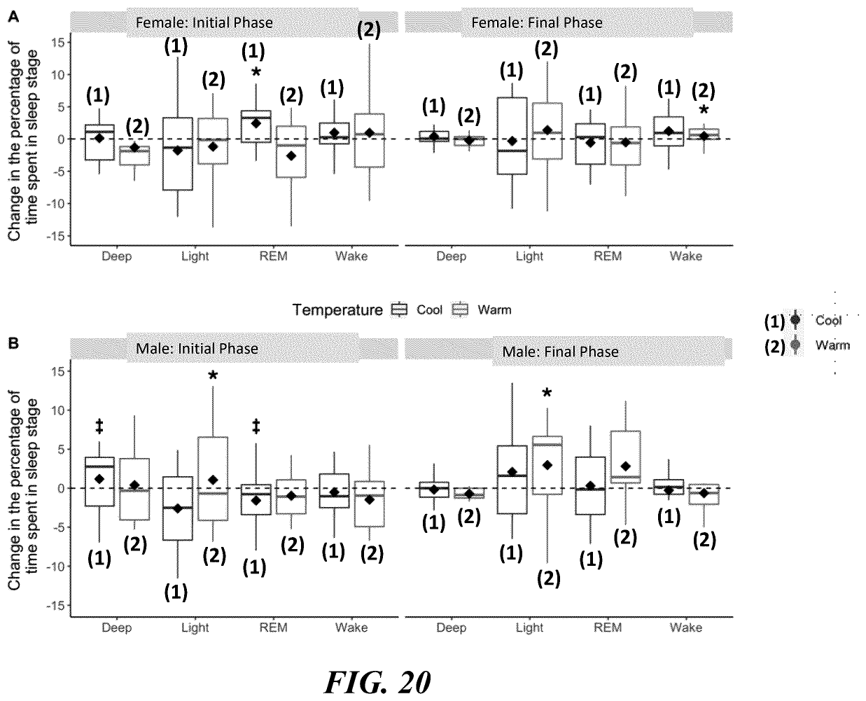

For men sleeping at cooler temperatures during the initial Phase, minimum HR was significantly lower by 1.23 bpm (p=0.020) and median HRV was significantly higher by 2.87 ms (p=0.039). Similar effects were not observed when men slept at warmer temperatures during initial Phase (all p>0.05). During the final Phase, men's minimum HR at both warm and cool temperatures was significantly lower on average by 1.06 bpm (p=0.046) and 1.02 bpm (p=0.045), respectively. However, temperature had no effect on HRV during final phase. Changes in HR and HRV for women were unaffected by sleeping temperature for the initial and final Phases (all p>0.05). Effect of bed device Use on Sleep Metrics Men Sleeping at cool Bedtime temperatures (1) led to a significantly faster deep sleep onset latency vs. bed device OFF (mean change=−6.70 min, p=0.045), whereas sleeping at warm Bedtime temperatures (2) did not (p=0.066) (FIG. 18). Sleeping at cool initial Phase (1) temperatures increased deep sleep by 14.3 min on average (p=0.003), which equated to a 22% increase in initial Phase compared to bed device OFF (FIG. 19A). This equated to a 2.78% increase in the percentage of total sleep time spent in deep sleep (p=0.051) (FIG. 20A). This increase in deep sleep for men sleeping at cooler temperatures came as a tradeoff for REM sleep (−6.7 min or −18% on average) and light sleep (−8.39 min or −7% on average; both p=0.07). Sleeping at warm initial Phase temperatures did not change sleep architecture compared to bed device OFF (all p>0.05) (Table 3). Men who had lower baseline deep sleep (bed device OFF) had larger deep sleep improvements with bed device ON (r 2=0.40, p=0.018). The relationship is inverse, so that for every 1-minute less of baseline deep sleep, there was a 0.4% increase in the total sleep time spent in deep sleep with bed device ON. Sleeping at Warm final Phase temperatures increased light sleep by 23.4 min on average (˜19% increase; p=0.023), but also slightly increased the number of awakenings (+3 awakenings, p=0.025) compared to bed device OFF (Table 3). Sleeping at cool final Phase temperatures did not alter sleep architecture relative to bed device OFF (all p>0.05) (Table 3). Overall, TST, SE, SOL, and REM onset latency were unchanged from bed device OFF to ON (TST mean change=10 min, 95% CI=[−0.14, 0.48], p=0.284; SE mean change=−0.01, 95% CI=[−0.009, 0.03], p=0.318; SOL mean change=−1.483, 95% CI=[−7.03, 4.04], p=0.599; REM onset latency mean change=6.45, 95% CI=[−14.24, 26.81], p=0.544).

Women who slept at warm Bedtime temperatures fell asleep 7 min faster on average compared to bed device OFF (mean change in SOL=−7.28 min; p=0.079). Sleeping at cool initial Phase temperatures (1) increased REM sleep by 9.2 min compared to bed device OFF (25% increase in initial Phase; p=0.033), which equated to a 2.48% increase in the percentage of total sleep time spent in REM sleep (FIGS. 19B and 20B; Table 3). This increase in REM sleep appeared to be a tradeoff for small, but statistically insignificant decreases in light sleep (mean change=−6.99 min or −6%, p=0.169) and deep sleep (mean change=−4.90 min or −7%, p=0.305) (Table 3). Women sleeping at warm final Phase temperatures (2) significantly increased wake time by 8.15 min (p=0.044), with no difference in the number of awakenings (p=0.123) vs. bed device OFF (Table 3). Pod temperatures did not affect TST, SE, REM and deep sleep onset latencies (TST mean change=−0.24 min, 95% CI=[−0.30, 0.29], p=0.981; SE mean change=−0.01, 95% CI=[0.029, 0.011], p=0.359; REM sleep latency mean change=0.12, 95% CI=[−24.39, 24.70], p=0.993; deep sleep latency mean change=−0.03, 95% CI=[−8.721, 8.884), p=0.995) (FIG. 18 and Table 3). Effect of bed device Use on the PSQI Components All PSQI components, except SE (p=0.915), significantly improved from bed device OFF to ON (all p<0.05) (Table 4). When the 12 prior bed device owners were excluded from the analyses, effects were muted and only SOL (p=0.014), daytime dysfunction (p=0.031), and sleep medication use (p<0.001) remained significantly improved with bed device ON. SOL After sleeping on the bed device, subjects reported a decrease in perceived SOL (p<0.001). Men sleeping at warm Bedtime temperatures reported improvements in perceived SOL compared to bed device OFF (p<0.001), while women perceived significantly faster SOL regardless of their Bedtime temperature (cool: p=0.002; warm: p<0.001).

TABLE 5
Odds ratios and 95% confidence intervals evaluating changes in responses
to daily perceptual questions from bed device OFF to bed device ON.

| | Odds | Std | | z | p- |
|---|---|---|---|---|---|
| Variable | Ratio | Err | 95% CI | value | value |
| How would you rate the calmness of your sleep last night? | 1.283 | 0.1 [symbol] | (1.013, | 2.0 [symbol] 3 | 0.039 |
| | | | 1.640) | | |
| How easy was it to fall asleep last night? | 1.257 | 0.127 | (1.059, | 2.41 | 0.01 [symbol] |
| | | | 1.739) | | |
| How easy was it to wake up this morning? | 1.144 | 0.125 | (0.895, | 1.076 | 0.282 |
| | | | 1.461) | | |
| How refreshed do you feel after waking? | 1.091 | 0.123 | (0.859, | 0.712 | 0.477 |
| | | | 1.386) | | |
| How satisfied are you with your sleep last night? | 1.24 [symbol] | 0.121 | (0.98 [symbol] , | 1.838 | 0.066 |
| | | | 1. [symbol] ) | | |
| On average throughout the night, what was your | 1.78 [symbol] | 0.122 | (1.401, | 4.726 | <0.001 |
| thermal sensation? | | | 2.2 [symbol] ) | | |
| On average, how comfortable were you with your body | 1.803 | 0.148 | (1.349, | [symbol] .981 | <0.001 |
| temperature throughout the night? | | | 2.410) | | |

Note:
P < 0.05 defined significance. [symbol]
indicates data missing or illegible when filed

Sleep Efficiency Perceived SE significantly improved for men sleeping at warm bed device temperatures across all phases (initial Phase: p=0.009; final Phase: p=0.013). There was a trend for women sleeping at cooler initial Phase temperatures (p=0.070) and warm final Phase temperatures (p=0.084) to perceive better SE. Medication Use & Daytime Dysfunction Medication use significantly decreased from 15 to 9 subjects with bed device ON (p<0.001) vs. bed device OFF, with no effect modification by sex (p=0.556). Daytime dysfunction was significantly reduced in men regardless of final Phase temperatures, and for women regardless of initial or final Phase temperatures (all p<0.05). Sleep Quality Perceived sleep quality significantly improved in men for bed device ON vs. OFF, independent of initial Phase temperature (cool: p<0.001; warm: p=0.008). However, only men sleeping at cool final Phase temperatures reported improved sleep quality compared to bed device OFF (p<0.001). Women reported significantly improved sleep quality with bed device ON regardless of initial Phase temperatures vs. bed device OFF (cool: p=0.001; warm: p<0.001) or final Phase (cool: p<0.001; warm: p<0.001). Effect of bed device Use on Daily Perceptual Questions Changes in daily perceptual ratings from bed device OFF to ON During bed device ON, subjects had increased odds of reporting that 1) their sleep was calmer (p=0.039), 2) it was easier to fall asleep (p=0.016), 3) they felt cooler during sleep (p<0.001), and 4) they were more comfortable with their body temperature (p<0.001). When the 12 prior bed device owners were removed from the analyses, only two key ratings were still statistically significant. Those that did not previously own a bed device were still significantly more comfortable with their body temperature (p=0.039) and felt cooler during sleep (p<0.001) with bed device ON vs. OFF.

TABLE 6
Odds ratios and 955 confidence intervals evaluating how
improvement in the sleep metrics from bed device OFF to
bed device ON affect responses on perceptual questions.

| | | | | | |
|---|---|---|---|---|---|
| Variable | Odds Ratio | Std Err | 95% CI | z value | p-value |

How would you rate the calmness of your sleep last night?

| | | | | | |
|---|---|---|---|---|---|
| Deep Sleep | 1.061 | 0.204 | (0.711, 1. [symbol] 83) | 0.291 | 0.771 |
| REM Sleep | 1.178 | 0.200 | (0.7 [symbol] 6, 1.744) | 0.818 | 0.41 [symbol] |
| Light Sleep | 1.164 | 0.203 | (0.781, 1. [symbol] 32) | 0.747 | 0.455 |
| Wake | 1. [symbol] 4 | 0.216 | (1.109, 2.58 [symbol] ) | 2.4 [symbol] 1 | 0. [symbol] |
| Total Sleep Time | 1.297 | 0.220 | (0. [symbol] 43, 1.996) | 1.183 | 0.237 |
| Number of Awakenings | 1.037 | 0.208 | (0. [symbol] 0, 1.55 [symbol] ) | 0.1 [symbol] 9 | 0.866 |
| REM Sleep Onset | 0. [symbol] 24 | 0.205 | (0.617, 1.382) | −0.16 [symbol] | 0.6 [symbol] |
| Deep Sleep Onset | 0.942 | 0.222 | (0. [symbol] 10, 1.457) | −0.267 | 0.700 |
| Sleep Efficiency | 1.358 | 0.129 | (1.050, 1.7 [symbol] 0) | 2.365 | 0.018 |

How easy was it to fall asleep last night?

| | | | | | |
|---|---|---|---|---|---|
| Deep Sleep | 0.887 | 0.22 [symbol] | (0.573, 1.375) | −0.535 | 0.592 |
| REM Sleep | 1.4 [symbol] 4 | 0.221 | (0.930, 2.212) | [symbol] .63 [symbol] | 0.10 [symbol] |
| Light Sleep | 0.995 | 0.22 [symbol] | (0.845, 1.5 [symbol] ) | −0.023 | 0.981 |
| Wake | 2.027 | 0.264 | (1.2 [symbol] 8, 3.400) | −2. [symbol] 78 | 0.007 |
| Total Sleep Time | 1.24 [symbol] | 0.702 | (0.315, 4.941) | 0.316 | 0.752 |
| Number of Awakenings | 1.438 | 0.234 | (0.908, 2.276) | 1.54 [symbol] | 0.121 |
| Sleep Onset | 1.246 | 0.232 | (0.791, 1.9 [symbol] 2) | 0.950 | 0.342 |
| REM Sleep Onset | 0.491 | 0.225 | (0.316, 0.763) | −3.160 | 0.002 |
| Deep Sleep Onset | 0. [symbol] 15 | 0.240 | (0.509, 1.303) | 0. [symbol] 55 | 0.3 [symbol] |
| Sleep Efficiency | 1.557 | 0.136 | (1.194, 2.030) | 3.267 | 0.001 |

How easy was it to wake up this morning?

| | | | | | |
|---|---|---|---|---|---|
| Deep Sleep | 0.842 | 0.2 [symbol] | ( [symbol] , 1.2 [symbol] 2) | −0.832 | 0.406 |
| REM Sleep | 1.275 | 0.200 | (0. [symbol] 3, 1.885) | 3.219 | 0.223 |
| Light Sleep | 1.20 [symbol] | 0.205 | (0.8 [symbol] 9, 1.504) | 0.92 [symbol] | 0.355 |
| Wake | 1.247 | [symbol] .220 | (0.521, 1.236) | [symbol] .0 [symbol] 0 | 0.317 |
| Total Sleep Time | 1.5 [symbol] 8 | 0.216 | (1.020, 2. [symbol] ) | 2.050 | 0.040 |
| Number of Awakenings | 1.15 [symbol] | 0.211 | (0.7 [symbol] 6, 1.750) | 0.6 [symbol] 5 | 0.487 |
| REM Sleep Onset | 0. [symbol] | 0.206 | (0.438, 0.982) | −2.048 | 0.04 [symbol] |
| Deep Sleep Onset | 1.083 | 0.222 | (0.70 [symbol] , 1.674) | 0.3 [symbol] 0 | 0. [symbol] 19 |
| Sleep Efficiency | 1.173 | 0.132 | (0.906, 1.521) | 1.21 [symbol] | 0.22 [symbol] |

How refreshed do you feel after waking?

| | | | | | |
|---|---|---|---|---|---|
| Deep Sleep | 1.177 | 0.214 | (0.761, 1.78 [symbol] ) | 0.7 [symbol] 1 | 0.447 |
| REM Sleep | 1.436 | 0.205 | (0. [symbol] 60, 2.149) | 1.7 [symbol] 2 | 0.078 |
| Light Sleep | 1.434 | 0.20 [symbol] | (0.95 [symbol] , 2.15 [symbol] ) | 1.730 | 0.084 |
| Wake | 1.578 | 0.238 | (0.990, 2.514) | 1.917 | 0.05 [symbol] |
| Total Sleep Time | 1.5 [symbol] 1 | 0.225 | (1.002, 2.43 [symbol] ) | 1.968 | 0.049 |
| Number of Awakenings | 0.898 | 0.217 | (0.587, 1.375) | [symbol] .492 | 0. [symbol] 23 |
| REM Sleep Onset | 0.771 | 0.213 | (0.508, 1.171) | −1.21 [symbol] | 0.223 |
| Deep Sleep Onset | 1.356 | 0.244 | (0.541, 2.187) | [symbol] .249 | 0.212 |
| Sleep Efficiency | 1.442 | 0.36 [symbol] | (1.116, 1. [symbol] 6 [symbol] ) | 2.7 [symbol] 6 | 0.005 |

How satisfied are you with your sleep last night?

| | | | | | |
|---|---|---|---|---|---|
| Deep Sleep | 1.129 | 0.204 | (0.757, 1.683) | 0.595 | 0. [symbol] 2 |
| REM Sleep | 1.512 | 0.212 | (1.022, 2.235) | 2.071 | 0.038 |
| Light Sleep | 1.28 | 0.201 | (0. [symbol] 2, 1.899) | [symbol] .225 | 0.220 |
| Wake | 1.6 [symbol] | 0.225 | ( [symbol] .061, 2.57 [symbol] ) | 2.223 | 0.02 [symbol] |
| Total Sleep Time | 1.722 | 0.213 | (1.135, 2.612) | [symbol] .555 | 0.011 |
| Number of Awakenings | 0.875 | 0.208 | (0. [symbol] 2, 1.317) | −0.639 | 0. [symbol] 23 |
| REM Sleep Onset | 0.846 | 0.206 | (0.5 [symbol] 4, 1.269) | −0. [symbol] 08 | 0.419 |
| Deep Sleep Onset | 1.381 | 0.233 | (0. [symbol] 75, 2.179) | [symbol] . [symbol] 8 | 0.165 |
| Sleep Efficiency | 1.5 [symbol] 8 | 0. [symbol] 0 | (1.178, 1.957) | 3.222 | 0. [symbol] 01 |

On average throughout the night, what was your thermal a [symbol] tion?

| | | | | | |
|---|---|---|---|---|---|
| Deep Sleep | 1.083 | 0.149 | (0.809, 1.456) | 0.53 [symbol] | 0. [symbol] 91 |
| REM Sleep | 0.8 [symbol] 6 | 0.150 | (0.635, 1.146) | −1.035 | 0.301 |
| Light Sleep | 0.687 | 0.15 [symbol] | (0.501, 0.927) | −2.400 | 0.0 [symbol] 5 |
| Wake | 1.038 | 0.149 | (0.775, [symbol] .0 [symbol] 9) | 0. [symbol] 52 | 0.801 |
| Total Sleep Time | 0.759 | 0.163 | (0.547, [symbol] .041) | −1. [symbol] 93 | 0.091 |
| Number of Awakenings | 1.590 | 0.161 | (1.171, 2.20 [symbol] ) | −2.891 | 0.004 |
| REM Sleep Onset | 1.031 | 0.149 | (0.767, 1.385) | 0.201 | 0.841 |
| Deep Sleep Onset | 0. [symbol] 08 | 0.174 | (0.425, 0.845) | −2.854 | 0.004 |
| Sleep Efficiency | 0.9 [symbol] | 0.101 | (0.756, 1.123) | −0.805 | 0.4 [symbol] |

On average, how comfortable were you with your body temperature throughout the night?

| | | | | | |
|---|---|---|---|---|---|
| Deep Sleep | 1. [symbol] 8 [symbol] | 0.347 | (0.875, 3.458) | 1.507 | 0.132 |
| REM Sleep | 1.55 [symbol] | 0.344 | (0.810, 3.17 [symbol] ) | 1. [symbol] 8 [symbol] | 0.1 [symbol] 8 |
| Light Sleep | 2.3 [symbol] | 0.3 [symbol] 9 | (1.171, 5.220) | 2.2 [symbol] 7 | 0.023 |
| Wake | 2. [symbol] 56 | 0.384 | (1.306, 5.920) | 2.547 | 0.011 |
| Total Sleep Time | 2.2 [symbol] | 0.375 | (1.127, 4.94 [symbol] ) | 2.183 | 0.029 |
| Number of Awakenings | 0.643 | 0.344 | (0.315, 1.23 [symbol] ) | [symbol] .286 | 0.1 [symbol] |
| REM Sleep Onset | 0.576 | 0. [symbol] | (0.279, 1.116) | [symbol] .58 | 0. [symbol] |
| Deep Sleep Onset | 1.318 | 0. [symbol] | (0. [symbol] 47, 2.94 [symbol] ) | 0.727 | 0.467 |
| Sleep Efficiency | 1.831 | 0.1 [symbol] | (1.255, 2.713) | 3.084 | 0.002 |

[symbol]

indicates data missing or illegible when filed

Note: Improvement in sleep is indicated by an increase in minutes from bed device OFF Baseline to bed device ON of deep sleep, REM sleep, light sleep, total sleep time, REM sleep onset, deep sleep onset latency or sleep efficiency proportion. Improvements in number of awakenings and minutes of wake time were indicated by a decrease in these sleep metrics. p<0.05 for all comparisons). Women sleeping at warm initial (p=0.018) and final (p=0.004) Phase temperatures reported feeling significantly more refreshed upon waking, while men did not feel more refreshed with bed device ON vs. OFF (p>0.05 for all comparisons). Women sleeping at cooler initial (p<0.001) and Late (p<0.001) Phase temperatures reported a cooler thermal sensation. Thermal comfort was significantly higher for women sleeping at warm initial Phase (p=0.003) and cool final Phase (p<0.001) temperatures. Whereas for men, thermal comfort significantly improved, and they felt cooler during sleep with bed device ON regardless of their initial or final Phase temperatures (all p>0.05). Linking perceptual ratings with changes in sleep metrics: the mind-body connection Men with faster SOL (measured via HST) reported it was easier for them to fall asleep (p=0.019). However, women with faster SOL did not perceive falling asleep faster (p=0.301). Improved SE (measured via HST) was associated with feeling 1) calmer about their sleep (p=0.018), 2) more refreshed after waking (p=0.005), and 3) that it was easier to fall asleep (p=0.018) (Table 6). Subjects with more TST reported it was easier to wake up in the morning (p=0.040) and that they felt more refreshed upon waking (p=0.049). Those with more REM sleep, more TST, higher SE, and less wake time reported higher sleep satisfaction (REM: p=0.038; TST: p=0.011; SE: p=0.001; wake time: p=0.026). Subjects reporting higher thermal comfort had approximately two times higher odds for having more light sleep (p=0.023), more TST (p=0.029), and higher SE (p=0.002). Those with higher thermal comfort ratings were also more likely to experience less total wake time (p=0.011) (Table 6).

Overall, this study found three main findings. First, sleeping at cooler temperatures during the first half of the night increased deep sleep in men and REM sleep in women, whilst sleeping at warmer temperatures in the latter half of the night led to more light sleep in men and wake time in women. Second, HR and HRV are improved when sleeping with bed device ON, but this effect is transient, as HR and HRV return to baseline after temperature regulation is removed (bed device OFF). Third, subjects rated their body temperature as significantly more comfortable with bed device ON, thus qualitatively and quantitatively improving sleep quality. Women who were warm Bedtime (2) sleepers fell asleep 7 minutes faster on average than those sleeping at cooler temperatures (1) (FIG. 18). For men, there was no quantitative improvement in SOL (FIG. 18), but there was a perceived improvement in SOL (Table 4). The lack of physiological change from bed device OFF to ON in men may be due to individual variability in core and skin temperatures as a result of acclimatization state, age, disease, or race, which would modify the bed device temperature needed for optimal SOL. Overall, the 7 min decrease in SOL for women and perceived improvement in SOL for men may be beneficial given SOL-insomnia is prevalent in the general population. Men sleeping at cool Pod temperatures (1) averaged 14 more minutes of deep sleep vs. bed device OFF Baseline, while women sleeping at cool bed device temperatures (1) averaged 9 more minutes of REM sleep vs. bed device OFF Baseline (FIG. 19). These improvements in deep and REM sleep came at the cost of reduced time in other sleep stages: increased deep sleep in men was traded for REM and light sleep, while increased REM sleep in women was traded for slight reductions in deep and light sleep (FIGS. 18 & 19). In men, the changes in deep and REM sleep with Pod ON promoted a similar amount of time spent in both sleep stages (˜19-21% in each stage), which is a characteristic of good sleep quality. Cooler temperatures during the second half of the night (final Phase) did not modify sleep compared to bed device OFF; however, sleeping at warmer temperatures (2) during final Phase led to an average increase of 8 min of wake time for women and three additional awakenings per night for men. It was found that men sleeping at warmer bed device temperatures (2) had 23 more minutes of light sleep vs. bed device OFF (FIGS. 18 & 19).

Sleeping on the bed device led to improvements in both HR (−2%) and HRV (+7%) in men and women, indicating improved restoration and recovery. Furthermore, men who slept at cool initial Phase temperatures had larger decreases in HR compared to those who slept at warm initial Phase temperatures. However, the same temperature dependent changes were not true for women as HR was lower regardless of bed device temperatures during the night. Since there was not a clear pattern in our data between the reduction in HR and temperature, it is likely that the bed device temperature required to achieve the lower HR is specific to each individual. For men, cool initial Phase temperatures led to higher HRV, while HRV improvements in final Phase occurred independent of temperature. Although not statistically significant, the mean change scores indicate that women's HRV improvements occurred when sleeping at warm bed device temperatures. This could be due to a similar reason mentioned above whereby Pod temperatures have different effects based on the individual's unique body temperature. Changes in HR and HRV with bed device ON were acute (transient), because HR and HRV went back to baseline (bed device OFF Baseline) during Post-bed device OFF (FIG. 17).

Women and men both prefer a similar skin temperature range during sleep 64; however, women need ˜1-2° C. warmer ambient temperatures vs. men to achieve the preferred skin temperature range for sleep 65. On average, women's bed device temperatures were ˜1-2° C. warmer than men's temperatures across all temperature Phases, which likely helped facilitate women maintaining this tight skin temperature range to optimize their sleep. In fact, women and men reported sleeping on the bed device was better than sleeping on their normal mattress 64.3% and 75.7% of the time, respectively. It was found that meaningful physiological changes in sleep did not always correspond with perceived improvements in sleep. For example, men who had significantly improved SOL reported that it was easier for them to fall asleep, whereas the same was not true for women. It was found that increased deep sleep did not lead to higher perceived sleep quality or satisfaction. Instead, subjects reported feeling more refreshed upon waking and satisfied with their sleep when getting more TST and REM sleep, and less wake time. SE improvements (measured via HST) were the most strongly related variable to perceived improvements in sleep quality (Table 6). Interestingly, despite no actual quantitative improvements in TST or SE with bed device ON (vs. OFF), subjects perceived their TST, SE, SOL, sleep quality, and daytime dysfunction improved with bed device ON (assessed via PSQI; see Table 4). Both sexes reported feeling more comfortable with their body temperature during sleep with bed device ON vs. OFF, and their body temperature felt cooler overall with bed device ON (Table 5). As each person has different temperature needs based on their individual body temperature rhythm, the bed device allowed for each individual to sleep at the temperature they found most comfortable. In turn, this led to subjects perceiving that it was easier to fall asleep and that their sleep was calmer (Table 6). Subjects who were more comfortable with their body temperature also had more light sleep, TST, and higher SE (Table 6). Thus, the bed device's continuous temperature regulation improved subjects' thermal comfort leading to increased quantitative and qualitative sleep quality.

### Example 6: Machine-Learning Algorithm for Sleep Stage Classification

A machine-learning algorithm was developed in order to determine the sleep stage a user is experiencing during use of the bed device. The algorithm used real-time physiological data from the sensors in the bed device to predict the current sleep stage a user is experiencing during use of the bed device.

To develop the algorithm, a dataset comprising over 5,700 subjects, each monitored with full polysomnography and professional sleep stage scoring, was used. Data from subjects lacking complete sleep data, subjects with moderate to severe obstructive sleep apnea, and subjects that recorded less than four hours of sleep were excluded by the final dataset. The final dataset was then segmented into training, validation, and test sets to ensure comprehensive learning and validation of the algorithm.

The machine-learning algorithm showed a 50% increase in accuracy in determining the sleep stage of the user compared to previous algorithms used with the bed device.

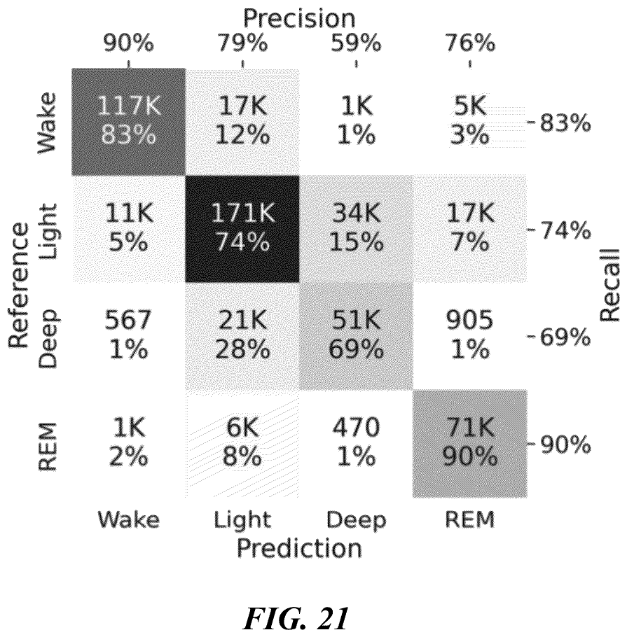

The accurate of the algorithm in determining the current sleep stage of the user was compared to electroencephalogram (EEG) brain wave recordings of subjects while in different sleep stages. This comparison showed that the algorithm was 78% accurate in classifying the correct sleep stages, as shown in FIG. 21.

While preferred embodiments of the present inventive concepts have been shown and described herein, it will be obvious to those skilled in the art that such embodiments are provided by way of example only. It is not intended that the inventive concepts be limited by the specific examples provided within the specification. While the inventive concepts have been described with reference to the aforementioned specification, the descriptions and illustrations of the embodiments herein are not meant to be construed in a limiting sense. Numerous variations, changes, and substitutions will now occur to those skilled in the art without departing from the inventive concepts. Furthermore, it shall be understood that all aspects of the inventive concepts are not limited to the specific depictions, configurations or relative proportions set forth herein which depend upon a variety of conditions and variables. It should be understood that various alternatives to the embodiments of the inventive concepts described herein may be employed in practicing the inventive concepts. It is therefore contemplated that the inventive concepts shall also cover any such alternatives, modifications, variations or equivalents. It is intended that the following claims define the scope of the inventive concepts and that methods and structures within the scope of these claims and their equivalents be covered thereby.

What is claimed is:

1. A method comprising:
(a) receiving an input from a user or a user device, wherein the input is indicative of a biological condition of a female individual selected from the group consisting of (i) schedule of menstrual cycle, a time within the menstrual cycle, or lack of menstrual cycle, (ii) use of birth control or lack thereof, (iii) menopause or lack thereof, (iv) use of hormonal therapy or lack thereof, (v) pregnancy or lack thereof, (vi) post-partum period, (vii) breast-feeding period, (viii) polycystic ovarian syndrome, (ix) experience of hot flash, and (x) other women-specific conditions or diseases; and
(b) based on the input, providing a control signal for a temperature regulator that is operatively coupled to an article of furniture, wherein the control signal comprises an instruction for the temperature regulator to change a temperature of a portion of the article of furniture to a target temperature specific for the biological condition of the female individual, wherein the target temperature is selected from a database comprising a plurality of different target temperatures associated with the plurality of different biological conditions selected from the group consisting of (i)-(x).

2. The method of claim 1, wherein the article of furniture is a bed device.

3. The method of claim 1, wherein the target temperature is for enhancing user experience while using the article of furniture.

4. The method of claim 1, wherein the target temperature is for enhancing sleep quality of the user during or subsequent to sleeping on the article of furniture at about the target temperature.

5. The method of claim 1, wherein the target temperature is for enhancing a health condition of the user during or subsequent to sleeping on the article of furniture at about the target temperature.

6. The method of claim 1, wherein the input is provided by the user via a graphical user interface (GUI) of a user device that is operatively coupled to the article of furniture.

7. The method of claim 6, further comprising permitting the user to modify the target temperature specific for the biological condition of the female individual via the graphical user interface (GUI) of the user device operatively coupled to the article of furniture, and storing the modified target temperature in a database operatively coupled to the article of furniture.

8. The method of claim 7, further comprising (i) receiving a feedback from the user via the graphical user interface (GUI) of the user device operatively coupled to the article of furniture subsequent to the user's use of the article of furniture at the target temperature, and (ii) automatically modifying the target temperature based on the feedback for the user's future use of the article of furniture.

9. The method of claim 6, wherein a biological sex of the user is provided by the user via the graphical user interface (GUI) of the user device operatively coupled to the article of furniture.

10. The method of claim 1, wherein the biological condition of the female individual comprises a plurality of conditions selected from the group consisting of (i)-(x).

11. The method of claim 1, wherein the instruction is for the temperature regulator to change the temperature of the portion of the article of furniture to the target temperature (i) for a predetermined duration of time and/or (ii) at a predetermined time.

12. The method of claim 11, wherein the predetermined duration of time is retrieved from the database.

13. The method of claim 11, wherein the predetermined duration of time is at most 1 hour.

14. The method of claim 1, wherein at least a portion of the temperature regulator is contained within the portion of the article of furniture.

15. The method of claim 1, wherein the article of furniture further comprises a sensor disclosed in a surface of the article of furniture.

16. The method of claim 1, wherein the biological condition of the female individual is a schedule of a menstrual cycle, a time within the menstrual cycle, or a lack of a menstrual cycle.

17. The method of claim 1, wherein the biological condition of the female individual is use of birth control or lack thereof.

18. The method of claim 1, wherein the biological condition of the female individual is menopause or lack thereof.

19. The method of claim 1, wherein the biological condition of the female individual is use of hormonal therapy or lack thereof.

20. The method of claim 1, wherein the biological condition of the female individual is pregnancy or lack thereof.

21. The method of claim 1, wherein the biological condition of the female individual is post-partum period.

22. The method of claim 1, wherein the biological condition of the female individual is breast-feeding period.

23. The method of claim 1, wherein the biological condition of the female individual is polycystic ovarian syndrome.

24. The method of claim 1, wherein the biological condition of the female individual is experience of hot flashes.

25. The method of claim 1, wherein the biological condition of the female individual is other women-specific conditions or diseases.

26. The method of claim 1, wherein the temperature of the portion of the article of furniture and the target temperature are different by at least 0.5 degrees Celsius (° C.).

27. The method of claim 1, wherein the temperature of the portion of the article of furniture and the target temperature are different by at most 5° C.

28. The method of claim 1, wherein the instruction of (b) is for the temperature regulator to change the temperature of the portion of the article of furniture to the target temperature, and wherein the temperature and the target temperature are based on a predetermined temperature profile set by the user.

29. The method of claim 28, further comprising, subsequent to (b), directing the temperature regulator to change the temperature of the at least the portion of the article of furniture to an additional target temperature of the predetermined temperature profile.

30. A system comprising one or more computer processors and computer memory coupled thereto, the computer memory comprising a machine executable code that, upon execution by the one or more computer processors, implements the method of claim 1.
